(19) United States
(12) Patent Application Publication
(10) Pub. No.: US 2025/0213712 A1
(43) Pub. Date: Jul. 3, 2025

(54) ANTIBODY DRUG CONJUGATES COMPRISING CAMPTOTHECIN DERIVATIVES AND USES THEREOF

(71) Applicant: IntoCell, Inc., Daejeon (KR)

(72) Inventors: Sangkwang Lee, Daejeon (KR); Doohwan Jung, Daejeon (KR); Sunyoung Kim, Daejeon (KR); CheolMin Jeon, Daejeon (KR); MyeongHwa Jeong, Daejeon (KR); Jihye Choi, Daejeon (KR); Ya Gob Kim, Daejeon (KR); Juseong Kim, Daejeon (KR); Sangbin Lim, Daejeon (KR); Jae Do Yoo, Daejeon (KR); Da Hee Lee, Daejeon (KR); Hee Young Kang, Daejeon (KR); Sena Kim, Daejeon (KR); Hyein Noh, Daejeon (KR); Hyang Sook Lee, Daejeon (KR); Sung-Ju Moon, Daejeon (KR); Yosup Rew, Daejeon (KR); Taekyo Park, Daejeon (KR)

(21) Appl. No.: 19/002,904

(22) Filed: Dec. 27, 2024

Related U.S. Application Data
(60) Provisional application No. 63/614,939, filed on Dec. 27, 2023.

(51) Int. Cl. A61K 47/68; A61P 35/00

(52) U.S. Cl. CPC A61K 47/68037; A61K 47/6851; A61K 47/6889; A61P 35/00

(57) ABSTRACT

The present disclosure is directed toward drugs or toxins; drug conjugates comprising said drugs or toxins and a cleavable linker; and conjugates comprising said drugs or toxins, cleavable linkers, and cell-binding groups. The present disclosure also relates to methods of treating cancers, autoimmune diseases, and inflammatory diseases using the compounds and conjugates of the disclosure.

### RELATED APPLICATION

This application claims the benefit of U.S. Provisional Application No. 63/614,939, filed Dec. 27, 2023, the contents of which are fully incorporated by reference herein.

### BACKGROUND

Cleavable drug conjugates have the potential to combine the binding specificity of antibodies or other cell-binding agents with the potency of chemotherapeutic agents. Since binding to a cell allows a drug to be accurately delivered to a target cancer cell and released under specific conditions while minimizing collateral damage to healthy cells, this technology increases the efficacy of a therapeutic agent and decreases the risk of an adverse reaction. However, conventional treatments show non-selective uptake of drugs into normal cells and cancer cells, and their therapeutic effects are not significant. The non-selective uptake is mainly due to the hydrophobicity of linker-drugs, and although studies are being conducted to lower the hydrophobicity of linker-drugs, so far their success is limited. Thus, there is a need for selective therapeutic agents that target cancer cells. One such class of compounds are toxins, such as enzymatically active toxins of bacterial, fungal, plant, or animal origin, or fragments thereof. Further development of toxins, and conjugates of toxins, as anti-cancer agents is a promising approach to cancer treatment.

### SUMMARY OF THE DISCLOSURE

Tecans are a class of compounds that can provide some therapeutic effects, but as a class they generally suffer from problems of solubility and stability. Known tecan structures require amine functional groups in order to introduce a cleavable linker. Accordingly, additional Tecans are needed, as well as additional Tecan derivatives, prodrugs, and conjugates (e.g. antibody-drug conjugates).

In certain aspects, provided herein are compounds of Formula (I):

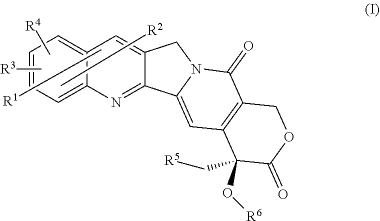

or a pharmaceutically acceptable salt thereof, wherein:
R¹ is selected from —(CH₂)ₜ(Ar¹¹)_{y}OR⁷—, —(CH₂)ₜNHC(═O)(CH₂)ₜOR⁷, —NHC(═O)CR₅=N—OR⁷, —NHC(═O)(CH₂)ₜ(Ar¹¹)_{y}OR⁷, —NH(CH₂)ₜ(Ar¹¹)_{y}OR⁷, and —NHC(═O)-alkyl-OR⁷;
R⁷ is H, -L^{C}-L^{B}-L^{A}, or -L^{C}-L^{B}-L^{D}-TM;
Ar¹¹ is aryl or heteroaryl;
y is 0 or 1;
L^{C} is a cleavage group;
L^{B} is a spacer group;
L^{A} is a reactive group;
L^{D} is a coupling group;
TM is a targeting moiety;
t is independently at each occurrence an integer from 0-5;
R², R³, and R⁴ are each independently selected from H, halo, hydroxy, cyano, nitro, amino, hydroxyamino, aminoacyl, amido, imino, alkyl, heteroalkyl, alkenyl, alkynyl, acyl, acyloxy, carboxyl, alkoxy, hydroxyalkyl, cycloalkyl, heterocycloalkyl, aryl, heteroaryl, (CH₂)ₜGlyc, (CH₂)ₜN(R⁸)₂, (CH₂)ₜC(═O)(CH₂)ₜOR⁸, (CH₂)ₜNHC(═O)(CH₂)ₜOR⁸, NHC(═O)C(═O)R⁸, NH(CH₂)ₜOR⁸, and (CH₂)ₜNHOR⁸; or any two of R¹, R², R³, and R⁴ combine with those carbons to which there are attached to complete an optionally substituted carbocyclyl or heterocyclyl, wherein the carbocyclyl or heterocyclyl, when substituted, is substituted with at least one R¹ or R⁹ substituent;
R⁵ is selected from H, halo, hydroxy, cyano, nitro, amino, hydroxyamino, aminoacyl, amido, imino, alkyl, heteroalkyl, alkenyl, alkynyl, acyl, acyloxy, alkoxy, hydroxyalkyl, cycloalkyl, heterocycloalkyl, aryl, heteroaryl, (CH₂)ₜGlyc, (CH₂)ₜNHC(═O)(CH₂)ₜOR⁸, NHC(═O)C(═O)R⁸, and (CH₂)ₜNHOR⁸;
Glyc is a monosaccharide, disaccharide, or oligosaccharide;
R⁶ is selected from H, C(═O)(CH₂)ₜGlyc, C(═O)(CH₂)ₜOR⁸, phosphonic acid (—P(═O)(OH)₂), sulfonic acid (—SO₃H), and C(═O)-alkyl;
each R⁸ is independently selected from H, alkyl, acyl, cycloalkyl, heterocyclyl, aryl, heteroaryl, and Glyc; and
R⁹ is selected from H, halo, hydroxy, carboxy, oxo, cyano, nitro, hydroxyamino, amino, aminoacyl, amido, imino, hydroxyalkyl, alkyl, heteroalkyl, alkenyl, alkynyl, acyl, acyloxy, alkoxy, cycloalkyl, heterocyclyl, aryl, heteroaryl, (CH₂)ₜAr¹¹OR⁸, (CH₂)ₜNH(CH₂)ₜOR⁸, (CH₂)ₜGlyc, (CH₂)ₜNHC(═O)(CH₂)ₜOR⁸, —NHC(═O)CR⁸═N—OR⁸, NHC(═O)C(═O)R⁸, and (CH₂)ₜNHOR⁸.

In certain embodiments, provided herein are methods of treating a cancer, comprising administering the compound or the pharmaceutical composition as described above to a subject in need thereof.

### BRIEF DESCRIPTION OF THE FIGURES

FIG. 1A shows the effect of exemplary ADCs (first method, Example 41 on tumor volume in NCI-N87 xenograft (Gastric CDX).

FIG. 1B shows the effect of exemplary ADCs (first method, Example 41) on body weight in NCI-N87 xenograft (Gastric CDX). No treatment related body weight loss was observed.

FIG. 2A shows the effect of exemplary ADCs (second method, Example 41) on tumor volume in NCI-N87 xenograft (Gastric CDX). Increased tumor growth inhibition was observed at higher doses. CR denotes the number of mice that exhibited a complete response.

FIG. 2B shows the effect of exemplary ADCs (second method, Example 41) on body weight in NCI-N87 xenograft (Gastric CDX). No treatment related body weight loss was observed.

FIG. 3A shows the results of an enzymatic cleavage assay of Compound T1-2 (Example 40).

FIG. 3B shows the results of an enzymatic cleavage assay of Compound T1-2 (Example 40).

FIG. 4A shows the effect of exemplary ADCs (third method, Example 41) on tumor volume in NCI-N87 xenograft (Gastric CDX).

FIG. 4B shows the effect of exemplary ADCs (third method, Example 41) on body weight in NCI-N87 xenograft (Gastric CDX). No treatment related body weight loss was observed.

FIG. 5 depicts in vivo efficacy of exemplary ADCs (fourth method, Example 41) on tumor size in NCI-N87 xenograft (Gastric CDX).

### DETAILED DESCRIPTION OF THE DISCLOSURE

Provided herein are compounds and drug conjugates which are useful for the treatment of cancer and/or autoimmune diseases or inflammatory diseases.

Some components of the technology disclosed herein, including cleavable linker technologies and cell-binding groups, are further described in WO 2019/008441, WO 2019/229536, WO 2020/141459, WO 2020/141460, WO 2021/260438, U.S. Pat. Nos. 16,472,983, 14,898,932, and 11,996,009 each of which is incorporated herein by reference in its entirety.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen atoms in a given structure with a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen atoms in a given structure with the substituents mentioned above. More preferably, one to three hydrogen substituents are replaced by the substituents as mentioned above. It is understood that the substituent can be further substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer. The term “lower alkyl” refers to the alkyl group with 1-6 carbon atoms. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like.

Moreover, the term “alkyl” as used throughout the specification, examples, and claims is intended to include both unsubstituted and substituted alkyl groups, the latter of which refers to alkyl groups having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, e.g., acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

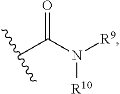

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure. The amide group may be bivalent (i.e., form part of a longer chain), e.g.,

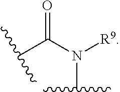

The terms “amine” and “amino” are art-recognized and refer to both unsubstituted and substituted amines and salts thereof, e.g., a moiety that can be represented by

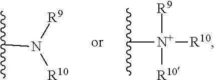

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

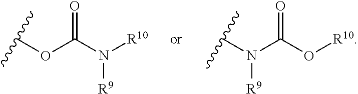

wherein R⁹ and R¹⁰ independently represent hydrogen or a hydrocarbyl group.

The term “carbocyclylalkyl”, as used herein, refers to an alkyl group substituted with a carbocycle group.

The term “cycloalkyl”, as used herein refers to a monocyclic or polycyclic non-aromatic ring, wherein each of the atoms forming the ring (i.e. skeletal atoms) is a carbon atom. Cycloalkyls may be saturated or partially unsaturated. Cycloalkyls may be fused with one or more aromatic rings (in which case the cycloalkyl is bonded through a non-aromatic ring carbon atom) or with one or more rings containing heteroatom(s) (in which case the cycloalkyl is bonded through a ring that does not contain a heteroatom). Representative cycloalkyls include, but are not limited to, cycloalkyls having from three to ten carbon atoms, from three to eight carbon atoms, from three to six carbon atoms, or from three to five carbon atoms. Monocyclic cycloalkyl rings include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. Polycyclic rings include, for example, adamantyl, norbornyl, decalinyl, and 3,4-dihydronaphthalen-1 (2H)-one. Unless otherwise stated specifically in the specification, a cycloalkyl group may be optionally substituted. The terms “cycloalkyl” also include polycyclic ring systems having two or more cyclic rings in which two or more carbons are common to two adjoining rings wherein at least one of the rings is cycloalkyl, e.g., the other cyclic rings can be cycloalkyls, cycloalkenyls, cycloalkynyls, aryls, heteroaryls, and/or heterocyclyls.

The term “carbocycle” or “carbocyclyl” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro (Cl), fluoro (F), bromo (Br), and iodo (I).

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, triazole, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are six or fewer atoms in the substituent. A “lower alkyl”, for example, refers to an alkyl group that contains six or fewer carbon atoms. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

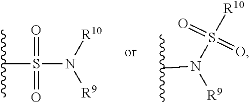

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “bisulfite” is art-recognized and refers to the group —OS(O)OH, or a pharmaceutically acceptable salt thereof.

The term “sulfate” is art-recognized and refers to the group —OSO₃H, or a pharmaceutically acceptable salt thereof.

The term “sulfone” is art-recognized and refers to the group —S(O)₂—.

The term “substituted” refers to groups having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, aphosphoryl, aphosphate, aphosphonate, aphosphinate, an amino, an amido, an amidine, an iminec a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the groups substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹, or —SC(O)R⁹, wherein R⁹ represents a hydrocarbyl.

The term “thioether”, as used herein, is equivalent to an ether, wherein the oxygen is replaced with a sulfur.

The term “urea” is art-recognized and may be represented by the general formula

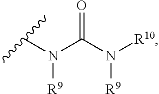

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of the disclosure for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

In certain embodiments, compounds of the disclosure may be racemic. In certain embodiments, compounds of the disclosure may be enriched in one enantiomer. For example, a compound of the disclosure may have greater than about 30% ee, 40% ee, 50% ee, 60% ee, 70% ee, 80% ee, 90% ee, 95% ee, 96% ee, 97% ee, 98% ee, 99% ee, or greater ee.

As is generally understood in the art, single bonds drawn without stereochemistry do not indicate the stereochemistry of the compound. The compound of formula I provides an example of a compound for which no stereochemistry is indicated.

In certain embodiments, a composition or compound of the disclosure may be enriched to provide predominantly one enantiomer of a compound. An enantiomerically enriched composition or compound may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound contains 98 grams of a first enantiomer and 2 grams of a second enantiomer, it would be said to contain 98 mol percent of the first enantiomer and only 2 mol % of the second enantiomer.

Furthermore, certain compounds which contain alkenyl groups may exist as Z (zusammen) or E (entgegen) isomers. In each instance, the disclosure includes both mixtures and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

The term “glycosyl” as used herein refers to a monovalent substituent formed from any natural sugar, a metabolite/catabolite thereof, a prodrug thereof, or a combination thereof. The term includes both linear and branched forms of oligosaccharides and polysaccharides, as well as alpha and beta configurations or any combination thereof. Preferred chain lengths of polysaccharides are one or two (i.e., mono- or disaccharides). In certain preferred embodiments, a glycosyl refers to a substituent formed from a glucose, a fucose, a galactose, a mannose, a xylose, a galatosamine, a glucuronic acid, a galacturonic acid, a manuric acid, a sialic acid, iduronic acid, neuraminic acid, derivatives thereof, or a combination thereof.

### Drugs and Drug Conjugates

In certain aspects, provided herein are compounds of Formula (I):

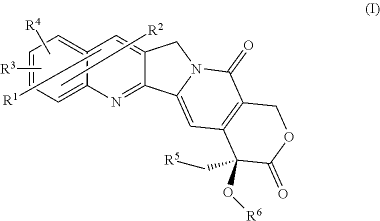

or a pharmaceutically acceptable salt thereof, wherein:
R¹ is selected from —(CH₂)ₜ(Ar¹¹)_{y}OR⁷—, —(CH₂)ₜNHC(═O)(CH₂)ₜOR⁷, —NHC(═O)CR₅═N—OR⁷, —NHC(═O)(CH₂)ₜ(Ar¹¹)_{y}OR⁷, —NH(CH₂)ₜ(Ar¹¹)_{y}OR⁷, and —NHC(═O)-alkyl-OR⁷;
R⁷ is H, -L^{C}-L^{B}-L^{A}, or -L^{C}-L^{B}-L^{D}-TM;
Ar¹¹ is aryl or heteroaryl;
y is 0 or 1;
L^{C} is a cleavage group;
L^{B} is a spacer group;
L^{A} is a reactive group;
L^{D} is a coupling group;
TM is a targeting moiety;
t is independently at each occurrence an integer from 0-5;
R², R³, and R⁴ are each independently selected from H, halo, hydroxy, cyano, nitro, amino, hydroxyamino, aminoacyl, amido, imino, alkyl, heteroalkyl, alkenyl, alkynyl, acyl, acyloxy, carboxyl, alkoxy, hydroxyalkyl, cycloalkyl, heterocycloalkyl, aryl, heteroaryl, (CH₂)ₜGlyc, (CH₂)ₜN(R⁸)₂, (CH₂)ₜC(═O)(CH₂)ₜOR⁸, (CH₂)ₜNHC(═O)(CH₂)ₜOR⁸, NHC(═O)C(═O)R⁸, NH(CH₂)ₜOR⁸, and (CH₂)ₜNHOR⁸; or any two of R¹, R², R³, and R⁴ combine with those carbons to which there are attached to complete an optionally substituted carbocyclyl or heterocyclyl, wherein the carbocyclyl or heterocyclyl, when substituted, is substituted with at least one R¹ or R⁹ substituent;
R⁵ is selected from H, halo, hydroxy, cyano, nitro, amino, hydroxyamino, aminoacyl, amido, imino, alkyl, heteroalkyl, alkenyl, alkynyl, acyl, acyloxy, alkoxy, hydroxyalkyl, cycloalkyl, heterocycloalkyl, aryl, heteroaryl, (CH₂)ₜGlyc, (CH₂)ₜNHC(═O)(CH₂)ₜOR⁸, NHC(═O)C(═O)R⁸, and (CH₂)ₜNHOR⁸;
Glyc is a monosaccharide, disaccharide, or oligosaccharide;
R⁶ is selected from H, C(═O)(CH₂)ₜGlyc, C(═O)(CH₂)ₜOR⁸, phosphonic acid (—P(═O)(OH)₂), sulfonic acid (—SO₃H), and C(═O)-alkyl;
each R⁸ is independently selected from H, alkyl, acyl, cycloalkyl, heterocyclyl, aryl, heteroaryl, and Glyc; and
R⁹ is selected from H, halo, hydroxy, carboxy, oxo, cyano, nitro, hydroxyamino, amino, aminoacyl, amido, imino, hydroxyalkyl, alkyl, heteroalkyl, alkenyl, alkynyl, acyl, acyloxy, alkoxy, cycloalkyl, heterocyclyl, aryl, heteroaryl, (CH₂)ₜAr¹¹OR⁸, (CH₂)ₜNH(CH₂)ₜOR⁸, (CH₂)ₜGlyc, (CH₂)ₜNHC(═O)(CH₂)ₜOR⁸, —NHC(═O)CR⁸═N—OR⁸, NHC(═O)C(═O)R⁸, and (CH₂)ₜNHOR⁸.

In certain aspects, only one pair of R¹, R², R³, and R⁴ combine to complete an optionally substituted carbocyclyl or heterocyclyl.

In certain aspects, two pairs of R¹, R², R³, and R⁴ combine to complete an optionally substituted carbocyclyl or heterocyclyl.

In certain aspects, at least one pair of R¹, R², R³, and R⁴ combine to to complete an optionally substituted 5 or 6-membered carbocyclyl or heterocyclyl.

In certain aspects, provided herein are compounds of Formula (I)

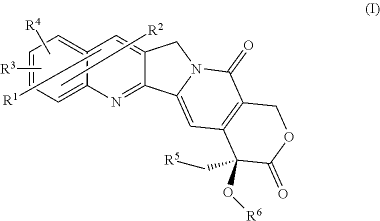

and pharmaceutically acceptable salts thereof, wherein:
R¹ is selected from —(CH₂)ₜ(Ar¹¹)_{y}OR⁷— —(CH₂)ₜNHC(═O)(CH₂)ₜOR⁷, and —NHC(═O)CR₅═N—OR⁷;
R⁷ is H, -L^{C}-L^{B}-L^{A}, or -L^{C}-L^{B}-L^{D}-TM;
Ar¹¹ is aryl or heteroaryl;
y is 0 or 1;
L^{C} is a cleavage group;
L^{B} is a spacer group;
L^{A} is a reactive group;
L^{D} is a coupling group;
t is independently at each occurrence an integer from 0-5;
R², R³, and R⁴ are each independently selected from H, halo, hydroxy, cyano, nitro, amino, hydroxyamino, aminoacyl, amido, imino, alkyl, heteroalkyl, alkenyl, alkynyl, acyl, acyloxy, alkoxy, hydroxyalkyl, cycloalkyl, heterocycloalkyl, aryl, heteroaryl, (CH₂)ₜGly, (CH₂)ₜNHC(═O)(CH₂)ₜOR⁸, NHC(═O)C(═O)R⁸, NH(CH₂)ₜOR⁸, and (CH₂)ₜNHOR⁸; or two of R², R³, and R⁴ are present on vicinal carbons and combine with those carbons to complete a carbocyclyl or heterocyclyl, which may bear the R¹ substituent;
R⁵ is selected from H, halo, hydroxy, cyano, nitro, amino, hydroxyamino, aminoacyl, amido, imino, alkyl, heteroalkyl, alkenyl, alkynyl, acyl, acyloxy, alkoxy, hydroxyalkyl, cycloalkyl, heterocycloalkyl, aryl, heteroaryl, (CH₂)ₜGlyc, (CH₂)ₜNHC(═O)(CH₂)ₜOR⁸, NHC(═O)C(═O)R⁸, and (CH₂)ₜNHOR⁸;
Glyc is a monosaccharide, disaccharide, or oligosaccharide;
each R⁸ is independently selected from H, alkyl, acyl, cycloalkyl, heterocyclyl, aryl, and heteroaryl;
R⁶ is selected from H, C(═O)(CH₂)ₜGly, C(═O)(CH₂)ₜOR⁸, phosphonic acid (—P(═O)(OH)₂), sulfonic acid (—SO₃H), and C(═O)-alkyl; and
TM is a targeting moiety.

As will be recognized by those of skill in the art, the compounds of Formula (I), as variously described herein, represent active agents, conjugates, and intermediates. For example, when R⁷ is H, the compound of Formula (I) is an active agent. In particularly preferred embodiments, where R⁷ is -L^{C}-L^{B}-L^{D}-TM, the compound is a conjugate of an active agent with the targeting moiety TM, which as discussed in more detail below may be any suitable targeting moiety, such as an antibody.

In certain embodiments, two of R², R³, and R⁴ are present on vicinal carbons and combine with those carbons to complete a carbocyclyl or heterocyclyl, which may be unsubstituted or substituted, preferably with the R¹ substitutent.

In certain embodiments, the compounds have a structure of Formula (IIA) or (IIB)

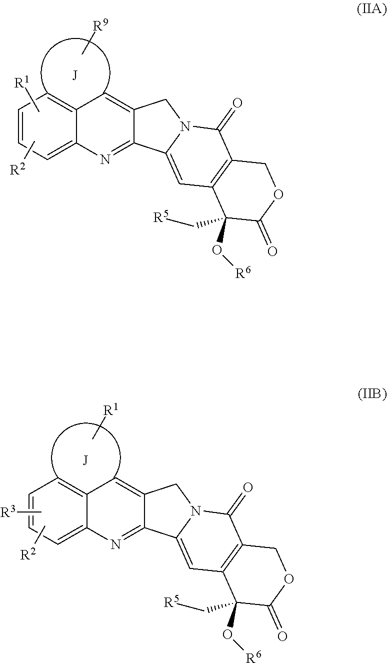

or pharmaceutically acceptable salts thereof, wherein:
ring J is a 5-membered or 6-membered cycloalkenyl or heterocycloalkenyl; and
R⁹ is selected from H, halo, hydroxy, carboxyl, oxo, cyano, nitro, hydroxyamino, amino, aminoacyl, amido, imino, alkyl, heteroalkyl, alkenyl, alkynyl, acyl, acyloxy, alkoxy, cycloalkyl, heterocyclyl, aryl, heteroaryl, (CH₂)ₜAr¹¹OR⁸, (CH₂)ₜNH(CH₂)ₜOR⁸, (CH₂)ₜGly, (CH₂)ₜNHC(═O)(CH₂)ₜOR⁸, —NHC(═O)CR⁸═N—OR⁸, NHC(═O)C(═O)R⁸, and (CH₂)ₜNHOR⁸.

In certain embodiments, the compounds have a structure of Formula (IIA) or (IIB):

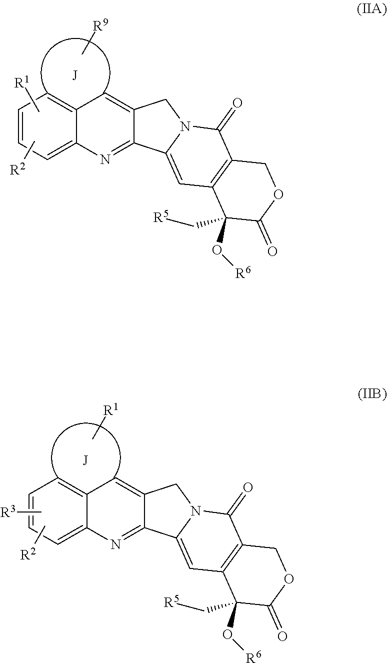

or a pharmaceutically acceptable salt thereof, wherein:
ring J is a 5-membered or 6-membered cycloalkenyl or heterocycloalkenyl.

In certain embodiments, the R¹ and R² or R² and R³ combine to complete an optionally substituted carbocyclyl or heterocyclyl.

In certain embodiments, the compounds have a structure of Formula (III)

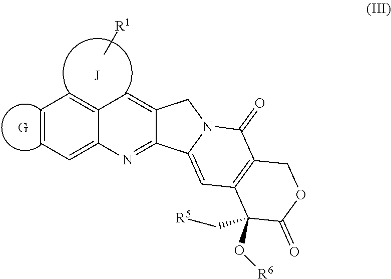

or pharmaceutically acceptable salts thereof, wherein ring G is a 5- or 6-membered carbocyclyl or heterocyclyl ring.

In certain embodiments, R², R³, and R⁴ are each independently selected from H, halo, hydroxy, cyano, nitro, amino, hydroxyamino, aminoacyl, amido, imino, alkyl, heteroalkyl, alkenyl, alkynyl, acyl, acyloxy, alkoxy, hydroxyalkyl, cycloalkyl, heterocycloalkyl, aryl, heteroaryl, (CH₂)ₜGly, (CH₂)ₜNHC(═O)(CH₂)ₜOR⁸, NHC(═O)C(═O)R⁸, NH(CH₂)ₜOR⁸, and (CH₂)ₜNHOR⁸.

In certain preferred embodiments, the compounds have a structure of Formula (IVA), (IVB), or (IVC)

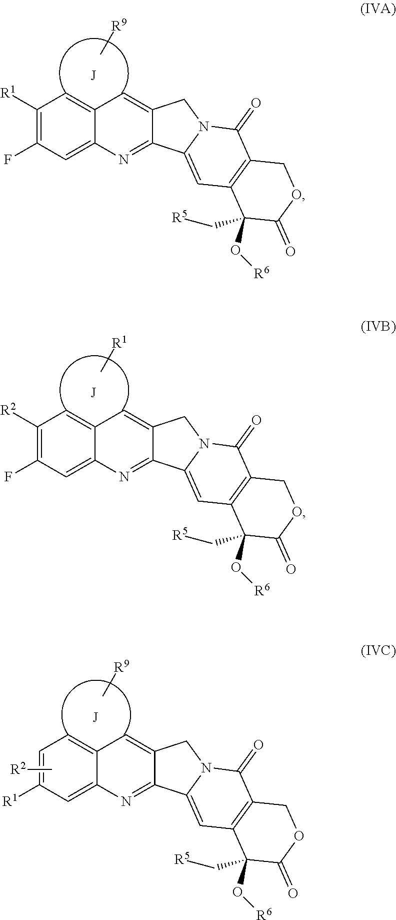

and pharmaceutically acceptable salts thereof, wherein:
ring J is a 6-membered carbocyclyl or heterocyclyl.

In certain preferred embodiments, ring J is a 6-membered carbocyclyl.

In certain especially preferred embodiments, the compound has a structure of Formula (VA):

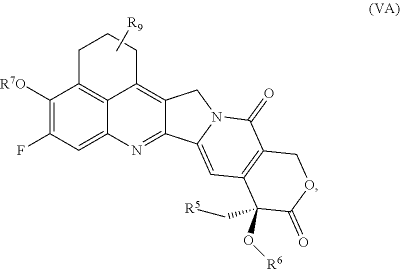

or pharmaceutically acceptable salts thereof.

In certain preferred embodiments, R⁹ is H.

In certain embodiments, the compounds have a structure of Formula (VIA) or (VIB)

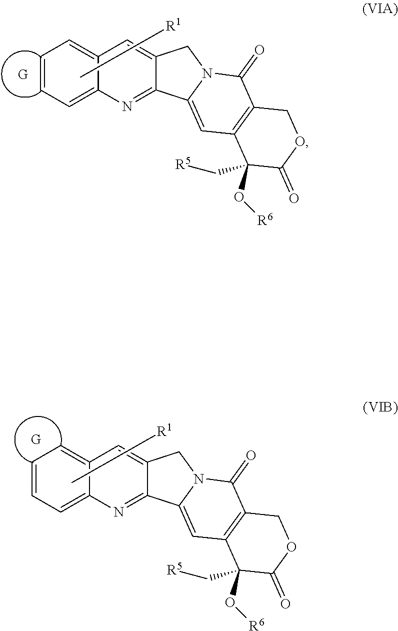

and pharmaceutically acceptable salts thereof, wherein:
ring G is a 5- or 6-membered carbocyclyl or heterocyclyl.

In certain embodiments, ring G is a 5-membered heterocyclyl.

In certain preferred embodiments, the compounds have a structure of Formula (VIIA) or (VIIB):

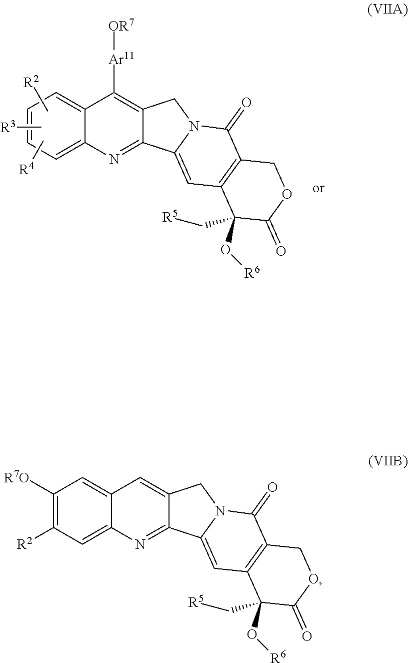

and pharmaceutically acceptable salts thereof.

In certain especially preferred embodiments, the compounds have a structure of Formula (VIIIA)

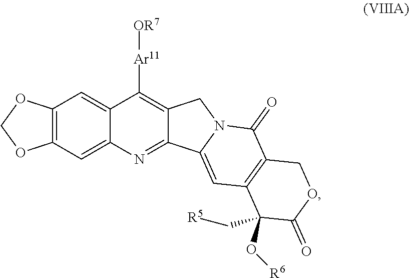

or pharmaceutically acceptable salts thereof.

In certain embodiments, R¹ is Ar¹¹OR⁷. In certain preferred embodiments, Ar¹¹ is aryl. In certain embodiments, Ar¹¹ is naphthyl. In certain embodiments, Ar¹¹ is biphenyl. In certain embodiments, Ar¹¹ is phenanthrenyl. In certain especially preferred embodiments, Ar¹¹ is phenyl.

In certain embodiments, Ar¹¹ is heteroaryl. In certain embodiments, Ar¹¹ is pyrrolyl. In certain embodiments, Ar¹¹ is thiophenyl. In certain embodiments, Ar¹¹ is pyrazinyl. In certain embodiments, Ar¹¹ is pyrimidinyl. In certain embodiments, Ar¹¹ is pyradizinyl. In certain embodiments, Ar¹¹ is indolyl. In certain embodiments, Ar¹¹ is pyridinyl or triazolyl

In certain embodiments, R⁷ is H. In certain embodiments, R⁷ is L^{C}-L^{B}-L^{A}.

In certain embodiments, L^{A} is selected from isocyanide, isothiocyanide, 2-pyridyl disulfide, haloacetamide (—NHC(═O)CH₂-halo), maleimide, diene, alkene, halide, tosylate, aldehyde, sulfonate,

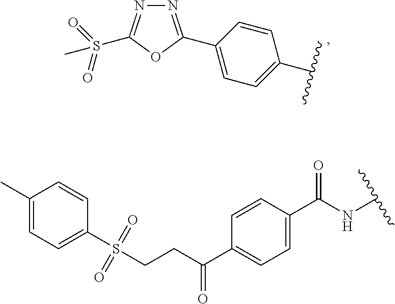 [SMILES: *c1ccc(-c2nnc(S(C)(=O)=O)o2)cc1 ; *NC(=O)c1ccc(C(=O)CCS(=O)(=O)c2ccc(C)cc2)cc1]

phosphonic acid (—P(═O)(OH)₂), ketone, C₈-C₁₀ cycloalkynyl, —OH, —NHOH, —NHNH₂, —SH, carboxylic acid, alkyne, azide, amino, sulfonic acid, an alkynone derivative (—C(O)C═C—Rₐ, wherein Rₐ is H or alkyl), and dihydrogen phosphate (—OP(═O)(OH)₂). In certain embodiments, L^{A} is

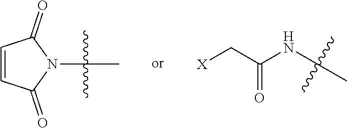

wherein X is halo. In yet further embodiments, X is I, Br or Cl.

In certain embodiments, R⁷ is L^{C}-L^{B}-L^{D}-TM.

In certain embodiments, L^{C} is selected from

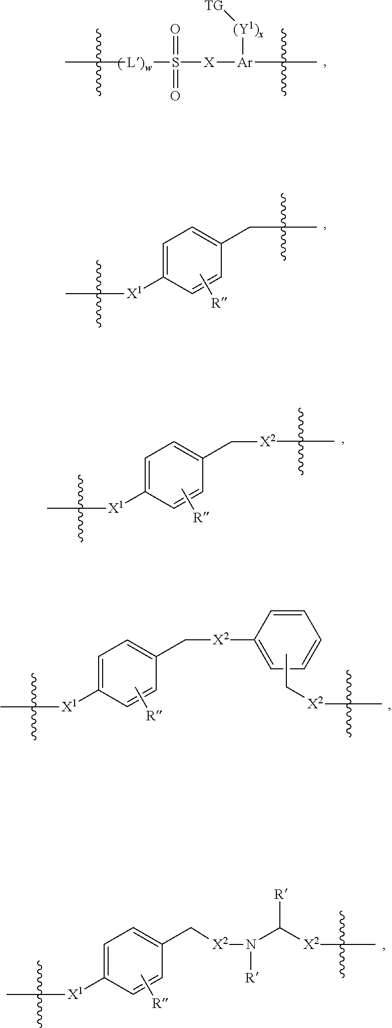

or a combination thereof:
wherein:
R′ is H or alkyl;
L′ is a spacer group attached to the SO₂ via a heteroatom selected from O, S, and N, preferably O or N, and selected such that cleavage of the bond between L′ and SO₂ promotes cleavage of the bond between L′ and the remainder of the compound;
w is 0 or 1;
x is 0 or 1;
X is selected from —O—, —CR^{b}₂, —S— and —NR^{a}—, where R^{a} and R^{b} are each independently at each occurrence H or alkyl;
Y¹ is —(CR^{b}₂)_{z}N(R^{a})—, —(CR^{b}₂)_{z}O—, or —(CR^{b}₂)_{z}S—, wherein z is an integer having a value of 0-5 and if z is 1-5, the N, O, or S atom is attached to TG, and wherein R^{a} and R^{b} are each independently for each occurrence H or alkyl;
at least one X is positioned in an ortho relationship or a para relationship to Y¹ on Ar;
Ar is aryl, heteroaryl, cycloalkyl, or heterocycloalkyl;
TG is a triggering group that, when activated, generates an O atom capable of reacting with the SO₂ to displace the remainder of the compound and form a 5-6-membered ring including X—SO₂ and the intervening atoms of Ar;
X¹ is —O— or NR′;
X² is —C(O), —C(O)O, —C(O)NR′—, or —NR′C(O)O—;
R″ is —SO₄, —(CH₂)ₜ(OCH₂CH₂)_{g}—R′, or —(CH₂)ₜGlyc; and
g is an integer having a value of 0-10.

In certain preferred embodiments, L^{C} is

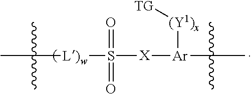

In yet further embodiments, X is O. In still further embodiments, Ar is aryl. In certain embodiments, Ar is C₆₋₁₀ aryl. In further embodiments, Ar is phenyl or naphthyl. In yet further embodiments, Ar is heteroaryl. In still further embodiments, Ar is a 5- to 10-membered heteroaryl comprising one to four heteroatoms selected from N, O, and S. In certain embodiments, Y¹ is —O—. In further embodiments, w and x are each 1. In yet further embodiments, L^{C} is

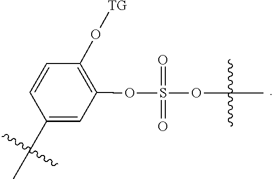

In certain embodiments, TG is a monosaccharide. In further embodiments, TG is a monosaccharide selected from glucose, glucuronic acid, fucose, and galactose. In yet further embodiments, TG is

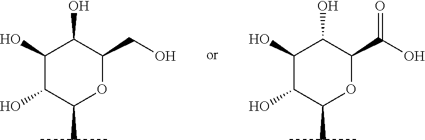

optionally wherein one or more of the —OH groups is masked by a protecting group. In still further embodiments, TG is

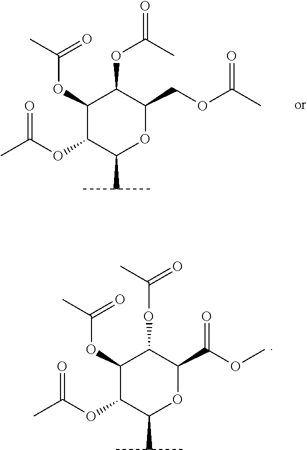

In certain embodiments, TG is a disaccharide. In further embodiments, TG is a disaccharide comprising glucose, glucuronic acid, fucose, galactose, or a combination thereof. In yet further embodiments, TG is

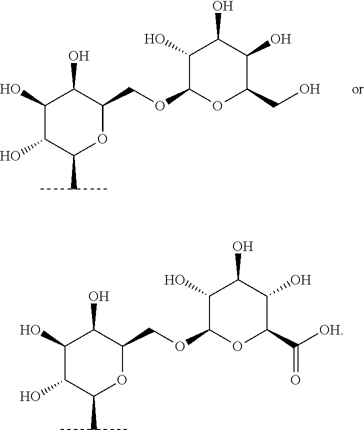

optionally wherein one or more of the —OH groups is masked with a protecting group. In certain embodiments, TG is

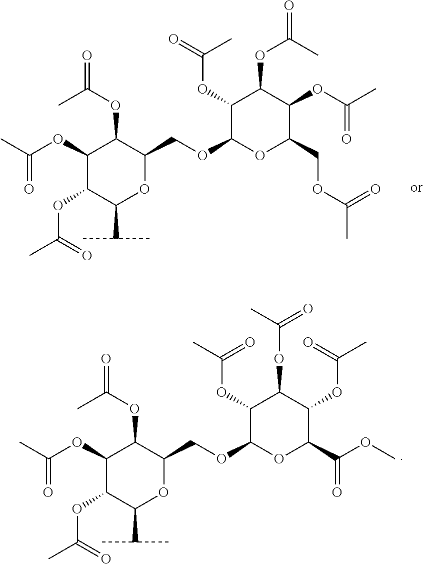

In certain embodiments, TG is a triggering group selected from —NO₂, —OC(═O)(CH₂)ᵣC(═O)R^{1A}, —NHOH, —BR^{1A}R^{1A}—NHNH₂, nitrobenzyl,

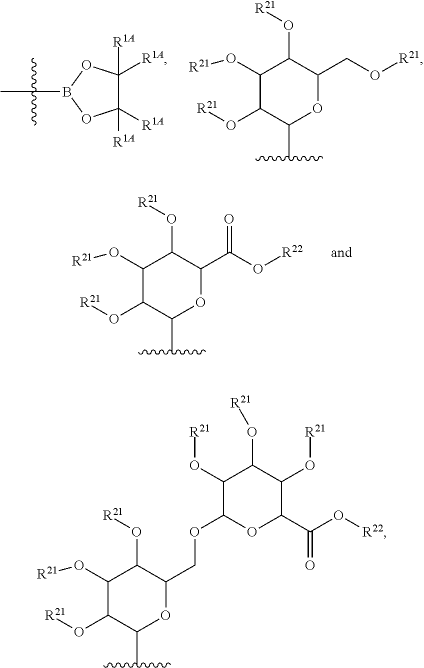

wherein:
R^{1A} is alkyl;
each R²¹ is independently hydrogen or acetyl;
R²² is hydrogen or C₁₋₆ alkyl; and
r is an integer from 0-5.

In certain embodiments, TG is selected from glucuronide, β-galactoside, and a disaccharide of β-galactoside and β-glucuronide. In yet further embodiments, TG is β-glucuronide or a disaccharide of β-galactoside and β-glucuronide.

In certain embodiments, L^{B} is selected from a C₇-C₁₀₀ linear or branched, saturated or unsaturated alkylene moiety comprising at least two of the following:
(i) at least one moiety selected from —NH—, —C(═O), —O—, —S— and —P—;
(ii) at least one heteroarylene, wherein the heteroarylene is selected from

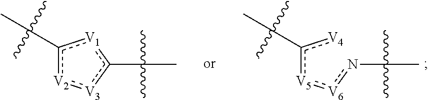

wherein:
V₁, V₂, and V₃ are independently selected from CH and N; and
V₄, V₅, and V₆ are each independently selected form C, CH, O, S, N, and NH; preferably a triazolene;
provided that the heteroarylene may be fused to a saturated or unsaturated ring;
(iii) at least one amino acid moiety, sugar bond, peptide bond, or amide bond; and
(iv) one or more substitutents selected from the group consisting of C₁-C₂₀ alkyl, C₆-C₂₀ aryl C₁-C₈ alkyl, (CH₂)ₛCOOH, ((OCH₂CH₂)ₙ)ₚR′ and —(CH₂)ₚNH₂, wherein each s and n independently is an integer having a value of 0 to 10, R′ is H or alkyl, and p is an integer having a value of 1 to about 10.

In certain embodiments, L^{B} comprises at least one amide bond and is selected from:

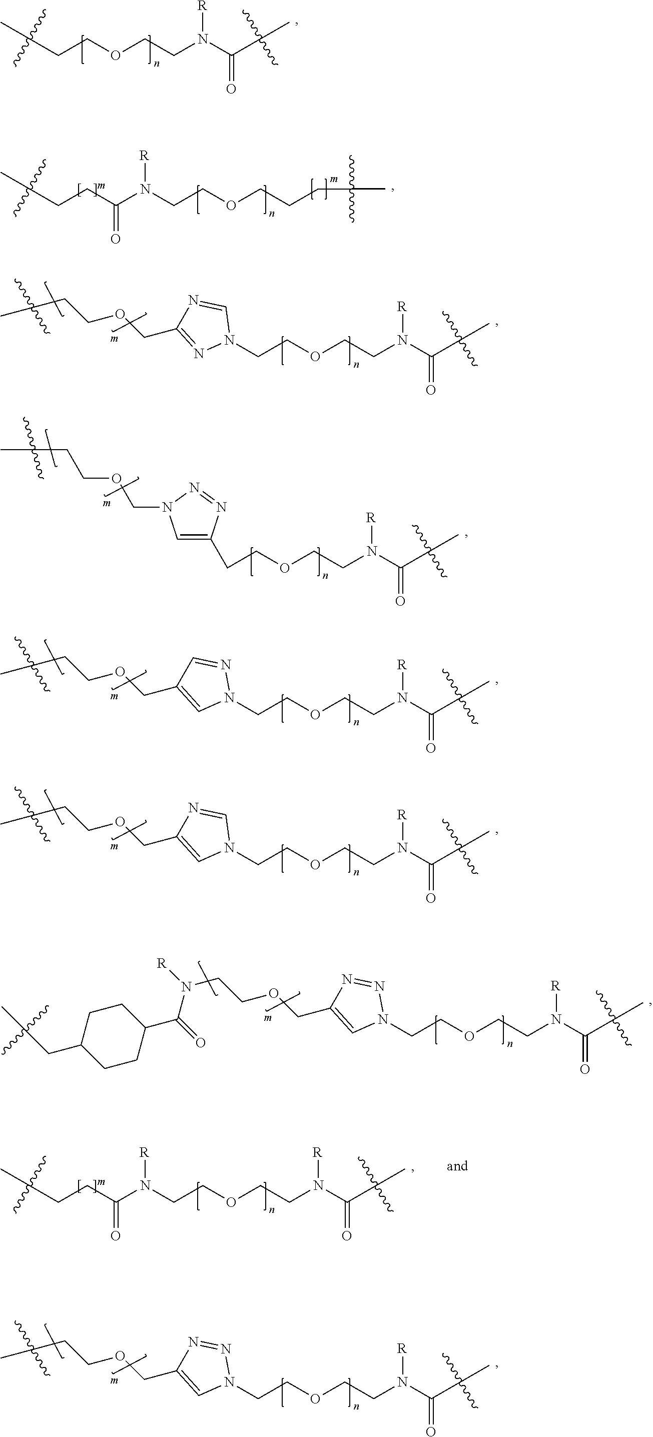

wherein each R is independently (CH₂)ₐₐR′;
aa is an integer from 0 to 10;
R′ is selected from H, hydroxy, aryl, cycloalkyl, nitro, amino, cyano, halo, (CH₂CH₂O)_{bb}R″, and C(═O)OR″;
R″ is selected from H, alkyl, and hydroxyl;
bb is an integer from 1 to 50;
n is an integer from 0 to 10; and
   m is an integer from 0 to 10. In certain embodiments, aa is an integer from 0 to 6, and R′ is selected from H, hydroxy, (CH₂CH₂O)_{bb}R″, and C(═O)OR″ and bb is an integer from 1 to 20.

In certain embodiments, L^{B} comprises at least one (CH₂CH₂O)_{bb}R″ at R.

In certain embodiments, L^{B} is:

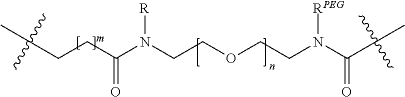

wherein
m is an integer from 0 to 10;
n is an integer from 0 to 10;
R is (CH₂)ₐₐR′;
aa is 0;
R′ is H;
R^{PEG} is

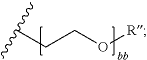

R″ is selected from H, alkyl, and hydroxyl; and
bb is an integer from 0 to 50.

In certain embodiments, m is 0; n is an integer from 2 to 4; R″ is alkyl; and bb is an integer from 1 to 20.

In certain embodiments, L^{B} is:

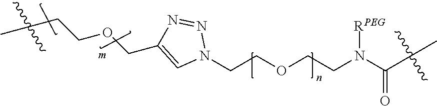

wherein:
m is an integer from 0 to 10;
n is an integer from 0 to 10;
R^{PEG} is

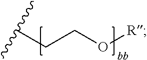

R″ is selected from H, alkyl, and hydroxyl; and
bb is an integer from 0 to 50.

In certain embodiments, m is 0; n is an integer from 2 to 4; R″ is alkyl; and bb is an integer from 1 to 20.

In certain embodiments, R″ is methyl.

In certain embodiments, L^{D} comprises a group that can be produced through a coupling reaction, e.g. the reaction of (a) a maleimide and a thiol; (b) a reaction between an azide and an alkyne, or (c) a haloacetamide and a thiol.

In certain embodiments, L^{D} comprises a linking unit formed from a precursor selected from isocyanide, isothiocyanide, 2-pyridyl disulfide, haloacetamide (e.g., —NHC(═O)CH₂-halo), maleimide, diene, alkene, halide, tosylate, aldehyde, sulfonate,

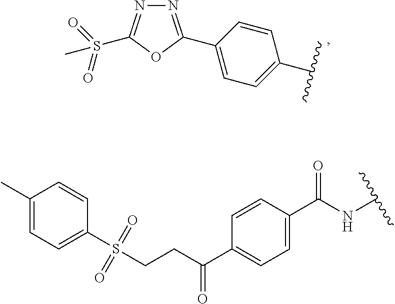 [SMILES: *c1ccc(-c2nnc(S(C)(=O)=O)o2)cc1 ; *NC(=O)c1ccc(C(=O)CCS(=O)(=O)c2ccc(C)cc2)cc1]

phosphonic acid (—P(O)(OH)₂), ketone, C₈-C₁₀ cycloalkynyl, —OH, —NHOH, —NHNH₂, —SH, carboxylic acid, alkyne, azide, amino, sulfonic acid, an alkynone derivative (C(O)C═C—Rₐ where Rₐ is H or alkyl), and dihydrogen phosphate (—OP(O)(OH)₂). In further embodiments, L^{D} comprises a triazole, thiosuccinimide, tetrazole, thioacetamide,

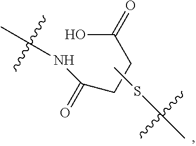

or thioether. In yet further embodiments, t is 0. In still further embodiments, t is an integer from 1 to 5.

In certain embodiments, R², R³, and R⁴ are each independently selected from halo, hydroxy, cyano, alkyl, heteroalkyl, amino, amido, acyloxy, and alkoxy. In further embodiments, R², R³, and R⁴ are each independently selected from halo, hydroxy, and alkyl.

In certain embodiments, R² is selected from H, halo, hydroxy, amino, amido, alkyl, acyloxy, (CH₂)ₜGlyc, and (CH₂)ₜNHR⁵. In further embodiments, R² is selected from H, amino, and amido. In yet further embodiments, R² is F.

In certain embodiments, R³ and R⁴ are each independently selected from H, alkyl, alkenyl, alkynyl, cycloalkyl, (CH₂)ₜGlyc, and (CH₂)ₜNHR⁵. In further embodiments, R³ and R⁴ are each independently selected from H and alkyl.

In certain embodiments, two of R², R³, and R⁴ are present and combine with those carbons to complete a carbocyclyl or heterocyclyl, which may bear the R¹ substituent and is optionally further substituted with one or more substituents selected from oxo (═O), oxime, ═N—OH, —NHC(═O)C(═O)R⁸, —NHC(═O)C(═N—OH)R⁸, —OH, cyano, and halo.

In certain embodiments, R⁵ and R⁶ are H.

In certain embodiments, R⁸ is selected from H and alkyl. In further embodiments, Glyc is selected from beta-glucuronide (BG), beta-galactoside (BGal), and beta-galactoside+beta-glucuronide:disaccharide type. In further embodiments, R⁶ is selected from H, —C(═O)—CH₂OH and

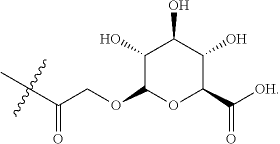

In yet further embodiments, R⁶ is H. In still further embodiments, R¹ is —(Ar¹¹)_{y}OH.

In certain embodiments, TM is selected from a nanoparticle, an immunoglobulin, a nucleic acid, a protein, an oligopeptide, a polypeptide, an antibody, a fragment of an antigenic polypeptide, or a repebody. In preferred embodiments, TM is an antibody selected from an intact polyclonal antibody, an intact monoclonal antibody, an antibody fragment, a single chain Fv (scFv) mutant, a multispecific antibody, a bispecific antibody, a chimeric antibody, a humanized antibody, a human antibody, a fusion protein comprising an antigenic determinant portion of an antibody, and other modified immunoglobulin molecules comprising antigen recognition sites. In yet further embodiments, TM is an intact monoclonal antibody. In still further embodiments, TM is selected from Muromonab-CD3, Abciximab, Rituximab, Daclizumab, Palivizumab, Infliximab, Trastuzumab (herceptin), Rosopatamab, Sacituzumab, Patritumab, Etanercept, Basiliximab, Gemtuzumab ozogamicin, Alemtuzumab, Ibritumomab tiuxetan, Adalimumab, Alefacept, Omalizumab, Efalizumab, Tositumomab-I¹³¹, Cetuximab, Bevacizumab, Natalizumab, Ranibizumab, Panitumumab, Eculizumab, Rilonacept, Certolizumab pegol, Romiplostim, AMG-531, CNTO-148, CNTO-1275, ABT-874, LEA-29Y, Belimumab, TACI-Ig, Second generation anti-CD20, ACZ-885, Tocilizumab, Atlizumab, Mepolizumab, Pertuzumab, Humax CD20, Tremelimumab (CP-675 206), Ticilimumab, MDX-010, IDEC-114, Inotuzumab ozogamycin, HuMax EGFR, Aflibercept, HuMax-CD4, Ala-Ala, ChAglyCD3, TRX4, Catumaxomab, IGN101, MT-201, Pregovomab, CH-14.18, WX-G250, AMG-162, AAB-001, Motavizumab, MEDI-524, Efumgumab, Aurograb, Raxibacumab, Third generation anti-CD20, LY2469298, and Veltuzumab.

In certain embodiments, TM binds to an antigen selected from HER2, HER3, B7H3, B7H4, TROP2, BCMA, CA6, CA9, CA15-3, CA19-9, CA27-29, CA125, CA242, CAIX, CCR2, CCR5, CD2, CD19, CD20, CD22, CD24, CD30, CD33, CD37, CD38, CD40, CD44, CD47, CD56, CD70, CD71, CD73, CD74, CD79, CD115, CD123, CD138, CD203c, CD303, CD333, CDCP1, CEA, CEACAM, Claudin 4, Claudin 7, CLCA-1, CLL 1, c-MET, Cripto, DLL3, EGFL, EGFR, EPCAM, EphA2, EPhB3, ETBR, FAP, FcRL5, FGFR3, FOLR1, FRbeta, GCC, GD2, GITR, GLOBO H, GPA33, GPC3, GPNMB, HMW-MAA, integrin α, IGF1R, TM4SF1, Lewis A like carbohydrate, Lewis X, Lewis Y, LGR5, LIV1, mesothelin, MN, MUC1, MUC16, NaPi2b, Nectin-4, Notch3, PD-1, PD-L1, PSMA, PTK7, SLC44A4, STEAP-1, 5T4, TF, TF-Ag, Tag72, TNFalpha, TNFR, uPAR, VEGFR and VLA. In further embodiments, the antigen is selected from HER2, HER3, and TROP2. In yet further embodiments, the antigen is located on a cancer cell.

In certain embodiments, the compound is of one of the following structures, or is a pharmaceutically acceptable salt thereof:

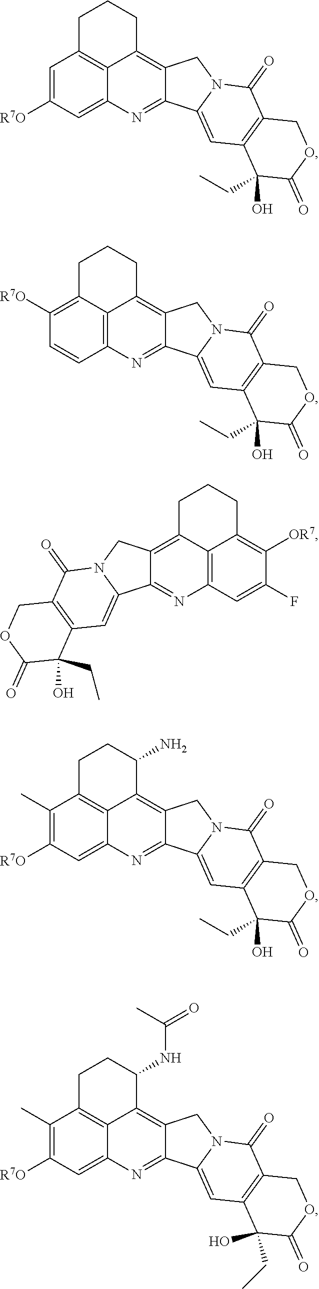

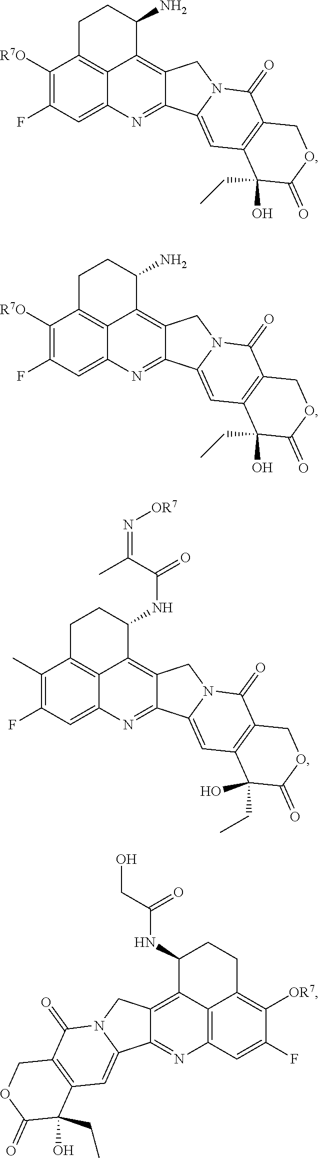

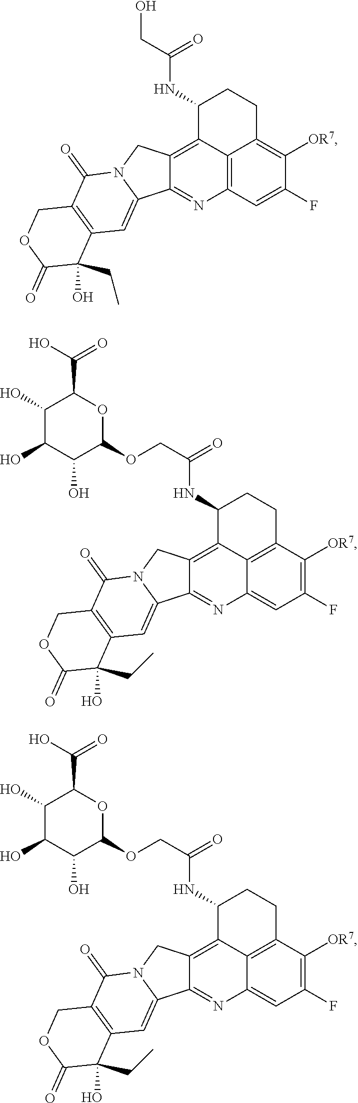

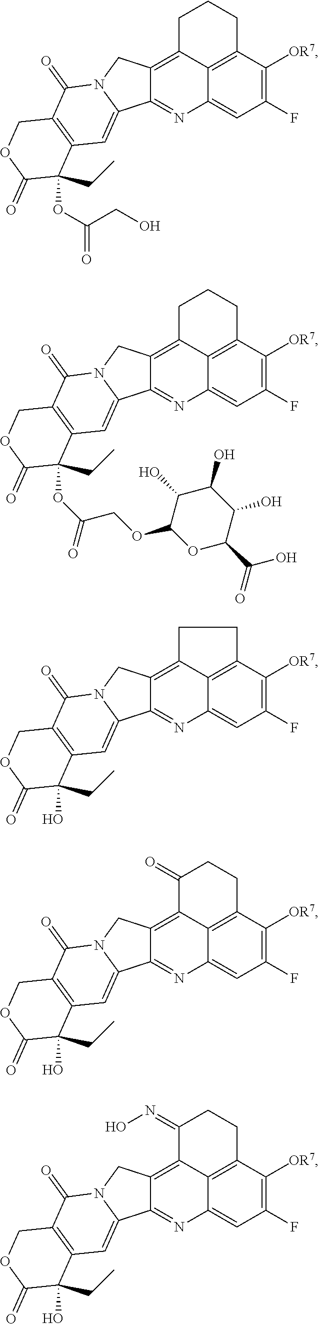

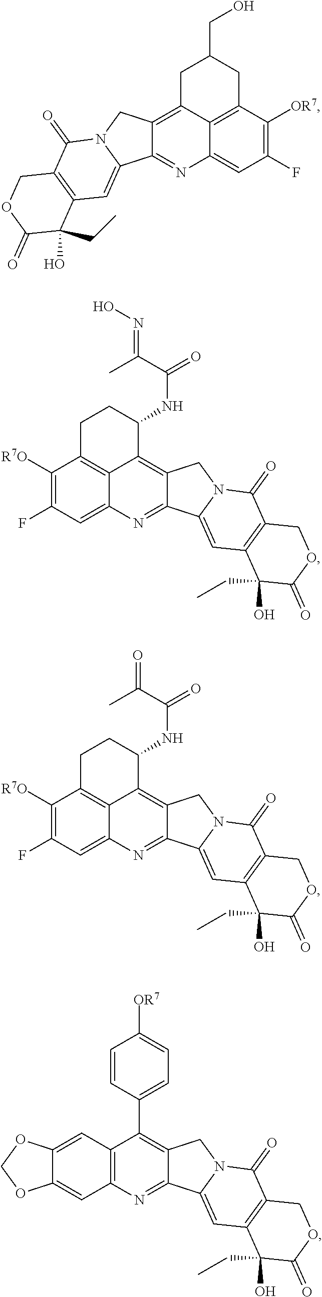

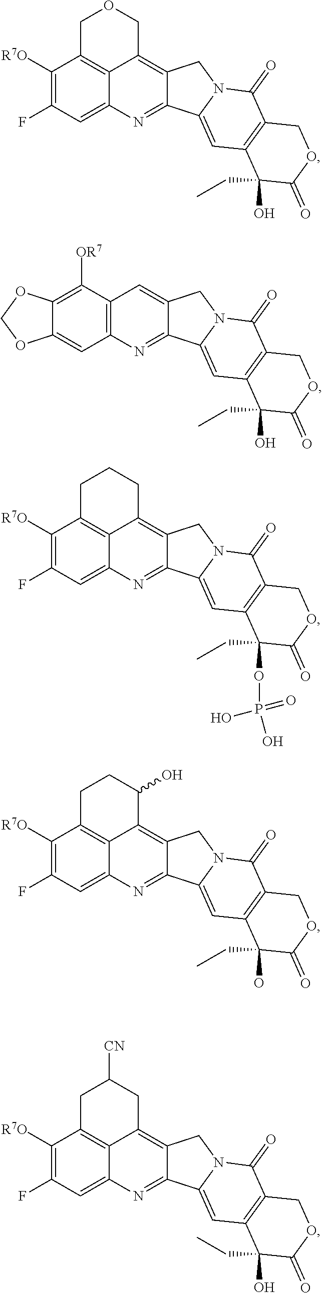

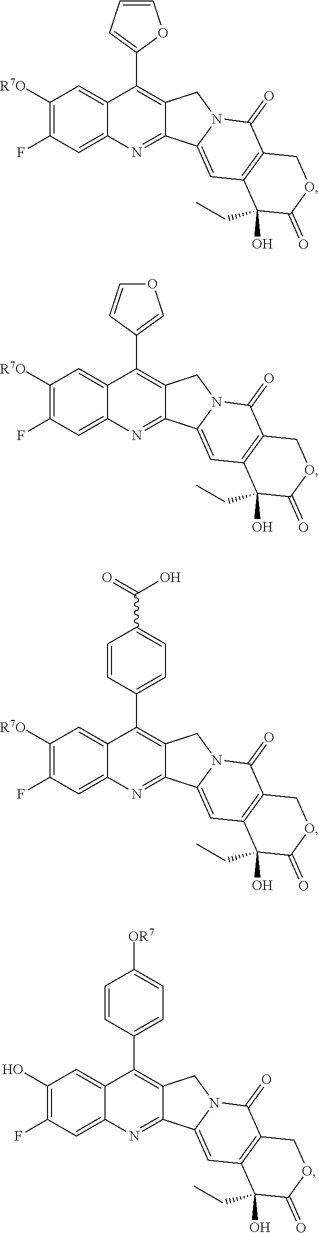

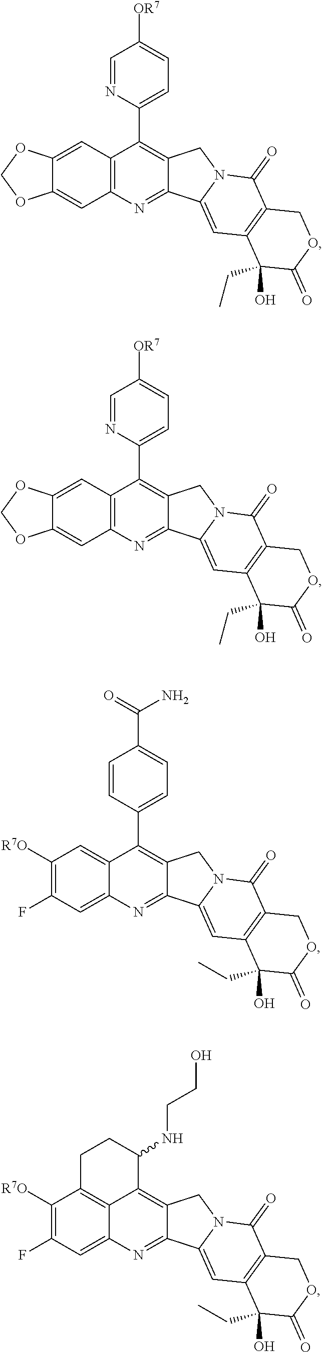

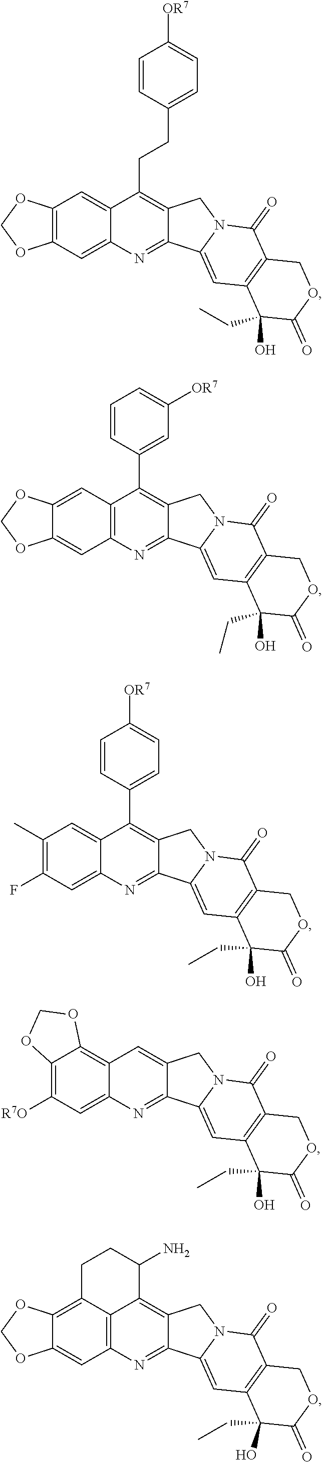

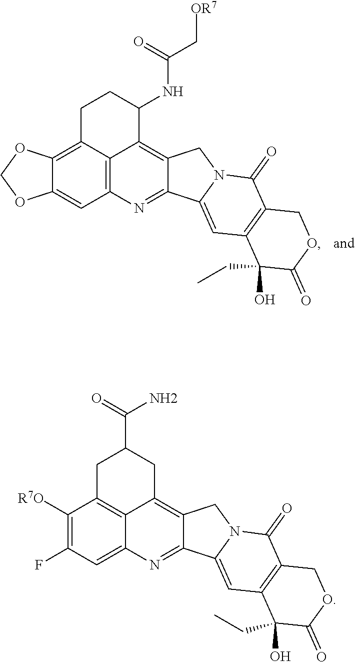

In certain embodiments, the compound is of one of the following structures, or is a pharmaceutically acceptable salt thereof:

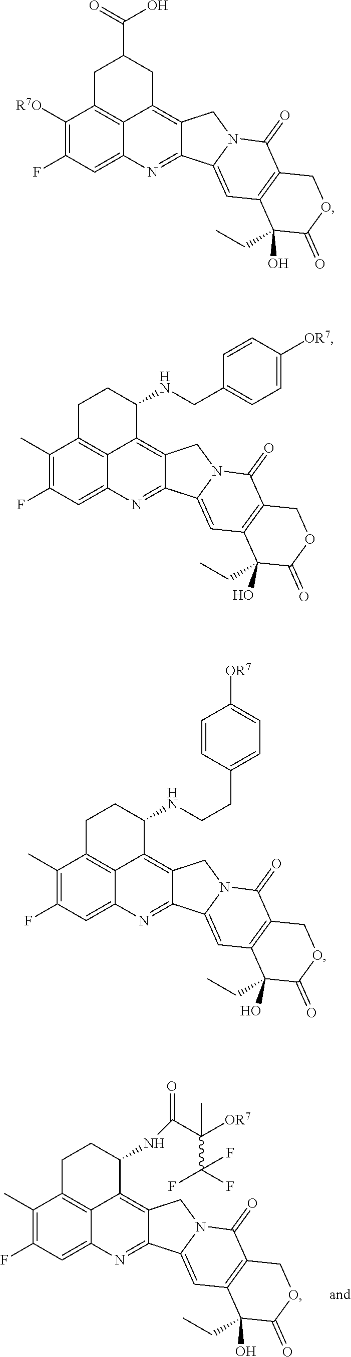

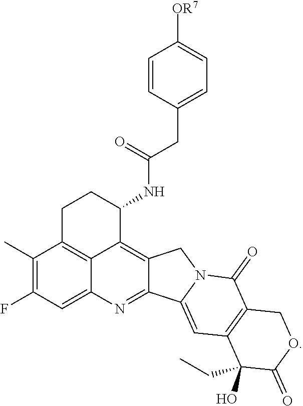

In certain embodiments, the compound is of one of the following structures, or is a pharmaceutically acceptable salt thereof:

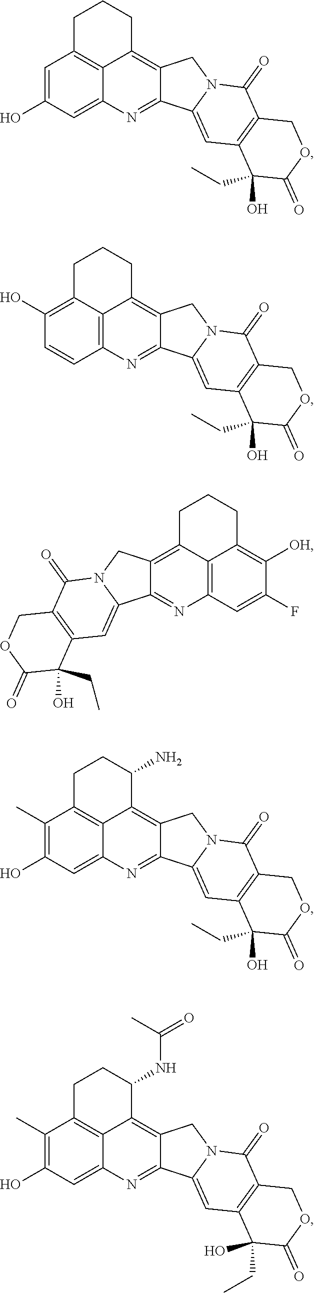 [SMILES: CC[C@@]1(O)C(=O)OCc2c1cc1n(c2=O)Cc2c-1nc1cc(O)cc3c1c2CCC3 ; CC[C@@]1(O)C(=O)OCc2c1cc1n(c2=O)Cc2c-1nc1ccc(O)c3c1c2CCC3 ; CC[C@@]1(O)C(=O)OCc2c1cc1n(c2=O)Cc2c-1nc1cc(F)c(O)c3c1c2CCC3 ; CC[C@@]1(O)C(=O)OCc2c1cc1n(c2=O)Cc2c-1nc1cc(O)c(C)c3c1c2[C@@H](N)CC3 ; CC[C@@]1(O)C(=O)OCc2c1cc1n(c2=O)Cc2c-1nc1cc(O)c(C)c3c1c2[C@@H](NC(C)=O)CC3]

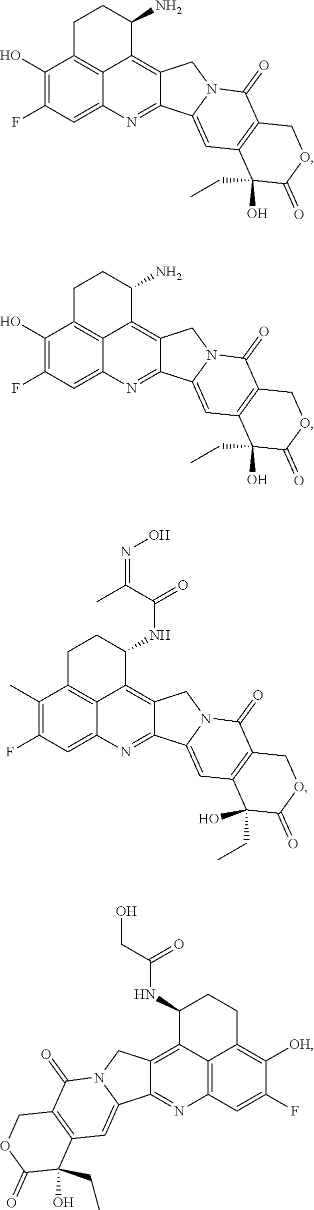 [SMILES: CC[C@@]1(O)C(=O)OCc2c1cc1n(c2=O)Cc2c-1nc1cc(F)c(O)c3c1c2[C@H](N)CC3 ; CC[C@@]1(O)C(=O)OCc2c1cc1n(c2=O)Cc2c-1nc1cc(F)c(O)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)/C(C)=N\O)CC3 ; CC[C@@]1(O)C(=O)OCc2c1cc1n(c2=O)Cc2c-1nc1cc(F)c(O)c3c1c2[C@@H](NC(=O)CO)CC3]

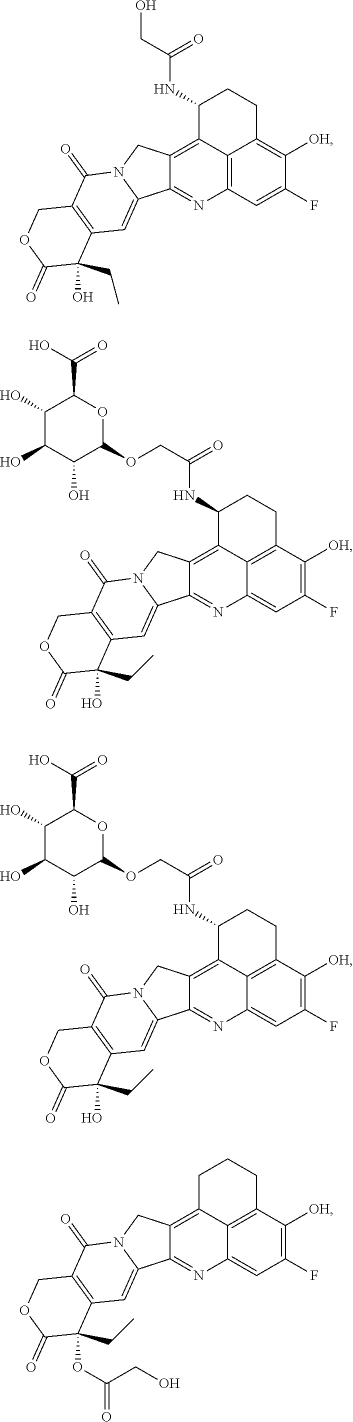

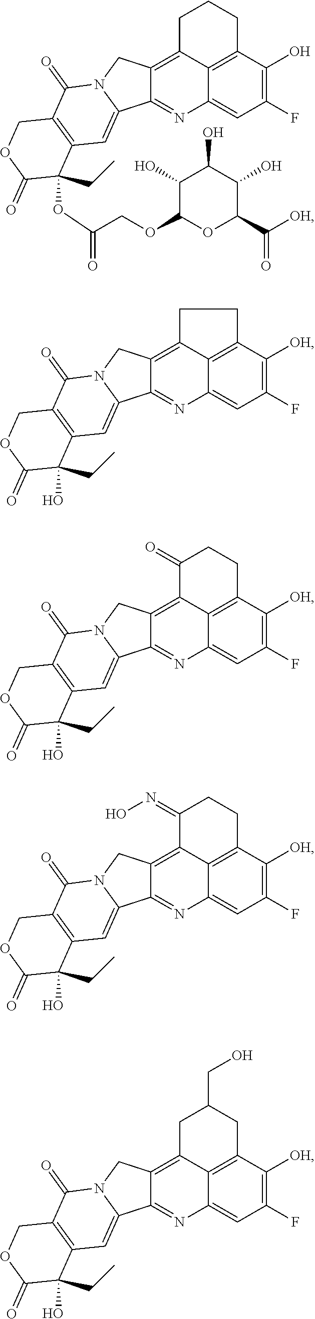

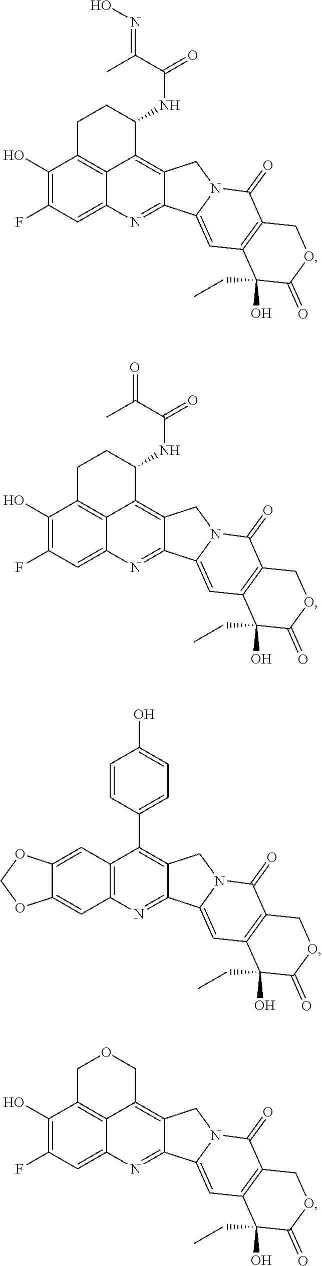 [SMILES: CC[C@@]1(O)C(=O)OCc2c1cc1n(c2=O)Cc2c-1nc1cc(F)c(O)c3c1c2[C@@H](NC(=O)/C(C)=N/O)CC3 ; CC[C@@]1(O)C(=O)OCc2c1cc1n(c2=O)Cc2c-1nc1cc(F)c(O)c3c1c2[C@@H](NC(=O)C(C)=O)CC3 ; CC[C@@]1(O)C(=O)OCc2c1cc1n(c2=O)Cc2c-1nc1cc3c(cc1c2-c1ccc(O)cc1)OCO3 ; CC[C@@]1(O)C(=O)OCc2c1cc1n(c2=O)Cc2c-1nc1cc(F)c(O)c3c1c2COC3]

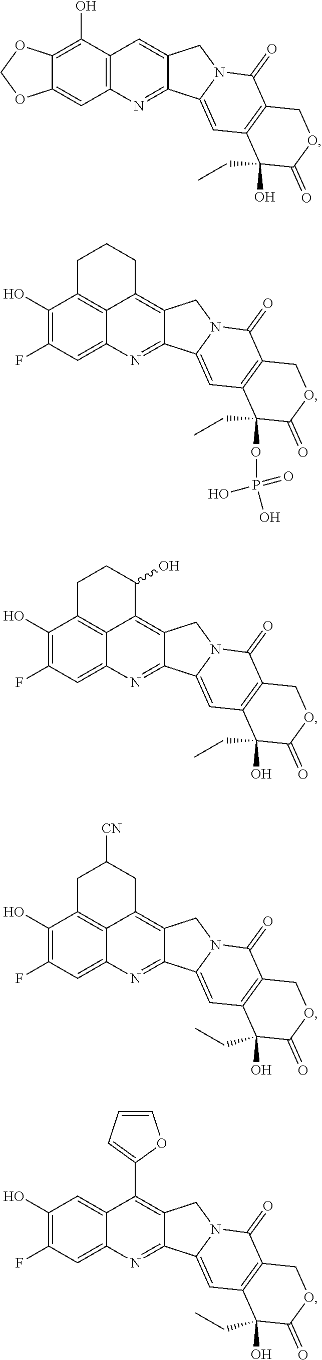 [SMILES: CC[C@@]1(O)C(=O)OCc2c1cc1n(c2=O)Cc2cc3c(O)c4c(cc3nc2-1)OCO4 ; CC[C@@]1(OP(=O)(O)O)C(=O)OCc2c1cc1n(c2=O)Cc2c-1nc1cc(F)c(O)c3c1c2CCC3 ; CC[C@@]1(O)C(=O)OCc2c1cc1n(c2=O)Cc2c-1nc1cc(F)c(O)c3c1c2C(O)CC3 ; CC[C@@]1(O)C(=O)OCc2c1cc1n(c2=O)Cc2c-1nc1cc(F)c(O)c3c1c2CC(C#N)C3 ; CC[C@@]1(O)C(=O)OCc2c1cc1n(c2=O)Cc2c-1nc1cc(F)c(O)cc1c2-c1ccco1]

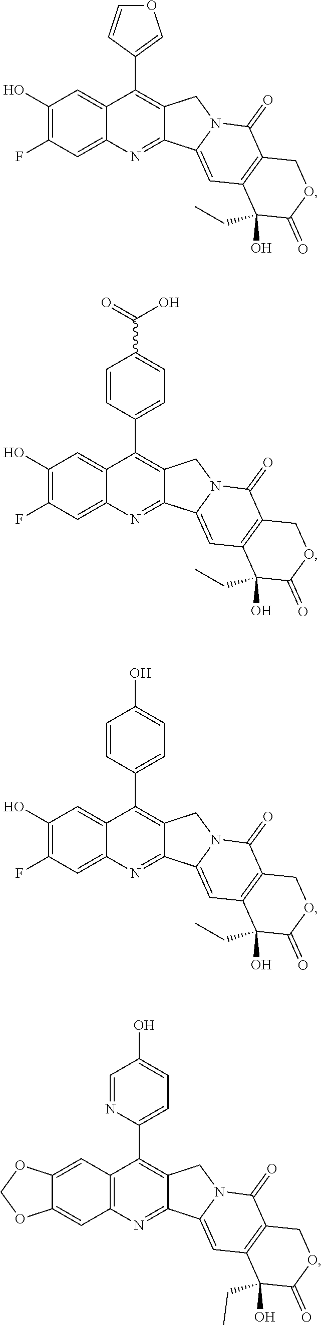 [SMILES: CC[C@@]1(O)C(=O)OCc2c1cc1n(c2=O)Cc2c-1nc1cc(F)c(O)cc1c2-c1ccoc1 ; CC[C@@]1(O)C(=O)OCc2c1cc1n(c2=O)Cc2c-1nc1cc(F)c(O)cc1c2-c1ccc(C(=O)O)cc1 ; CC[C@@]1(O)C(=O)OCc2c1cc1n(c2=O)Cc2c-1nc1cc(F)c(O)cc1c2-c1ccc(O)cc1 ; CC[C@@]1(O)C(=O)OCc2c1cc1n(c2=O)Cc2c-1nc1cc3c(cc1c2-c1ccc(O)cn1)OCO3]

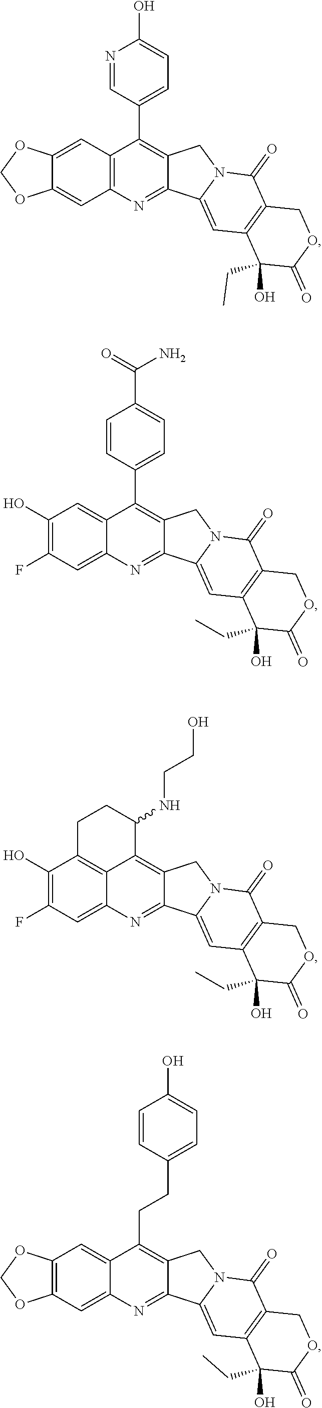 [SMILES: CC[C@@]1(O)C(=O)OCc2c1cc1n(c2=O)Cc2c-1nc1cc3c(cc1c2-c1ccc(O)nc1)OCO3 ; CC[C@@]1(O)C(=O)OCc2c1cc1n(c2=O)Cc2c-1nc1cc(F)c(O)cc1c2-c1ccc(C(N)=O)cc1 ; CC[C@@]1(O)C(=O)OCc2c1cc1n(c2=O)Cc2c-1nc1cc(F)c(O)c3c1c2C(NCCO)CC3 ; CC[C@@]1(O)C(=O)OCc2c1cc1n(c2=O)Cc2c-1nc1cc3c(cc1c2CCc1ccc(O)cc1)OCO3]

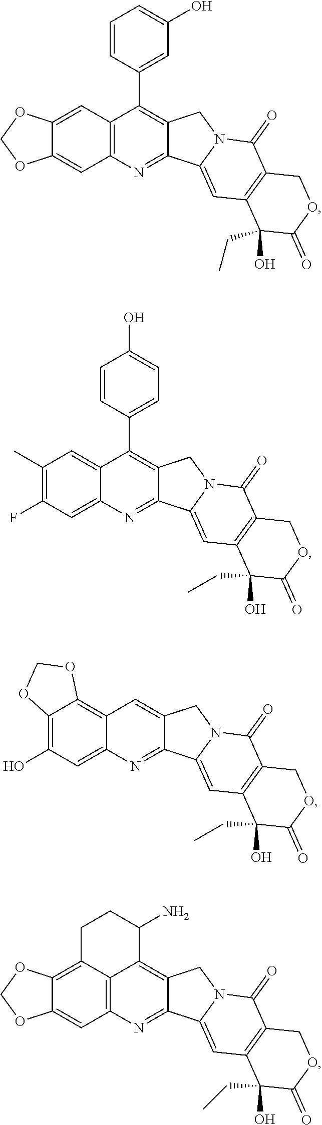 [SMILES: CC[C@@]1(O)C(=O)OCc2c1cc1n(c2=O)Cc2c-1nc1cc3c(cc1c2-c1cccc(O)c1)OCO3 ; CC[C@@]1(O)C(=O)OCc2c1cc1n(c2=O)Cc2c-1nc1cc(F)c(C)cc1c2-c1ccc(O)cc1 ; CC[C@@]1(O)C(=O)OCc2c1cc1n(c2=O)Cc2cc3c4c(c(O)cc3nc2-1)OCO4 ; CC[C@@]1(O)C(=O)OCc2c1cc1n(c2=O)Cc2c-1nc1cc3c(c4c1c2C(N)CC4)OCO3]

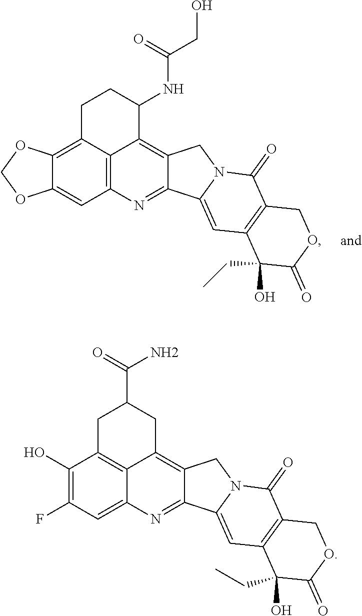 [SMILES: CC[C@@]1(O)C(=O)OCc2c1cc1n(c2=O)Cc2c-1nc1cc3c(c4c1c2C(NC(=O)CO)CC4)OCO3 ; CC[C@@]1(O)C(=O)OCc2c1cc1n(c2=O)Cc2c-1nc1cc(F)c(O)c3c1c2CC(C(N)=O)C3]

In certain embodiments, the compound is of one of the following structures, or is a pharmaceutically acceptable salt thereof:

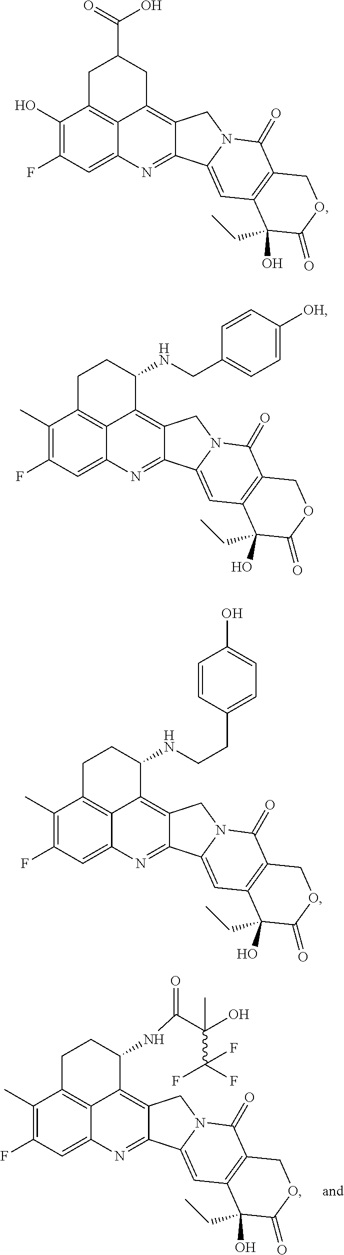

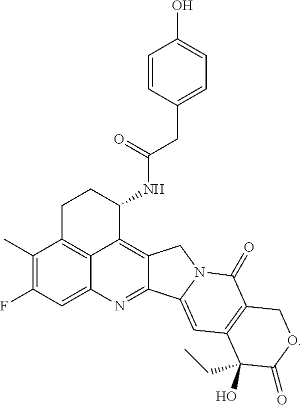 [SMILES: CC[C@@]1(O)C(=O)OCc2c1cc1n(c2=O)Cc2c-1nc1cc(F)c(C)c3c1c2[C@@H](NC(=O)Cc1ccc(O)cc1)CC3]

In certain embodiments, provided herein are pharmaceutical compositions comprising the compounds described above, and a pharmaceutically acceptable carrier.

In certain embodiments, provided herein are methods of treating a cancer, comprising administering the compound or the pharmaceutical composition as described above to a subject in need thereof. In further embodiments, the cancer is selected from leukemia, lymphoma, breast cancer, gastric cancer, colon cancer, ovarian cancer, bladder cancer, prostate cancer, glioma, lung cancer, bronchial cancer, colorectal cancer, pancreatic cancer, esophageal cancer, liver cancer, urinary bladder cancer, kidney cancer, renal pelvis cancer, oral cavity cancer, pharynx cancer, uterine corpus cancer, or melanoma. In yet further embodiments, the cancer is selected from breast cancer, gastric cancer, lung cancer and colon cancer. In certain embodiments, provided herein are methods of treating an autoimmune disease or an inflammatory disease, comprising administering the compound, or the pharmaceutical composition as described above to a subject in need thereof. In further embodiments, the autoimmune disease or the inflammatory disease is selected from B-cell mediated autoimmune diseases or inflammatory diseases, for example, systemic lupus erythematosus (SLE), rheumatoid arthritis (RA), idiopathic thrombocytopenic purpura (ITP), Waldenstrom's hypergammaglobulinaemia, Sjogren's syndrome, multiple sclerosis (MS), or lupus nephritis.

### Release of the Active Agent

As described above, in certain embodiments, the compounds and conjugates disclosed herein contain a cleavage group L^{C}, which cleaves to release the active agent under certain conditions. Many suitable cleavage groups are known in the art. Some, which in certain embodiments are particularly preferred, are capable of dissociating one or more active agents through an intramolecular cyclization reaction following a chemical reaction that activates the triggering group. In certain embodiments, the chemical reaction is a physicochemical reaction and/or a biochemical reaction.

In some embodiments, the compounds and conjugates disclosed herein comprise a nucleophilic functional group (Y¹) introduced at an atom on Ar adjacent to X (e.g., O). Typically, the nucleophilic functional group is masked by a triggering group (TG), as further detailed below. For example, as described in detail herein, the structure of L^{C} may be as follows:

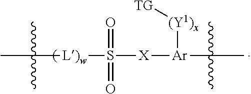

As further described herein, according to these embodiments, Ar is coupled to L^{B} and L′ (or the S) is coupled to the drug moiety.

Upon activation, the triggering group releases the nucleophilic functional group to react with the nearby SO₂ moiety in an intramolecular cyclization, ultimately releasing the one or more compounds of Formula (XII) or (XIII). In some such embodiments, one or more active agents are released through an intramolecular cyclization reaction after a chemical reaction, a physicochemical reaction and/or a biochemical reaction (see, for example, Reaction Scheme 1), or the active agent is released through 1,6-elimination or 1,4-elimination after the intramolecular cyclization reaction (see, for example, Reaction Scheme 2).

As an example, when Y is —Y¹ and Q is an active agent directly conjugated to the SO₂ group, the active agent may be released by the mechanism shown in Reaction Scheme 1:

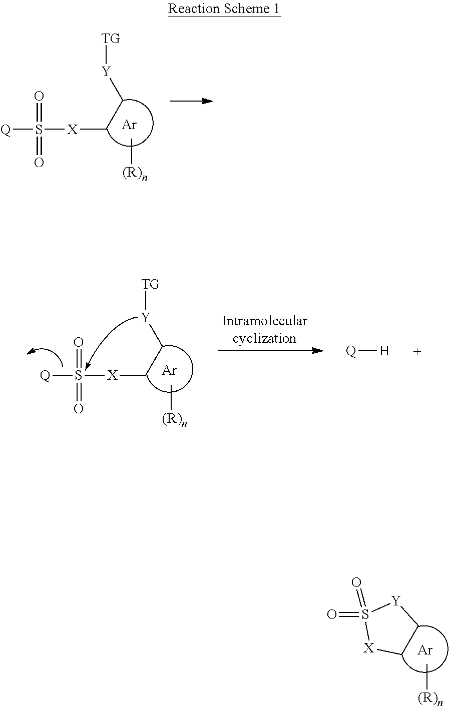

When Q comprises a spacer moiety (e.g., when Q is

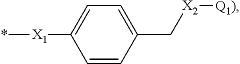

active agent Q¹ may be released by the mechanism shown in Reaction Scheme 2:

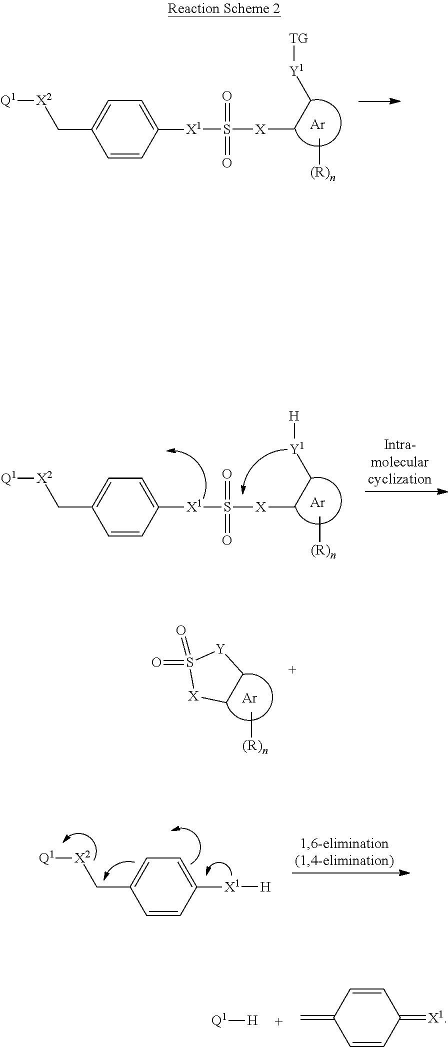

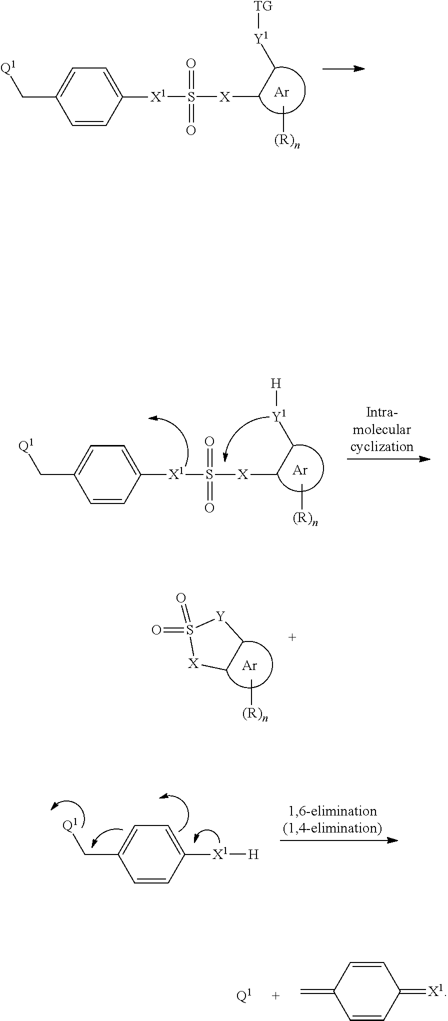

In some embodiments, Q¹ when released is an active agent comprising at least one functional group selected from, e.g., —C(O)—, —OH, —NH—, —SH, —COH, and —COOH. According to these embodiments, as further described herein, Q¹ is conjugated to a compound as described herein by, e.g., the —C(O)—, —OH, —NH—, —SH, —COH, or —COOH, for instance through a functional group selected from ester, amide, thioester, carbamate, urea, oxime, hydrazone, etc. In some such embodiments, Q² is used in place of Q¹, and Q² is an amine group-containing drug. In other embodiments, Q² is an active agent capable of binding with an ammonium unit. In still other embodiments, Q² is capable of being dissociated in its original form having an amine group upon release of Q² release, wherein the active agent may be a drug, a toxin, an affinity ligand, a probe for detection, or a combination thereof.

In some embodiments, the compounds and conjugates disclosed herein are chemically and physiologically stable. In some such embodiments, the compounds and conjugates disclosed herein reach a desired target cell in a state wherien there is little dissociation of the active agent in the blood, thereby selectively releasing the drug.

### Triggering Groups (TGs)

In some embodiments, the conjugates of the present disclosure include a triggering group (TG). TGs are groups capable of being cleaved, preferably selectively cleaved, by a chemical reaction, such as a biological reaction. Generally, triggering groups serve to mask the nucleophilic nature of the Y′ group, thereby providing stability (e.g., by preventing self-immolation or intramolecular cyclization prior to the conjugate reaching a target location or experiencing a predetermined trigger condition) to the compounds and conjugates disclosed herein. Upon activation, the triggering group releases the nucleophilic Y group and allows for self-immolation or intramolecular cyclization to occur, as described above.

In some embodiments, the TG comprises a sequence (such as a peptide sequence) or a moiety recognized by TEV, trypsin, thrombin, cathepsin B, cathespin D, cathepsin K, caspase 1, matrix metalloproteinase (MMP), and the like, which can be hydrolyzed by an enzyme (e.g., an oxidoreductase, a transferase, a hydrolase, a lyase, an isomerase, a ligase, etc.) and/or may include a moiety selected from a sulfate, a phosphodiester, a phospholipid, an ester, a β-galactose, a β-glucose, a fucose, an oligosugar, and the like.

In some embodiments, the TG comprises a reactive chemical moiety or functional group that can be cleaved under nucleophilic reagent conditions (e.g., a silyl ether, a 2-N-acyl nitrobenzenesulfonamide, an unsaturated vinyl sulfide, a sulfonamide after activation, a malondialdehyde-indole derivative, a levulinoyl ester, a hydrazone, or an acyl hydrazone).

In some embodiments, the TG may comprise a reactive chemical moiety or functional group that can be cleaved under basic reagent conditions (e.g., a 2-cyanoethyl ester, an ethylene glycolyl disuccinate, a 2-sulfonylethyl ester, an alkyl thioester, or a thiophenyl ester).

In some embodiments, the TG may comprise a reactive chemical moiety or functional group that can be cleaved by photo-irradiation (e.g., 2-nitrobenzyl derivative, phenacyl ester, 8-quinolinyl benzenesulfonate, coumarin, phosphotriester, bis-arylhydrazone, or bimane bi-thiopropionic acid derivative).

In some embodiments, the TG may comprise a reactive chemical moiety or functional group that can be cleaved by reducing agent conditions (e.g., hydroxylamine, disulfide, levulinate, nitro, or 4-nitrobenzyl derivative).

In some embodiments, the TG may comprise a reactive chemical moiety or a functional group that can be cleaved using acidic conditions (e.g., saccharides, tert-butylcarbamate analogue, dialkyl or diaryl dialkoxysilane, orthoester, acetal, aconityl, hydrazone, β-thiopropionate, phosphoramidate, imine, trityl, vinyl ether, polyketal, and alkyl 2-(diphenylphosphino)benzoate derivative; alkyl ester, 8-hydroxyquinoline ester, and picolinate ester).

In some embodiments, the TG may comprise a reactive chemical moiety or functional group that can be cleaved under oxidative conditions (e.g., a boronate, a vicinal diol, paramethoxybenzyl derivative, or a selenium compound).

In certain preferred embodiments, the TG comprises a saccharide, which can be cleaved under acidic or enzymatic conditions. In certain preferred embodiments, the triggering group is —NO₂, which can be cleaved under reducing conditions. In certain preferred embodiments, the triggering group is a boronate, which can be cleaved under oxidative conditions. In certain preferred embodiments, the triggering group is an ester, which can be cleaved under acidic, basic, or enzymatic conditions. In certain preferred embodiments, the triggering group is a hydrazone, which can be cleaved under nucleophilic conditions or under acidic conditions. In certain preferred embodiments, the triggering group is a hydroxylamine, which can be cleaved under reducing conditions.

### Saccharide Triggering Groups

In some embodiments, the compounds and conjugates disclosed herein comprise a saccharide triggering group, for instance a triggering group selected from:

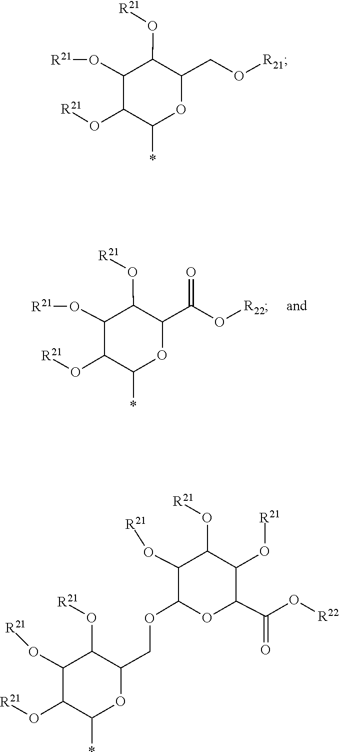

wherein each R²¹ is independently hydrogen or is selected such that O—R²¹ is a hydroxy protecting group (e.g., acetyl); and R²² is hydrogen or lower alkyl (e.g., C₁-C₆-alkyl). In certain embodiments, the hydroxy protecting group is capable of being used in organic synthesis, including but not limited to: methyl ether, methoxymethyl ether, methylthiomethyl ether, 2-methoxyethoxymethyl ether, bis(2-chloroethoxy)methyl ether, tetrahydropyranyl ether, tetrahydrothiopyranyl ether, 4-methoxytetrahydropyranyl ether, 4-methoxytetrahydrothiopyranyl ether, tetrahydrofuranyl ether, 1-ethoxyethyl ether, 1-methyl-1-methoxyethyl ether, 2-(phenylselenyl)ethyl ether, t-butyl ether, allyl ether, benzyl ether, o-nitrobenzyl ether, triphenyl methyl ether, α-naphthyldiphenyl methyl ether, p-methoxyphenyldiphenylmethyl ether, 9-(9-phenyl-10-oxo)anthryl ether, trimethylsilyl ether, isopropyldimethylsilyl ether, t-butyldimethylsilyl ether, t-butyldiphenylsilyl ether, tribenzylsilyl ether, triisopropylsilyl ether, formate ester, acetate ester, trichloroacetate ester, phenoxyacetate ester, isobutyrate ester, pivaloate ester, adamantoate ester, benzoate ester, 2,4,6-trimethylbenzoate ester, methyl carbonate, 2,2,2-trichloroethyl carbonate, allyl carbonate, p-nitrophenyl carbonate, benzyl carbonate, p-nitrobenzyl carbonate, S-benzylthiocarbonate, N-phenylcarbamate, nitrate ester, 2,4-dinitrophenylsulfenate ester, etc., but is not limited thereto.

### Protecting Groups as Triggering Groups

In some embodiments, TG is a group that is capable of being cleaved by a chemical reaction, a physicochemical reaction, and/or a biological reaction. In certain embodiments, TG is a protecting group. In some such embodiments, the protecting group is an amine group protecting group, an alcohol protecting group, or a thiol protecting group.

### Amine Protecting Groups

In certain embodiments, the amine protecting group is a general protecting group that is capable of being used in organic synthesis, including but not limited to: m-nitrophenyl carbamate, 3,5-dimethoxybenzyl carbamate, o-nitrobenzyl carbamate, phenyl(o-nitrophenyl)methyl carbamate, alkyl carbamate, 9-fluorenylmethyl carbamate, 2,2,2-trichloroethyl carbamate, 2-trimethylsilylethyl carbamate (Teoc), t-butyl carbamate (Boc), vinyl carbamate (Voc), allyl carbamate (Alloc), 1-isopropylallyl carbamate (Ipaoc), 8-quinolyl carbamate, N-hydroxypiperidinyl carbamate, benzyl carbamate, p-methoxybenzyl carbamate, p-nitrobenzyl carbamate, diphenyl methyl carbamate, acetamide, chloroacetamide, trichloroacetamide, phenylacetamide, benzamide, N-phthalimide, N-2,3-diphenylmaleimide, N-2,5-dimethylpyrrole, N-1,1-dimethylthiomethyleneamine, N-benzylideneamine, benzenesulfenamide, o-nitrobenzenesulfenamide, triphenylmethylsulfenamide, p-toluenesulfonamide, methanesulfonamide, etc., but is not limited thereto.

### Alcohol Protecting Groups

In certain embodiments, the alcohol protecting group is a general protecting group that is capable of being used in organic synthesis, including but not limited to: methyl ether, methoxymethyl ether (MOM ether), benzyloxymethyl ether (BOM ether), 2-(trimethylsilyl)ethoxymethyl ether (SEM ether), phenylthiomethyl ether (PTM ether), 2,2-dichloro-1,1-difluoroethyl ether, p-bromophenacyl ether, chloropropylmethyl ether, isopropyl ether, cyclohexyl ether, 4-methoxybenzyl, 2,6-dichlorobenzyl ether, 4-(dimethylaminocarbonyl)benzyl ether, 9-anthrylmethyl ether, 4-picolyl ether, methylthiomethyl ether (MTM ether), 2-methoxyethoxymethyl ether (MEM ether), bis(2-chloroethoxy)methyl ether, tetrahydropyranyl ether (THP ether), tetrahydrothiopyranyl ether, 4-methoxytetrahydropyranyl ether, 4-methoxytetrahydrothiopyranyl ether, tetrahydrofuranyl ether, 1-ethoxyethyl ether, 1-methyl-1-methoxyethyl ether, 2-(phenylselenyl)ethyl ether), t-butyl ether, allyl ether, benzyl ether, o-nitrobenzyl ether, triphenylmethyl ether, α-naphthyldiphenylmethyl ether, p-methoxyphenyldiphenylmethyl ether, 9-(9-phenyl-10-oxo)anthryl ether, trimethylsilyl ether (TMS ether), isopropyldimethylsilyl ether, t-butyldimethylsilyl ether (TBDMS ether), t-butyldiphenyl silyl ether, tribenzylsilyl ether, triisopropylsilyl ether, formate ester, acetate ester, trichloroacetate ester, phenoxyacetate ester, isobutyrate ester, pivaloate ester, adamantoate ester, benzoate ester, 2,4,6-trimethylbenzoate (Mesitoate) ester, methyl carbonate, 2,2,2-trichloroethyl carbonate, allyl carbonate, p-nitrophenyl carbonate, benzyl carbonate, p-nitrobenzyl carbonate, S-benzyl thiocarbonate, N-phenylcarbamate, nitrate ester, 2,4-dinitrophenylsulfenate ester, dimethylphosphinyl ester (DMP ester), dimethylthiophosphinyl ester (MPT ester), aryl methanesulfonate, aryl toluenesulfonate, etc., but is not limited thereto.

### Thiol Protecting Groups

In certain embodiments, the thiol protecting group is capable of being used in organic synthesis, including but not limited to: S-benzyl thioether, S-p-methoxybenzyl thioether, S-o- or p-hydroxyl or acetoxybenzyl thioether, S-p-nitrobenzyl thioether, S-4-picolyl thioether, S-2-picolyl N-oxide thioether, S-9-anthrylmethyl thioether, S-9-fluorenylmethyl thioether, S-methoxymethyl monothioacetal, A-acetyl derivative, S-benzoyl derivative, S—(N-ethylcarbamate), S—(N-methoxymethylcarbamate), etc., but is not limited thereto.

Coupling Groups from the Drug Conjugate to the Cell-Binding Group (Linking Group)

In some embodiments, the compounds and conjugates disclosed herein comprise a linking group connecting each CB and Ar through covalent bonds. Preferably, the linking group may be an OHPAS linker as described in U.S. patent Ser. No. 11/753,431. It should be noted that any cleavable linker that can be linked to an alcohol group may be used. Typical linking groups are stable, non-hydrolyzable groups, such as, for example a C₇-C₁₀₀ linear or branched, saturated or unsaturated alkylene, optionally comprising one or more double bonds and/or triple bonds. In certain embodiments, the linking unit satisfies at least two, and more preferably at least three, out of four of the following criteria:
(i) at least one —CH₂— in the alkylene moiety is replaced by one or more heteroatoms selected from —NH—, —C(═O), —O—, —S— and —P—;
(ii) at least one heteroarylene is included in the alkylene moiety;
(iii) at least one amino acid moiety, sugar bond, peptide bond, or amide bond is included in the alkylene moiety; and
(iv) the alkylene may be further substituted with one or more substituents selected from the group consisting of C₁-C₂₀ alkyl, C₆-C₂₀ aryl C₁-C₈ alkyl, —(CH₂)ₛCOOH, and —(CH₂)ₚNH₂, wherein s is an integer having a value of 0 to 10, and p is an integer having a value of 1 to about 10.

In certain embodiments, the linking unit comprises at least two, and more preferably at least three, of the following:
(i) at least one heteroatom selected from —NH—, —C(═O), —O—, —S— and —P—;
(ii) at least one heteroarylene;
(iii) at least one amino acid moiety, sugar bond, peptide bond, or amide bond; and
(iv) the alkylene may be further substituted with one or more substituents selected from the group consisting of C₁-C₂₀ alkyl, C₆-C₂₀ aryl C₁-C₈ alkyl, —(CH₂)ₛCOOH, and —(CH₂)ₚNH₂, wherein s is an integer having a value of 0 to 10, and p is an integer having a value of 1 to about 10.

In other embodiments, the linking group connecting each CB and Ar comprises a functional group produced through a click chemical reaction.

In alternative embodiments, the linking unit comprises a reactive functional group capable of participating in a click chemical reaction.

A click chemical reaction is a reaction that can be performed under mild conditions, and is extremely selective for functional groups that are not commonly found in biological molecules (e.g., an azide group, an acetylene group, etc.). Accordingly, this reaction can be carried out in the presence of complex triggering groups, cell-binding groups, etc. Further, click chemistry has high reaction specificity. For example, the click chemical reaction between an azide group and an acetylene group proceeds selectively without interference from other functional groups present in the molecule. For example, azide-acetylene click chemistry may afford a triazole moiety in high yield.

Thus, in some embodiments, the linking group connecting each CB and Ar comprises

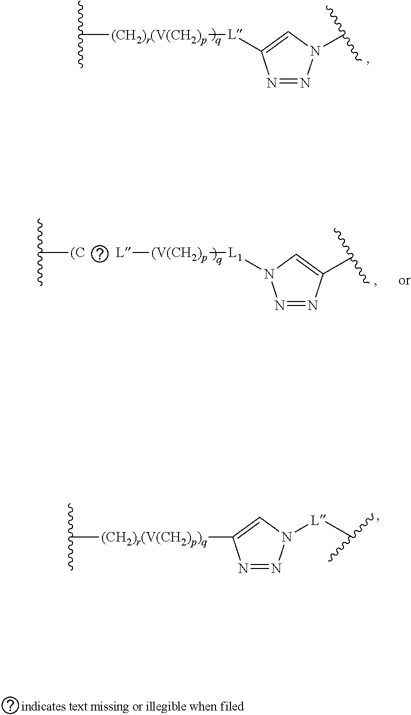

V may be a single bond, —O—, —S—, —NR²¹—, —C(O)NR²²—, —NR²³C(O)—, —NR²⁴SO₂—, or —SO₂NR²⁵—, R²¹ to R²⁵ may be each independently hydrogen, (C₁-C₆)alkyl, (C₁-C₆)alkyl(C₆-C₂₀)aryl, or (C₁-C₆)alkyl(C₃-C₂₀)heteroaryl, r may be an integer having a value of 1 to about 10, p may be an integer having a value of 0 to about 10, q may be an integer having a value of 1 to about 10, and L″ may be a single bond.

Various linking groups (or spacer groups) L^{B} are suitable for use with the presently disclosed drug conjugates, and are bonded either to a reactive group L^{A} (which is capable of reacting with a targeting moiety TM) or a coupling group L^{D}, which is further bonded to the targeting moiety. When a TM is not present, the linking group comprises a terminal reactive functional moiety that can react with a cell-binding group. In some embodiments, L^{B}-L^{A} comprises, or is selected from:

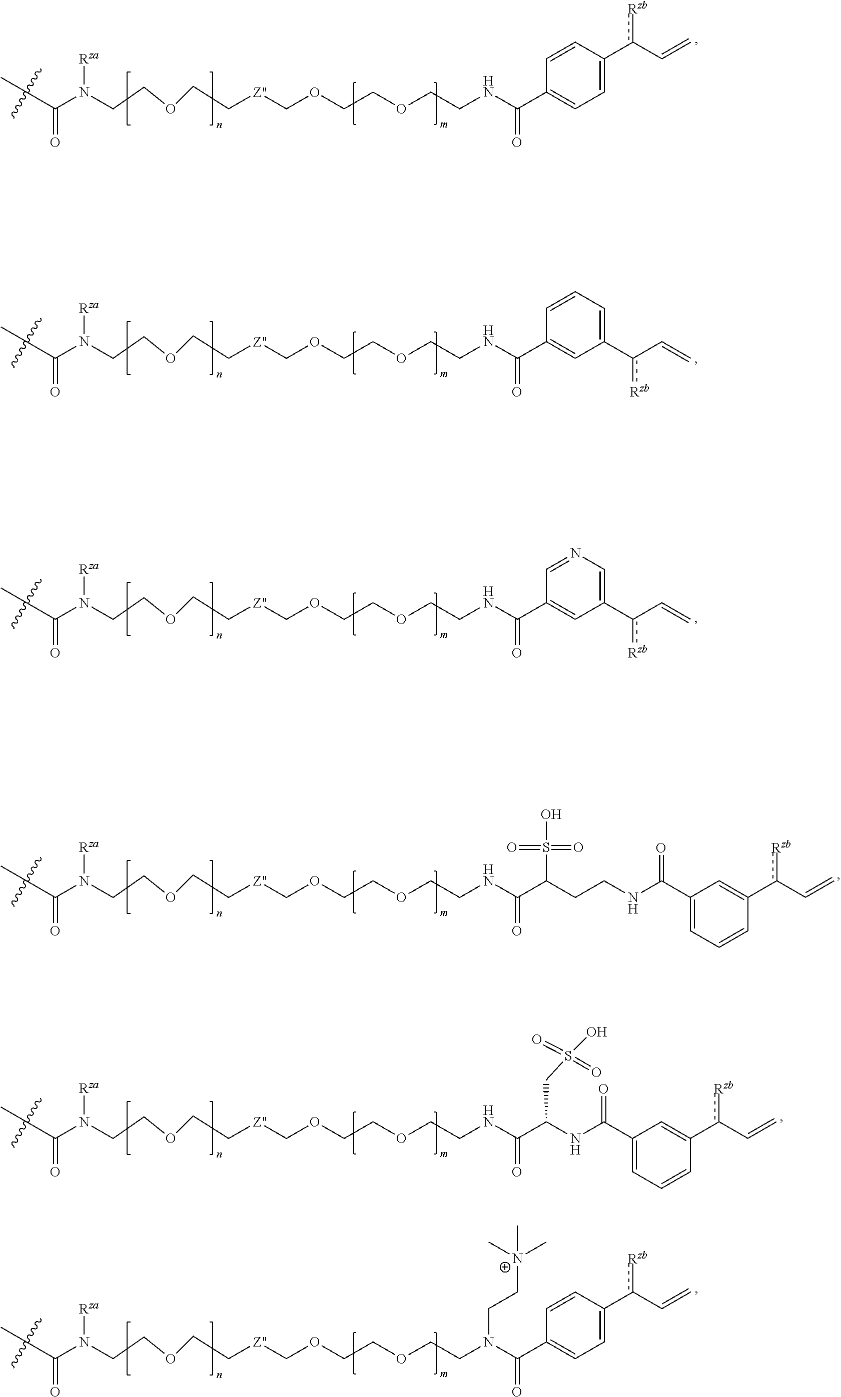

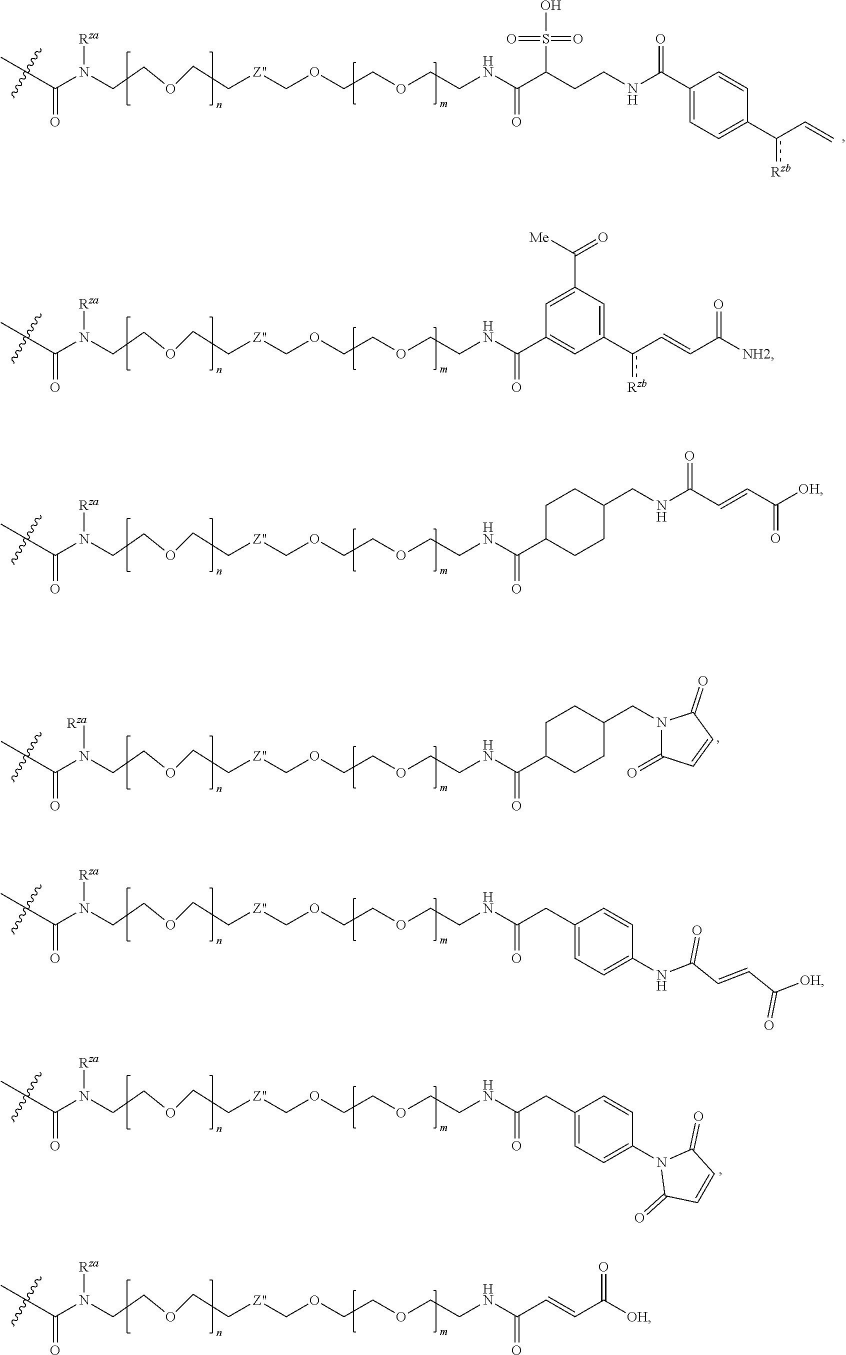

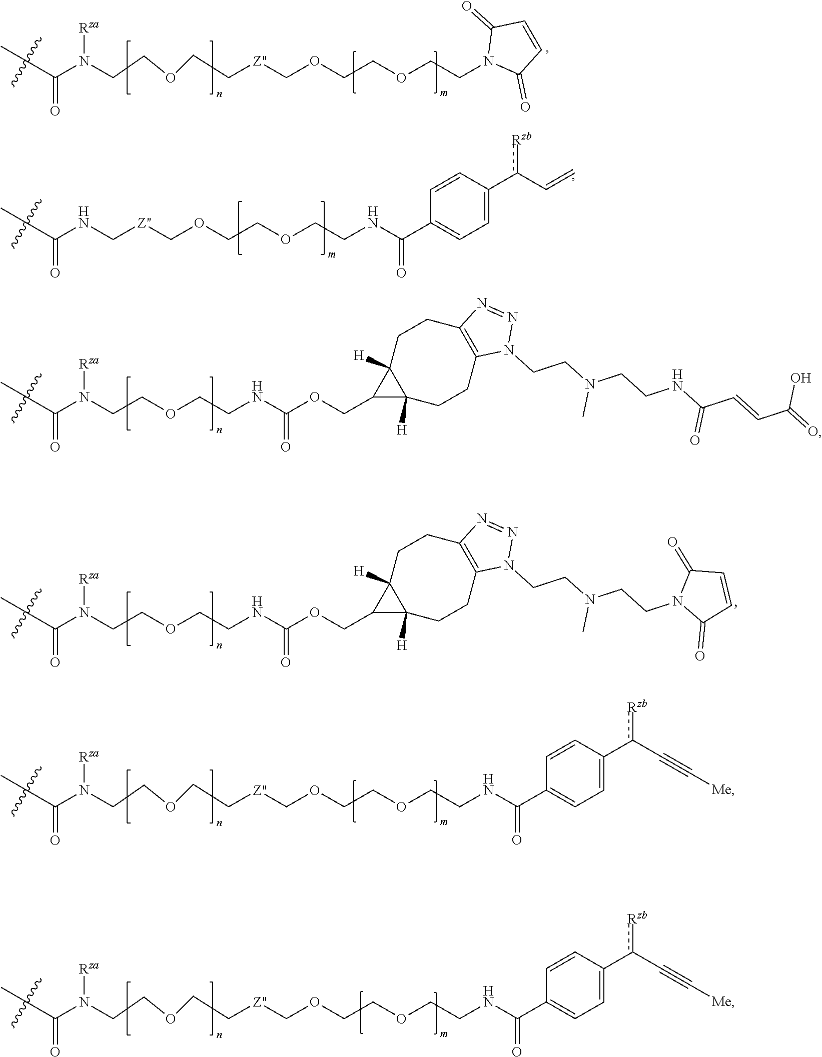

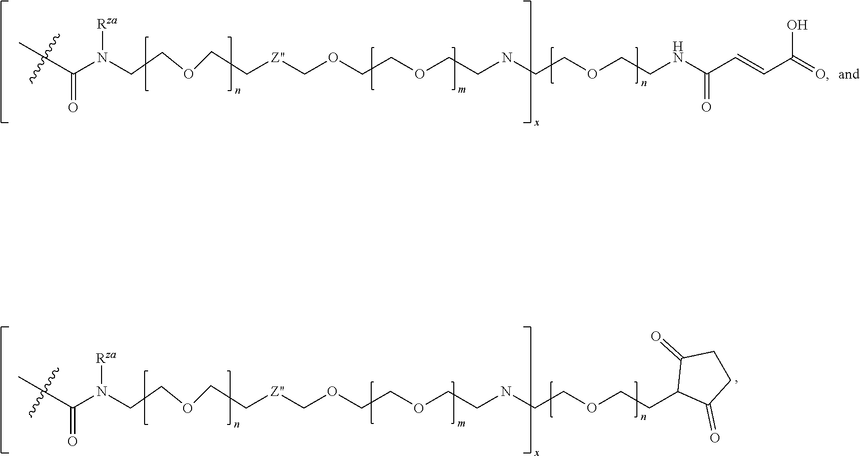

wherein:
R^{a} is (CH₂)ₐₐR′;
aa is an integer from 0 to 10;
R′ is selected from H, hydroxy, aryl, heteroaryl, cycloalkyl, heterocyclyl, nitro, amino; cyano,
halo, and C(═O)OR″;
R″ is selected from H and alkyl;
R^{zb} is —OH, ═O, or ═NHOH;
n and m are each independently integer selected from 1-10;
x is integer selected from 1-2;

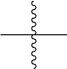

represents the bond between L^{B}-L^{A} and the drug conjugate;
[symbol] a single bond or a double bond;
Z″ is selected from

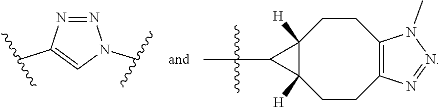 [SMILES: *c1cn(*)nn1 ; *C1[C@H]2CCc3nnn(C)c3CC[C@@H]12]

In other embodiments, the L^{B}-L^{A} comprises, or is selected from:

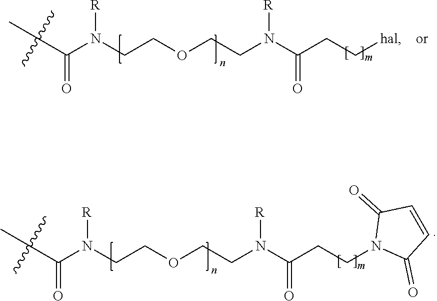

wherein hal is halo;
R is (CH₂)ₐₐR′;
aa is an integer from 0 to 10;
R′ is selected from H, hydroxy, aryl, cycloalkyl, nitro, amino, cyano, halo, (OCH₂CH₂)ₙ)_{bb}R″, and C(═O)OR″, preferably at least one is (OCH₂CH₂)ₙ)_{bb}R″;
R″ is selected from H, alkyl, and hydroxyl;

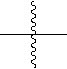

represents the bond between L^{B}-L^{A} and the remainder of the compound;
bb is an integer from 1 to 20, preferably 6 to 12;
n is an integer from 0 to 10; and
m is an integer from 0 to 10.

In certain embodiments, when a cell-binding group is present, the group L^{B}-L^{D} links the conjugate to the targeting moiety, TM. In some embodiments, L^{B}-L^{D} is selected from:

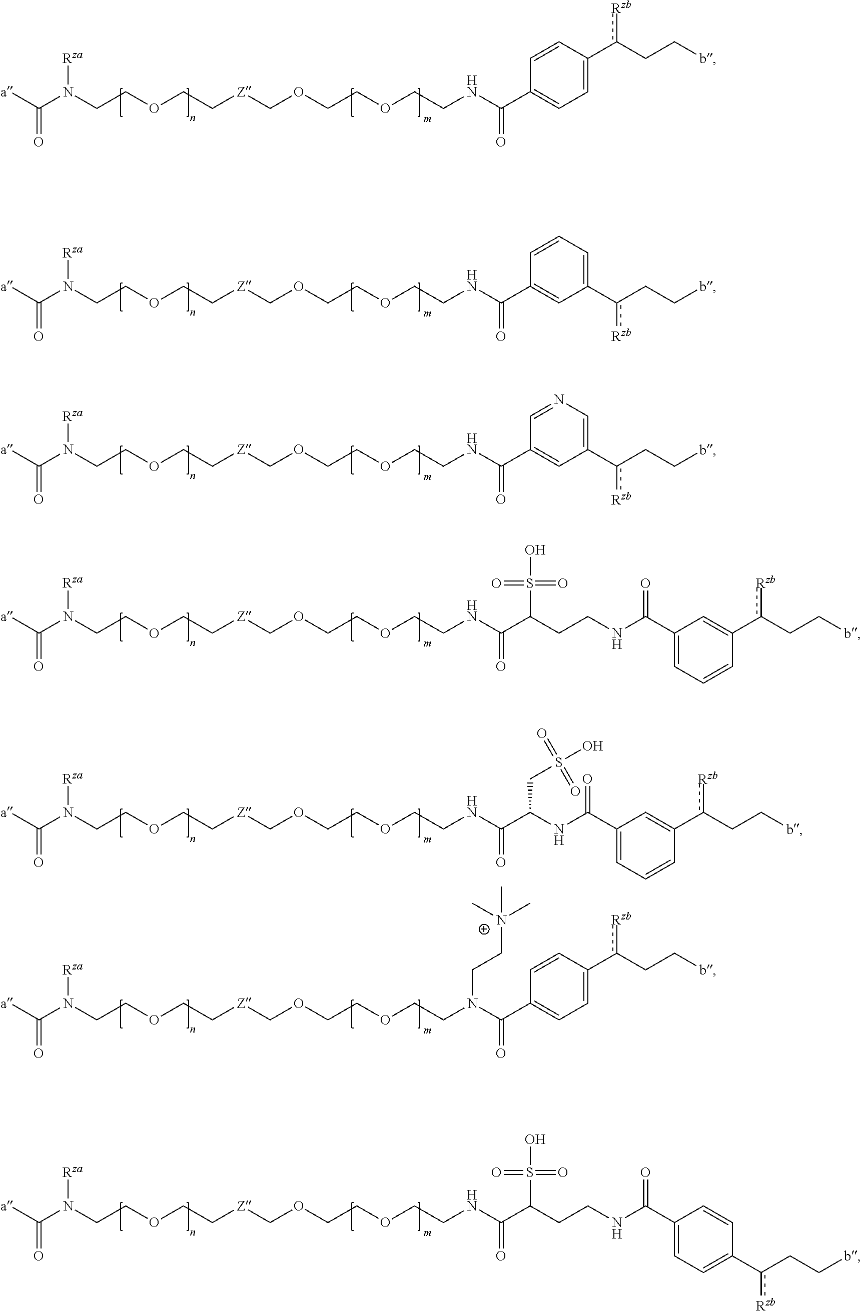

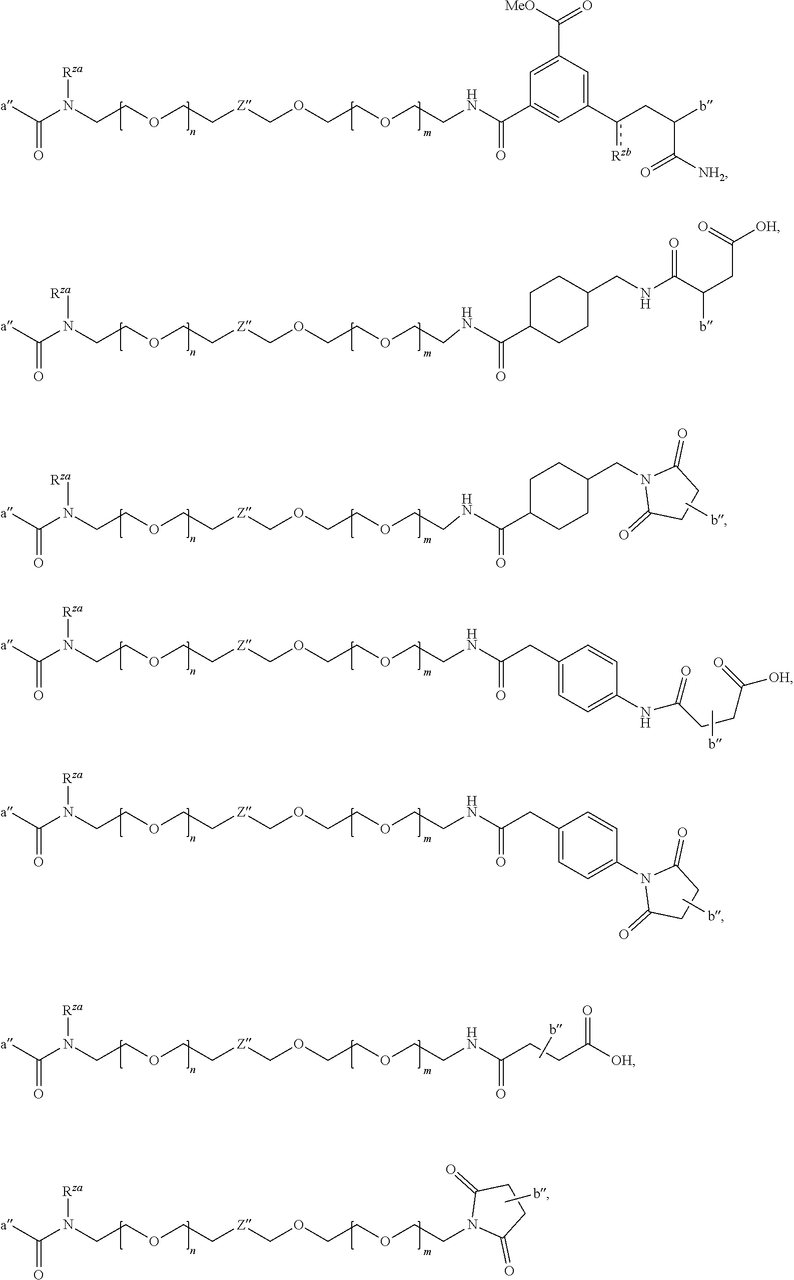

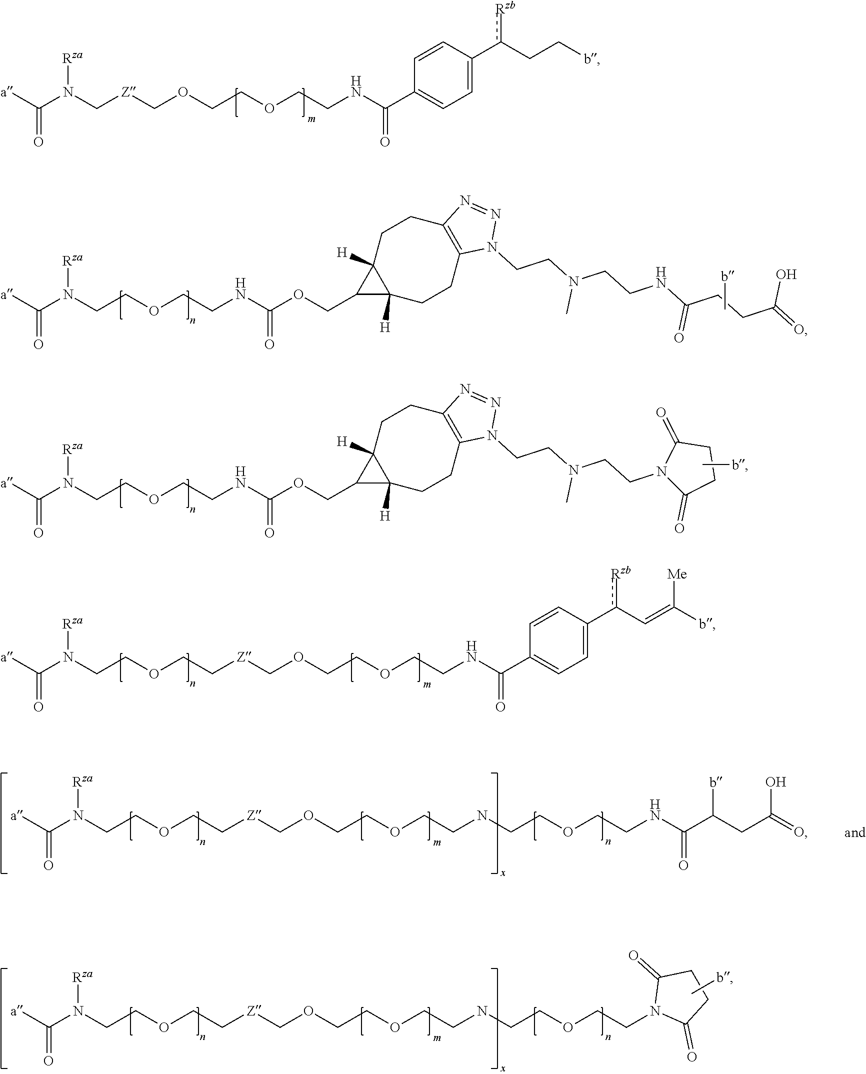

wherein
R^{za} is (CH₂)ₐₐR′;
aa is an integer from 0 to 10;
R′ is selected from H, hydroxy, aryl, heteroaryl, cycloalkyl, heterocyclyl, nitro, amino; cyano, halo, and C(═O)OR″;
R″ is selected from H and alkyl;
R^{zb} is —OH, ═O, or ═NHOH;
[symbol] a single bond or a double bond;
n and m are each independently an integer selected from 1-10;
x is an integer selected from 1-2;
b″ represents the bond between L^{B}-L^{D} and TM; and
a″ represents the bond between L^{B}-L^{D} and the remainder of the compound;
Z″ is selected from

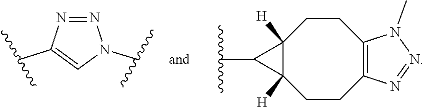 [SMILES: *c1cn(*)nn1 ; *C1[C@H]2CCc3nnn(C)c3CC[C@@H]12]

In certain embodiments, the linkage group between L^{C} and TM (L^{B}-L^{D}) is selected from

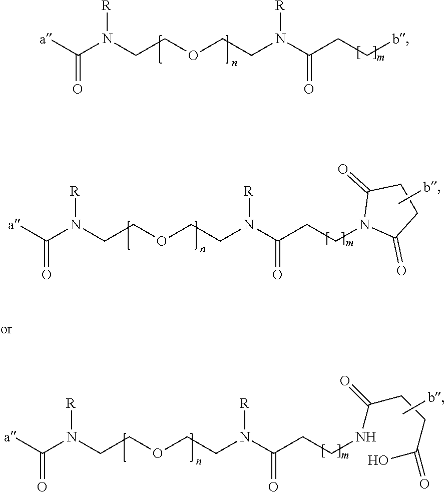

wherein R is (CH₂)ₐₐR′;
aa is an integer from 0 to 10;
R′ is selected from H, hydroxy, aryl, cycloalkyl, nitro, amino, cyano, halo, (OCH₂CH₂)ₙ)_{bb}R″, and C(═O)OR″;
R″ is selected from H, alkyl, and hydroxyl;
bb is an integer from 1 to 20;
b″ represents the bond between L^{B}-L^{D} and TM;
a″ represents the bond between L^{B}-L^{D} and the remainder of the compound;
n is an integer from 0 to 10; and
m is an integer from 0 to 10.

### Cell-Binding Groups as Targeting Moieties

The compounds and conjugates of the present disclosure can further comprise one or more ligand or cell-binding groups, CB. In some embodiments, the ligand or cell-binding group is any molecular recognition element, which can undergo a specific interaction with at least one other molecular through, e.g., noncovalent bonding such as hydrogen bonding, metal coordination, hydrophobic forces, van der Waals forces, π-π interactions, halogen bonding, electrostatic, and/or electromagnetic effects. In certain embodiments, CB is selected from a nanoparticle, an immunoglobulin, a nucleic acid, a protein, an oligopeptide, a polypeptide, an antibody, a fragment of an antigenic polypeptide, a repebody, and the like.

The compounds and conjugates of the present disclosure may comprise one or more cell-binding groups. In certain embodiments, the cell-binding group is a nanoparticle, an immunoglobulin, a nucleic acid, a protein, an oligopeptide, a polypeptide, an antibody, a fragment of an antigenic polypeptide, or a repebody. In further embodiments, the cell-binding group is an antibody selected from an intact polyclonal antibody, an intact monoclonal antibody, an antibody fragment, a single chain Fv (scFv) mutant, a multispecific antibody, a bispecific antibody, a chimeric antibody, a humanized antibody, a human antibody, a fusion protein comprising an antigenic determinant portion of an antibody, and other modified immunoglobulin molecules comprising antigen recognition sites.

In yet further embodiments, the antibody is selected from Muromonab-CD3, Abciximab, Rituximab, Daclizumab, Palivizumab, Infliximab, Trastuzumab (herceptin), Rosopatamab, Sacituzumab, Patritumab, Etanercept, Basiliximab, Gemtuzumab ozogamicin, Alemtuzumab, Ibritumomab tiuxetan, Adalimumab, Alefacept, Omalizumab, Efalizumab, Tositumomab-I¹³¹, Cetuximab, Bevacizumab, Natalizumab, Ranibizumab, Panitumumab, Eculizumab, Rilonacept, Certolizumab pegol, Romiplostim, AMG-531, CNTO-148, CNTO-1275, ABT-874, LEA-29Y, Belimumab, TACI-Ig, Second generation anti-CD20, ACZ-885, Tocilizumab, Atlizumab, Mepolizumab, Pertuzumab, Humax CD20, Tremelimumab (CP-675 206), Ticilimumab, MDX-010, IDEC-114, Inotuzumab ozogamycin, HuMax EGFR, Aflibercept, HuMax-CD4, Ala-Ala, ChAglyCD3, TRX4, Catumaxomab, IGN101, MT-201, Pregovomab, CH-14.18, WX-G250, AMG-162, AAB-001, Motavizumab, MEDI-524, Efumgumab, Aurograb, Raxibacumab, Third generation anti-CD20, LY2469298, and Veltuzumab.

In some embodiments, CB comprises two or more independently selected natural amino acids or non-natural amino acids conjugated by covalent bonds (e.g., peptide bonds), and the peptide may include 2, 3, 4, 5, 6, 7, 8, 9, 10, 11, 12, 13, 14, 15, 16, 17, 18, 19, 20, or more natural amino acids or non-natural amino acids that are conjugated by peptide bonds. In some embodiments, the ligand comprises shorter amino acid sequences (e.g., fragments of natural proteins or synthetic polypeptide fragments) as well as full-length proteins (e.g., pre-engineered proteins).

In some embodiments, CB is selected from an antibody, a hormone, a drug, an antibody analogue (e.g., non-IgG), protein, an oligopeptide, a polypeptide, etc., which bind to a receptor. In certain embodiments, CB selectively targets the drug in a specific organ, tissue, or cell. In other embodiments, CB specifically binds to a receptor over-expressed in cancer cells as compared to normal cells, and may be classified into a monoclonal antibody (mAb) or an antibody fragment and a low-molecular non-antibody. Preferably, CB is selected from peptides, tumor cell-specific peptides, tumor cell-specific aptamers, tumor cell-specific carbohydrates, tumor cell-specific monoclonal antibodies, polyclonal antibodies, and antibody fragments that are identified in a library screen.

Exemplary ligands or cell-binding groups include, but are not limited to, carnitine, inositol, lipoic acid, pyridoxal, ascorbic acid, niacin, pantothenic acid, folic acid, riboflavin, thiamine, biotin, vitamin B₁₂, other water-soluble vitamins (vitamin B), fat-soluble vitamins (vitamin A, D, E, K), RGD (Arg-Gly-Asp), NGR (Asn-Gly-Arg), transferein, VIP (vasoactive intestinal peptide) receptor, APRPG (Ala-Pro-Arg-Pro-Gly) peptide, TRX-20 (thioredoxin-20), integrin, nucleolin, aminopeptidase N (CD13), endoglin, vascular epithelial growth factor receptor, low density lipoprotein receptor, transferrin receptor, somatostatin receptor, bombesin, neuropeptide Y, luteinizing hormone releasing hormone receptor, folic acid receptor, epidermal growth factor receptor, transforming growth factor, fibroblast growth factor receptor, asialoglycoprotein receptor, galectin-3 receptor, E-selectin receptor, hyaluronic acid receptor, prostate-specific membrane antigen (PSMA), cholecystokinin A receptor, cholecystokinin B receptor, discoidin domain receptor, mucin receptor, opioid receptor, plasminogen receptor, bradykinin receptor, insulin receptor, insulin-like growth factor receptor, angiotensin AT1 receptor, angiotensin AT2 receptor, granulocyte macrophage colony stimulating factor receptor (GM-CSF receptor), galactosamine receptor, sigma-2 receptor, delta-like 3 (DLL-3), aminopeptidase P, melanotransferrin, leptin, tetanus toxin Tet1, tetanus toxin G23, RVG (Rabies Virus Glycoprotein) peptide, HER2 (human epidermal growth factor receptor 2), GPNMB (glycoprotein non-metastatic b), Ley, CA6, CanAng, SLC44A4 (Solute carrier family 44 member 4), CEACAM5 (Carcinoembryonic antigen-related cell adhesion molecule 5), Nectin-4, Carbonic Anhydrase 9, TNNB2, 5T4, CD30, CD37, CD74, CD70, PMEL17, EphA2 (EphrinA2 receptor), Trop-2, SC-16, Tissue factor, ENPP-3 (AGS-16), SLITRK6 (SLIT and NTRK like family member 6), CD27, Lewis Y antigen, LIV1, GPR161 (G Protein-Coupled Receptor 161), PBR (peripheral-type benzodiazeoine receptor), MERTK (Mer receptor tyrosine kinase) receptor, CD71, LLT1 (Lectin-like transcript 1 or CLED2D), interleukin-22 receptor, sigma 1 receptor, peroxisome proliferator-activated receptor, DLL3, C4.4a, cKIT, EphrinA, CTLA4 (Cytotoxic T-Lymphocyte Associated Protein 4), FGFR2b (fibroblast growth factor receptor 2b), N-acetylcholine receptor, gonadotropin releasing hormone receptor, gastrin-releasing peptide receptor, bone morphogenetic protein receptor-type 1B (BMPR1B), E16 (LAT1, SLC7A5), STEAP1 (six transmembrane epithelial antigen of prostate), 0772P (CA125, MUC16), MPF (MSLN, mesothelin), Napi3b (SLC34A2), Sema5b (semaphorin 5b), ETBR (Endothelin type B receptor), MSG783 (RNF124), STEAP2 (six transmembrane epithelial antigen of prostate 2), TrpM4 (transient receptor potential cation 5 channel, subfamily M, member 4), CRIPTO (teratocarcinoma-derived growth factor), CD21, CD79b, FcRH2 (IFGP4), HER2 (ErbB2), NCA (CEACM6), MDP (DPEP1), IL20R-alpha (IN20Ra), Brevican (BCAN), EphB2R, ASLG659 (B7h), CD276, PSCA (prostate stem cell antigen precursor), GEDA, BAFF-R (BR3), CD22 (BL-CAM), CD79a, CXCR5, HLA-DOB, P2X5, CD72, LY64, FcRH1, IRTA2, TENB2, SSTR2, SSTR5, SSTR1, SSTR3, SSTR4, ITGAV (Integrin, alpha 5), ITGB6 (Integrin, beta 6), MET, MUC1, EGFRvIII, CD33, CD19, IL2RA (interleukin 2 receptor, alpha), AXL, BCMA, CTA (cancer tetis antigens), CD174, CLEC14A, GPR78, CD25, CD32, LGR5 (GPR49), CD133 (Prominin), ASG5, ENPP3 (ectonucleotide Pyrophosphatase/Phosphodiesterase 3), PRR4 (proline-rich protein 4), GCC (guanylate cyclase 2C), Liv-1 (SLC39A6), CD56, CanAg, TIM-1, RG-1, B7-H4, PTK7, CD138, Claudins, Her3 (ErbB3), RON (MST1R), CD20, TNC (Tenascin C), FAP, DKK-1, CD52, CS1 (SLAMF7), Annexin A1, V-CAM, gp100, MART-1, MAGE-1 (melanoma antigen-encoding gene-1), MAGE-3 (melanoma-associated antigen 3), BAGE, GAGE-1, MUM-1 (multiple myeloma oncogene 1), CDK4, TRP-1 (gp75), TAG-72 (tumor-associated glycoprotein-72), ganglioside GD2, GD3, GM2, GM3, VEP8, VEP9, Myl, VIM-D5, D156-22, OX40, RNAK, PD-L1, TNFR1, TNFR2, etc.

### Targets

In some embodiments, the target or targets of the molecular recognition element are specifically associated with one or more particular cell or tissue types. In some embodiments, targets are specifically associated with one or more particular disease states. In some embodiments, targets are specifically associated with one or more particular developmental stages. For example, a cell type specific marker is typically expressed at levels at least 2 fold greater in that cell type than in a reference population of cells. In some embodiments, the cell type specific marker is present at levels at least 3 fold, at least 4 fold, at least 5 fold, at least 6 fold, at least 7 fold, at least 8 fold, at least 9 fold, at least 10 fold, at least 50 fold, at least 100 fold, or at least 1,000 fold greater than its average expression in a reference population. Detection or measurement of a cell type specific marker may make it possible to distinguish the cell type or types of interest from cells of many, most, or all other types. In some embodiments, a target can comprise a protein, a carbohydrate, a lipid, and/or a nucleic acid, as described herein.

In some embodiments, a substance is considered to be “targeted” if it specifically binds to a cell-binding group, such as a nucleic acid cell-binding group. In some embodiments, a cell-binding group, such as a nucleic acid cell-binding group, specifically binds to a cell under stringent conditions.

In certain embodiments, the conjugates and compounds described herein comprise a cell-binding group that specifically binds to one or more targets (e.g., antigens) associated with an organ, tissue, cell, extracellular matrix component, and/or intracellular compartment. In some embodiments, the conjugates and compounds described herein comprise a cell-binding group that specifically binds to targets associated with a particular organ or organ system. In some embodiments, the conjugates and compounds described herein comprise a cell-binding group that specifically binds to one or more intracellular targets (e.g., organelle, intracellular protein). In some embodiments, the conjugates and compounds described herein comprise a cell-binding group which specifically binds to targets associated with diseased organs, tissues, cells, extracellular matrix components, and/or intracellular compartments. In some embodiments, the conjugates and compounds described herein comprise a cell-binding group that specifically binds to targets associated with particular cell types (e.g., endothelial cells, cancer cells, malignant cells, prostate cancer cells, etc.).

In some embodiments, the conjugates and compounds described herein comprise a cell-binding group that binds to a target that is specific for one or more particular tissue types (e.g., liver tissue vs. prostate tissue). In some embodiments, the conjugates and compounds described herein comprise a cell-binding group that binds to a target that is specific for one or more particular cell types (e.g., T cells vs. B cells). In some embodiments, the conjugates and compounds described herein comprise a cell-binding group that binds to a target that is specific for one or more particular disease states (e.g., tumor cells vs. healthy cells). In some embodiments, the conjugates and compounds described herein comprise a cell-binding group that binds to a target that is specific for one or more particular developmental stages (e.g., stem cells vs. differentiated cells).

In some embodiments, a cell-binding group may be a marker that is exclusively or primarily associated with one or a few cell types, with one or a few diseases, and/or with one or a few developmental stages. A cell type specific marker is typically expressed at levels at least 2 fold greater in that cell type than in a reference population of cells which may consist, for example, of a mixture containing cells from a plurality (e.g., 5-10 or more) of different tissues or organs in approximately equal amounts. In some embodiments, the cell type specific marker is present at levels at least 3 fold, at least 4 fold, at least 5 fold, at least 6 fold, at least 7 fold, at least 8 fold, at least 9 fold, at least 10 fold, at least 50 fold, at least 100 fold, or at least 1000 fold greater than its average expression in a reference population. Detection or measurement of a cell type specific marker may make it possible to distinguish the cell type or types of interest from cells of many, most, or all other types.

In some embodiments, a target comprises a protein, a carbohydrate, a lipid, and/or a nucleic acid. In some embodiments, a target comprises a protein and/or characteristic portion thereof, such as a tumor marker, integrin, cell surface receptor, transmembrane protein, intercellular protein, ion channel, membrane transporter protein, enzyme, antibody, chimeric protein, glycoprotein, etc. In some embodiments, a target comprises a carbohydrate and/or characteristic portion thereof, such as a glycoprotein, sugar (e.g., monosaccharide, disaccharide, polysaccharide), glycocalyx (i.e., the carbohydrate-rich peripheral zone on the outside surface of most eukaryotic cells), etc. In some embodiments, a target comprises a lipid and/or characteristic portion thereof, such as an oil, fatty acid, glyceride, hormone, steroid (e.g., cholesterol, bile acid), vitamin (e.g., vitamin E), phospholipid, sphingolipid, lipoprotein, etc. In some embodiments, a target comprises a nucleic acid and/or characteristic portion thereof, such as a DNA nucleic acid; RNA nucleic acid; modified DNA nucleic acid; modified RNA nucleic acid; nucleic acid that includes any combination of DNA, RNA, modified DNA, and modified RNA.

Numerous markers are known in the art. Typical markers include cell surface proteins, e.g., receptors. Exemplary receptors include, but are not limited to, the transferrin receptor; LDL receptor; growth factor receptors such as epidermal growth factor receptor family members (e.g., EGFR, Her2, Her3, Her4) or vascular endothelial growth factor receptors, cytokine receptors, cell adhesion molecules, integrins, selectins, and CD molecules. The marker can be a molecule that is present exclusively or in higher amounts on a malignant cell, e.g., a tumor antigen.

### Antibody-Drug Conjugates (ADCs)

In some embodiments, CB is an antibody, and Q is a drug. Accordingly, the compounds and conjugates disclosed herein may be used to conjugate an antibody to a drug moiety to form antibody-drug conjugates (ADC). Antibody-drug conjugates (ADCs), like other drug conjugates, may increase therapeutic efficacy in treating disease, e.g., cancer, due to the ability of the ADC to selectively deliver one or more drug moiety(s) to target tissues, such as a tumor-associated antigen. Thus, in certain embodiments, the disclosure provides ADCs for therapeutic use, e.g., treatment of cancer.

ADCs of the disclosure comprise an antibody linked to one or more drug moieties. The specificity of the ADC is defined by the specificity of the antibody. In one embodiment, an antibody is linked to one or more cytotoxic drug(s), which is delivered internally to a cancer cell.

Examples of drugs that may be used in the ADC of the disclosure are provided below. The terms “drug”, “agent”, and “drug moiety” are used interchangeably herein. The terms “linked” and “conjugated” are also used interchangeably herein and indicate that the antibody and moiety are covalently linked.

In certain aspects, the present disclosure is directed to ADCs, compositions comprising ADCs, methods of treating, and methods of formulating ADC compositions. ADCs comprise an antibody, or an antibody fragment, conjugated to a cytotoxic compound. In some embodiments, the cytotoxic compound is conjugated to an antibody via a linker. In other embodiments, the cytotoxic compound is linked directly to the antibody. The types of antibodies, linkers, and cytotoxic compounds encompassed by this disclosure are described below.

### Antibodies

The antibody of an ADC may be any antibody that binds, typically but not necessarily specifically, an antigen expressed on the surface of a target cell of interest. The antigen need not, but in some embodiments, is capable of internalizing an ADC bound thereto into the cell. Target cells of interest may include cells where induction of apoptosis is desirable. Target antigens may be any protein, glycoprotein, polysaccharide, lipoprotein, etc. expressed on the target cell of interest, but will typically be proteins that are either uniquely expressed on the target cell and not on normal or healthy cells, or that are over-expressed on the target cell as compared to normal or healthy cells, such that the ADCs selectively target specific cells of interest, such as, for example, tumor cells. As will be appreciated by skilled artisans, the specific antigen, and hence antibody, selected will depend upon the identity of the desired target cell of interest. In specific embodiments, the antibody of the ADC is an antibody suitable for administration to humans.

Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

References to “VH” refer to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab.

The term “antibody” herein is used in the broadest sense and refers to an immunoglobulin molecule that specifically binds to, or is immunologically reactive with, a particular antigen, and includes polyclonal, monoclonal, genetically engineered and otherwise modified forms of antibodies, including but not limited to murine, chimeric antibodies, humanized antibodies, heteroconjugate antibodies (e.g., bispecific antibodies, diabodies, triabodies, and tetrabodies), and antigen binding fragments of antibodies, including e.g., Fab′, F(ab′)₂, Fab, Fv, rIgG, and scFv fragments. The term “scFv” refers to a single chain Fv antibody in which the variable domains of the heavy chain and the light chain from a traditional antibody have been joined to form one chain.

Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “antibody fragment” refers to a portion of a full-length antibody, generallythe target binding or variable region. Examples of antibody fragments include Fab, Fab′, F(ab′)₂ and Fv fragments. An “Fv” fragment is the minimum antibody fragment that contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. “Single-chain Fv” or “scFv” antibody fragments comprise the VH and VL domains of an antibody in a single polypeptide chain. Generally, the Fv polypeptide further comprises a polypeptide linker between the VH and VL domains which enables the scFv to form the desired structure for target binding. “Single domain antibodies” are composed of a single VH or VL domains which exhibit sufficient affinity to the target. In a specific embodiment, the single domain antibody is a camelized antibody (see, e.g., Riechmann, 1999, Journal of Immunological Methods 231:25-38).

The Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)2 pepsin digestion product. Additional chemical couplings of antibody fragments are known to those of ordinary skill in the art.

Both the light chain and the heavy chain variable domains have complementarity determining regions (CDRs), also known as hypervariable regions. The more highly conserved portions of variable domains are called the framework (FR). As is known in the art, the amino acid position/boundary delineating a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The CDRs in each chain are held together in close proximity by the FR regions and, with the CDRs from the other chain, contribute to the formation of the target binding site of antibodies (see Kabat et al., Sequences of Proteins of Immunological Interest (National Institute of Health, Bethesda, Md. 1987). As used herein, numbering of immunoglobulin amino acid residues is done according to the immunoglobulin amino acid residue numbering system of Kabat et al., unless otherwise indicated.

In certain embodiments, the antibodies of the ADCs of the present disclosure are monoclonal antibodies. The term “monoclonal antibody” (mAb) refers to an antibody that is derived from a single copy or clone, including e.g., any eukaryotic, prokaryotic, or phage clone, and not the method by which it is produced. Preferably, a monoclonal antibody of the disclosure exists in a homogeneous or substantially homogeneous population. Monoclonal antibody includes both intact molecules, as well as, antibody fragments (such as, for example, Fab and F(ab′)2 fragments), which are capable of specifically binding to a protein. Fab and F(ab′)2 fragments lack the Fc fragment of intact antibody, clear more rapidly from the circulation of the animal, and may have less non-specific tissue binding than an intact antibody (Wahl et al., 1983, J. Nucl. Med 24:316). Monoclonal antibodies useful with the present disclosure can be prepared using a wide variety of techniques known in the art including the use of hybridoma, recombinant, and phage display technologies, or a combination thereof. The antibodies of the disclosure include chimeric, primatized, humanized, or human antibodies.

While in most instances antibodies are composed of only the genetically-encoded amino acids, in some embodiments non-encoded amino acids may be incorporated at specific. Examples of non-encoded amino acids that may be incorporated into antibodies for use in controlling stoichiometry and attachment location, as well as methods for making such modified antibodies are discussed in Tian et al., 2014, Proc Nat'l Acad Sci USA 111(5):1766-1771 and Axup et al., 2012, Proc Nat'l Acad Sci USA 109(40):16101-16106 the entire contents of which are incorporated herein by reference.

In certain embodiments, the antibody of the ADCs described herein is a chimeric antibody. The term “chimeric” antibody as used herein refers to an antibody having variable sequences derived from a non-human immunoglobulin, such as rat or mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816,397, which are incorporated herein by reference in their entireties.

In certain embodiments, the antibody of the ADCs described herein is a humanized antibody. “Humanized” forms of non-human (e.g., murine) antibodies are chimeric immunoglobulins, immunoglobulin chains or fragments thereof (such as Fv, Fab, Fab′, F(ab′)2 or other target-binding subdomains of antibodies), which contain minimal sequences derived from non-human immunoglobulin. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad Sci. USA 91:969-973; and U.S. Pat. No. 5,565,332, all of which are hereby incorporated by reference in their entireties.

In certain embodiments, the antibody of the ADCs described herein is a human antibody. Completely “human” antibodies can be desirable for therapeutic treatment of human patients. As used herein, “human antibodies” include antibodies having the amino acid sequence of a human immunoglobulin and include antibodies isolated from human immunoglobulin libraries or from animals transgenic for one or more human immunoglobulin and that do not express endogenous immunoglobulins. Human antibodies can be made by a variety of methods known in the art including phage display methods using antibody libraries derived from human immunoglobulin sequences. See U.S. Pat. Nos. 4,444,887 4,716,111, 6,114,598, 6,207,418, 6,235,883, 7,227,002, 8,809,151 and U.S. Published Application No. 2013/189218, the contents of which are incorporated herein by reference in their entireties. Human antibodies can also be produced using transgenic mice which are incapable of expressing functional endogenous immunoglobulins, but which can express human immunoglobulin genes. See, e.g., U.S. Pat. Nos. 5,413,923; 5,625,126; 5,633,425; 5,569,825; 5,661,016; 5,545,806; 5,814,318; 5,885,793; 5,916,771; 5,939,598; 7,723,270; 8,809,051 and U.S. Published Application No. 2013/117871, which are incorporated by reference herein in their entireties. In addition, companies such as Medarex (Princeton, N.J.), Astellas Pharma (Deerfield, Ill.), and Regeneron (Tarrytown, N.Y.) can be engaged to provide human antibodies directed against a selected antigen using technology similar to that described above. Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1988, Biotechnology 12:899-903).

In certain embodiments, the antibody of the ADCs described herein is a primatized antibody. The term “primatized antibody” refers to an antibody comprising monkey variable regions and human constant regions. Methods for producing primatized antibodies are known in the art. See, e.g., U.S. Pat. Nos. 5,658,570; 5,681,722; and 5,693,780, which are incorporated herein by reference in their entireties.

In certain embodiments, the antibody of the ADCs described herein is a bispecific antibody or a dual variable domain antibody (DVD). Bispecific and DVD antibodies are monoclonal, often human or humanized, antibodies that have binding specificities for at least two different antigens. DVDs are described, for example, in U.S. Pat. No. 7,612,181, the disclosure of which is incorporated herein by reference.

In certain embodiments, the antibody of the ADCs described herein is a derivatized antibody. For example, but not by way of limitation, derivatized antibodies are typically modified by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formylation, metabolic synthesis of tunicamycin, etc. Additionally, the derivative can contain one or more non-natural amino acids, e.g., using ambrx technology (see, e.g., Wolfson, 2006, Chem. Biol. 13(10):1011-2).

In certain embodiments, the antibody of the ADCs described herein has a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fc receptor (FcR). FcR binding can be reduced by mutating the immunoglobulin constant region segment of the antibody at particular regions necessary for FcR interactions (see, e.g., Canfield and Morrison, 1991, J. Exp. Med 173:1483-1491; and Lund et al., 1991, J. Immunol. 147:2657-2662).

In certain embodiments, the antibody of the ADCs described herein is modified to acquire or improve at least one constant region-mediated biological effector function relative to an unmodified antibody, e.g., to enhance FcγR interactions (see, e.g., US 2006/0134709). For example, an antibody with a constant region that binds FcγRIIA, FcγRIIB and/or FcγRIIIA with greater affinity than the corresponding wild type constant region can be produced according to the methods described herein.

In certain specific embodiments, the antibody of the ADCs described herein is an antibody that binds tumor cells, such as an antibody against a cell surface receptor or a tumor-associated antigen (TAA). In attempts to discover effective cellular targets for cancer diagnosis and therapy, researchers have sought to identify transmembrane or otherwise tumor-associated polypeptides that are specifically expressed on the surface of one or more particular type(s) of cancer cell as compared to on one or more normal non-cancerous cell(s). Often, such tumor-associated polypeptides are more abundantly expressed on the surface of the cancer cells as compared to the surface of the no-cancerous cells. Such cell surface receptor and tumor-associated antigens are known in the art, and can prepared for use in generating antibodies using methods and information which are well known in the art.

### Exemplary Cell Surface Receptors and TAAs

Examples of cell surface receptor and TAAs to which the antibody of the ADCs described herein may be targeted include, but are not limited to, the various receptors and TAAs listed below in Table 1. For convenience, information relating to these antigens, all of which are known in the art, is listed below and includes names, alternative names, Genbank accession numbers and primary reference(s), following nucleic acid and protein sequence identification conventions of the National Center for Biotechnology Information (NCBI). Nucleic acid and protein sequences corresponding to the listed cell surface receptors and TAAs are available in public databases such as GenBank.

TABLE I

4-1BB
5AC
5T4
Alpha-fetoprtein
angiopoietin 2
ASLG659
TCL1
BMPR1B
Brevican (BCAN, BEHAB)
C2-42 antigen
C5
CA-125
CA-125 (imitation)
CA-IX (Carbonic anhydrase 9)
CCR4
CD140a
CD152
CD19
CD20
CD200
CD21 (C3DR) 1)
CD22 (B-cell receptor CD22-B isoform)
CD221
CD23 (gE receptor)
CD28
CD30 (TNFRSF8)
CD33
CD37
CD38 (cyclic ADP ribose hydrolase)
CD4
CD40
CD44 v6
CD51
CD52
CD56
CD70
CD72 (Lyb-2, B-cell differentiation antigen CD72)
CD74
CD79a (CD79A, CD79α, immunoglobulin-associated alpha)
Genbank accession No. NP_001774.10)
CD79b (CD79B, CD79B, B29)
CD80
CEA
CEA-related antigen
ch4D5
CLDN18.2
CRIPTO (CR, CR1, CRGF, TDGF1 teratocarcinoma-derived
growth factor)
CTLA-4
CXCR5
DLL4
DR5
E16 (LAT1, SLC7A5) EGFL7
EGFR
EpCAM
EphB2R (DRT, ERK, Hek5, EPHT3, Tyro5)
Episialin
ERBB3
ETBR (Endothelin type B receptor)
FCRH1 (Fc receptor-like protein 1)
FcRH2 (IFGP4, IRTA4, SPAP1, SPAP1B, SPAP1C, SH2 domain
containing phosphatase anchor protein
Fibronectin extra domain-B
Folate receptor 1
Frizzled receptor
GD2
GD3 ganglioside
GEDA
GPNMB
HER1
HER2 (ErbB2)
HER2/neu
HER3
HGF
HLA-DOB
HLA-DR
Human scatter factor receptor kinase
IGF-1 receptor
IgG4
IL-13
IL20Rα (IL20Ra, ZCYTOR7)
IL-6
ILGF2
ILFR1R
integrin α
integrin α5β1
integrin αvβ3
IRTA2 (Immunoglobulin superfamily receptor translocation
associated 2, Gene Chromosome 1q21)
Lewis-Y antigen
LY64 (RP105)
MCP-1
MDP (DPEP1)
MPF (MSLN, SMR, mesothelin, megkaryocyte potentiating
factor)
MS4A1
MSG783 (RNF124, hypothetical protein FLJ20315)
MUC1
Mucin CanAg
Napi3 (NAPI-3B, NPTIIb, SLC34A2, type II sodium-dependent
phosphate transporter 3b)
NCA (CEACAM6)
P2X5 (Purinergic receptor P2X ligand-gated ion channel 5)
PD-1
PDCD1
PDGF-R α
Prostate specific membrane antigen
PSCA (Prostate stem cell antigen precursor)
PSCA hlg
RANKL
RON
SDC1
Sema 5b
SLAMF7 (CS-1)
STEAP1
STEAP2 (HGNC_8639, PCANAP1, STAMP1, STEAP2, STMP,
prostate cancer associated gene 1)
TAG-72
TEM1
Tenascin C
TENB2, (TMEFF2, tomoregulin, TPEF, HPP1, TR)
TGF-β
TRAIL-E2
TRAIL-R1
TRAIL-R2
TrpM4 (BR22450, FLJ20041, TRPM4, TRPM4B, transient
receptor potential cation channel subfmlily M, member 4)
TROP2
TA CTAA16.88
TWEAK-R
TYRP1 (glycoprotein 75)
VEGF
VEGF-A
EGFR-1
VEGFR-2
Vimentin

### Exemplary Antibodies

Exemplary antibodies to be used with ADCs of the disclosure include but are not limited to 3F8 (GD2), Abagovomab (CA-125 (imitation)), Adecatumumab (EpCAM), Afutuzumab (CD20), Alacizumab pegol (VEGFR2), ALD518 (IL-6), Alemtuzumnab (CD52), Altumomab pentetate (CEA), Amatuximab (Mesothelin), Anatumomnab mafenatox (TAG-72), Apolizumab (HLA-DR), Arcitumomab (CEA), Bavituximab (Phosphatidylserine), Bectumomab (CD22), Belimumab (BAFF), Besilesomab (CEA-related antigen), Bevacizumab (VEGF-A), Bivatuzumab mertansine (CD44 v6), Blinatumomab (CD19), Brentuximab vedotin ((CD30 (TNFRSF8)), Cantuzumab mertansine (Mucin CanAg), Cantuzumab ravtansine (MUC1), Capromab pendetide (Prostatic carcinoma cells), Carlumab (MCP-1), Catumaxomab (EpCAM, CD3), CC49 (Tag-72), cBR96-DOX ADC (Lewis-Y antigen), Cetuximab (EGFR), Citatuzumab bogatox (EpCAM), Cixutumumab (IGF-1 receptor), Clivatuzumab tetraxetan(MUC1), Conatumumab (TRAIL-E2), Dacetuzumab (CD40), Dalotuzumab (Insulin-like growth factor 1 receptor), Deratumumab ((CD38 (cyclic ADP ribose hydrolase)), Demcizumab (DLL4), Denosumab (RANKL), Detumomab (B-lymphoma cell), Drozitumab (DR5), Dusigitumab (ILGF2), Ecromeximab (D3 ganglioside), Eculizumab (C5), Edrecolomab (EpCAM), Elotuzumab (SLAMF7), Elsilimomab (IL-6), Enavatuzumab (TWEAK receptor), Enoticumab (DLL4), Ensituximab (5AC), Epitumomab cituxetan (Episialin), Epratuzumab (CD22), Ertumaxomab ((HER2/neu, CD3)), Etancizumab (Integrin αvβ3), Farletuzumab (Folate receptor 1), FBTA05 (CD20), Ficlatuzumab (HGF), Figitumumab (IGF-1 receptor), Flanvotumab ((TYRP1 (glycoprotein 75)), Fresolimumab (TGF-1), Galiximab (CD80), Ganitumab (IGF-I), Gemtuzumab ozogamicin (CD33), Girentuximab ((Carbonic anhydrase 9 (CA-IX)), Glembatumumab vedotin (GPNMB), Ibritumomab tiuxetan (CD20) Icrucumab (VEGFR-1), Igovomab (CA-125), IMAB362 (CLDN18.2), Imgatuzumab (EGFR), Indatuximab ravtansine (SDC1), Intetumumab (CD51), Inotuzumab ozogamicin (CD22), Ipilimumab (CD152), Iratumumab ((CD30 (TNFRSF8)), Labetuzumab (CEA), Lambrolizumab (PDCD1), Lexatumumab (TRAIL-R2), Lintuzumab (CD33), Lorvotuzumab mertansine (CD56), Lucatumumab (CD40), Lumiliximab ((CD23 (IgE receptor)), Mapatumumab (TRAIL-R1), Margetuximab (ch4DS), Matuzumab (EGFR), Milatuzumab (CD74), Mitumomab (GD3 ganglioside), Mogamulizumab (CCR4), Moxetumomab pasudotox (CD22), Nacolomab tafenatox (C2-42 antigen), Naptumomab estafenatox (5T4), Narnatumab (RON), Natalizumab (integrin α4), Necitumumab (EGFR), Nesvacumab (angiopoietin 2), Nimotuzumab (EGFR), Nivolumab (IgG4), Ocaratuzumab (CD20), Ofatumumab (CD20), Olaratumab (PDGF-R a), Onartuzumab (Human scatter factor receptor kinase), Ontuxizumab (TEM1), Oportuzumab monato (EpCAM), Oregovomab (CA-125), Otlertuzumab (CD37), Panitumumab (EGFR) Pankomab (Tumor specific glycosylation of MUC1), Parsatuzumab (EGFL7), Patritumab (HER3), Pemtumomab (MUC1), Pertuzumab (HER2/neu), Pidilizumab (PD-1), Pinatuzumab vedotin (CD22), Pritumumab (Vimentin), Racotumomab (N-glycolylneuraminic acid), Radretumab (Fibronectin extra domain-B), Ramucirumab (VEGFR2), Rilotumumab (HGF), Rituximab (CD20), Robatumumab (IGF-1 receptor), Samalizumab (CD200), Satumomab pendetide (TAG-72), Seribantumab (ERBB3), Sibrotuzumab (FAP), SGN-CD19A (CD19), SGN-CD33A (CD33), Siltuximab (IL-6), Solitomab (EpCAM), Sonepcizumab (Sphingosine-1-phosphate), Tabalumb (BAFF), Tacatuzumab tetraxetan (Alpha-fetoprotein), Taplitumomab paptox (CD19), Tenatumomab (Tenascin C), Teprotumumab (CD221), TGN1412 (CD28), Ticilimumab (CTLA-4), Tigatuzumab (TRAIL-R2), TNX-650 (IL-13), Tovetumab (CD40a), Trastuzumab (HER2/neu), TRBS07 (GD2), Tremelimumab (CTLA-4), Tucotuzumab celmoleukin (EpCAM), Ublituximab (MS4A), Urelumab (4-1BB), Vandetanib (VEGF), Vantictumab (Frizzled receptor), Volociximab (integrin α5β1), Vorsetuzumab mafodotin (CD70), Votumumab (Tumor antigen CTAA16.88), Zalutumumab (EGFR), Zanolimumab (CD4), and Zatuximab (HER1).

### Methods of Making Antibodies

The antibody of an ADC can be prepared by recombinant expression of immunoglobulin light and heavy chain genes in a host cell. For example, to express an antibody recombinantly, a host cell is transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Molecular Cloning; A Laboratory Manual, Second Edition (Sambrook, Fritsch and Maniatis (eds), Cold Spring Harbor, N. Y., 1989), Current Protocols in Molecular Biology (Ausubel, F. M. et al., eds., Greene Publishing Associates, 1989) and in U.S. Pat. No. 4,816,397.

In one embodiment, the Fc variant antibodies are similar to their wild-type equivalents but for changes in their Fc domains. To generate nucleic acids encoding such Fc variant antibodies, a DNA fragment encoding the Fc domain or a portion of the Fc domain of the wild-type antibody (referred to as the “wild-type Fc domain”) can be synthesized and used as a template for mutagenesis to generate an antibody as described herein using routine mutagenesis techniques; alternatively, a DNA fragment encoding the antibody can be directly synthesized.

Once DNA fragments encoding wild-type Fc domains are obtained, these DNA fragments can be further manipulated by standard recombinant DNA techniques, for example, to convert the constant region genes to full-length antibody chain genes. In these manipulations, a CH-encoding DNA fragment is operatively linked to another DNA fragment encoding another protein, such as an antibody variable region or a flexible linker. The term “operatively linked,” as used in this context, is intended to mean that the two DNA fragments are joined such that the amino acid sequences encoded by the two DNA fragments remain in-frame.

To express the Fc variant antibodies, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. A variant antibody light chain gne and the antibody heavy chain gene can be inserted into separate vectors or, more typically, both genes are inserted into the same expression vector.

The antibody genes are inserted into the expression vector by standard methods (e.g., ligation of complementary restriction sites on the antibody gene fragment and vector, or blunt end ligation if no restriction sites are present). Prior to insertion of the variant Fc domain sequences, the expression vector can already carry antibody variable region sequences. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel, Gene Expression Technology: Methods in Enzymology 185 (Academic Press, San Diego, Calif., 1990). It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al., and U.S. Pat. No. 4,968,615 by Schaffner et al.

In addition to the antibody chain genes and regulatory sequences, the recombinant expression vectors can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all to Axel et al.). For example, typically the selectable marker gene confers resistance to drugs, such as G418, puromycin, blasticidin, hygromycin or methotrexate, on a host cell into which the vector has been introduced. Suitable selectable marker genes include the dihydrofolate reductase (DHFR) gene (for use in DHFR-host cells with methotrexate selection/amplification) and the neo gene (for G418 selection). For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, lipofection, calcium-phosphate precipitation, DEAE-dextran transfection, and the like.

It is possible to express the antibodies in either prokaryotic or eukaryotic host cells. In certain embodiments, expression of antibodies is performed in eukaryotic cells, e.g., mammalian host cells, for optimal secretion of a properly folded and immunologically active antibody. Exemplary mammalian host cells for expressing the recombinant antibodies include Chinese Hamster Ovary (CHO cells) (including DHFR-CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601-621), NSO myeloma cells, COS cells, 293 cells and SP2/0 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods. Host cells can also be used to produce portions of intact antibodies, such as Fab fragments or scFv molecules.

In some embodiments, the antibody of an ADC can be a bifunctional antibody. Such antibodies, in which one heavy and one light chain are specific for one antigen and the other heavy and light chain are specific for a second antigen, can be produced by crosslinking an antibody to a second antibody by standard chemical crosslinking methods. Bifunctional antibodies can also be made by expressing a nucleic acid engineered to encode a bifunctional antibody.

In certain embodiments, dual specific antibodies, i.e. antibodies that bind one antigen and a second, unrelated antigen using the same binding site, can be produced by mutating amino acid residues in the light chain and/or heavy chain CDRs. Exemplary second antigens include a proinflammatory cytokine (such as, for example, lymphotoxin, interferon-7, or interleukin-1). Dual specific antibodies can be produced, e.g., by mutating amino acid residues in the periphery of the antigen binding site (see, e.g., Bostrom et al., 2009, Science 323:1610-1614). Dual functional antibodies can be made by expressing a nucleic acid engineered to encode a dual specific antibody.

Antibodies can also be produced by chemical synthesis (e.g., by the methods described in Solid Phase Peptide Synthesis, 2nd ed., 1984 The Pierce Chemical Co., Rockford, Ill.). Antibodies can also be generated using a cell-free platform (see, e.g., Chu et al., Biochemia No. 2, 2001 (Roche Molecular Biologicals)).

Methods for recombinant expression of Fc fusion proteins are described in Flanagan et al., Methods in Molecular Biology, vol. 378: Monoclonal Antibodies: Methods and Protocols.

Once an antibody has been produced by recombinant expression, it can be purified by any method known in the art for purification of an immunoglobulin molecule, for example, by chromatography (e.g., ion exchange, affinity, particularly by affinity for antigen after Protein A or Protein G selection, and sizing column chromatography), centrifugation, differential solubility, or by any other standard technique for the purification of proteins.

Once isolated, an antibody can, if desired, be further purified, e.g., by high performance liquid chromatography (see, e.g., Fisher, Laboratory Techniques In Biochemistry And Molecular Biology (Work and Burdon, eds., Elsevier, 1980)), or by gel filtration chromatography on a Superdex™ 75 column (Pharmacia Biotech AB, Uppsala, Sweden).

### General Method for Preparing Antibodies

Various procedures known within the art may be used for the production of polyclonal or monoclonal antibodies directed against a given target, such as, for example, recognize HER2, HER3, or TROP2, a tumor associated antigen or other target, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

In some embodiments, the antibodies which may be used in embodiments disclosed herein are monoclonal antibodies. Monoclonal antibodies are generated, for example, by using the procedures set forth in the Examples provided herein. Antibodies are also generated, e.g., by immunizing BALB/c mice with combinations of cell transfectants expressing high levels of a given target on their surface. Hybridomas resulting from myeloma/B cell fusions are then screened for reactivity to the selected target.

Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody, or can be substituted for the variable domains of one antigen-combining site of an antibody to create a chimeric bivalent antibody.

Monoclonal antibodies which may be used in embodiments disclosed here include humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization is performed, e.g., by following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539). In some instances, Fv framework residues of the human immunoglobulin are replaced by corresponding non-human residues. Humanized antibodies also comprise, e.g., residues which are found neither in the recipient antibody nor in the imported CDR or framework sequences. In general, the humanized antibody includes substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also includes at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Fully human antibodies are antibody molecules in which the entire sequence of both the light chain and the heavy chain, including the CDRs arise from human genes. Such antibodies are termed “human antibodies” or “fully human antibodies” herein. Monoclonal antibodies can be prepared by using trioma technique; the human B-cell hybridoma technique (see Kozbor, et al., 1983 Immunol Today 4: 72); and the EBV hybridoma technique to produce monoclonal antibodies (see Cole, et al., 1985 In: MONOCLONAL ANTIBODIES AND CANCER THERAPY, Alan R. Liss, Inc., pp. 77-96). Monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. An example of such a nonhuman animal is a mouse termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds specifically to the relevant epitope with high affinity are disclosed in U.S. publication U.S. 2003/009212.

The antibody can be expressed by a vector containing a DNA segment encoding the single chain antibody described above.

These can include vectors, liposomes, naked DNA, adjuvant-assisted DNA. gene gun, catheters, etc. Vectors include chemical conjugates such as described in WO 93/64701, which has a cell-binding group (e.g., a ligand to a cellular surface receptor), and a nucleic acid binding moiety (e.g., polylysine), viral vector (e.g., a DNA or RNA viral vector), fusion proteins such as described in U.S. Pat. No. 7,186,697 which is a fusion protein containing a target group (e.g., an antibody specific for a target cell) and a nucleic acid binding moiety (e.g., a protamine), plasmids, phage, etc. The vectors can be chromosomal, non-chromosomal or synthetic.

Preferred vectors include viral vectors, fusion proteins and chemical conjugates. Retroviral vectors include moloney murine leukemia viruses. DNA viral vectors are preferred. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cells cytoplasm. Avipox virus vectors result in only a short-term expression of the nucleic acid. Adenovirus vectors, adeno-associated virus vectors and herpes simplex virus (HSV) vectors are preferred for introducing the nucleic acid into neural cells. The adenovirus vector results in a shorter-term expression (about 2 months) than adeno-associated virus (about 4 months), which in turn is shorter than HSV vectors. The particular vector chosen will depend upon the target cell and the condition being treated. The introduction can be by standard techniques, e.g., infection, transfection, transduction or transformation. Examples of modes of gene transfer include e.g., naked DNA, CaPO₄ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g., adenovirus, HSV) to a desired location. Additionally, the particles can be delivered by intracerebroventricular (icy) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other suitable routes of administration.

Bispecific antibodies are antibodies that have binding specificities for at least two different antigens. In the present case, one of the binding specificities is for a target such as recognize HER2, HER3, or TROP2 or any fragment thereof. The second binding target is any other antigen, and advantageously is a cell-surface protein or receptor or receptor subunit.

Many methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Bispecific and/or monovalent antibodies which may be used in embodiments disclosed herein can be made using any of a variety of art-recognized techniques, including those disclosed in application WO 2012/023053, filed Aug. 16, 2011, the contents of which are hereby incorporated by reference in their entirety. The methods described in WO 2012/023053 generate bispecific antibodies that are identical in structure to a human immunoglobulin. This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions. However it is also possible to obtain bispecific antibodies which may be used in embodiments disclosed herein by fusing a Kappa light chain variable domain to a constant Lambda domain for a first specificity and fusing a Lambda light chain variable domain to a constant Kappa domain for the second specificity. The bispecific antibodies described in WO 2012/023053 are referred to as IgGκλ antibodies or “κλ bodies,” a new fully human bispecific IgG format. This κλ-body format allows the affinity purification of a bispecific antibody that is undistinguishable from a standard IgG molecule with characteristics that are undistinguishable from a standard monoclonal antibody and, therefore, favorable as compared to previous formats.

An essential step of the method is the identification of two antibody Fv regions (each composed by a variable light chain and variable heavy chain domain) having different antigen specificities that share the same heavy chain variable domain. Numerous methods have been described for the generation of monoclonal antibodies and fragments thereof. (See, e.g., Antibodies: A Laboratory Manual, Harlow E, and Lane D, 1988, Cold Spring Harbor Laboratory Press, Cold Spring Harbor, NY, incorporated herein by reference). Fully human antibodies are antibody molecules in which the sequence of both the light chain and the heavy chain, including the CDRs 1 and 2, arise from human genes. The CDR3 region can be of human origin or designed by synthetic means. Such antibodies are termed “human antibodies”, or “fully human antibodies” herein. Human monoclonal antibodies can be prepared by using the trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

Monoclonal antibodies are generated, e.g., by immunizing an animal with a target antigen or an immunogenic fragment, derivative or variant thereof. Alternatively, the animal is immunized with cells transfected with a vector containing a nucleic acid molecule encoding the target antigen, such that the target antigen is expressed and associated with the surface of the transfected cells. A variety of suitable techniques for producing xenogenic non-human animals are well-known in the art. For example, see U.S. Pat. Nos. 6,075,181 and 6,150,584, which is hereby incorporated by reference in its entirety.

Alternatively, the antibodies are obtained by screening a library that contains antibody or antigen binding domain sequences for binding to the target antigen. This library is prepared, e.g., in bacteriophage as protein or peptide fusions to a bacteriophage coat protein that is expressed on the surface of assembled phage particles and the encoding DNA sequences contained within the phage particles (i.e., “phage displayed library”).

Hybridomas resulting from myeloma/B cell fusions are then screened for reactivity to the target antigen. Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Although not strictly impossible, the serendipitous identification of different antibodies having the same heavy chain variable domain but directed against different antigens is highly unlikely. Indeed, in most cases the heavy chain contributes largely to the antigen binding surface and is also the most variable in sequence. In particular the CDR3 on the heavy chain is the most diverse CDR in sequence, length and structure. Thus, two antibodies specific for different antigens will almost invariably carry different heavy chain variable domains.

The methods disclosed in application U.S. Pat. No. 9,926,382 overcomes this limitation and greatly facilitates the isolation of antibodies having the same heavy chain variable domain by the use of antibody libraries in which the heavy chain variable domain is the same for all the library members and thus the diversity is confined to the light chain variable domain. Such libraries are described, for example, in U.S. Pat. No. 8,921,281 and Application WO 2011/084255, each of which is hereby incorporated by reference in its entirety. However, as the light chain variable domain is expressed in conjunction with the heavy variable domain, both domains can contribute to antigen binding. To further facilitate the process, antibody libraries containing the same heavy chain variable domain and either a diversity of Lambda variable light chains or Kappa variable light chains can be used in parallel for in vitro selection of antibodies against different antigens. This approach enables the identification of two antibodies having a common heavy chain but one carrying a Lambda light chain variable domain and the other a Kappa light chain variable domain that can be used as building blocks for the generation of a bispecific antibody in the full immunoglobulin format. The bispecific antibodies which may be used in embodiments disclosed herein can be of different Isotypes and their Fc portion can be modified in order to alter the bind properties to different Fc receptors and in this way modify the effectors functions of the antibody as well as it pharmacokinetic properties. Numerous methods for the modification of the Fc portion have been described and are applicable to antibodies which may be used in embodiments disclosed herein. (see for example Strohl, WR Curr Opin Biotechnol 2009 (6):685-91; U.S. Pat. No. 6,528,624; PCT/US2009/0191199 filed Jan. 9, 2009).

The common heavy chain and two different light chains are co-expressed into a single cell to allow for the assembly of a bispecific antibody which may be used in embodiments disclosed herein. If all the polypeptides get expressed at the same level and get assembled equally well to form an immunoglobulin molecule then the ratio of monospecific (same light chains) and bispecific (two different light chains) should be 50%. However, it is likely that different light chains are expressed at different levels and/or do not assemble with the same efficiency. Therefore, a means to modulate the relative expression of the different polypeptides is used to compensate for their intrinsic expression characteristics or different propensities to assemble with the common heavy chain. This modulation can be achieved via promoter strength, the use of internal ribosome entry sites (IRES) featuring different efficiencies or other types of regulatory elements that can act at transcriptional or translational levels as well as acting on mRNA stability. Different promoters of different strength could include CMV (Immediate-early Cytomegalovirus virus promoter); EF1-1α (Human elongation factor 1α-subunit promoter); Ubc (Human ubiquitin C promoter); SV40 (Simian virus 40 promoter). Different IRES have also been described from mammalian and viral origin. (See e.g., Hellen CU and Sarnow P. Genes Dev 2001 15: 1593-612). These IRES can greatly differ in their length and ribosome recruiting efficiency. Furthermore, it is possible to further tune the activity by introducing multiple copies of an IRES (Stephen et al. 2000 Proc Natl Acad Sci USA 97: 1536-1541). The modulation of the expression can also be achieved by multiple sequential transfections of cells to increase the copy number of individual genes expressing one or the other light chain and thus modify their relative expressions. The Examples provided herein demonstrate that controlling the relative expression of the different chains is critical for maximizing the assembly and overall yield of the bispecific antibody.

The co-expression of the heavy chain and two light chains generates a mixture of three different antibodies into the cell culture supernatant: two monospecific bivalent antibodies and one bispecific bivalent antibody. The latter has to be purified from the mixture to obtain the molecule of interest. The method described herein greatly facilitates this purification procedure by the use of affinity chromatography media that specifically interact with the Kappa or Lambda light chain constant domains such as the CaptureSelect Fab Kappa and CaptureSelect Fab Lambda affinity matrices (BAC BV, Holland). This multi-step affinity chromatography purification approach is efficient and generally applicable to antibodies which may be used in embodiments disclosed herein. This is in sharp contrast to specific purification methods that have to be developed and optimized for each bispecific antibodies derived from quadromas or other cell lines expressing antibody mixtures. Indeed, if the biochemical characteristics of the different antibodies in the mixtures are similar, their separation using standard chromatography technique such as ion exchange chromatography can be challenging or not possible at all.

Other suitable purification methods include those disclosed in US2013/0317200, the contents of which are hereby incorporated by reference in their entirety.

In other embodiments of producing bispecific antibodies, antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface includes at least a part of the CH₃ region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen which may be used in embodiments disclosed herein. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate antibodies are also within the scope of the present disclosure. Heteroconjugate antibodies are composed of two covalently joined antibodies. Such antibodies have, for example, been proposed to target immune system cells to unwanted cells (see U.S. Pat. No. 4,676,980), and for treatment of HIV infection (see WO 91/00360; WO 92/200373; EP 03089). It is contemplated that the antibodies can be prepared in vitro using synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It can be desirable to modify the antibodies which may be used in embodiments disclosed herein with respect to effector function, so as to enhance, e.g., the effectiveness of the antibody in treating cancer and/or other diseases and disorders associated with aberrant recognize HER2, HER3, or TROP2expression and/or activity. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)).

### Conjugated Antibodies

The disclosure also pertains to conjugated antibodies, also referred to herein as immunoconjugates, comprising an antibody or antigen-binding fragment thereof conjugated to a cytotoxic agent such as a toxin (e.g., an enzymatically active toxin of bacterial, fungal, plant, or animal origin, or fragments thereof), or a radioactive isotope (i.e., a radioconjugate).

In some embodiments, the toxin is a microtubule inhibitor or a derivative thereof. In some embodiments, the toxin is a dolastatin or a derivative thereof. In some embodiments, the toxin is auristatin E, auristatin F, AFP, MMAF, MMAE, MMAD, DMAF, or DMAE. In some embodiments, the toxin is a maytansinoid or maytansinoid derivative. In some embodiments, the toxin is DM1 or DM4. In some embodiments, the toxin is a nucleic acid damaging toxin. In some embodiments, the toxin is a duocarmycin or derivative thereof. In some embodiments, the toxin is a calicheamicin or a derivative thereof. In some embodiments, the agent is a pyrrolobenzodiazepine or a derivative thereof. In some embodiments, the agent is an exatecane or a derivative thereof. In some embodiments, the agent is an amanitin or a derivative thereof.

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent can be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis-(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies which may be used in embodiments disclosed herein. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present disclosure, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

Suitable linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl) carbodiimide hydrochloride; (ii) SMPT (4-succinimidyloxycarbonyl-alpha-methyl-alpha-(2-pyridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody can be prepared by any suitable methods, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present disclosure can be conjugated to the liposomes as described in Martin et al., J. Biol. Chem., 257: 286-288 (1982) via a disulfide-interchange reaction.

### Use of Antibodies

It will be appreciated that administration of therapeutic entities in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203 (1-2):1-60 (2000), Charman WN “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the disclosure, which include a conjugate of the disclosure, are used to treat or alleviate a symptom associated with a cancer, such as, by way of non-limiting example, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma The present disclosure also provides methods of treating or alleviating a symptom associated with a cancer. A therapeutic regimen can include identifying a subject, e.g., a human patient suffering from (or at risk of developing) a cancer, e.g., using standard methods.

Therapeutic formulations of the disclosure, which include a conjugate of the disclosure that recognize HER2, HER3, or TROP2 and, optionally, a second target can be used to treat or alleviate a symptom associated with an autoimmune disease and/or inflammatory disease, such as, for example, B-cell mediated autoimmune diseases and/or inflammatory diseases, including by way of non-limiting example, systemic lupus erythematosus (SLE), rheumatoid arthritis (RA), idiopathic thrombocytopenic purpura (ITP), Waldenstrom's hypergammaglobulinaemia, Sjogren's syndrome, multiple sclerosis (MS), and/or lupus nephritis.

Efficaciousness of treatment can be determined in association with any suitable method for diagnosing or treating the particular immune-related disorder. Alleviation of one or more symptoms of the immune-related disorder indicates that the conjugate confers a clinical benefit.

Conjugates directed against a target may be used in methods relating to the localization and/or quantitation of these targets, e.g., for use in measuring levels of these targets within appropriate physiological samples, for use in diagnostic methods, for use in imaging the protein, and the like). For example, conjugates specific for any of these targets, or derivative, fragment, analog or homolog thereof, that contain the antibody derived antigen-binding domain, can be utilized as pharmacologically active compounds (referred to hereinafter as “Therapeutics”).

A conjugate of the disclosure can be used to isolate a particular target using standard techniques, such as immunoaffinity, chromatography or immunoprecipitation. Conjugates of the disclosure can be used diagnostically to monitor protein levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Conjugates of the disclosure may be used as therapeutic agents. Such agents will generally be employed to treat or prevent a disease or pathology associated with aberrant expression or activation of a given target in a subject. A conjugate preparation, preferably one having high specificity and high affinity for its target antigen, is administered to the subject and will generally have an effect due to its binding with the target. Administration of the conjugate may abrogate or inhibit or interfere with the signaling function of the target. Administration of the conjugate may abrogate or inhibit or interfere with the binding of the target with an endogenous ligand to which it naturally binds.

A therapeutically effective amount of a conjugate of the disclosure relates generally to the amount needed to achieve a therapeutic objective. As noted above, this may be a binding interaction between the antibody and its target antigen that, in certain cases, interferes with the functioning of the target and/or the effect of an active agent conjugated to the antibody. The amount required to be administered will furthermore depend on the binding affinity of the antibody for its specific antigen and/or the potency of the active agent, and will also depend on the rate at which an administered antibody is depleted from the free volume other subject to which it is administered. Common ranges for therapeutically effective dosing of a conjugate of the disclosure may be, by way of nonlimiting example, from about 0.1 mg/kg body weight to about 50 mg/kg body weight. Common dosing frequencies may range, for example, from twice daily to once a week.

Conjugates of the disclosure can be administered for the treatment of a variety of diseases and disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration are preferably sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

A conjugate according to the disclosure can be used as an agent for detecting the presence of a given target (or a protein fragment thereof) in a sample. In some embodiments, the conjugate contains a detectable label. Antibodies can be polyclonal, or more preferably, monoclonal. An intact antibody, or a fragment thereof (e.g., F_{ab}, scFv, or F_{(ab)2}) can be used. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the disclosure can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte conjugate. For example, the antibody can be labeled with a radioactive marker whose presence and location in a subject can be detected by standard imaging techniques.

### Pharmaceutical Compositions

In certain aspects, provided herein are pharmaceutical compositions comprising the compounds or drug conjugates of the present disclosure.

The antibody-drug conjugate may be used to transfer the active agent to a target cell of a subject to treat the subject using any suitable method of preparing a composition. In some aspects, the disclosure relates to a composition (e.g., a pharmaceutical composition) comprising an antibody-drug conjugate as described herein.

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as lyophile for reconstitution, powder, solution, injection or the like.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self emulsifying drug delivery system or a self microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A pharmaceutical composition (preparation) can be administered to a subject by any of a number of routes of administration. For example, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any suitable method in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the disclosure. A larger total dose can be delivered by multiple administrations of the agent. Many methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

The patient receiving this treatment may be any animal in need, including primates, in particular humans; and other mammals such as equines, cattle, swine, sheep, cats, and dogs; poultry; and pets in general.

In certain embodiments, compounds of the disclosure may be used alone or conjointly administered with another type of therapeutic agent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Compositions may be prepared in an injectable form, either as a liquid solution or as a suspension. Solid forms suitable for injection may also be prepared, e.g., as emulsions, or with the antibody-drug conjugate encapsulated in liposomes. Antibody-drug conjugates may be combined with a pharmaceutically acceptable carrier, which includes any carrier that does not induce the production of antibodies harmful to the subject receiving the carrier. Suitable carriers typically comprise large macromolecules that are slowly metabolized, for example, proteins, polysaccharides, polylactic acids, polyglycolic acids, polymeric amino acids, amino acid copolymers, lipid aggregates, and the like.

The compositions may also contain diluents, for example, water, saline, glycerol, and ethanol. Auxiliary substances, for example, wetting or emulsifying agents, pH buffering substances, and the like may also be present therein. The compositions may be parenterally administered by injection, wherein such injection may be either subcutaneous or intramuscular injection. In some embodiments, a composition may be administered into a tumor. The composition may be inserted (e.g., injected) into a tumor. Additional formulations are suitable for other forms of administration, such as suppository or oral administration. Oral compositions may be administered as a solution, suspension, tablet, pill, capsule, or sustained release formulation.

The compositions may be administered in a manner compatible with a dose and a formulation. The composition preferably comprises a therapeutically effective amount of the antibody-drug conjugate. A dose may vary, depending on the subject to be treated, the subject's health and physical conditions, a degree of protection to be desired, and other relevant factors. The exact amount of an active ingredient (e.g., the antibody-drug conjugate) may depend on the judgment of a doctor. For example, a therapeutically effective amount of the antibody-drug conjugate or composition containing the same may be administered to a patient suffering from a cancer or tumor to treat the cancer or tumor.

The antibody-drug conjugate according to the present disclosure or the composition containing the same may be administered in the form of a pharmaceutically acceptable salt thereof. In some embodiments, the antibody-drug conjugate according to the present disclosure or the composition containing the same may be administered with a pharmaceutically acceptable carrier, a pharmaceutically acceptable excipient, and/or a pharmaceutically acceptable additive. The effective amount and the type of the pharmaceutically acceptable salt, excipient and additive may be measured using standard methods (see, e.g., Remington's Pharmaceutical Sciences, Mack Publishing Co., Easton, PA, 18th Edition, 1990).

In some embodiments, the disclosure relates to a method of treating cancer in a subject, comprising administering a pharmaceutical composition comprising an antibody-drug conjugate as described herein to the subject. In preferred embodiments, the subject is a mammal. For example, the subject may be selected from rodents, lagomorphs, felines, canines, porcines, ovines, bovines, equines, and primates. In certain preferred embodiments, the subject is a human.

The conjugates of the disclosure (also referred to herein as “active compounds”), and derivatives, fragments, analogs and homologs thereof, can be incorporated into pharmaceutical compositions suitable for administration. Such compositions typically comprise the conjugate and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intradermal, and subcutaneous administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In certain embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to suitable methods, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### Methods of Treatment

The compounds and conjugates disclosed herein may be used in methods to induce apoptosis in cells.

Dysregulated apoptosis has been implicated in a variety of diseases, including, for example, autoimmune disorders (e.g., systemic lupus erythematosus, rheumatoid arthritis, graft-versus-host disease, myasthenia gravis, or Sjögren's syndrome), chronic inflammatory conditions (e.g., psoriasis, asthma or Crohn's disease), hyperproliferative disorders (e.g., breast cancer, lung cancer), viral infections (e.g., herpes, papilloma, or HIV), and other conditions, such as osteoarthritis and atherosclerosis. The compounds, conjugates, and compositions described herein may be used to treat or ameliorate any of these diseases. Such treatments generally involve administering to a subject suffering from the disease an amount of a compound, conjugate, or composition described herein sufficient to provide therapeutic benefit. The identity of the antibody of the compound, conjugate, or composition administered will depend upon the disease being treated—thus the antibody should bind a cell-surface antigen expressed in the cell type where inhibition would be beneficial. The therapeutic benefit achieved will also depend upon the specific disease being treated. In certain instances, the compounds and compositions disclosed herein may treat or ameliorate the disease itself, or symptoms of the disease, when administered as monotherapy. In other instances, the compounds and compositions disclosed herein may be part of an overall treatment regimen including other agents that, together with the inhibitor or the compounds and compositions disclosed herein, treat or ameliorate the disease being treated, or symptoms of the disease. Agents useful to treat or ameliorate specific diseases that may be administered adjunctive to, or with, the compounds and compositions disclosed herein will be apparent to those of skill in the art.

Although absolute cure is always desirable in any therapeutic regimen, achieving a cure is not required to provide therapeutic benefit. Therapeutic benefit may include halting or slowing the progression of the disease, regressing the disease without curing, and/or ameliorating or slowing the progression of symptoms of the disease. Prolonged survival as compared to statistical averages and/or improved quality of life may also be considered therapeutic benefit.

One particular class of diseases that involve dysregulated apoptosis and that are significant health burden world-wide are cancers. In a specific embodiment, the the compounds and compositions disclosed herein may be used to treat cancers. The cancer may be, for example, solid tumors or hematological tumors. Cancers that may be treated with the compounds and compositions disclosed herein include, but are not limited to bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, myeloma, prostate cancer, small cell lung cancer and spleen cancer. The compounds and compositions disclosed herein may be especially beneficial in the treatment of cancers because the antibody can be used to target the tumor cell specifically, thereby potentially avoiding or ameliorating undesirable side-effects and/or toxicities that may be associated with systemic administration of unconjugated inhibitors. One embodiment pertains to a method of treating a disease involving dysregulated intrinsic apoptosis, comprising administering to a subject having a disease involving dysregulated apotosis an amount of a compound and composition disclosed herein effective to provide therapeutic benefit, wherein the ligand of the compounds and compositions disclosed herein binds a cell surface receptor on a cell whose intrinsic apoptosis is dysregulated. One embodiment pertains to a method of treating cancer, comprising administering to a subject having cancer a compound and composition disclosed herein, wherein the ligand is capable of binding a cell surface receptor or a tumor associated antigen expressed on the surface of the cancer cells, in an amount effective to provide therapeutic benefit.

In the context of tumorigenic cancers, therapeutic benefit, in addition to including the effects discussed above, may also specifically include halting or slowing progression of tumor growth, regressing tumor growth, eradicating one or more tumors and/or increasing patient survival as compared to statistical averages for the type and stage of the cancer being treated. In one embodiment, the cancer being treated is a tumorigenic cancer.

The compounds and conjugates disclosed herein may be administered as monotherapy to provide therapeutic benefit, or may be administered adjunctive to, or with, other chemotherapeutic agents and/or radiation therapy. Chemotherapeutic agents to which the compounds and compositions disclosed herein may be utilized as adjunctive therapy may be targeted (for example, ADCs, protein kinase inhibitors, etc.) or non-targeted (for example, non-specific cytotoxic agents such as radionucleotides, alkylating agents and intercalating agents). Non-targeted chemotherapeutic agents with which the compounds and compositions disclosed herein may be adjunctively administered include, but are not limited to, methotrexate, taxol, L-asparaginase, mercaptopurine, thioguanine, hydroxyurea, cytarabine, cyclophosphamide, ifosfamide, nitrosoureas, cisplatin, carboplatin, mitomycin, dacarbazine, procarbizine, topotecan, nitrogen mustards, Cytoxan, etoposide, 5-fluorouracil, BCNU, irinotecan, camptothecins, bleomycin, doxorubicin, idarubicin, daunorubicin, dactinomycin, plicamycin, mitoxantrone, asperaginase, vinblastine, vincristine, vinorelbine, paclitaxel, calicheamicin, and docetaxel.

The compounds and conjugates disclosed herein that may not be effective as monotherapy to treat cancer may be administered adjunctive to, or with, other chemotherapeutic agents or radiation therapy to provide therapeutic benefit. One embodiment pertains to a method in which a compound or composition disclosed herein is administered in an amount effective to sensitize the tumor cells to standard chemotherapy and/or radiation therapy. Accordingly, in the context of treating cancers, “therapeutic benefit” includes administering the compounds and compositions disclosed herein adjunctive to, or with, chemotherapeutic agents and/or radiation therapy, either in patients who have not yet begin such therapy or who have but have not yet exhibited signs of resistance, or in patients who have begun to exhibit signs of resistance, as a means of sensitizing the tumors to the chemo and/or radiation therapy.

In some aspects, the present disclosure provides pharmaceutical compositions comprising an antibody drug conjugate as described herein, optionally further comprising a therapeutically effective amount of a chemotherapeutic agent.

In certain aspects, provided herein are methods of treating a cancer, comprising administering one or more of the compounds, drug conjugates, or pharmaceutical compositions of the present disclosure to a subject in need thereof.

In certain embodiments, the cancer is selected from leukemia, lymphoma, breast cancer, colon cancer, ovarian cancer, bladder cancer, prostate cancer, glioma, lung cancer, bronchial cancer, colorectal cancer, pancreatic cancer, esophageal cancer, liver cancer, urinary bladder cancer, kidney cancer, renal pelvis cancer, oral cavity cancer, pharynx cancer, uterine corpus cancer, or melanoma.

In further aspects, provided herein are methods of treating autoimmune diseases or inflammatory diseases, comprising administering one or more of the compounds, drug conjugates, or pharmaceutical compositions of the present disclosure to a subject in need thereof.

In certain embodiments, the autoimmune disease or the inflammatory disease is selected from B-cell mediated autoimmune diseases or inflammatory diseases, for example, systemic lupus erythematosus (SLE), rheumatoid arthritis (RA), idiopathic thrombocytopenic purpura (ITP), Waldenstrom's hypergammaglobulinaemia, Sjogren's syndrome, multiple sclerosis (MS), or lupus nephritis.

Hereinafter, configurations of the present disclosure will be described in detail through Examples, but the following Examples are only to assist in understanding of the present disclosure. The scope of the present disclosure is not limited thereto. Further, unless specifically described otherwise, the reagent, solvent, and starting material described in the specification can be easily obtained from a commercial supplier.

### EXAMPLES

### Example 1: Preparation of Compound D1

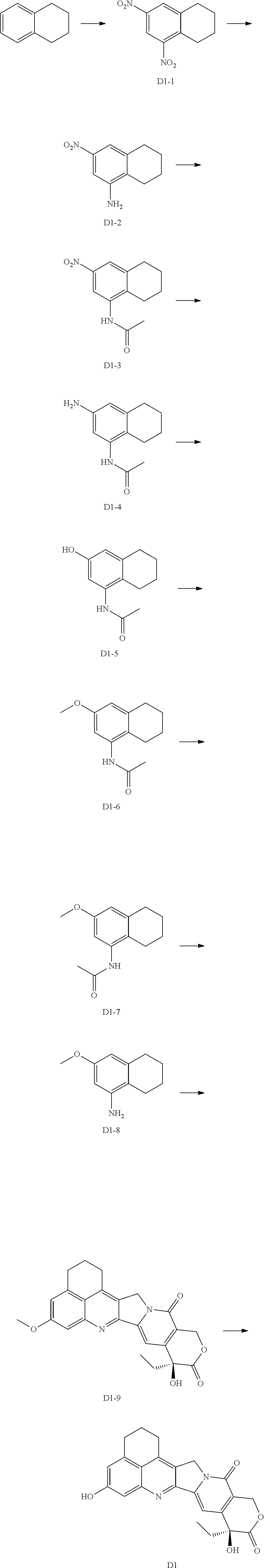

### Preparation of Compound D1-1

To a solution of compound 1,2,3,4-tetrahydronaphthalene (10.0 g, 75.6 mmol) in H₂SO₄ (55 mL) was added HNO₃ (55 mL) at 0° C. under N₂ atmosphere. The reaction mixture was stirred at same temperature for 30 minutes. After the reaction was completed, the reaction mixture was diluted with distilled water and extracted with ethyl acetate (EA). The combined organic layer was washed with saturated aqueous NaHCO₃. The organic layer was dried over anhydrous Na₂SO₄, and filtered and concentrated under reduced pressure. The residue was purified by column chromatography to obtain compound D1-1 (3.00 g, 18%).

¹H NMR (400 MHz, CDCl₃) δ 8.49 (d, J=6.4 Hz, 1H), 8.18 (d, J=2.0 Hz, 1H), 3.08-2.97 (m, 4H), 1.91-1.84 (m, 4H)

### Preparation of Compound D1-2

To a solution of compound D1-1 (6.30 g, 28.4 mmol) in ethanol (44 mL) was added acetic acid (44 mL), conc HCl (44 mL) and SnCl₂·2H₂O (19.2 g, 85.1 mmol) at room temperature under N₂ atmosphere. The reaction mixture was refluxed for 7 hours. The reaction mixture was quenched with saturated aqueous Na₂CO₃ and extracted with EA. The combined organic layer was dried over anhydrous Na₂SO₄, filtered and concentrated under reduced pressure. The crude compound was purified by column chromatography to obtain compound D1-2 (2.39 g, 44%).

¹H NMR (400 MHz, CDCl₃) δ 7.40 (s, 1H), 7.32 (d, J=2.0 Hz, 1H), 3.85 (brs, 1H), 2.80 (t, J=6.0 Hz, 2H), 2.48 (t, J=6.4 Hz, 2H), 1.92-1.89 (m, 2H), 1.82-1.78 (m, 2H)

### Preparation of Compound D1-3

To a solution of compound D1-2 (2.39 g, 12.4 mol) in anhydrous dichloromethane (DCM, 41 mL) was added acetic anhydride (2.35 mL) at 0° C. under N₂ atmosphere. The reaction mixture was stirred at room temperature for 3 hours. The reaction mixture was quenched with saturated aqueous Na₂CO₃ and extracted with EA. The combined organic layer was dried over anhydrous Na₂SO₄, filtered and concentrated under reduced pressure. The crude compound D1-3 (2.91 g) was used in the next step without further purification.

¹H NMR (400 MHz, CDCl₃) δ 8.57 (s, 1H), 7.80 (s, 1H), 6.97 (brs, 1H), 2.87 (t, J=6.4 Hz, 2H), 2.63 (t, J=6.4 Hz, 2H), 1.89-1.81 (m, 4H)

ESI-MS m/z: 235 (M⁺+1).

### Preparation of Compound D1-4

To a solution of compound D1-3 (6.1 g, 0.026 mol) in MeOH (200 mL) was added 5% Pd/C wetted with ca. 55% water (277 mg, 0.0026 mol) at room temperature. The reaction mixture was degassed 5 times with a hydrogen gas balloon at room temperature. The reaction mixture was stirred at room temperature for 3 hours under H₂ atmosphere. After the reaction was completed, the reaction mixture was celite filtered washed with MeOH. The mixture was filtered and concentrated under reduced pressure. The crude compound was vacuum-dried for 15 hours to obtain crude compound D1-4 (5.28 g).

¹H NMR (400 MHz, CDCl₃) δ 7.21 (s, 1H), 6.99 (brs, 1H), 6.27 (s, 1H), 3.54 (s, 2H), 2.66 (t, J=6.0 Hz, 2H), 2.47 (t, J=6.2 Hz, 2H), 2.17 (s, 3H), 1.83-1.68 (m, 4H)

ESI-MS m/z: 205 (M⁺+1).

### Preparation of Compound D1-5

To a solution of crude compound D1-4 (5.28 g, 0.0258 mol) in 1% H₂SO₄ solution (129 mL) at 0° C. A solution of NaNO₂ (2.67 g, 0.0387 mol) in distilled water (10 mL) at room temperature. The reaction mixture was added dropwise NaNO₂ solution at 0° C. After stirring at 0° C. for 20 minutes, the reaction mixture was added 100° C. distilled water (129 mL) at room temperature. After stirring at 75° C. for 20 minutes. The reaction mixture was added EA (700 mL) and then extracted. The water layer was washed with 5% MeOH/EA (500 mL) and then 10% MeOH/EA (500 mL). The organic layer was dried over anhydrous Na₂SO₄, filtered and concentrated under reduced pressure. The crude compound was vacuum-dried for 15 hours to obtain crude compound D1-5 (5.43 g).

¹H NMR (400 MHz, CDCl₃) δ 7.20 (d, J=2.4 Hz, 1H), 6.44 (d, J=2.0 Hz, 1H), 3.41 (brs, 1H), 2.70 (t, J=6.0 Hz, 2H), 2.48 (t, J=6.4 Hz, 2H), 2.18 (s, 3H), 1.84-1.71 (m, 4H) ESI-MS m/z: 206 (M⁺+1).

### Preparation of Compound D1-6

To a solution of crude compound D1-5 (5.4 g, 0.0263 mol) in anhydrous N,N-dimethylformamide (DMF, 60 mL) at room temperature under N₂ atmosphere. The reaction mixture was added K₂CO₃ (18.2 g, 0.132 mol) at room temperature under N₂ atmosphere. The reaction mixture was added dropwise Mel (8.2 mL, 0.132 mol) at 0° C. under N₂ atmosphere. The reaction mixture was stirred at 0° C. for 15 minutes, and then warmed up to room temperature under N₂ atmosphere for 1 hour 30 minutes. The reaction mixture was stirred at 40° C. under N₂ atmosphere for 21 hours. The reaction mixture was added EA (600 mL) and distilled water (200 mL). After extracted, organic layer was washed with brine (150 mL×5). The organic layer was dried over anhydrous Na₂SO₄, filtered and concentrated under reduced pressure. The crude compound was triturated with 20% ether/hexane and then one more triturated with only hexane (Hex). The solution was filtrated wash with Hex. The solid was vacuum-dried for 15 hours to obtain compound D1-6 (4.5 g, 4 steps yield=75%).

¹H NMR (400 MHz, CDCl₃) δ 7.41 (s, 1H), 6.89 (brs, 1H), 6.47 (s, 1H), 3.77 (s, 3H), 2.74 (t, J=6.0 Hz, 2H), 2.51 (t, J=6.2 Hz, 2H), 2.19 (s, 3H), 1.83-1.73 (m, 4H) ESI-MS m/z: 220 (M⁺+1).

### Preparation of Compound D1-7

To a solution of compound D1-6 (4.5 g, 0.0205 mol) in acetone (100 mL) at room temperature under N₂ atmosphere. The reaction mixture was added 15% MgSO₄ in distilled water (11 mL, 0.0133 mol) at 0° C. The reaction mixture was added KMnO₄ (8.1 g, 0.0513 mol) at 0° C. The reaction mixture was stirred at 0° C. for 3.5 hours. The reaction mixture was diluted with distilled water (300 mL) and extracted with EA (500 mL). The organic layer was washed with brine (150 mL×2). The organic layer was dried over anhydrous Na₂SO₄, filtered and concentrated under reduced pressure. The residue was purified by column chromatography to obtain compound D1-7 (2.6 g, 54%).

¹H NMR (400 MHz, CDCl₃) δ 8.29 (d, J=2.8 Hz, 1H), 6.43 (d, J=2.4 Hz, 1H), 3.86 (s, 3H), 2.90 (t, J=6.2 Hz, 2H), 2.64 (t, J=6.4 Hz, 2H), 2.23 (s, 3H), 2.04 (p, J=12.6 Hz, 6.3 Hz, 2H) ESI-MS m/z: 234 (M⁺+1).

### Preparation of Compound D1-8

To a solution of compound D1-7 (2.6 g, 0.0111 mol) in 6N HCl solution (124 mL) at room temperature. The reaction mixture was stirred at 90° C. for 1 hour. After the reaction was completed, the reaction mixture concentrated under reduced pressure. The reaction was quenched by addition of distilled water (20 mL) and saturated aqueous NaHCO₃ (20 mL) and 15% NaOH (25 mL) at 0° C. (pH 10). The reaction mixture was extracted with EA (300 mL). The organic layer was dried over anhydrous Na₂SO₄, filtered and concentrated under reduced pressure. Producing compound D1-8 (2.21 g, 104%) as brown solid, which was used without further purification.

¹H NMR (400 MHz, CDCl₃) δ 6.56 (brs, 2H), 6.05 (d, J=2.4 Hz, 1H), 5.92 (d, J=2.0 Hz, 1H), 3.78 (s, 3H), 2.81 (t, J=6.0 Hz, 2H), 2.57 (t, J=6.4 Hz, 2H), 2.00 (p, J=12.4 Hz, 6.3 Hz, 2H) ESI-MS m/z: 192 (M⁺+1).

### Preparation of Compound D1-9

To a solution of compound D1-8 (2.21 g, 0.0116 mol) and (S)-4-ethyl-4-hydroxy-7,8-dihydro-1H-pyrano[3,4-f]indolizine-3,6,10 (4H)-trione (3.66 g, 0.0139 mol) in toluene (18 mL) at room temperature under N₂ atmosphere. The reaction mixture was added pyridinium p-toluenesulfonate (PPTS, 2.92 g, 0.0116 mol) at room temperature under N₂ atmosphere. The reaction mixture was stirred at 130° C. with dean-stark for 7 hours. After the reaction was completed, the high-temperature reaction solution was decanted and separated from tar. The decanted solution was cooled to room temperature to filter the resulting solid (pure product). The filtrate was combined with tar, concentrated under reduced pressure. The residue was purified by column chromatography to obtain compound D1-9 (1.7 g, 36%).

¹H NMR (400 MHz, CDCl₃) δ 7.64 (s, 1H), 7.37 (d, J=2.8 Hz, 1H), 7.04 (d, J=2.4 Hz, 1H), 5.76 (d, J=16.4 Hz, 1H), 5.31 (d, J=12.4 Hz, 1H), 5.16 (s, 2H), 3.98 (s, 2H), 3.75 (s, 1H), 3.13 (q, J=6.8 Hz, 4H), 2.18 (p, J=12.2 Hz, 6.2 Hz, 2H), 1.93-1.85 (m, 2H), 1.03 (t, J=7.4 Hz, 3H) ESI-MS m/z: 419 (M⁺+1).

### Preparation of Compound D1

To a solution of compound D1-9 (1.7 g, 0.00406 mol) in 48% HBr solution (40.6 mL) at room temperature under N₂ atmosphere. The reaction mixture was stirred at 130° C. under N₂ atmosphere for 17 hours. The reaction was quenched by addition of distilled water (41 mL) and saturated aqueous NaHCO₃ (400 mL) at 0° C. (pH 7-8). The reaction mixture was filtrated washed with anhydrous DCM and distilled water. The filtrate was extracted organic layer was dried over anhydrous Na₂SO₄, filtered and concentrated under reduced pressure. The residue was purified by column chromatography to obtain compound D1 (1.03 g, 63%).

¹H NMR (400 MHz, CDCl₃) δ 7.63 (s, 1H), 7.21 (d, J=2.4 Hz, 1H), 7.04 (d, J=2.0 Hz, 1H), 5.71 (d, J=16.0 Hz, 1H), 5.31 (d, J=16.4 Hz, 1H), 5.14 (s, 2H), 3.42-3.40 (m, 2H), 3.15-3.09 (m, 4H), 2.21-2.15 (m, 2H), 1.96-1.86 (m, 2H), 1.03 (t, J=7.4 Hz, 3H) ESI-MS m/z: 405 (M⁺+1).

### Example 2: Preparation of Compound D2

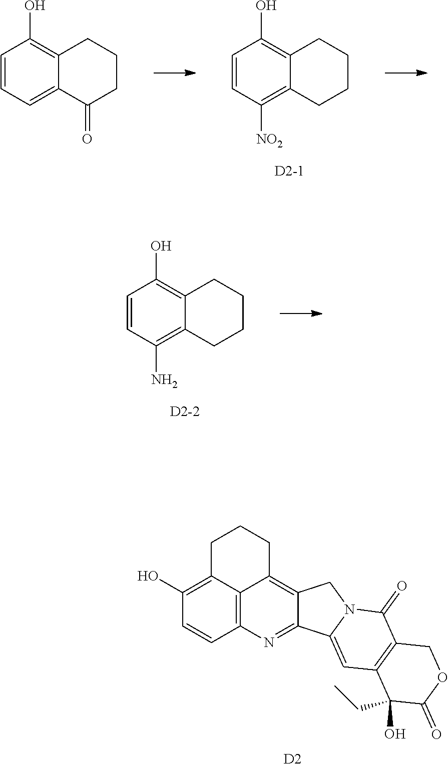

### Preparation of Compound D2-1

To a solution of compound 5-hydroxy-1-tetralone (30 mg, 0.185 mmol) in acetic acid (1.2 mL) was added HNO₃ (10.0 L, 0.241 mmol) under N₂ atmosphere. The reaction mixture was stirred at 120° C. for 25 minutes. The reaction mixture was quenched with saturated aqueous NaHCO₃ and extracted with EA. The combined organic layer was dried over anhydrous Na₂SO₄, filtered and concentrated under reduced pressure. The residue was purified by prep TLC to obtain compound D2-1 (12.1 mg, 32%).

¹H NMR (400 MHz, CDCl₃) δ 7.35 (d, J=8.4 Hz, 1H), 6.97 (d, J=8.8 Hz, 1H), 2.91 (t, J=6.4 Hz, 2H), 2.67 (t, J=6.4 Hz, 2H), 2.17-2.11 (m, 2H) ESI-MS m/z: 207 (M⁺).

### Preparation of Compound D2-2

To a solution of compound D2-1 (90 mg, 0.434 mmol) in ethanol (0.68 mL) was added acetic acid (0.68 mL), conc HCl (0.68 mL) and SnCl₂·2H₂O (294 mg, 1.30 mmol) under N₂ atmosphere. The reaction mixture was stirred at 120° C. for 1.5 hours. The reaction mixture was quenched with saturated aqueous Na₂CO₃ and extracted with EA. The combined organic layer was dried over anhydrous Na₂SO₄, filtered and concentrated under reduced pressure. The crude compound was purified by column chromatography to obtain compound D2-2 (77.0 mg, 100%).

¹H NMR (400 MHz, CDCl₃) δ 6.86 (d, J=9.2 Hz, 1H), 6.49 (d, J=8.8 Hz, 1H), 4.62 (brs, 1H), 2.83 (t, J=6.0 Hz, 2H), 2.57 (t, J=6.0 Hz, 2H), 2.04-1.96 (m, 2H) ESI-MS m/z: 178 (M⁺+1).

### Preparation of Compound D2

To a solution of compound D2-2 (50.0 mg, 0.282 mmol) and (4S)-4-ethyl-7,8-dihydro-4-hydroxy-1H-pyrano[3,4-f]indolizine-3,6,10 (4H)-trione (81.7 mg, 0.310 mmol) in acetic acid (5.0 mL) was heated at 110° C. under N₂ atmosphere. The reaction mixture was stirred at same temperature for 2 hours. The reaction mixture was concentrated and the residue was triturated by EA/DCM. The solid was filtered and washed with anhydrous DCM to afford the compound D2 (74.7 mg, 66%).

ESI-MS m/z: 405 (M⁺+1).

### Example 3: Preparation of Compound D3

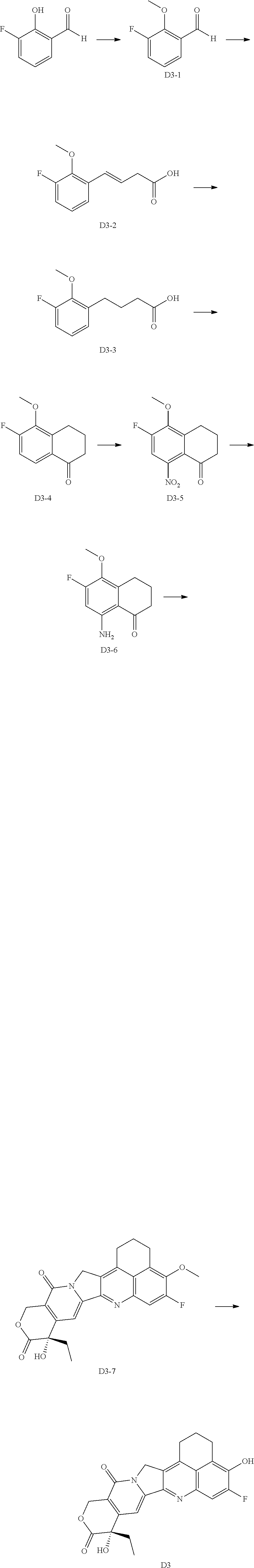

### Preparation of Compound D3-1

To a solution of 3-fluoro-2-hydroxybenzaldehyde (2.8 g, 20.0 mmol) in acetone (35 mL) was added K₂CO₃ (4.14 g, 30.0 mmol) and dimethyl sulfate (1.93 ml, 20.38 mmol) at room temperature under N₂ atmosphere. The reaction mixture was stirred at 60° C. for 90 minutes. After the reaction was completed, reaction mixture was allowed to cool, concentrated, and diluted with distilled water (50 mL) and extracted with anhydrous DCM (70 mL). The combined organic layer was washed with saturated aqueous NaHCO₃ (60 mL). The organic layer was dried over anhydrous Na₂SO₄, filtered and concentrated under reduced pressure. The crude compound D3-1 (2.92 g) was used in the next step without further purification.

¹H NMR (400 MHz, CDCl₃) δ 10.41 (s, 1H), 7.61 (dd, J=7.8 Hz, 2.6 Hz, 1H), 7.34-7.26 (m, 1H), 7.34-7.26 (m, 1H), 4.10 (s, 3H) ESI-MS m/z: 155 (M⁺+1).

### Preparation of Compound D3-2

To a solution of (2-carboxyethyl)triphenylphosphonium bromide (9.14 g, 22.0 mmol) in dry tetrahydrofuran (THF, 42 mL), dimethyl sulfoxide (DMSO, 18 mL) was added NaH 60% dispersion in mineral oil (1.76 g, 44.0 mmol) at room temperature under N₂ atmosphere. The mixture was stirred at room temperature under for 5 minutes and added crude compound D3-1 (2.92 g, 20.0 mmol) in THF (10 mL) solution slowly. The mixture was stirred at room temperature under for 17 hours and quenched with saturated aqueous NaHCO₃ (80 mL), extracted with EA (100 mL×2). The water layer quenched by 6N HCl (˜pH 2) and extracted with EA (100 mL×2). The organic layer was dried over anhydrous Na₂SO₄, filtered and concentrated under reduced pressure. The residue was purified by column chromatography to obtain compound D3-2 (2.47 g, 2 steps yield=59%).

¹H NMR (400 MHz, CDCl₃) δ 7.25-7.22 (m, 1H), 6.83-6.78 (m, 1H), 6.38-6.31 (m, 1H), 3.90 (s, 3H), 3.34 (d, J=6.8 Hz, 2H)

### Preparation of Compound D3-3

To a solution of compound D3-2 (2.47 g, 11.75 mmol) in MeOH (90 mL) was added 5% Pd/C wetted with ca. 55% water (2.5 g, 1.175 mmol) at room temperature. The reaction mixture was degassed 5 times with a hydrogen gas balloon at room temperature. The reaction was stirred at room temperature under H₂ atmosphere for 2 hours. After the reaction was completed, reaction mixture was celite filtered washed with MeOH. The mixture was filtered and concentrated under reduced pressure. The crude compound was vacuum-dried for 3 hours to obtain crude compound D3-3 (2.14 g).

¹H NMR (400 MHz, CDCl₃) δ 6.95-6.90 (m, 3H), 3.91 (s, 3H), 2.69 (t, J=7.6 Hz, 2H), 2.39 (t, J=7.6 Hz, 2H), 1.93 (p, J=12.5 Hz, 7.5 Hz, 2H)

### Preparation of Compound D3-4

To a solution of crude compound D3-3 (2.14 g, 11.75 mmol) in polyphosphoric acid (4 g) was stirred at 110° C. under N₂ atmosphere for 30 minutes. After the reaction was completed, reaction mixture was allowed to cool, diluted with distilled water (50 mL) and extracted with EA (70 mL) and washed with brine (60 mL). The organic layer was dried over anhydrous Na₂SO₄, filtered and concentrated under reduced pressure. The residue was purified by column chromatography to obtain compound D3-4 (1.21 g, 2 steps yield=53%).

¹H NMR (400 MHz, CDCl₃) δ 7.79 (q, J=4.6 Hz, 1H), 7.03 (q, J=6.5 Hz, 1H), 3.94 (s, 3H), 2.95 (t, J=6.2 Hz, 2H), 2.61 (t, J=6.4 Hz, 2H), 2.12 (p, J=12.8 Hz, 6.4 Hz, 2H) ESI-MS m/z: 195 (M⁺+1).

### Preparation of Compound D3-5

To a solution of compound D3-4 (1.21 g, 6.23 mmol) in H₂SO₄ (6 mL) was added HNO₃ (314 L, 7.48 mmol) solution in H₂SO₄ (3 mL) at 0° C. under N₂ atmosphere. The reaction was stirred at 0° C. for 30 minutes and quenched ice-water (30 mL), extracted with EA (50 mL), washed with saturated aqueous NaHCO₃ (40 mL). The organic layer was dried over anhydrous Na₂SO₄, filtered and concentrated under reduced pressure. The residue was purified by column chromatography to obtain compound D3-5 (707 mg, 47%).

¹H NMR (400 MHz, CDCl₃) δ 7.17 (d, J=10.8 Hz, 1H), 4.05 (s, 3H), 2.96 (t, J=6.2 Hz, 2H), 2.68 (t, J=6.6 Hz, 2H), 2.15 (p, J=13.4 Hz, 6.8 Hz, 2H) ESI-MS m/z: 240 (M⁺+1).

### Preparation of Compound D3-6

D3-6 was obtained by performing a reaction in a similar method as described in Example 3 compound D1-2 (598 mg, 97%).

¹H NMR (400 MHz, CDCl₃) δ 6.43 (brs, 2H), 6.21 (d, J=12.8 Hz, 1H), 3.76 (s, 3H), 2.91 (t, J=6.2 Hz, 2H), 2.59 (t, J=6.4 Hz, 2H), 2.01 (p, J=12.8 Hz, 6.4 Hz, 2H) ESI-MS m/z: 210 (M⁺+1).

### Preparation of Compound D3-7

D3-7 was obtained by performing a reaction in a similar method as described in Example 3 compound D1-9 (1.02 g, 100%).

ESI-MS m/z: 437 (M⁺+1).

### Preparation of Compound D3

D3 was obtained by performing a reaction in a similar method as described in Example 3 compound D1 (883 mg, 95%).

¹H NMR (400 MHz, DMSO-d6) δ 7.72 (d, J=12.0 Hz, 1H), 7.23 (s, 1H), 5.41 (s, 2H), 5.20 (s, 2H), 3.09 (t, J=6.2 Hz, 2H), 3.01 (t, J=6.2 Hz, 2H), 2.00 (t, J=6.4 Hz, 2H), 1.85 (p, J=12.4 Hz, 7.4 Hz, 2H)

ESI-MS m/z: 423 (M⁺+1).

### Example 4: Preparation of Compound D4

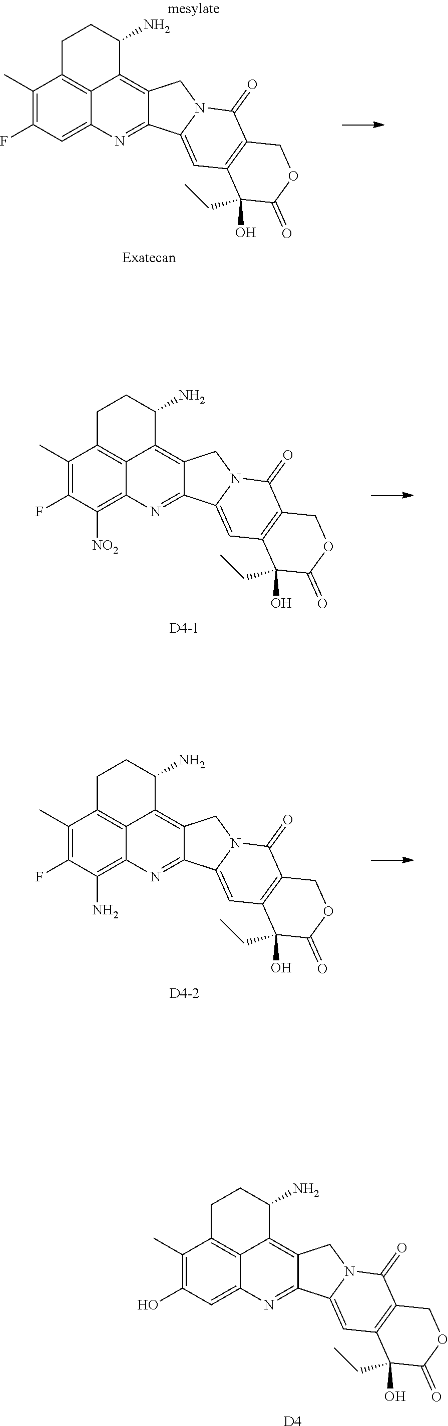

### Preparation of Compound D4-1

To a solution of exatecan mesylate (20.0 mg, 0.0376 mmol) in H₂SO₄ (1.06 mL) at −5° C. under N₂ atmosphere was treated with HNO₃ (12 μL) and stirred at room temperature for 3.5 hours. After reaction was completed, the mixture was basified with saturated aqueous NaHCO₃, adjusted to pH 4. The organic layer was washed with distilled water, dried over anhydrous Na₂SO₄, filtered, and concentrated under reduced pressure. The residue was purified by prep HPLC to obtain compound D4-1 (6.9 mg, 18%).

ESI-MS m/z: 481 (M⁺+1).

### Preparation of Compound D4-2

To a solution of compound D4-1 (6.9 mg, 0.014 mmol) in EtOH (0.7 mL)/dioxane (0.7 mL) was added Pt₂O (1.6 mg, 0.0072 mmol). H₂ gas was introduced via a balloon, and the mixture was stirred at room temperature for 1.5 hours. The mixture was filtered using celite and concentrated under reduced pressure. The crude D4-2 was in situ next step without further purification (6.4 mg, crude).

ESI-MS m/z: 451 (M⁺+1).

### Preparation of Compound D4

To a solution of compound D4-2 (6.4 mg, 0.014 mmol) in 20% H₂SO₄ (0.56 mL) was added NaNO₂ (1.16 mg, 0.0168 mmol) at room temperature under N₂ atmosphere. The reaction mixture was stirred at room temperature for 15 minutes. After the reaction was completed, hot distilled water (5.6 mL) was added and then the reaction mixture was stirred for 4 hours at 100° C. The residue was purified by prep HPLC to obtain compound D4 (0.2 mg, 3.3%).

ESI-MS m/z: 434 (M⁺+1).

### Example 5: Preparation of Compound D5

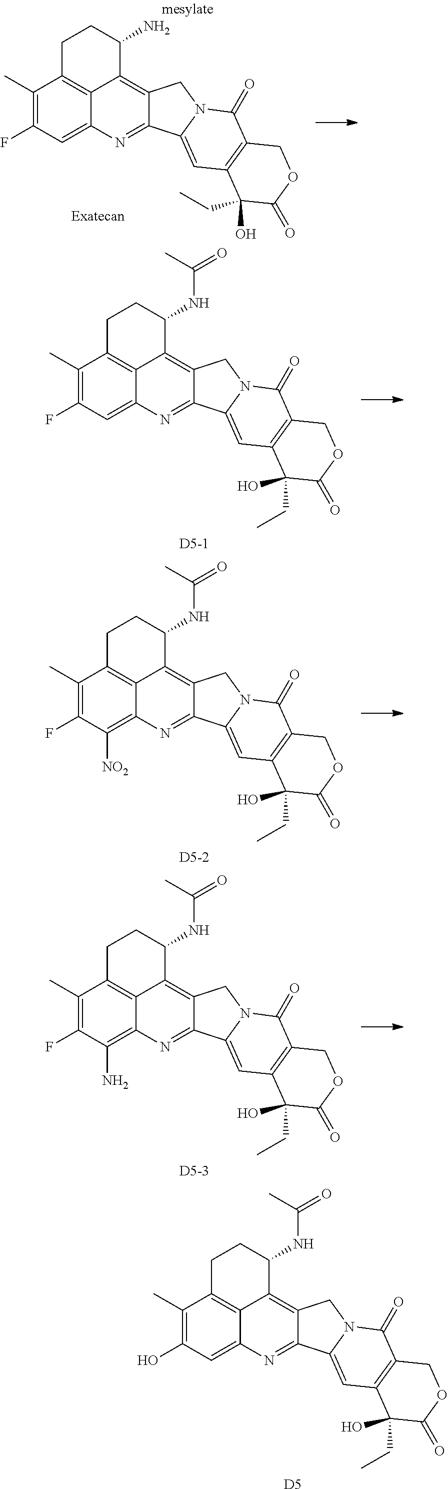

### Preparation of Compound D5-1

To a solution of exatecan mesylate (50.0 mg, 0.094 mmol) in DCM/DMF (1/1, 0.5 mL) was added acetic anhydride (9.8 μL, 0.103 mmol) and trimethylamine (14.4 μL, 0.103 mmol) at 0° C. under N₂ atmosphere. The reaction mixture was stirred at room temperature for 2 hours. The reaction mixture was extracted with EA and distilled water, the organic layer was dried over anhydrous Na₂SO₄, filtered and concentrated under reduced pressure. The crude compound was washed with Hex and then purified by column chromatography to obtain compound D5-1 (37.7 mg, 84%).

ESI-MS m/z: 478 (M⁺+1).

### Preparation of Compound D5-2

D5-2 was obtained by performing a reaction in a similar method as described in Example 4 compound D4-1 (26.4 mg, 64%).

ESI-MS m/z: 523 (M⁺+1).

### Preparation of Compound D5-3

D5-3 was obtained by performing a reaction in a similar method as described in Example 4 compound D4-2 (34.9 mg, crude).

ESI-MS m/z: 493 (M⁺+1).

### Preparation of Compound D5

To a solution of crude compound D5-3 (34.9 mg, 0.0505 mmol) in 6 N HCl solution (758 μL) and NaNO₂ (8.7 mg, 0.126 mmol) at −5° C. The reaction mixture was stirred at −5° C. for 2 hours. The reaction mixture was added H₃PO₂ (44.7 μL, 0.505 mmol) and stirred at 70° C. for 2 hours. The reaction mixture was concentrated under reduced pressure. The crude compound was purified by prep TLC to obtain compound D5 (2 mg, 2 steps yield=8.3%).

ESI-MS m/z: 476 (M⁺+1).

### Example 6: Preparation of Compound D6 and D7

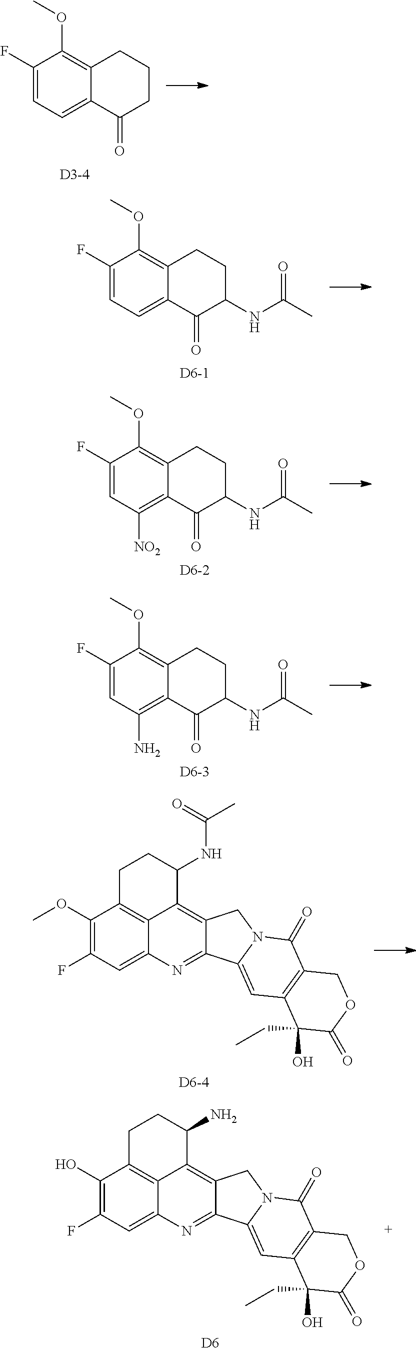

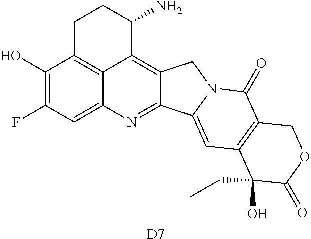 [SMILES: CC[C@@]1(O)C(=O)OCc2c1cc1n(c2=O)Cc2c-1nc1cc(F)c(O)c3c1c2[C@@H](N)CC3 ; "D7"]

### Preparation of Compound D6-1

To a mixed solution of THF (191 mL) and t-BuOH (19.1 mL) containing t-BuOK (3.77 g, 33.6 mmol) was added slowly in at 0° C. under N₂ atmosphere. The compound D3-4 (2.97 g, 15.3 mmol) dissolved in THF (95 mL) solution was added to t-BuOK mix solution at 0° C. under N₂ atmosphere. The reaction mixture was stirred at 0° C. for 10 minutes. The reaction mixture was dropwise added t-butyl nitrite (2.73 mL, 22.9 mmol) and stirred for 1.5 hours, during which the temperature was slowly raised to 20° C. The reaction mixture was adjusted to pH 1 with the addition of 1 N HCl solution, extracted with chloroform and washed with brine. The organic layer was dried over anhydrous Na₂SO₄, filtered and concentrated under reduced pressure. The residue was added acetic acid (190 mL) and acetic anhydride (190 mL), zinc powder (20 g, 306 mmol) followed by stirring at 20° C. for 30 minutes. Insoluble substances were removed by filtration. The filtrate was evaporated and the residue was cooled with ice-bath, basified with saturated aqueous NaHCO₃. The basic mixture solution was extracted with anhydrous DCM. The organic layer was dried over anhydrous Na₂SO₄, filtered and concentrated under reduced pressure. The residue was purified by column chromatography to obtain compound D6-1 (2.48 g, 65%).

¹H NMR (400 MHz, CDCl₃) δ 7.74 (q, J=4.6 Hz, 1H), 7.06 (q, J=6.6 Hz, 1H), 6.57 (brs, 1H), 4.60 (td, J=11.3 Hz, 6.8 Hz, 1H), 3.96 (d, J=2.0 Hz, 3H), 3.25-3.20 (m, 1H), 3.02-2.92 (m, 1H), 2.84-2.80 (m, 1H), 2.09 (s, 3H) ESI-MS m/z: 252 (M⁺+1).

### Preparation of Compound D6-2

D6-2 was obtained by performing a reaction in a similar method as described in Example 3 compound D3-5 (2.36 g, 81%).

¹H NMR (400 MHz, CDCl₃) δ 7.29 (s, 1H), 6.57 (brs, 1H), 4.81 (td, J=11.4 Hz, 6.7 Hz, 1H), 4.09 (d, J=3.6 Hz, 3H), 3.26 (dd, J=18.4 Hz, 4.4 Hz, 1H), 3.17-2.96 (m, 1H), 2.87-2.85 (m, 1H), 2.06 (s, 3H), 1.86-1.78 (m, 1H) ESI-MS m/z: 297 (M⁺+1).

### Preparation of Compound D6-3

To a solution of NH₄Cl (2.56 g, 47.8 mmol) in distilled water/EtOH (1/1, 40 mL) at room temperature was treated with Iron powder (2.67 g, 47.8 mmol) and stirred at 60° C. for 30 minutes. The reaction mixture was added compound D6-2 (2.36 g, 7.97 mmol) at 60° C. The reaction mixture was stirred at 80° C. for 90 minutes. The reaction was cooled with ice-bath, basified with saturated aqueous NaHCO₃ to pH˜12. The reaction mixture was filtered to remove solid residue. The filtrate was extracted with EA. The organic layer was dried over anhydrous Na₂SO₄, filtered and concentrated under reduced pressure. The residue was purified by column chromatography to obtain compound D6-3 (2.02 g, 95%).

¹H NMR (400 MHz, CDCl₃) δ 6.56 (d, J=3.6 Hz, 1H), 6.29 (brs, 2H), 6.25 (d, J=12.4 Hz, 1H), 4.50 (td, J=10.93 Hz, 6.7 Hz, 1H), 3.78 (s, 3H), 3.26-3.20 (m, 1H), 2.91-2.82 (m, 1H), 2.75-2.70 (m, 1H), 2.09 (s, 3H), 1.77-1.66 (m, 1H)

ESI-MS m/z: 267 (M⁺+1).

### Preparation of Compound D6-4

To a solution of compound D6-3 (621.37 mg, 2.33 mmol), (S)-4-ethyl-4-hydroxy-7,8-dihydro-1H-pyrano[3,4-f]indolizine-3,6,10 (4H)-trione (798.6 mg, 3.03 mmol) in toluene (47 mL) at room temperature under N₂ atmosphere was treated with PPTS (586.4 mg, 2.33 mmol) and stirred at 130° C. for 18.5 hours. When the reaction was completed, the reaction mixture was concentrated under reduced pressure. The residue was purified by column chromatography to obtain compound D6-4 (839.9 mg, 73%).

¹H NMR (400 MHz, DMSO-d6) δ 8.47 (t, J=8.0 Hz, 1H), 7.92 (d, J=12.4 Hz, 1H), 7.30 (s, 1H), 6.50 (s, 1H), 5.55 (p, J=8.8 Hz, 4.5 Hz, 1H), 5.42 (s, 2H), 5.20 (q, J=16.4 Hz, 2H), 3.97 (s, 3H), 3.20-3.15 (m, 2H), 2.12-2.08 (m, 2H), 1.90-1.81 (m, 2H), 0.87 (t, J=7.4 Hz, 3H) ESI-MS m/z: 494 (M⁺+1).

### Preparation of Compound D6 and D7

To a solution of compound D6-4 (839.9 mg, 3.15 mmol), in 44% HBr solution (5 mL) at room temperature under N₂ atmosphere was stirred for 3.5 hours at 130° C. When the reaction was completed, the reaction mixture was concentrated under reduced pressure. The residue was purified by C18 MPLC and prep HPLC to obtain D6 (more polar, 249.6 mg, 26.6%), D7 (less polar, 269.47 mg, 28.7%).

### Compound D6

¹H NMR (400 MHz, DMSO-d6) δ 10.74 (brs, 1H), 8.40 (brs, 2H), 7.93 (d, J=11.6 Hz, 1H), 7.30 (s, 1H), 6.53 (brs, 1H), 5.68 (d, J=19.6 Hz, 1H), 5.44 (s, 2H), 5.39 (d, J=19.6 Hz, 1H), 5.07 (brs, 1H), 2.97-2.88 (m, 2H), 2.17-2.07 (m, 2H), 1.93-1.82 (m, 2H), 0.88 (t, J=7.4 Hz, 3H)

ESI-MS m/z: 438 (M⁺+1) free form

### Compound D7

¹H NMR (400 MHz, DMSO-d6) δ 10.74 (brs, 1H), 8.40 (brs, 2H), 7.93 (d, J=11.6 Hz, 1H), 7.30 (s, 1H), 6.51 (brs, 1H), 5.68 (d, J=19.6 Hz, 1H), 5.44 (s, 2H), 5.39 (d, J=19.2 Hz, 1H), 5.08 (brs, 1H), 2.99-2.88 (m, 2H), 2.15-2.06 (m, 2H), 1.94-1.80 (m, 2H), 0.87 (t, J=7.4 Hz, 3H)

ESI-MS m/z: 438 (M⁺+1) free form.

### Example 7: Preparation of Compound D8

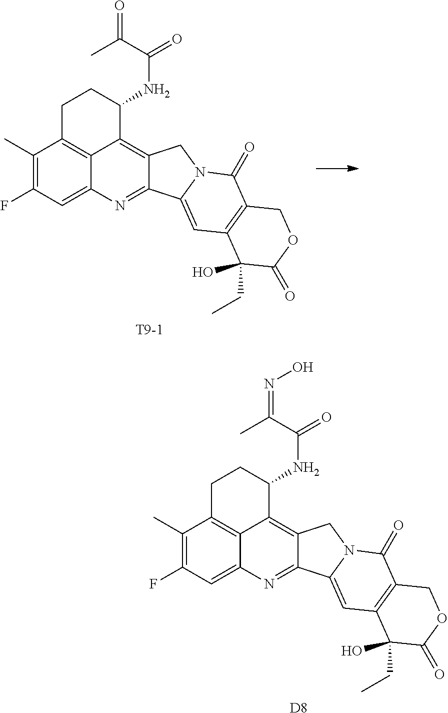 [SMILES: CC[C@@]1(O)C(=O)OCc2c1cc1n(c2=O)Cc2c-1nc1cc(F)c(C)c3c1c2[C@@H](NC(=O)C(C)=O)CC3 ; "T9-1" ; CC[C@@]1(O)C(=O)OCc2c1cc1n(c2=O)Cc2c-1nc1cc(F)c(C)c3c1c2[C@@H](NC(=O)/C(C)=N\O)CC3 ; "D8"]

### Preparation of Compound D8

To a solution of T9-1 (3.0 mg, 0.006 mmol), hydroxylamine hydrochloride (1.26 mg, 0.018 mmol) in anhydrous DMF (0.5 mL) at room temperature under N₂ atmosphere was treated with triethylamine (TEA, 2.48 μL, 0.018 mmol) and stirred for 2 hours at 50° C. When the reaction was completed, the reaction mixture was purified by prep HPLC to obtain compound D8 (0.61 mg, 20%).

ESI-MS m/z: 521 (M⁺+1).

### Example 8: Preparation of Compound D21

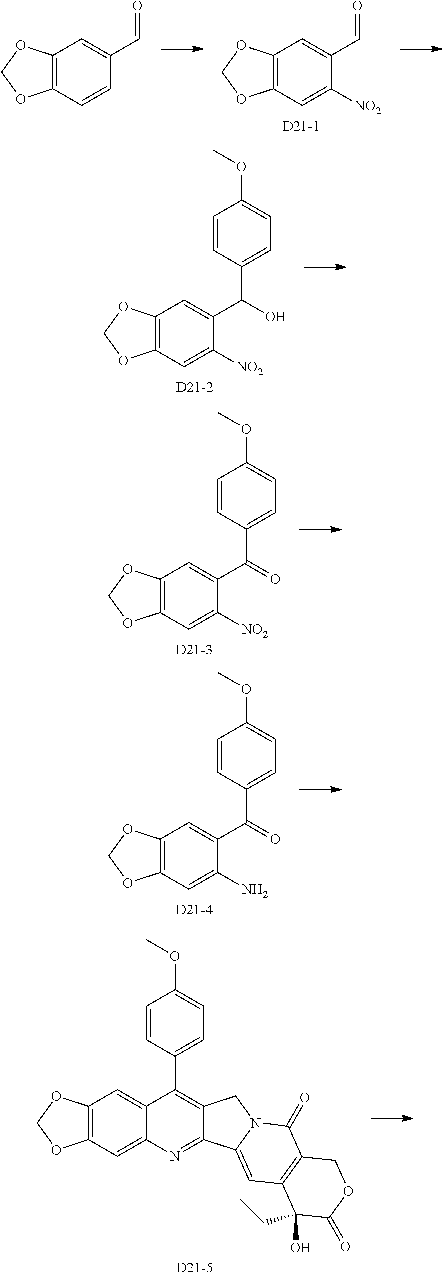

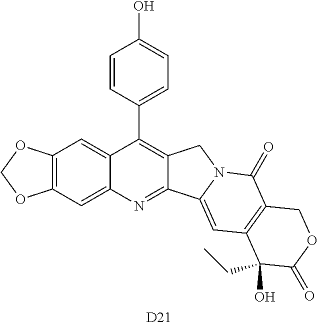 [SMILES: CC[C@@]1(O)C(=O)OCc2c1cc1n(c2=O)Cc2c-1nc1cc3c(cc1c2-c1ccc(O)cc1)OCO3 ; "D21"]

### Preparation of Compound D21-1

To a solution of piperonal (1.0 g, 6.66 mmol) in HNO₃ (3.3 mL) at r.t under N₂ atmosphere. The reaction mixture was stirred at r.t for 20 minutes. After reaction completed, cold water was added. The solid was filtered and washed by water and hexane. The filtered solid was dried over using high vacuum to obtain D21-1 (1.2 g, 95%, yellow solid).

¹H NMR (400 MHz, DMSO-d6) δ 10.12 (s, 1H), 7.79 (s, 1H), 7.16 (s, 1H), 6.38 (s, 1H)

### Preparation of Compound D21-2

To a solution of D21-1 (500 mg, 2.56 mmol) in THF (4 mL) was added (4-Methylphenyl) magnesium bromide solution 0.5 M in THF (10.2 mL, 5.12 mmol) at r.t. for 2.5 hr. After reaction completed, the mixture was quenched with 2N HCl adjusted to pH 2.0 and extracted with EA and washed with brine. The organic layer was dried over anh.Na2SO4, filtered, concentrated under reduced pressure. The residue was purified by column chromatography (EA/HEX 1/3) to obtain D21-2 (447 mg, 57%, black solid).

¹H NMR (400 MHz, CDCl₃) δ 7.49 (s, 1H), 7.27 (d, 1H, 1.6 Hz), 7.21 (s, 1H), 6.86 (m, 2H), 6.41 (d, 2H, 4.4 Hz), 6.12 (m, 2H), 3.79 (s, 3H)

ESI-MS m/z: 629 (2M⁺+Na).

### Preparation of Compound D21-3

To a solution of D21-2 (440 mg, 1.45 mmol) in Acetone (5.3 mL) was added jone's reagent (0.8 mL) at 0° C. for 30 min. After reaction was completed, reagent was quenched with IPA and solid was removed by celite filter and filterate was concentrated under reduced pressure, The mixture was extracted with EA and washed with brine. The organic layer was dried over anh.Na2SO4, filtered, concentrated under reduced pressure to obtain D21-3 (390 mg, 89%, brown solid).

¹H NMR (400 MHz, CDCl₃) δ 7.72 (d, 2H, 8.8 Hz), 7.66 (s, 1H), 6.91 (d, 2H, 9.2 Hz), 6.81 (s, 1H), 6.20 (s, 2H), 3.86 (s, 3H)

ESI-MS m/z: 302.65 (M⁺+H).

### Preparation of Compound D21-4

To a solution of D21-3 (390 mg, 1.21 mmol) in EtOH/H₂O (1:1, 6.1 mL) was added Fe powder (407 mg, 7.32 mmol), NH4Cl (392 mg, 7.32 mmol) at 60-80° C. for 1 hr. After reaction was completed, Fe powder was removed by celite filter and filterate was concentrated under reduced pressure, The mixture was extracted with EA and washed with brine. The organic layer was dried over anh.Na₂SO₄, filtered, concentrated under reduced pressure to obtain D21-4 (310.4 mg, 88%, yellow solid).

¹H NMR (400 MHz, CDCl₃) δ 7.66 (s, 1H), 7.41 (s, 1H), 7.30 (m, 1H), 7.26 (m, 1H), 7.19 (m, 1H), 6.83 (s, 1H), 6.22 (s, 2H), 3.86 (s, 3H) ESI-MS m/z: 272.65 (M⁺+H).

### Preparation of Compound D21-5

To a solution of D21-4 (100 mg, 0.37 mmol) in toluene (6.6 mL) was added (4S)-4-ethyl-7,8-dihydro-4-hydroxy-1H-pyrano[3,4-f]indolizine-3,6,10 (4H)-trione (80.7 mg, 0.307 mmol), PPTS (77 mg, 0.307 mmol) at 120° C. for 19 hr. After reaction was completed, the mixture was concentrated under reduced pressure, the residue was purified by column chromatography (EA/HEX 1/1) to obtain D21-5 (117.4 mg, 77%, yellow solid).

¹H NMR (400 MHz, CDCl₃) δ 7.63 (s, 1H), 7.60 (d, 2H, 8.8 Hz), 7.32 (s, 1H), 7.25 (d, 2H, 8.8 Hz), 7.10 (s, 1H), 6.54 (s, 1H), 6.31 (s, 2H), 5.44 (s, 2H), 5.01 (s, 2H), 3.93 (s, 3H), 1.91 (m, 2H), 0.91 (m, 3H)

ESI-MS m/z: 499.85 (M⁺+H)

### Preparation of Compound D21

To a solution of D21-5 (96 mg, 0.19 mmol) in 48w % HBr solution (3.5 mL) at 120° C. for 1 hr. After reaction was completed, the mixture was cool down to 0° C. and ice water was added. The mixture was extracted with EA and washed with brine. The organic layer was dried over anh.Na2SO4, filtered, concentrated under reduced pressure. The residue was purified by prepHPLC to obtain D21 [1.99 mg (99% purity, yellow solid), 12.1 mg (88% purity, yellow solid), 38.9 mg (purity 76%, brown solid), 15 mg (purity 78.7%, orange solid), 12.1 mg (purity 78.7%, yellow solid), total 86%]

¹H NMR (400 MHz, DMSO-d6) δ 7.57 (s, 1H), 7.43 (d, 2H, 8.8 Hz), 7.27 (s, 1H), 7.08 (s, 1H), 7.02 (d, 2H, 8.8 Hz), 6.26 (m, 2H), 5.39 (s, 2H), 5.04 (s, 2H), 5.01 (s, 2H), 1.85 (m, 2H), 0.87 (m, 3H)

ESI-MS m/z: 485.75 (M⁺+H).

### Example 9: Preparation of Compound D27

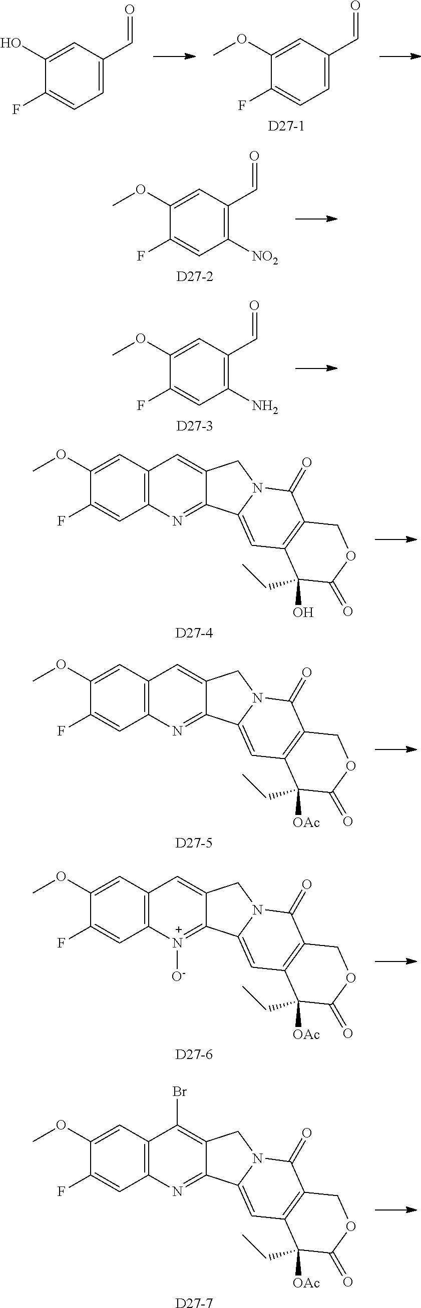

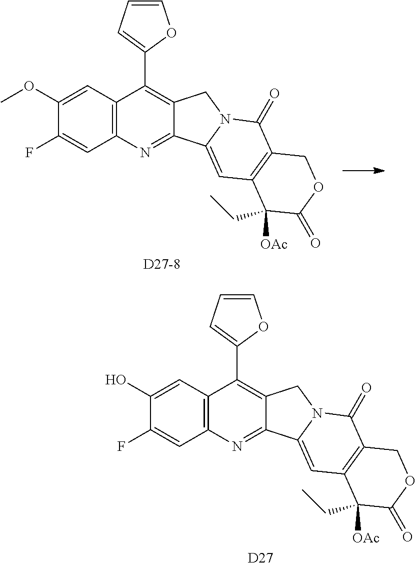 [SMILES: CC[C@@]1(OC(C)=O)C(=O)OCc2c1cc1n(c2=O)Cc2c-1nc1cc(F)c(OC)cc1c2-c1ccco1 ; "D27-8" ; CC[C@@]1(OC(C)=O)C(=O)OCc2c1cc1n(c2=O)Cc2c-1nc1cc(F)c(O)cc1c2-c1ccco1 ; "D27"]

### Preparation of Compound D27-1

To a solution of 4-fluoro-3-hydroxy benzaldehyde (10.0 g, 71.4 mmol) in acetone (79 mL) was added K₂CO₃ (14.8 g, 107.1 mmol) and dimethyl sulfate (6.9 mL, 72.8 mmol) at room temperature under N₂ atmosphere. The reaction mixture was stirrer at 60° C. for 3 hours. After the reaction was completed, reaction mixture was cooled to room temperature, and diluted with distilled water (200 mL) and extracted with DCM (100 mL×4). The combined organic layer was washed with saturated aqueous NaHCO₃ (150 mL). The organic layer was dried over anhydrous Na₂SO₄, filtered and concentrated under reduced pressure. The crude compound D27-1 (10.06 g) was used in the next step without further purification.

¹H NMR (400 MHz, CDCl₃) δ 7.52 (dd, J=8.4 Hz, 2.0 Hz, 1H), 7.47-7.43 (m, 1H), 7.24 (dd, J=10.4 Hz, 8.0 Hz, 1H), 3.96 (s, 3H)

### Preparation of Compound D27-2

To a solution of crude compound D27-1 (9.65 g, 62.6 mmol) in TFAA (70 mL) was added Cu(NO₃)₂·2.5H₂O (15.29 g, 65.7 mmol) at 0° C. under N₂ atmosphere. The reaction mixture was stirred at 0° C. for 90 minutes. After the reaction was completed, reaction mixture was diluted with distilled water (150 mL) and extracted with EA (100 mL×3). The combined organic layer was dried over anhydrous Na₂SO₄, filtered and concentrated under reduced pressure. The residue was purified by column chromatography to obtain compound D27-2 (3.37 g, 27%).

¹H NMR (400 MHz, CDCl₃) δ 10.49 (s, 1H), 7.95 (d, J=10.4 Hz, 1H), 7.50 (d, J=8.0 Hz, 1H), 4.06 (s, 3H)

### Preparation of Compound D27-3

To a solution of compound D27-2 (1.5 g, 7.53 mmol) in H₂O/ethanol (19 mL/19 mL) was added Fe (2.52 g, 45.19 mmol) and NH₄Cl (1.60 g, 45.19 mmol) at room temperature. The reaction mixture was stirred at 80° C. for 1 hours. After the reaction was completed, reaction mixture was cooled to room temperature, and filtered through celite and washed with EA. The filtrate was diluted with distilled water (200 mL) and extracted with EA (100 mL×3). The combined organic layer was dried over anhydrous Na₂SO₄, filtered and concentrated under reduced pressure. The residue was purified by column chromatography to obtain compound D27-3 (734 mg, 58%).

¹H NMR (400 MHz, CDCl₃) δ 9.77 (s, 1H), 7.04 (d, J=8.8 Hz, 1H), 6.41 (d, J=12.0 Hz, 1H), 6.04 (br s, 2H), 3.86 (s, 3H) ESI-MS m/z: 170 (M⁺+1).

### Preparation of Compound D27-4

The solution of compound D27-3 (734 mg, 4.34 mmol), (S)-4-ethyl-4-hydroxy-7,8-dihydro-1H-pyrano[3,4-f]indolizine-3,6,10 (4H)-trione (1.37 g, 5.21 mmol), pyridinium p-toluenesulfonate (PPTS, 1.09 g, 4.34 mmol) in anhydrous toluene (87 mL) was stirred at 120° C. for 3 hours under N₂ atmosphere. After the reaction was completed, reaction mixture was concentrated under reduced pressure. The crude mixture was diluted with MeOH/MC (=1/49, 10 mL), and added ACN (30 mL). The mixture was filtered and washed with ACN (10 mL) to filter the resulting solid (pure product). The filtrate was combined with tar, concentrated under reduced pressure. The residue was purified by column chromatography to obtain compound D27-4 (1.22 g, 76%).

¹H NMR (400 MHz, CDCl₃) δ 8.26 (s, 1H), 7.85 (d, J=12.0 Hz, 1H), 7.61 (s, 1H), 7.23 (d, J=8.1 Hz, 1H), 5.75 (d, J=16.4 Hz, 1H), 5.30 (d, J=16.4 Hz, 1H), 5.27 (s, 2H), 4.07 (s, 3H), 2.01 (s, 1H), 1.97-1.82 (m, 2H), 1.04 (t, J=14.8 Hz, 3H)

ESI-MS m/z: 397 (M⁺+1).

### Preparation of Compound D27-5

To a solution of compound D27-4 (150 mg, 0.38 mmol) in acetic anhydride (4.7 mL) was added pyridine (3.0 mL) at room temperature. The reaction mixture was stirred at room temperature for 7 hours. After the reaction was completed, reaction mixture was diluted with 1% CuSO₄ solution (w/w, in distilled water) (15 mL) and extracted with EA (20 mL×3). The combined organic layer was dried over anhydrous Na₂SO₄, filtered and concentrated under reduced pressure. The residue was purified by column chromatography to obtain compound D27-5 (162 mg, 98%).

¹H NMR (400 MHz, CDCl₃) δ 8.27 (s, 1H), 7.85 (d, J=11.6 Hz, 1H), 7.25 (d, J=9.2 Hz, 1H), 7.15 (s, 1H), 5.67 (d, J=17.2 Hz, 1H), 5.40 (d, J=17.2 Hz, 1H), 5.26 (s, 2H), 4.07 (s, 3H), 2.30-2.10 (m, 5H), 0.98 (t, J=14.8 Hz, 3H)

### Preparation of Compound D27-6

To a solution of compound D27-5 (162 mg, 0.37 mmol) in acetic acid (12 mL) was added 35% H₂O₂ solution (3.2 mL, 36.95 mmol) at room temperature. The reaction mixture was stirred at 75° C. for 2 hours. After the reaction was completed, reaction mixture was cooled to room temperature, and concentrated under reduced pressure. The residue was purified by column chromatography to obtain compound D27-6 (110 mg, 66%).

¹H NMR (400 MHz, CDCl₃) δ 8.45 (d, J=11.2 Hz, 1H), 7.92 (s, 1H), 7.77 (s, 1H), 7.28 (d, J=8.0 Hz, 1H), 5.65 (d, J=18.0 Hz, 1H), 5.38 (d, J=18.0 Hz, 1H), 5.27 (s, 2H), 4.08 (s, 3H), 2.32-2.10 (m, 5H), 0.97 (t, J=14.8 Hz, 3H)

### Preparation of Compound D27-7

To a solution of compound D27-6 (110 mg, 0.24 mmol) in DMF (3.0 mL) was oxalyl bromide (51.6 μL, 0.36 mmol) at 0° C. The reaction mixture was stirred at room temperature for 90 minutes. After the reaction was completed, reaction mixture was diluted with distilled water and extracted with EA. The combined organic layer was dried over anhydrous Na₂SO₄, filtered and concentrated under reduced pressure. The residue was purified by column chromatography to obtain compound D27-7 (67 mg, 54%).

¹H NMR (400 MHz, CDCl₃) δ 7.82 (d, J=11.2 Hz, 1H), 7.59 (d, J=8.8 Hz, 1H), 7.10 (s, 1H), 5.68 (d, J=16.8 Hz, 1H), 5.40 (d, J=17.2 Hz, 1H), 5.23 (d, J=2.0 Hz, 2H), 4.13 (s, 3H), 2.30-2.11 (m, 5H), 0.97 (t, J=14.8 Hz, 3H)

### Preparation of Compound D27-8

The solution of compound D27-7 (67 mg, 0.13 mmol), 2-(furan-2-yl)-4,4,5-trimethyl-1,3,2-dioxaborolane (50 mg, 0.26 mmol), CsF (39 mg, 0.26 mmol), Pd(PPh₃)₄ (15 mg, 0.01 mmol) in dioxane/ethanol/H₂O (=5/2/3, 16 mL) was stirred at 120° C. for 2 hours. After the reaction was completed, reaction mixture was diluted with distilled water and extracted with EA. The combined organic layer was dried over anhydrous Na₂SO₄, filtered and concentrated under reduced pressure. The crude compound D27-8 (75 mg) was used in the next step without further purification.

¹H NMR (400 MHz, CDCl₃) δ 7.91-7.83 (m, 4H), 7.15 (s, 1H), 7.10 (d, J=3.6 Hz, 1H), 6.77 (dd, J=3.6 Hz, 2.0 Hz, 1H), 5.68 (d, J=17.2 Hz, 1H), 5.43-5.39 (m, 3H), 4.07 (s, 3H), 2.31-2.13 (m, 5H), 0.98 (t, J=14.8 Hz, 3H)

ESI-MS m/z: 505 (M⁺+1).

### Preparation of Compound D27

The solution of crude compound D27-8 (75 mg) in 48% HBr solution (8 mL) was stirred at 120° C. for 21 hours. After the reaction was completed, reaction mixture was diluted with distilled water and extracted with EA. The combined organic layer was dried over anhydrous Na₂SO₄, filtered and concentrated under reduced pressure. The crude compound D27 (70 mg) was used in the next step without further purification.

¹H NMR (400 MHz, DMSO-d₆) δ 11.08 (br s, 1H), 8.20 (d, J=1.2 Hz, 1H), 8.05 (d, J=9.6 Hz, 1H), 7.98 (d, J=12.0 Hz, 1H), 7.36 (d, J=3.2 Hz, 1H), 7.29 (s, 1H), 6.94 (dd, J=3.6 Hz, 2.0 Hz, 1H), 5.43 (s, 2H), 5.39 (s, 2H), 1.99-1.81 (m, 2H), 0.88 (t, J=14.8 Hz, 3H)

ESI-MS m/z: 449 (M⁺+1).

### Example 10: Preparation of Compound D9 to D20, D22 to D26, and D28 to D49

Compounds were obtained by performing a reaction in a similar method as described in Example 6, Example 8 or Example 9.

TABLE 1

| Compounds | Structure | Analytical Data |
|---|---|---|
| D9 | 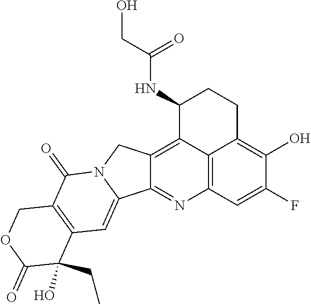 [SMILES: CC[C@@]1(O)C(=O)OCc2c1cc1n(c2=O)Cc2c-1nc1cc(F)c(O)c3c1c2[C@@H](NC(=O)CO)CC3] | ESI-MS m/z: 496(M⁺ + 1). |
| D10 | 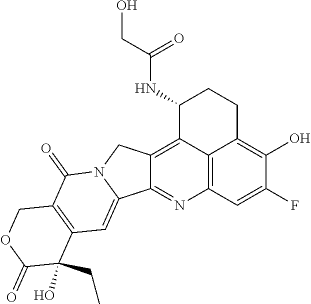 [SMILES: CC[C@@]1(O)C(=O)OCc2c1cc1n(c2=O)Cc2c-1nc1cc(F)c(O)c3c1c2[C@H](NC(=O)CO)CC3] | ESI-MS m/z: 496(M⁺ + 1). |
| D11 | 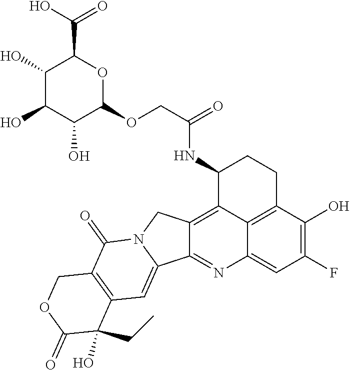 | ESI-MS m/z: 672(M⁺ + 1).- |
| D12 | 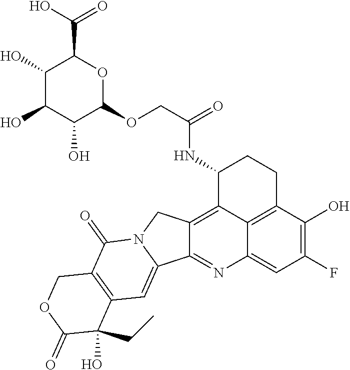 | ESI-MS m/z: 672(M⁺ + 1). |
| D13 | 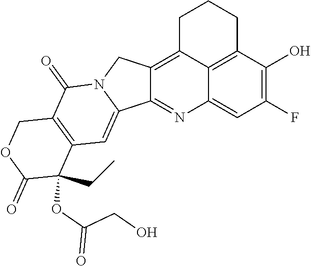 [SMILES: CC[C@@]1(OC(=O)CO)C(=O)OCc2c1cc1n(c2=O)Cc2c-1nc1cc(F)c(O)c3c1c2CCC3] | ESI-MS m/z: 481(M⁺ + 1). |
| D14 | 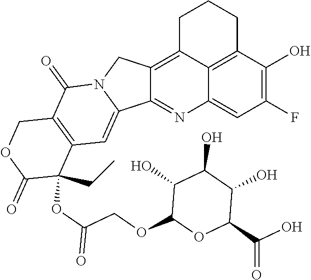 | ESI-MS m/z: 657(M⁺ + 1). |
| D15 | 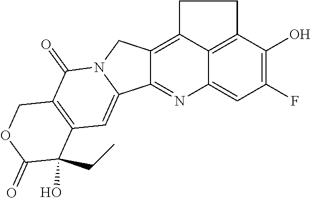 [SMILES: CC[C@@]1(O)C(=O)OCc2c1cc1n(c2=O)Cc2c-1nc1cc(F)c(O)c3c1c2CC3] | ESI-MS m/z: 409(M⁺ + 1). |
| D16 | 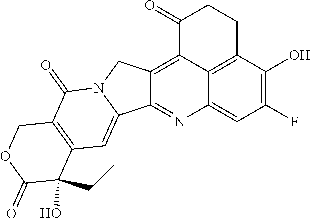 [SMILES: CC[C@@]1(O)C(=O)OCc2c1cc1n(c2=O)Cc2c-1nc1cc(F)c(O)c3c1c2C(=O)CC3] | ESI-MS m/z: 437(M⁺ + 1) |
| D17 | 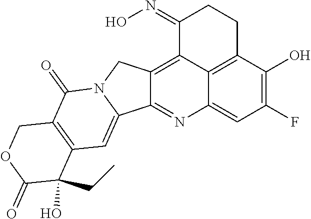 [SMILES: CC[C@@]1(O)C(=O)OCc2c1cc1n(c2=O)Cc2c-1nc1cc(F)c(O)c3c1c2/C(=N\O)CC3] | ESI-MS m/z: 452(M⁺ + 1) |
| D18 | 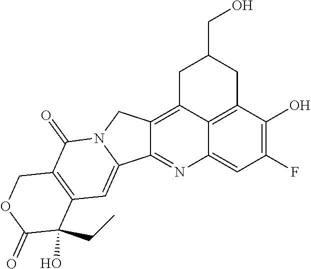 [SMILES: CC[C@@]1(O)C(=O)OCc2c1cc1n(c2=O)Cc2c-1nc1cc(F)c(O)c3c1c2CC(CO)C3] | ESI-MS m/z: 453(M⁺ + 1) |
| D19 | 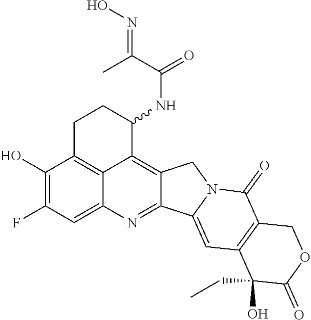 [SMILES: CC[C@@]1(O)C(=O)OCc2c1cc1n(c2=O)Cc2c-1nc1cc(F)c(O)c3c1c2C(NC(=O)/C(C)=N/O)CC3] | ESI-MS m/z: 523(M⁺ + 1) |
| D20 | 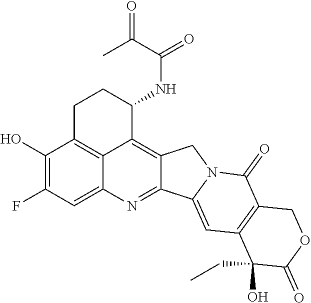 [SMILES: CC[C@@]1(O)C(=O)OCc2c1cc1n(c2=O)Cc2c-1nc1cc(F)c(O)c3c1c2[C@@H](NC(=O)C(C)=O)CC3] | ESI-MS m/z: 508(M⁺ + 1) |
| D21 | 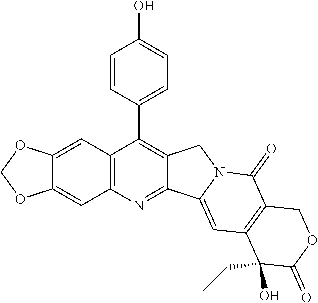 [SMILES: CC[C@@]1(O)C(=O)OCc2c1cc1n(c2=O)Cc2c-1nc1cc3c(cc1c2-c1ccc(O)cc1)OCO3] | ESI-MS m/z: 485(M⁺ + 1) |
| D22 | 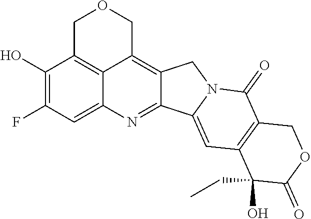 [SMILES: CC[C@@]1(O)C(=O)OCc2c1cc1n(c2=O)Cc2c-1nc1cc(F)c(O)c3c1c2COC3] | ESI-MS m/z: 425(M⁺ + 1) |
| D23 | 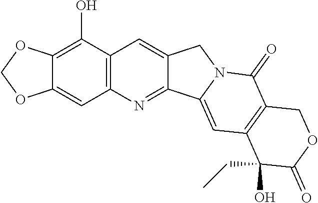 [SMILES: CC[C@@]1(O)C(=O)OCc2c1cc1n(c2=O)Cc2cc3c(O)c4c(cc3nc2-1)OCO4] | ESI-MS m/z: 409(M⁺ + 1) |
| D24 | 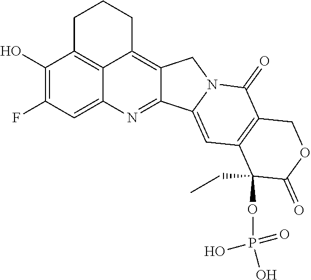 [SMILES: CC[C@@]1(OP(=O)(O)O)C(=O)OCc2c1cc1n(c2=O)Cc2c-1nc1cc(F)c(O)c3c1c2CCC3] | ESI-MS m/z: 503(M⁺ + 1) |
| D25 | 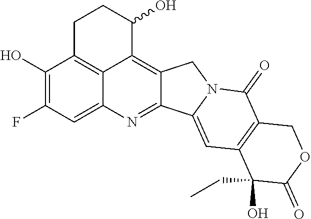 [SMILES: CC[C@@]1(O)C(=O)OCc2c1cc1n(c2=O)Cc2c-1nc1cc(F)c(O)c3c1c2C(O)CC3] | ESI-MS m/z: 439(M⁺ + 1) |
| D26 | 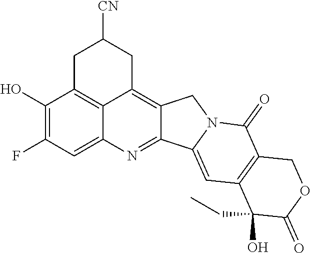 [SMILES: CC[C@@]1(O)C(=O)OCc2c1cc1n(c2=O)Cc2c-1nc1cc(F)c(O)c3c1c2CC(C#N)C3] | ESI-MS m/z: 448(M⁺ + 1) |
| D27 | 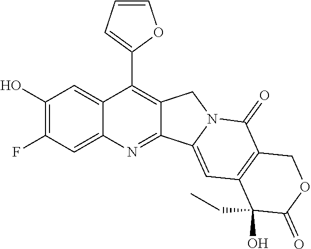 [SMILES: CC[C@@]1(O)C(=O)OCc2c1cc1n(c2=O)Cc2c-1nc1cc(F)c(O)cc1c2-c1ccco1] | ESI-MS m/z: 449(M⁺ + 1) |
| D28 | 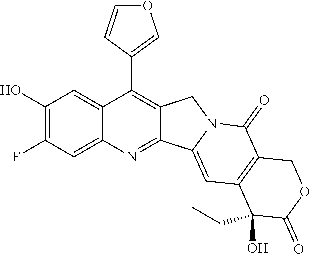 [SMILES: CC[C@@]1(O)C(=O)OCc2c1cc1n(c2=O)Cc2c-1nc1cc(F)c(O)cc1c2-c1ccoc1] | ESI-MS m/z: 449(M⁺ + 1) |
| D29 | 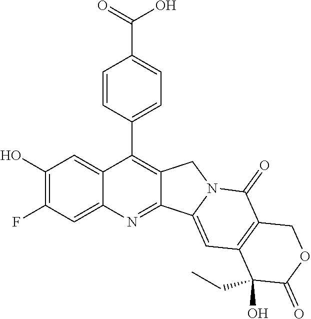 [SMILES: CC[C@@]1(O)C(=O)OCc2c1cc1n(c2=O)Cc2c-1nc1cc(F)c(O)cc1c2-c1ccc(C(=O)O)cc1] | ESI-MS m/z: 503(M⁺ + 1). |
| D30 | 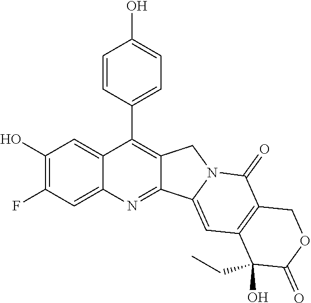 [SMILES: CC[C@@]1(O)C(=O)OCc2c1cc1n(c2=O)Cc2c-1nc1cc(F)c(O)cc1c2-c1ccc(O)cc1] | ESI-MS m/z: 475(M⁺ + 1) |
| D31 | 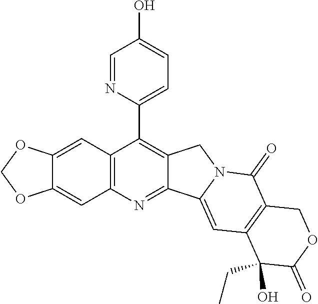 [SMILES: CC[C@@]1(O)C(=O)OCc2c1cc1n(c2=O)Cc2c-1nc1cc3c(cc1c2-c1ccc(O)cn1)OCO3] | ESI-MS m/z: 486(M⁺ + 1) |
| D32 | 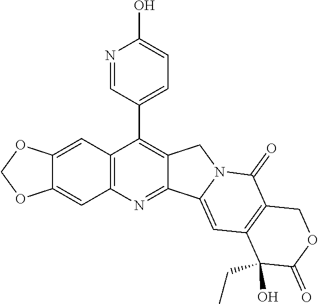 [SMILES: CC[C@@]1(O)C(=O)OCc2c1cc1n(c2=O)Cc2c-1nc1cc3c(cc1c2-c1ccc(O)nc1)OCO3] | ESI-MS m/z: 486(M⁺ + 1) |
| D33 | 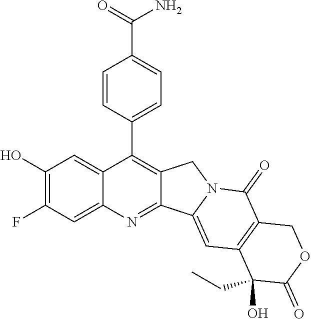 [SMILES: CC[C@@]1(O)C(=O)OCc2c1cc1n(c2=O)Cc2c-1nc1cc(F)c(O)cc1c2-c1ccc(C(N)=O)cc1] | ESI-MS m/z: 502(M⁺ + 1) |
| D34 | 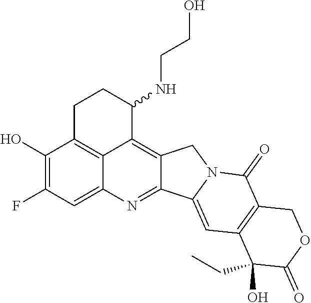 [SMILES: CC[C@@]1(O)C(=O)OCc2c1cc1n(c2=O)Cc2c-1nc1cc(F)c(O)c3c1c2C(NCCO)CC3] | ESI-MS m/z: 482(M⁺ + 1) |
| D35 | 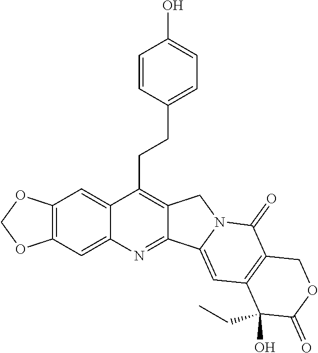 [SMILES: CC[C@@]1(O)C(=O)OCc2c1cc1n(c2=O)Cc2c-1nc1cc3c(cc1c2CCc1ccc(O)cc1)OCO3] | ESI-MS m/z: 513(M⁺ + 1) |
| D36 | 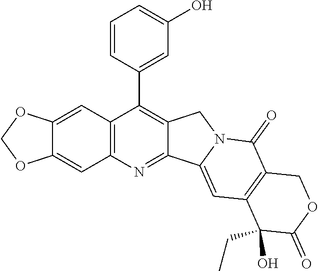 [SMILES: CC[C@@]1(O)C(=O)OCc2c1cc1n(c2=O)Cc2c-1nc1cc3c(cc1c2-c1cccc(O)c1)OCO3] | ESI-MS m/z: 485(M⁺ + 1) |
| D37 | 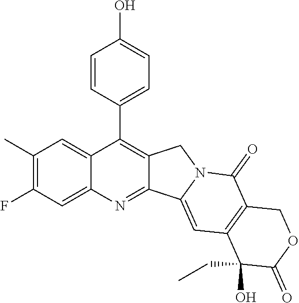 [SMILES: CC[C@@]1(O)C(=O)OCc2c1cc1n(c2=O)Cc2c-1nc1cc(F)c(C)cc1c2-c1ccc(O)cc1] | ESI-MS m/z: 473(M⁺ + 1) |
| D38 | 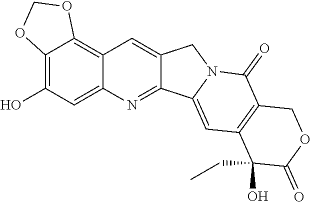 [SMILES: CC[C@@]1(O)C(=O)OCc2c1cc1n(c2=O)Cc2cc3c4c(c(O)cc3nc2-1)OCO4] | ESI-MS m/z: 409(M⁺ + 1) |
| D39 | 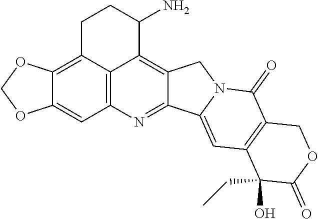 [SMILES: CC[C@@]1(O)C(=O)OCc2c1cc1n(c2=O)Cc2c-1nc1cc3c(c4c1c2C(N)CC4)OCO3] | ESI-MS m/z: 448(M⁺ + 1) |
| D40 | 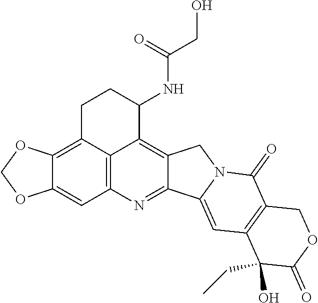 [SMILES: CC[C@@]1(O)C(=O)OCc2c1cc1n(c2=O)Cc2c-1nc1cc3c(c4c1c2C(NC(=O)CO)CC4)OCO3] | ESI-MS m/z: 506(M⁺ + 1) |
| D41 | 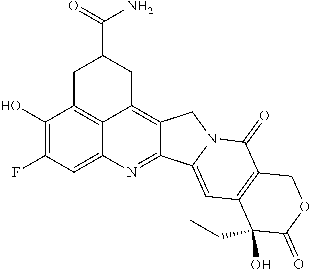 [SMILES: CC[C@@]1(O)C(=O)OCc2c1cc1n(c2=O)Cc2c-1nc1cc(F)c(O)c3c1c2CC(C(N)=O)C3] | ESI-MS m/z: 466(M⁺ + 1) |
| D42 | 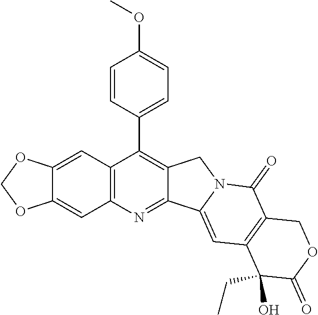 [SMILES: CC[C@@]1(O)C(=O)OCc2c1cc1n(c2=O)Cc2c-1nc1cc3c(cc1c2-c1ccc(OC)cc1)OCO3] | ESI-MS m/z: 499(M⁺ + 1). |
| D43 | 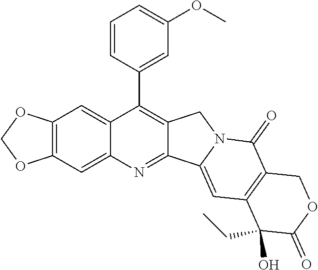 [SMILES: CC[C@@]1(O)C(=O)OCc2c1cc1n(c2=O)Cc2c-1nc1cc3c(cc1c2-c1cccc(OC)c1)OCO3] | ESI-MS m/z: 499(M⁺ + 1). |
| D44 | 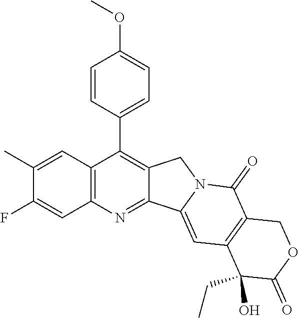 [SMILES: CC[C@@]1(O)C(=O)OCc2c1cc1n(c2=O)Cc2c-1nc1cc(F)c(C)cc1c2-c1ccc(OC)cc1] | ESI-MS m/z: 487(M⁺ + 1). |
| D45 | 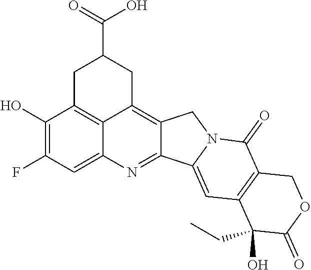 [SMILES: CC[C@@]1(O)C(=O)OCc2c1cc1n(c2=O)Cc2c-1nc1cc(F)c(O)c3c1c2CC(C(=O)O)C3] | ESI-MS m/z: 467(M⁺ + 1) |
| D46 | 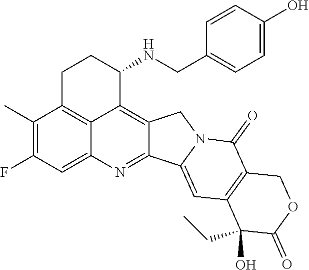 [SMILES: CC[C@@]1(O)C(=O)OCc2c1cc1n(c2=O)Cc2c-1nc1cc(F)c(C)c3c1c2[C@@H](NCc1ccc(O)cc1)CC3] | ESI-MS m/z: 542(M⁺ + 1). |
| D47 | 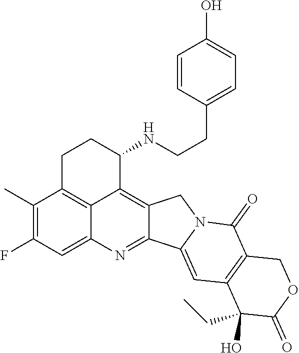 [SMILES: CC[C@@]1(O)C(=O)OCc2c1cc1n(c2=O)Cc2c-1nc1cc(F)c(C)c3c1c2[C@@H](NCCc1ccc(O)cc1)CC3] | ESI-MS m/z: 556(M⁺ + 1). |
| D48 | 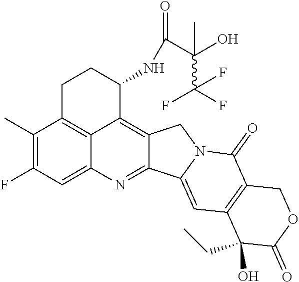 [SMILES: CC[C@@]1(O)C(=O)OCc2c1cc1n(c2=O)Cc2c-1nc1cc(F)c(C)c3c1c2[C@@H](NC(=O)C(C)(O)C(F)(F)F)CC3] | ESI-MS m/z: 576(M⁺ + 1). |
| D49 | 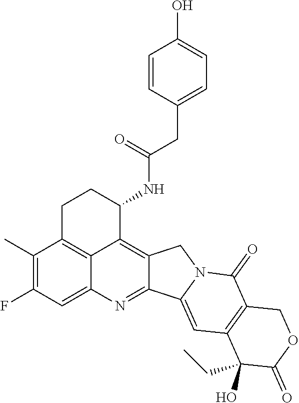 [SMILES: CC[C@@]1(O)C(=O)OCc2c1cc1n(c2=O)Cc2c-1nc1cc(F)c(C)c3c1c2[C@@H](NC(=O)Cc1ccc(O)cc1)CC3] | ESI-MS m/z: 570(M⁺ + 1). |

### Example 11: Preparation of Compound Int-L1

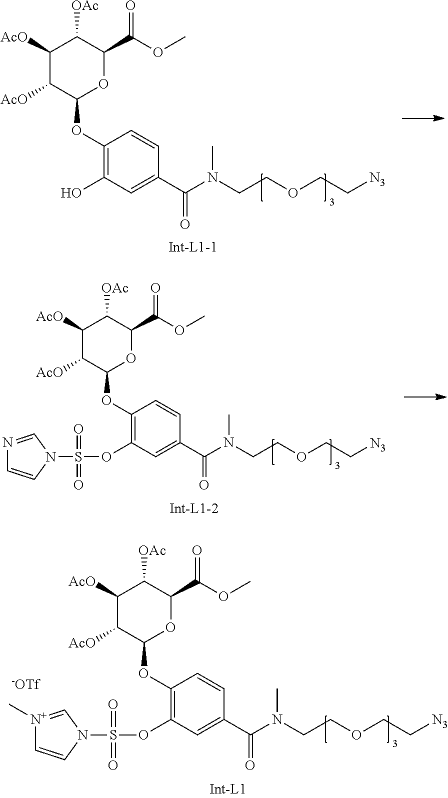

### Preparation of Compound Int-L1-1

Int-L1-1 was obtained by performing a reaction in a similar method as described in Example 77 Int-TG104-4 of document US 2023-0190939 A1 (15 g, 82%).

ESI-MS m/z: 685 (M⁺+1).

### Preparation of Compound Int-L1-2

To a solution of compound Int-L1-1 (15 g, 0.022 mol) and 1-((1H-imidazol-1-yl)sulfonyl)-3-methyl-1H-imidazol-3-ium trifluoromethanesulfonate (9.5 g, 0.026 mol) in anhydrous DCM (110 mL) at room temperature under N₂ atmosphere was treated with 2,6-lutidine (5.1 mL, 0.044 mol) and stirred for 1 hour. The reaction mixture was extracted with EA (700 mL) and saturated aqueous citric acid (500 mL). The organic layer was dried over anhydrous Na₂SO₄, filtered and concentrated under reduced pressure. The residue was purified by column chromatography to obtain compound Int-L1-2 (11.86 g, 67%).

ESI-MS m/z: 815 (M⁺+1).

### Preparation of Compound Int-L1

To a solution of compound Int-L1-2 (6.1 g, 7.48 mmol) in anhydrous DCM (15.0 mL) at 0° C. under N₂ atmosphere was treated with methyl triflate (982.9 μL, 8.98 mmol) and stirred for 1 hour. After 1 hour, the cool ether (100 mL) was added in reaction mixture. The solid was decantation and dry over with vacuum to obtain compound Int-L1 (7.26 g, 99%).

ESI-MS m/z: 829 (M⁺) free form.

### Example 12: Preparation of Compound Int-L2

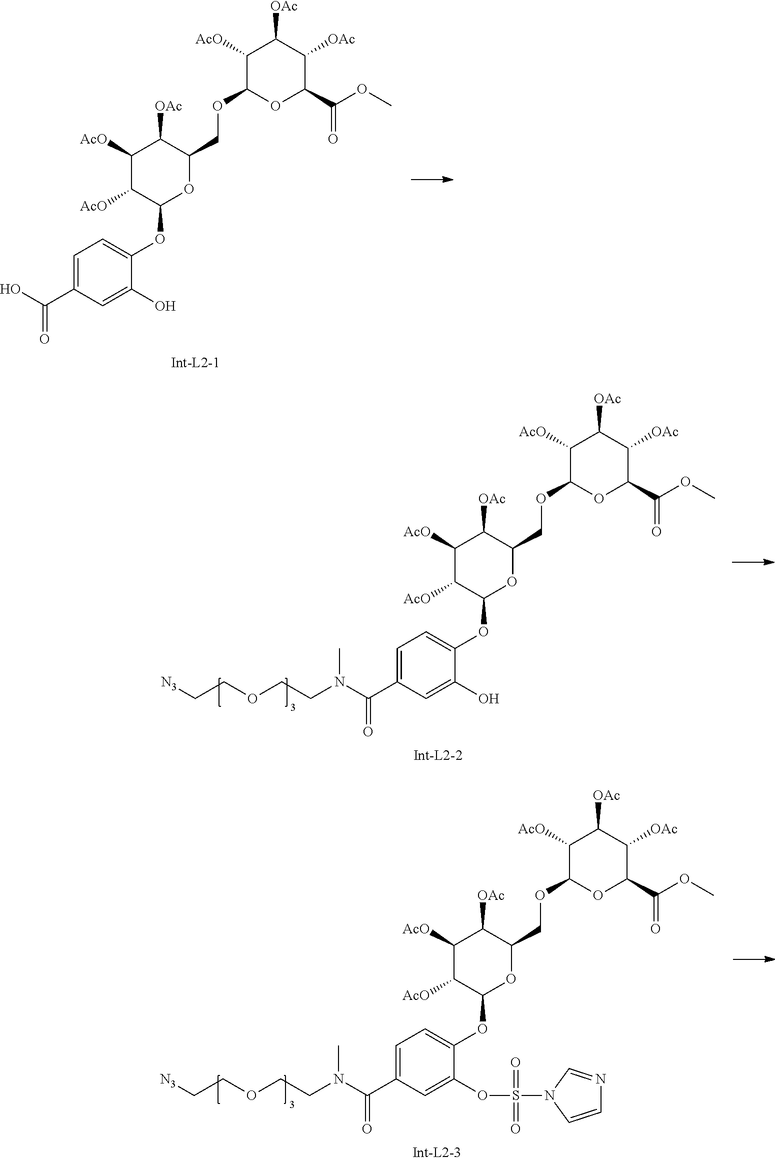

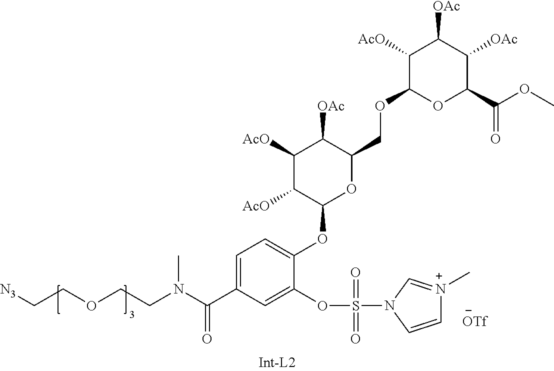

### Preparation of Compound Int-L2-1

Int-L2-1 was obtained by performing a reaction in a similar method as described in Example 11 Int-TG6-2 of document U.S. patent application Ser. No. 18/084,739 (27.3 mg, 81%).

ESI-MS m/z: 759 (M⁺+1).

### Preparation of Compound Int-L2-2

Int-L2-2 was obtained by performing a reaction in a similar method as described in Example 11 Int-TG7 of document U.S. patent application Ser. No. 18/084,739 (24.1 mg, 87%).

ESI-MS m/z: 973 (M⁺+1).

### Preparation of Compound Int-L2-3

Int-L2-3 was obtained by performing a reaction in a similar method as described in Example 11 compound Int-L1-2 (114 mg, 90%).

ESI-MS m/z: 1103 (M⁺).

### Preparation of Compound Int-L2

Int-L2 was obtained by performing a reaction in a similar method as described in Example 11 compound Int-L1 (130 mg, quantitative).

ESI-MS m/z: 1117 (M⁺).

### Example 13: Preparation of Compound Int-L3

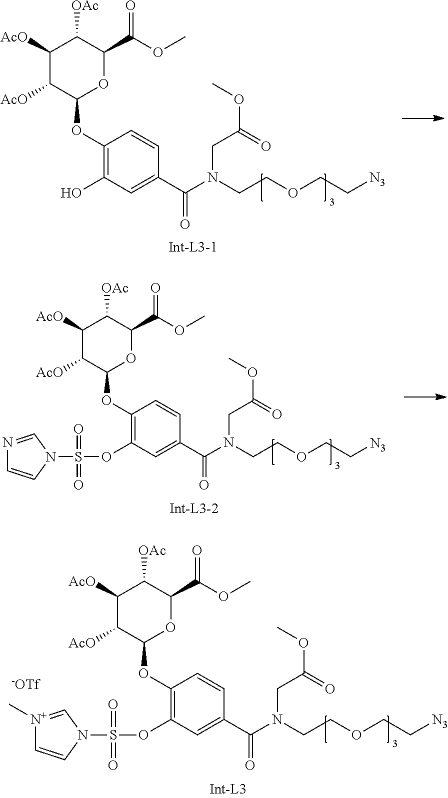

### Preparation of Compound Int-L3-1

Int-L3-1 was obtained by performing a reaction in a similar method as described in Example 11 for compound Int-L1-1 (100 mg, 27%).

ESI-MS m/z: 743 (M⁺+1).

### Preparation of Compound Int-L3-2

Int-L3-2 was obtained by performing a reaction in a similar method as described in Example 11 for compound Int-L1-2 (101 mg, 85%).

ESI-MS m/z: 873 (M⁺+1).

### Preparation of Compound Int-L3

Int-L3 was obtained by performing a reaction in a similar method as described in Example 11 for compound Int-L1 (104 mg, 87%).

ESI-MS m/z: 887 (M⁺+1).

### Example 14: Preparation of Compound Int-L4

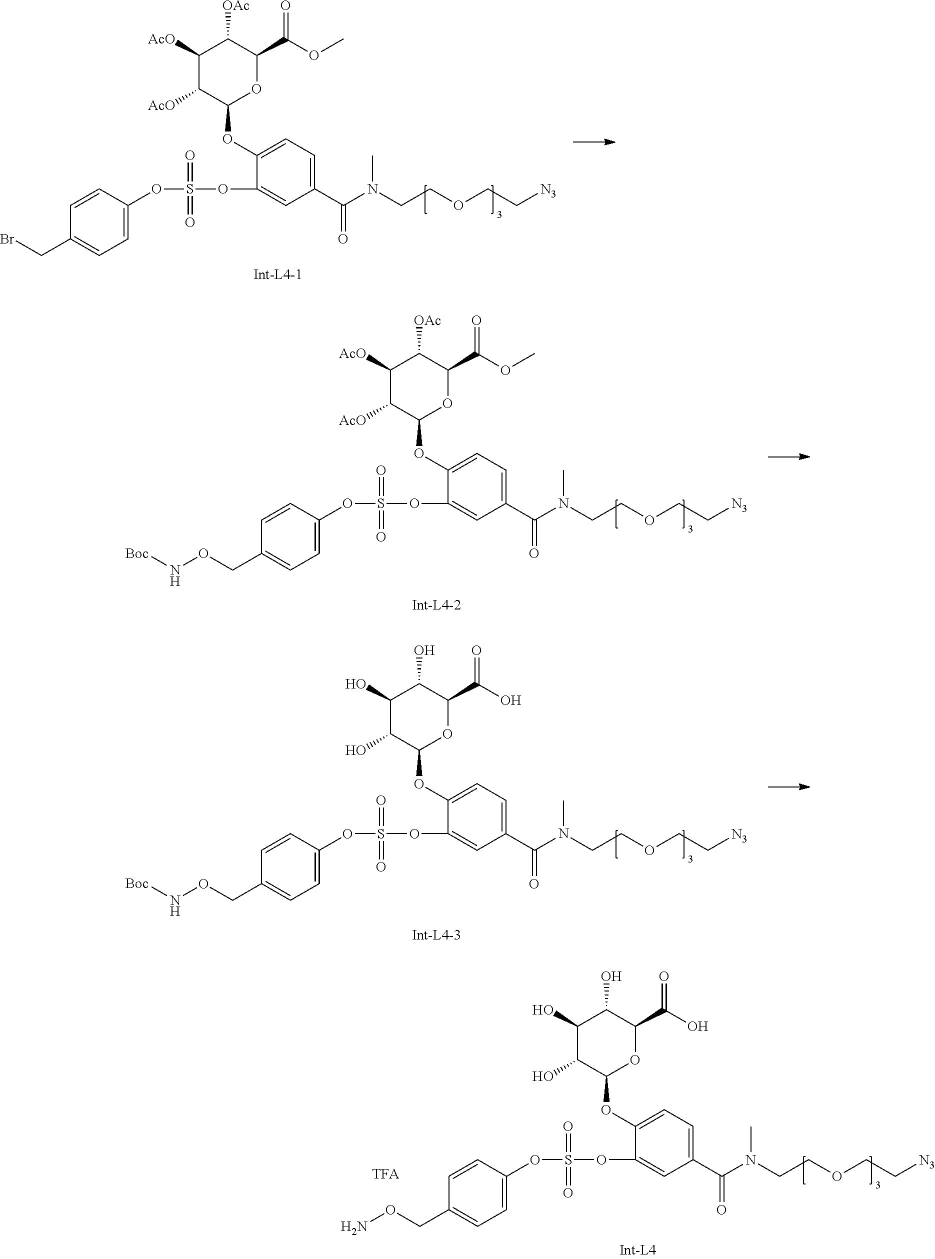

### Preparation of Compound Int-L4-1

Int-L4-1 was obtained by performing a reaction in a similar method as described in Example 3.7 OHPAS-D9 of document US 2022-0047717 A1 (50 mg, 97%).

ESI-MS m/z: 934 (M⁺+1).

### Preparation of Compound Int-L4-2

To a solution of Int-L4-1 (50 mg, 0.054 mmol), N-Boc-Hydroxylamine (8.6 mg, 0.064 mmol) in anhydrous ACN (0.54 mL) at room temperature under N₂ atmosphere was treated with K₂CO₃ (8.9 mg, 0.064 mmol) and stirred for 18 hours. The DMF (0.3 mL) was added in reaction mixture and stirred for 3 hours. When the reaction was completed, the mixture was extracted with EA and distilled water. The organic layer was dried over anhydrous Na₂SO₄, filtered and concentrated under reduced pressure. The residue was purified by prep TLC to obtain compound Int-L4-2 (25.6 mg, 48%).

ESI-MS m/z: 986 (M⁺+1).

### Preparation of Compound Int-L4-3

To a solution of Int-L4-2 (25.6 mg, 0.026 mmol) in THF (0.6 mL), MeOH (0.4 mL), distilled water (0.2 mL) at −30° C. under N₂ atmosphere was treated with Lithium hydroxide monohydrate (5.4 mg, 0.13 mmol) and stirred for 2.5 hours. When the reaction was completed, the mixture was quenched with 2N HCl, and concentrated under reduced pressure. The residue was purified by prep HPLC to obtain compound Int-L4-3 (7.3 mg, 33%).

ESI-MS m/z: 846 (M⁺+1).

### Preparation of Compound Int-L4

To a solution of Int-L4-3 (7.2 mg, 0.009 mmol) in anhydrous DCM (0.5 mL) at 0° C. under N₂ atmosphere was treated with TFA (0.15 mL) and stirred for 45 minutes. When the reaction was completed, the reaction mixture was concentrated under reduced pressure to obtain compound Int-L4 (7.3 mg, quant).

ESI-MS m/z: 746 (M⁺+1).

### Example 15: Preparation of Compound Int-L5

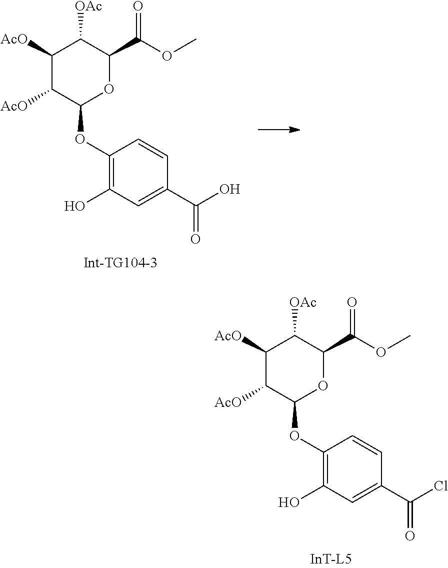 [SMILES: COC(=O)[C@H]1O[C@@H](Oc2ccc(C(=O)O)cc2O)[C@H](OC(C)=O)[C@@H](OC(C)=O)[C@@H]1OC(C)=O ; COC(=O)[C@H]1O[C@@H](Oc2ccc(C(=O)Cl)cc2O)[C@H](OC(C)=O)[C@@H](OC(C)=O)[C@@H]1OC(C)=O]

To a solution of Compound Int-TG104-3 (469 mg, 1.00 mmol, in Example 77 Int-TG104-3 of document US 2023-0190939 A1) in anhydrous THF (483 mL) at room temperature under N₂ atmosphere, Oxalyl chloride (153.9 uL, 1.80 mmol), DMF (1 drop) was added and stirred for 30 min. The reaction mixture was concentrated under reduced pressure to obtain compound Int-L5. The Int-L5 compound was used in the next step of the reaction.

### Example 16: Preparation of Compound Int-L6 and Int-L7

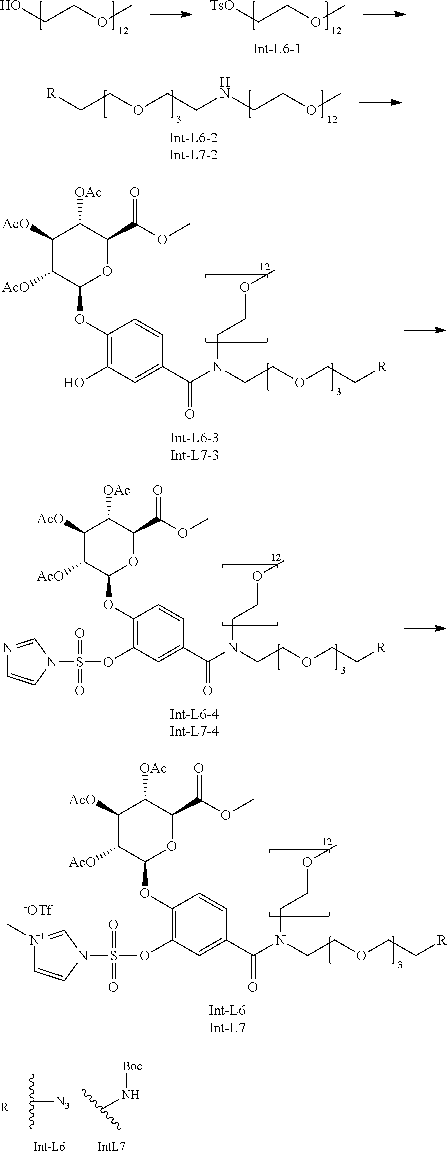

### Preparation of Compound Int-L6-1

To a solution of Dodecaethylene Glycol Monomethyl Ether (BLD, CAS: 2050595-03-2, 5.0 g, 8.90 mmol) in anhydrous DCM (180 mL) at room temperature under N₂ atmosphere, TEA (1.25 mL, 8.90 mmol), DMAP (653.7 mg, 5.35 mmol), p-TsCl (2.0 g, 10.7 mmol) was added and stirred for overnight. The reaction mixture was concentrated under reduced pressure. The residue was purified by column chromatography (MeOH in DCM 0%-5%) to obtain compound Int-L6-1 (5.26 g, 80%).

¹H NMR ¹H NMR (400 MHz, CDCl₃) δ 2.43 (s, 3H), 3.37 (s, 3H), 3.64 (m, 46H) 4.15 (t, 2H), 7.34 (d, 2H), 7.85 (d, 2H).

### Preparation of Compound Int-L6-2

To a solution of Compound Int-L6-1 (3.55 g, 4.97 mmol) in 1,4-Dioxane (99 mL) at room temperature under N₂ atmosphere, K₂CO₃ (2.75 g, 19.9 mmol) and 2-(2-(2-(2-azidoethoxy)ethoxy)ethoxy)ethan-1-amine hydrochloride (Achem Block, 777081-35-3, 3.80 g, 14.9 mmol) were added and stirred for overnight at 70° C. After overnight the TEA (2.76 mL, 19.9 mmol) was added in reaction mixture and stirred for 3 days at 70° C. After 3 days the reaction mixture was extracted with EA/H₂O. The organic layer was dried over with anhydrous Na₂SO₄, filtered and concentrated under reduced pressure. The residue was purified by column chromatography to obtain compound Int-L6-2 (1.46 g, 39%).

ESI-MS m/z: 761 (M⁺+1).

### Preparation of Compound Int-L6-3

To a solution of Compound Int-L6-2 (730 mg, 0.96 mmol) in anhydrous THF (19.2 mL) at room temperature under N₂ atmosphere, DIPEA (334.2 uL, 1.92 mmol) and solution of compound Int-L5 (469 mg, 1.06 mmol) in THF was added and stirred for 30 minutes. The reaction mixture was extracted with EA (400 mL×2)/H₂O (40 mL). The organic layer was dried over with anhydrous Na₂SO₄, filtered and concentrated under reduced pressure. The residue was purified by column chromatography (MeOH in DCM 0%-10%) to obtain compound Int-L6-3 (527 mg, 45%).

ESI-MS m/z: 1214 (M⁺+1).

### Preparation of Compound Int-L6-4

Int-L2-3 was obtained by performing a reaction in a similar method as described in Example 11 compound Int-L1-2 (259.65 mg, 55.4%).

ESI-MS m/z: 1343 (M⁺).

### Preparation of Compound Int-L6

Int-L2-3 was obtained by performing a reaction in a similar method as described in Example 11 compound Int-L1 (80.35 mg, 77.5%).

ESI-MS m/z: 1358 (M⁺) free form.

### Preparation of Compound Int-L7-2

Int-L2-3 was obtained by performing a reaction in a similar method as described in the preparation of compound Int-L6-2 (331 mg, 48%).

ESI-MS m/z: 836 (M⁺+1).

### Preparation of Compound Int-L7-3

Int-L2-3 was obtained by performing a reaction in a similar method as described in the preparation of compound Int-L6-3 (196.3 mg, 49%).

ESI-MS m/z: 1287 (M⁺).

### Preparation of Compound Int-L7-4

Int-L2-3 was obtained by performing a reaction in a similar method as described in the preparation of compound Int-L6-4 (149.7 mg, 57%).

ESI-MS m/z: 1417 (M⁺).

### Preparation of Compound Int-L7

Int-L2-3 was obtained by performing a reaction in a similar method as described in the preparation of compound Int-L6 (118.2 mg, 74.5%).

ESI-MS m/z: 1432 (M⁺) free form.

### Example 17: Preparation of Compound Mal-1

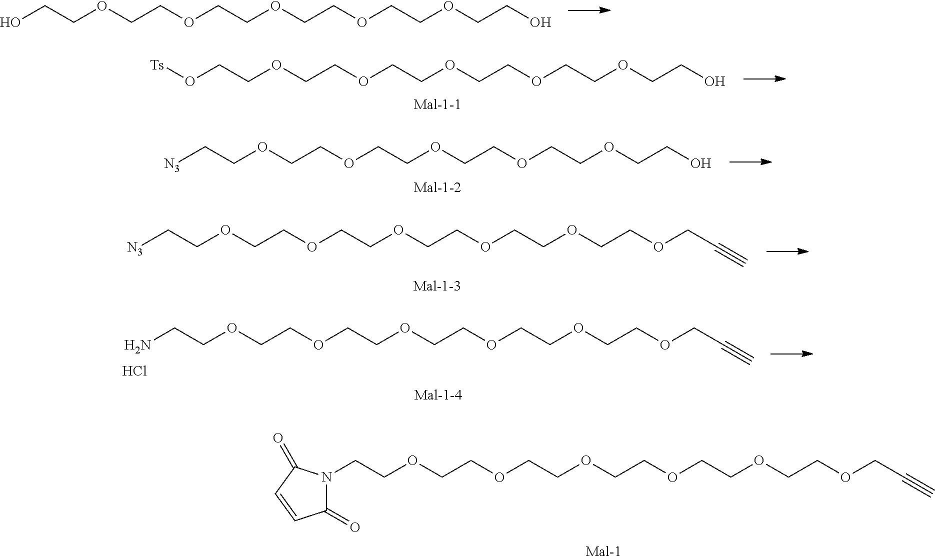

### Preparation of Compound Mal-1-1

To a solution of hexaethyleneglycol (10.0 g, 35.4 mmol) in anhydrous DCM (354 mL) was added Ag₂O (9.87 g, 42.5 mmol), p-TsCl (7.42 g, 38.9 mmol) and KI (588 mg, 3.54 mmol) at room temperature under N₂ atmosphere. The reaction mixture was stirred at room temperature under N₂ atmosphere for 16 hours. The reaction mixture was filtered through celite pad and filtrate was concentrate under reduced pressure. The crude product was purified by column chromatography to obtain compound Mal-1-1 (10.2 g, 66%).

¹H NMR (400 MHz, CDCl₃) δ 7.80 (d, J=8.4 Hz, 2H), 7.34 (d, J=8.4 Hz, 2H), 4.16 (t, J=4.8 Hz, 2H), 3.71-3.58 (m, 22H), 2.45 (s, 3H)

### Preparation of Compound Mal-1-2

To a solution of compound Mal-1-1 (58.1 g, 133 mmol) in anhydrous DMF (290 mL) was added sodium azide (13.0 g, 200 mmol) at room temperature under N₂ atmosphere. The reaction mixture was stirred at 110° C. under N₂ atmosphere for 90 minutes. After the reaction was completed, reaction mixture was concentrated under reduced pressure. The residue was purified by column chromatography to obtain compound Mal-1-2 (37.5 g, 92%).

¹H NMR (400 MHz, CDCl₃) δ 3.72-3.60 (m, 22H), 3.19 (t, J=5.0 Hz, 2H)

### Preparation of Compound Mal-1-3

To a solution of compound Mal-1-2 (37.5 g, 122 mmol) in anhydrous DMF (249 mL) was added NaH 60% dispersion in mineral oil (5.87 g, 147 mmol) at 0° C. under N₂ atmosphere. After 30 minutes, Propargyl bromide, 80 wt % solution in toluene (15.1 ml, 159 mmol) was added at 0° C. under N₂ atmosphere. The reaction mixture was stirred at 65° C. under N₂ atmosphere for 3 hours. The reaction mixture was extracted with EA and saturated aqueous NH₄Cl and washed with brine. The obtained organic layer was dried over anhydrous Na₂SO₄, filtered, and concentrated under reduced pressure. The residue was purified by column chromatography to obtain compound Mal-1-3 (35.0 g, 83%).

¹H NMR (400 MHz, CDCl₃) δ 4.20 (d, J=2.4 Hz, 2H), 3.70-3.65 (m, 22H), 3.39 (t, J=5.0 Hz, 2H)

### Preparation of Compound Mal-1-4

To a solution of compound Mal-1-3 (35.0 g, 101 mmol) in EA/ether (338 mL/338 mL) was added 5% HCl solution (676 mL) and triphenyhlphosphine (34.4 g, 131 mmol) at 0° C. under N₂ atmosphere. The reaction mixture was stirred at room temperature under N₂ atmosphere for 41 hours. The reaction mixture was concentrated and washed with anhydrous DCM. The aqueous layer was concentrated under reduced pressure. The crude compound Mal-1-4 (34.9 g) was used in the next step without further purification.

¹H NMR (400 MHz, CDCl₃) δ 7.96 (brs, 2H), 4.25 (d, J=2.4 Hz, 2H), 3.96 (t, J=4.8 Hz, 2H), 3.79-3.67 (m, 20H), 3.18 (q, J=5.0 Hz, 2H), 2.44 (t, J=2.4 Hz, 1H)

### Preparation of Compound Mal-1

To a solution of crude compound Mal-1-4 (34.9 g, 101 mmol) in saturated aqueous NaHCO₃ (337 mL) was stirred at 0° C. for 30 minutes. To a solution was added N-methoxycarbonyl maleimide (20.4 g, 132 mmol) at 0° C. The reaction mixture was stirred at room temperature for 5 hours. The reaction mixture was extracted with EA. The obtained organic layer was dried over anhydrous Na₂SO₄, filtered, and concentrated under reduced pressure. The residue was purified by column chromatography to obtain compound Mal-1 (30.3 g, 2 steps yield=75%).

¹H NMR (400 MHz, CDCl₃) δ 6.70 (s, 2H), 4.20 (d, J=2.4 Hz, 2H), 3.69-3.60 (m, 24H), 2.43 (t, J=2.4 Hz, 1H)

ESI-MS m/z: 400 (M⁺+1).

### Example 18: Preparation of Compound Mal-2

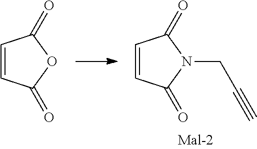

### Preparation of Compound Mal-2

To a solution of maleic anhydride (10 g, 0.102 mol) in acetone (50 ml) was added propargyl amine (5.4 ml, 0.085 mol) at room temperature under N₂ atmosphere. The reaction mixture was stirred at 70° C. under N₂ atmosphere for 20 hours. The reaction mixture was concentrated under reduced pressure. The crude compound in acetic anhydride (40 ml) was added NaOAc (3.48 g, 0.043 mol) at room temperature under N₂ atmosphere. The reaction mixture was stirred at 60° C. under N₂ atmosphere for 5 hours. The reaction mixture was extracted with ether and distilled water. The obtained organic layer was dried over anhydrous Na₂SO₄, filtered, and concentrated under reduced pressure. The compound was vacuum-dried for 15 hours to obtain compound Mal-2 (8.65 g, 2 steps yield=74%).

¹H NMR (400 MHz, CDCl₃) δ 6.77 (s, 2H), 4.30 (d, J=2.4 Hz, 2H), 2.22 (t, J=2.4 Hz, 1H) ESI-MS m/z: 136 (M⁺+1).

### Example 19: Preparation of Compound T1

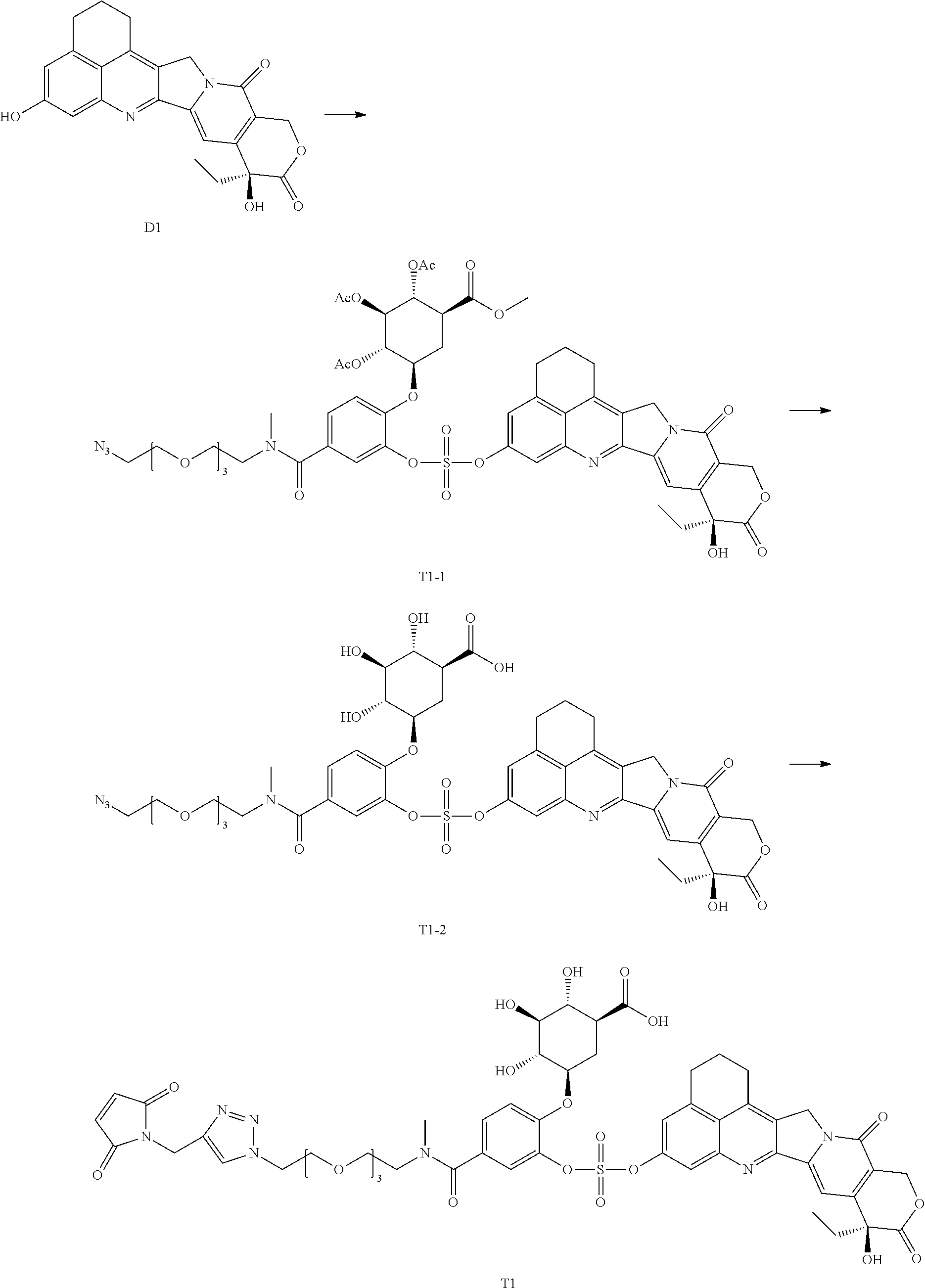

### Preparation of Compound T1-1

To a solution of compound D1 (620 mg, 1.53 mmol) in anhydrous DMF (70 mL) at room temperature under N₂ atmosphere was treated with TEA (1.09 mL, 7.66 mmol), Int-L1 (2.25 g, 2.3 mmol) and stirred for 30 minutes. When the reaction was completed, the mixture was extracted with EA and distilled water. The organic layer was dried over anhydrous Na₂SO₄, filtered and concentrated under reduced pressure. The residue was purified by column chromatography to obtain compound T1-1 (1.38 g, 78%).

ESI-MS m/z: 1151 (M⁺).

### Preparation of Compound T1-2

To a solution of compound T1-1 (980 mg, 0.85 mmol) in THF (6.0 mL), MeOH (5.0 mL), distilled water (4.0 mL) at −15° C. under N₂ atmosphere was treated with lithium hydroxide monohydrate (214.3 mg, 5.1 mmol) and stirred for 2.5 hours. When the reaction was completed, the mixture was quenched with 2 N HCl solution and concentrated under reduced pressure. The residue was purified by prep C18 column to produce compound T1-2 (1.36 g, 82%).

ESI-MS m/z: 1011 (M⁺+1).

### Preparation of Compound T1

To a solution of compound T1-2 (300 mg, 0.30 mmol), Mal-2 (52.12 mg, 0.39 mmol) in degassed DMSO (1.5 mL), distilled water (1.5 mL) at room temperature under N₂ atmosphere was treated with CuBr (55.3 mg, 0.39 mmol) and stirred for 40 minutes. The reaction mixture was purified by prep HPLC to obtain compound T1 (266.6 mg, 78%).

ESI-MS m/z: 1146 (M⁺).

### Example 20: Preparation of Compound T2

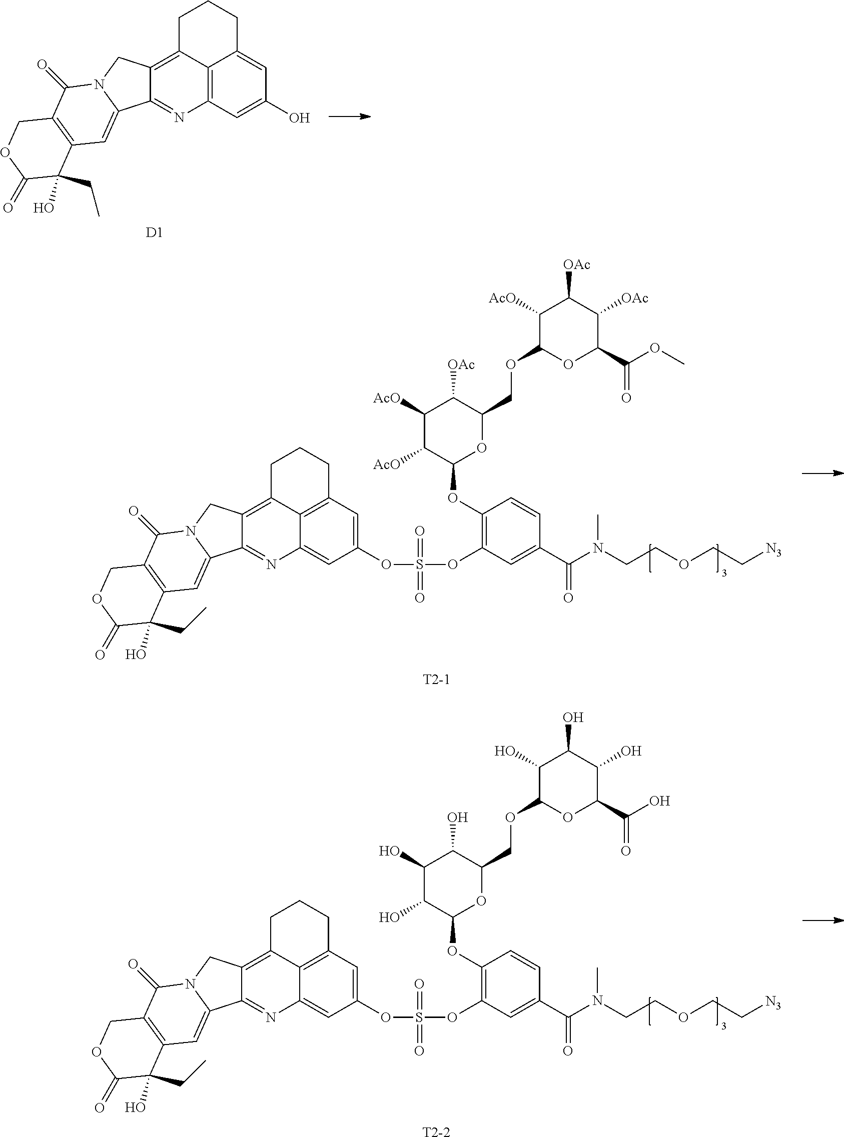

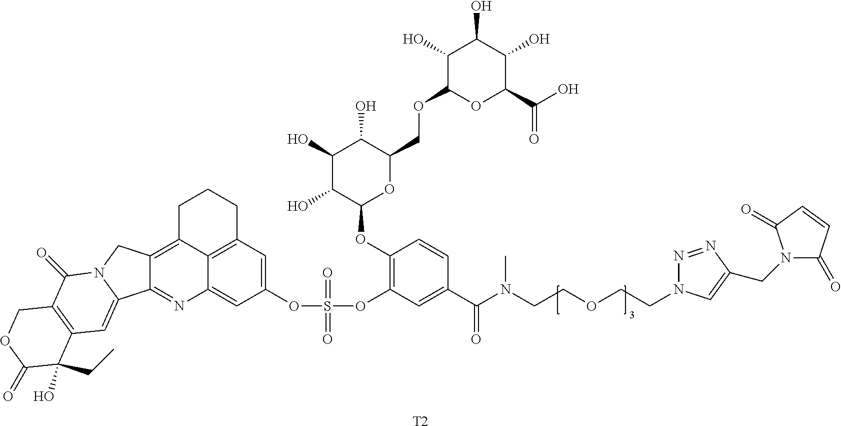

### Preparation of Compound T2-1

T2-1 was obtained by performing a reaction in a similar method as described in Example 19 compound T1-1 (12.3 mg, 71%).

ESI-MS m/z: 1439 (M+).

### Preparation of Compound T2-2

T2-2 was obtained by performing a reaction in a similar method as described in Example 19 compound T1-2 (6.4 mg, 61%).

ESI-MS m/z: 1173 (M⁺).

### Preparation of Compound T2

T2 was obtained by performing a reaction in a similar method as described in Example 19 compound T1 (7.2 mg, 92%).

ESI-MS m/z: 1308 (M⁺).

### Example 21: Preparation of Compound T3

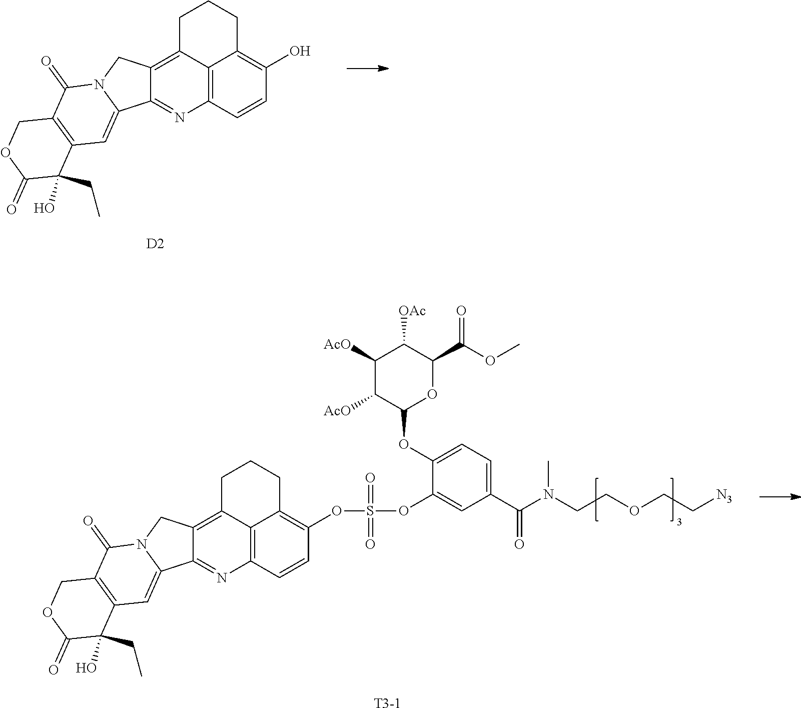

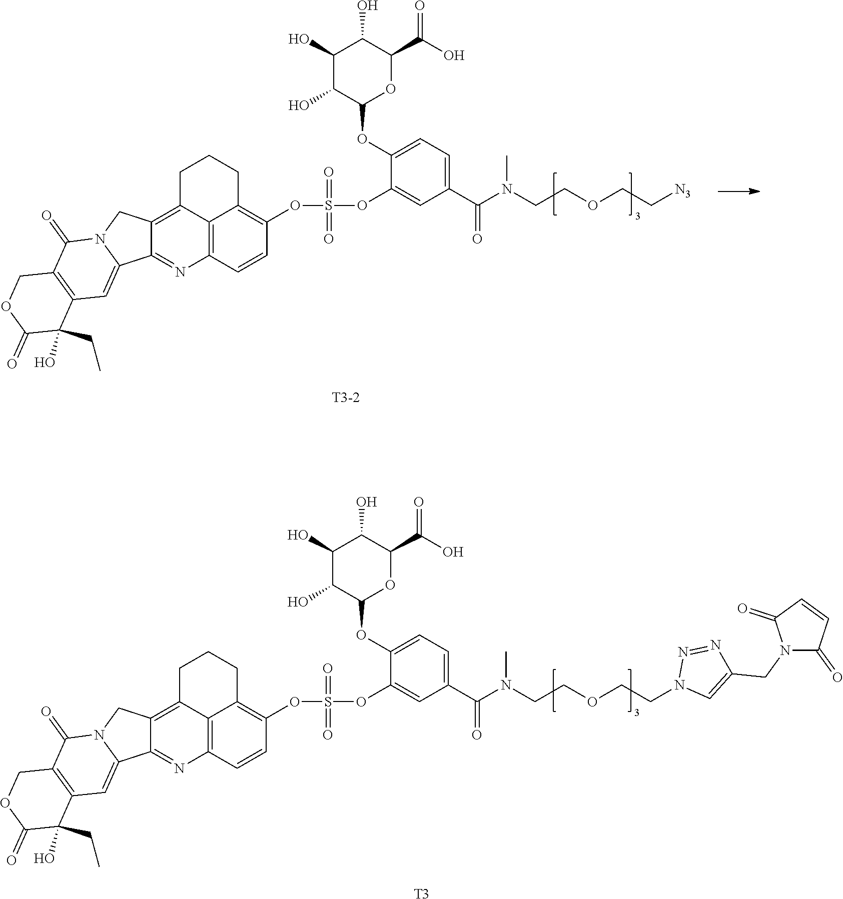

### Preparation of Compound T3-1

To a solution of compound D2 (10 mg, 0.025 mmol) in anhydrous DMF (2.0 mL) at room temperature under N₂ atmosphere was treated with TEA (17.2 L, 0.037 mmol), Int-L1 (36.3 mg, 0.12 mmol) and stirred for 15 hours. When the reaction was completed, the mixture was extracted with EA and distilled water. The organic layer was dried over anhydrous Na₂SO₄, filtered and concentrated under reduced pressure. The residue was purified by column chromatography to obtain compound T3-1 (14.4 mg, 50.6%).

ESI-MS m/z: 1151 (M⁺).

### Preparation of Compound T3-2

To a solution of compound T3-1 (14.4 mg, 0.012 mmol) in THF (0.4 mL), MeOH (0.4 mL), distilled water (0.2 mL) at −15° C. under N₂ atmosphere was treated with lithium hydroxide monohydrate (4.2 mg, 0.10 mmol) and stirred for 2.5 hours. When the reaction was completed, the mixture was quenched with 2N HCl solution and concentrated under reduced pressure. The residue purified by prep HPLC to obtain compound T3-2 (6.0 mg, 47.6%).

ESI-MS m/z: 1011 (M⁺+1).

### Preparation of Compound T3

To a solution of compound T3-2 (6.0 mg, 0.006 mmol), Mal-2 (1.2 mg, 0.009 mmol) in degassed DMSO (0.4 mL), distilled water (0.4 mL) at room temperature under N₂ atmosphere was treated with CuBr (1.28 mg, 0.009 mmol) and stirred for 15 minutes. The reaction mixture was purified by prep HPLC to obtain compound T3 (5.33 mg, 78%).

ESI-MS m/z: 1146 (M⁺).

### Example 22: Preparation of Compound T4

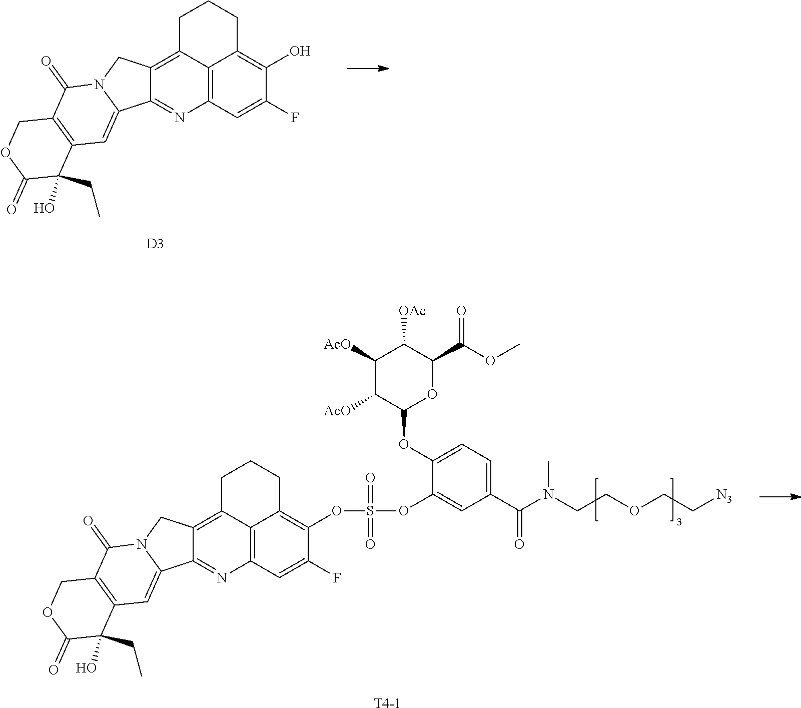

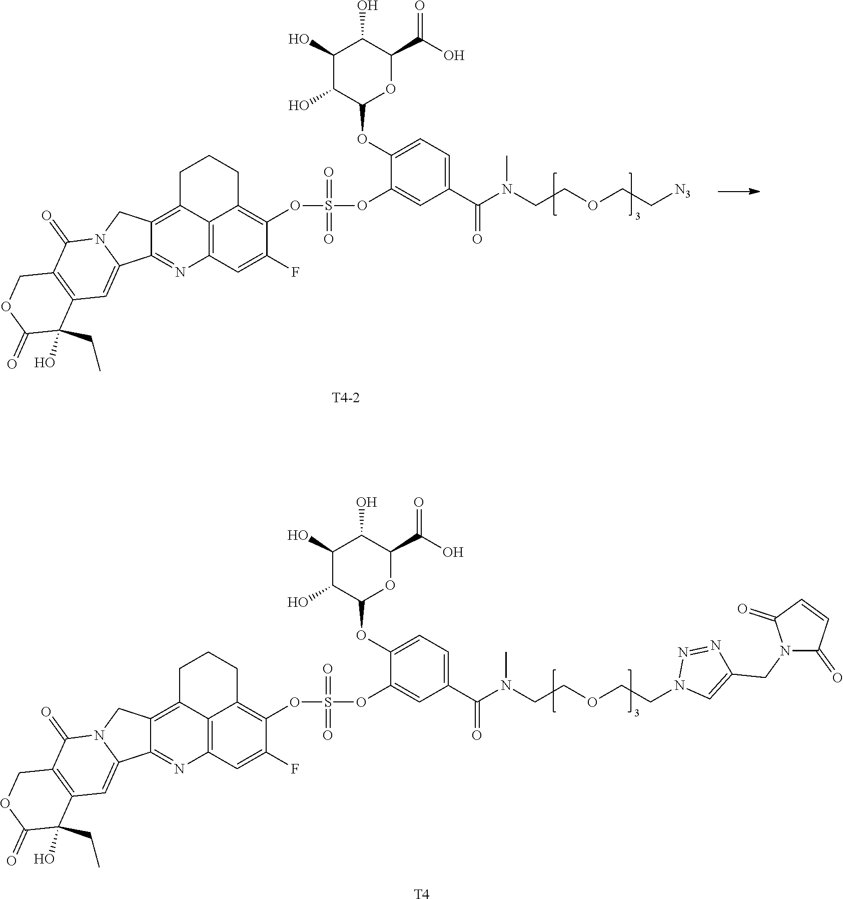

### Preparation of Compound T4-1

To a solution of compound D3 (100 mg, 0.24 mmol) in anhydrous DMF (10.0 mL) at room temperature under N₂ atmosphere was treated with TEA (165 L, 1.18 mmol), Int-L1 (348 mg, 0.36 mmol) and stirred for 1 hour. When the reaction was completed, the mixture was extracted with EA and distilled water. The organic layer was dried over anhydrous Na₂SO₄, filtered and concentrated under reduced pressure. The residue was purified by column chromatography to obtain compound T4-1 (263 mg, 95%).

ESI-MS m/z: 1169 (M⁺).

### Preparation of Compound T4-2

To a solution of compound T4-1 (263 mg, 0.22 mmol) in THF (2.8 mL), MeOH (2.8 mL), distilled water (1.4 mL) at −15° C. under N₂ atmosphere was treated with lithium hydroxide monohydrate (47.2 mg, 0.10 mmol) and stirred for 1.5 hours. When the reaction was completed, the mixture was quenched with 2 N HCl solution, and concentrated under reduced pressure. The residue purified by prep HPLC to obtain compound T4-2 (165.9 mg, 72%).

ESI-MS m/z: 1029 (M⁺+1).

### Preparation of Compound T4

To a solution of compound T4-2 (115 mg, 0.11 mmol), Mal-2 (22.6 mg, 0.17 mmol) in degassed DMSO (2.5 mL), distilled water (2.5 mL) at room temperature under N₂ atmosphere was treated with CuBr (24.05 mg, 0.17 mmol) and stirred for 15 minutes. The reaction mixture was purified by prep HPLC to obtain compound T4 (108.1 mg, 83%).

ESI-MS m/z: 1164 (M⁺).

### Example 23: Preparation of Compound IAA-1

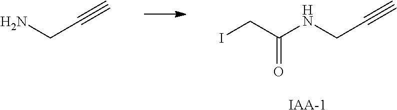

### Preparation of Compound IAA-1

To a solution of iodoacetic anhydride (300 mg, 0.85 mmol) and propargyl amine (39 mg, 0.71 mmol) in anhydrous DCM (3 ml) was added TEA (295.3 μL, 2.1 mmol) at 0° C. under N₂ atmosphere. The reaction mixture was extracted with EA and distilled water. The obtained organic layer was dried over anhydrous Na₂SO₄, filtered, and concentrated under reduced pressure. The compound was vacuum-dried for 15 hours to obtain compound IAA-1 (145 mg, 90%).

¹H NMR (400 MHz, CDCl₃) δ 4.08 (q, J=2.8 Hz, 2H), 3.72 (s, 2H), 2.29 (t, J=2.8 Hz, 1H) ESI-MS m/z: 224 (M⁺+1).

### Example 24: Preparation of Compound T5

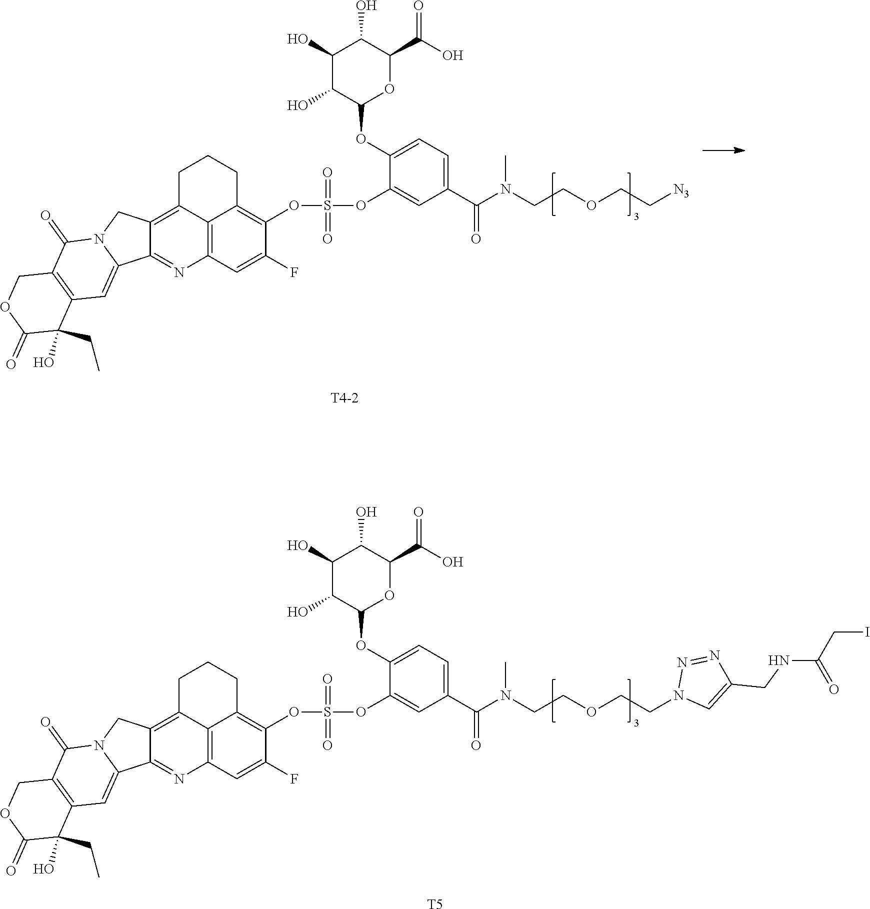

### Preparation of Compound T5

To a solution of compound T4-2 (10 mg, 0.01 mmol), IAA-1 (3.25 mg, 0.014 mmol) in degassed DMSO (0.5 mL), distilled water (0.5 mL) at room temperature under N₂ atmosphere was treated with CuBr (2.1 mg, 0.014 mmol) and stirred for 40 minutes. The reaction mixture was purified by prep HPLC to obtain compound T5 (9.46 mg, 77%).

ESI-MS m/z: 1252 (M⁺+1).

### Example 25: Preparation of Compound T6

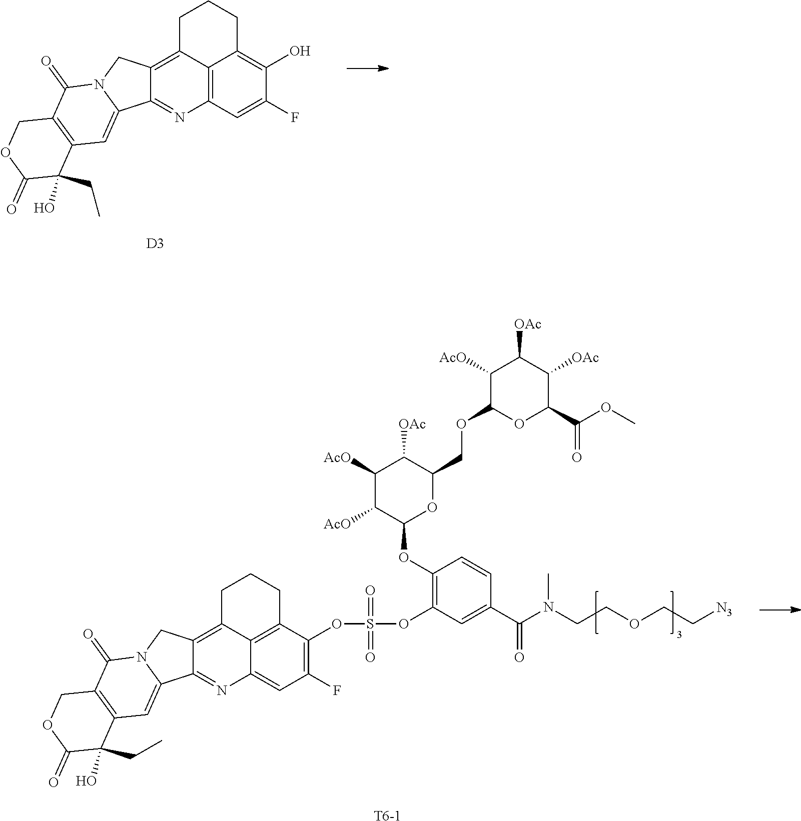

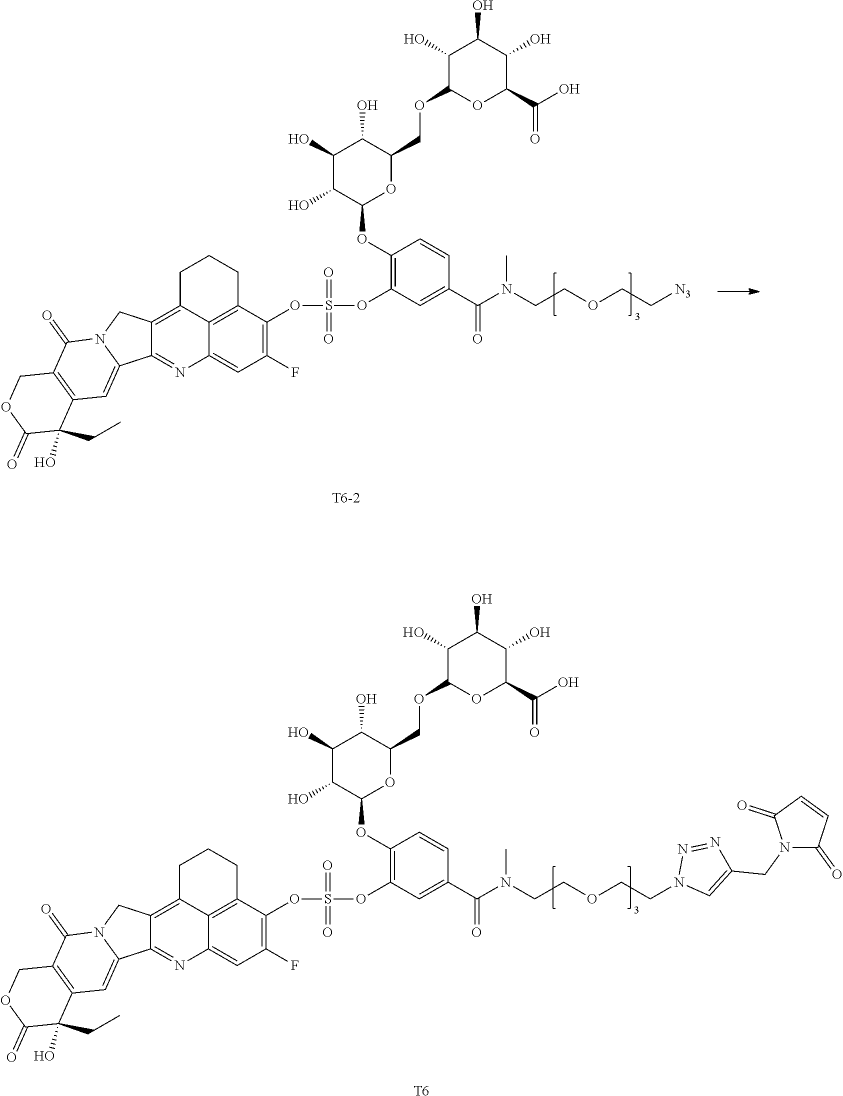

### Preparation of Compound T6-1

T6-1 was obtained by performing a reaction in a similar method as described in Example 19 compound T1-1 (87 mg, 87%).

ESI-MS m/z: 1457 (M′).

### Preparation of Compound T6-2

T6-2 was obtained by performing a reaction in a similar method as described in Example 19 compound T1-2 (52 mg, 73%).

ESI-MS m/z: 1191 (M⁺).

### Preparation of Compound T6

T6 was obtained by performing a reaction in a similar method as described in Example 19 compound T1 (12.5 mg, 75%).

ESI-MS m/z: 1326 (M⁺).

### Example 26: Preparation of Compound T7

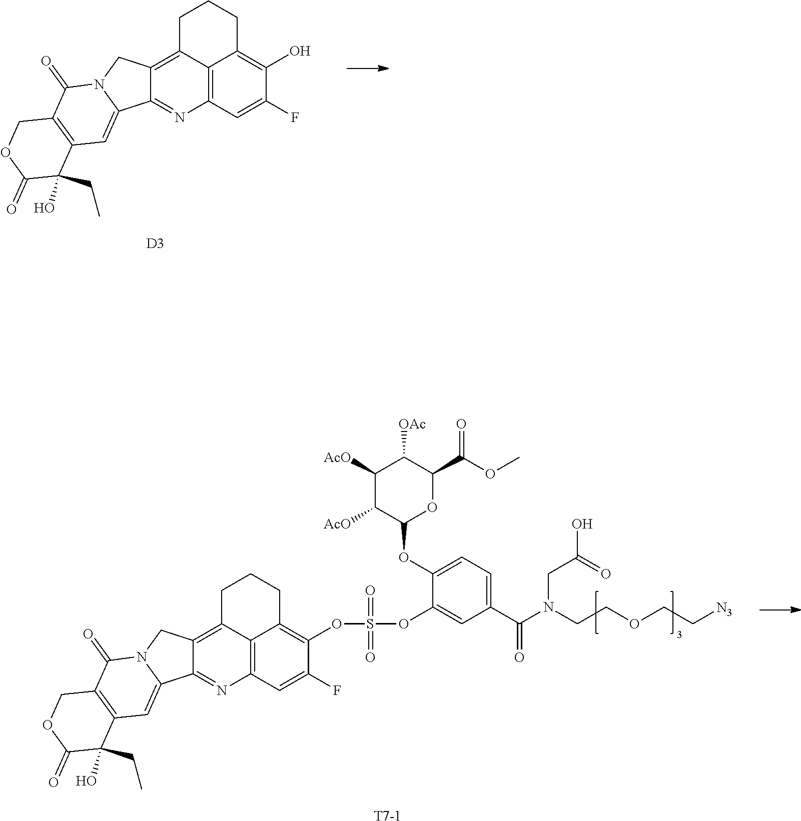

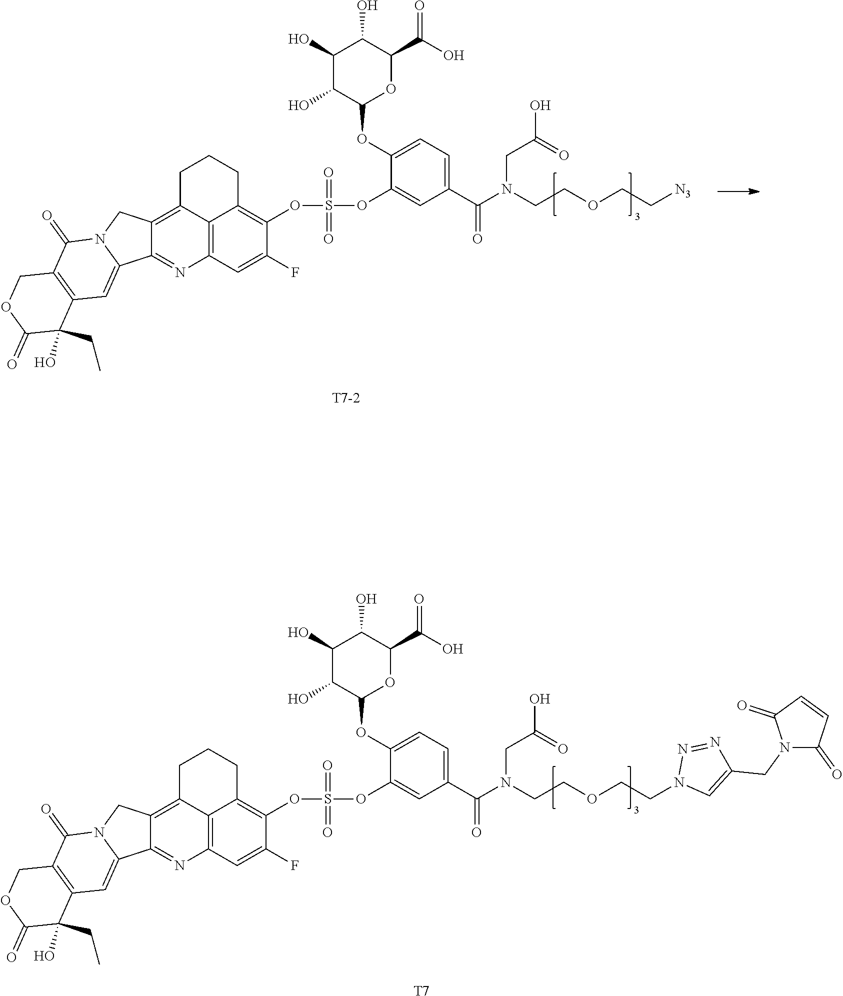

### Preparation of Compound T7-1

T7-1 was obtained by performing a reaction in a similar method as described in Example 19 compound T1-1 (24.1 mg, 83%).

ESI-MS m/z: 1228 (M⁺+1).

### Preparation of Compound T7-2

T7-2 was obtained by performing a reaction in a similar method as described in Example 19 compound T1-2 (15.6 mg, 74%).

ESI-MS m/z: 1073 (M⁺+1).

### Preparation of Compound T7

T7 was obtained by performing a reaction in a similar method as described in Example 19 compound T1 (13.5 mg, 77%).

ESI-MS m/z: 1209 (M⁺+1).

### Example 27: Preparation of Compound T8

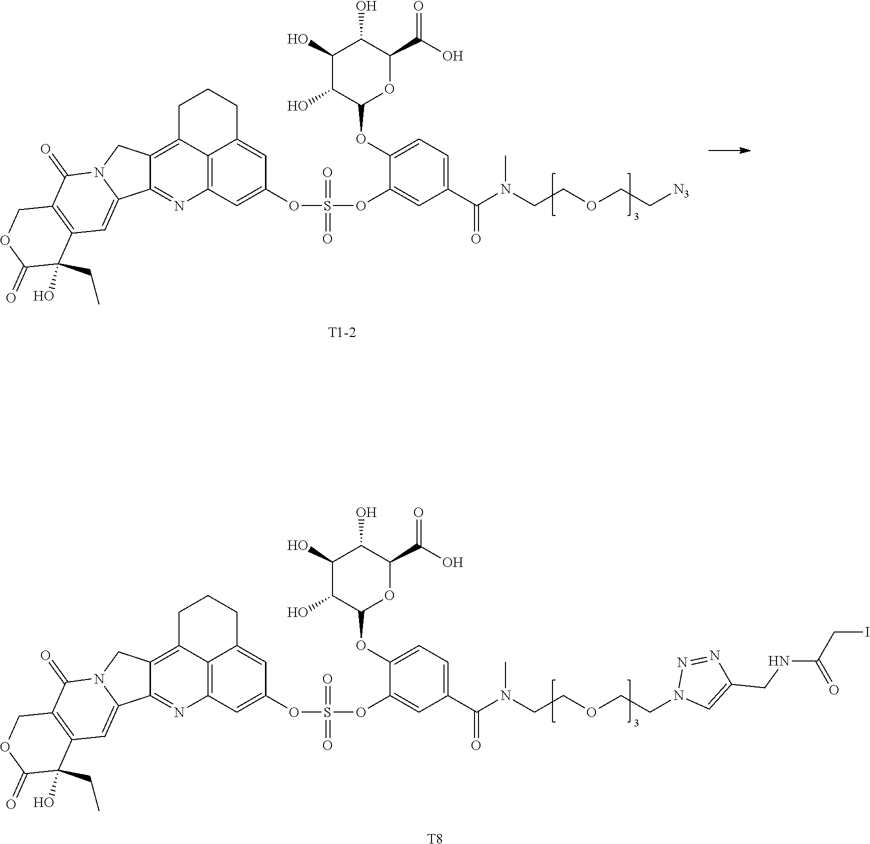

### Preparation of Compound T8

To a solution of compound T1-2 (10 mg, 0.01 mmol), IAA-1 (3.3 mg, 0.015 mmol) in degassed DMSO (0.2 mL), distilled water (0.2 mL) at room temperature under N₂ atmosphere was treated with CuBr (2.1 mg, 0.015 mmol) and stirred for 15 minutes. The reaction mixture was purified by prep HPLC to obtain compound T8 (9.84 mg, 80%).

ESI-MS m/z: 1234 (M⁺+1).

### Example 28: Preparation of Compound T9

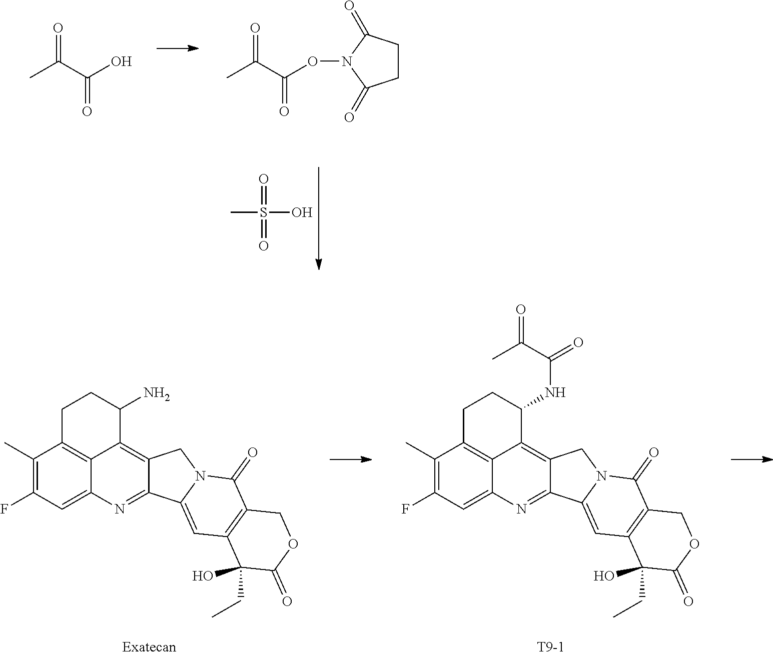

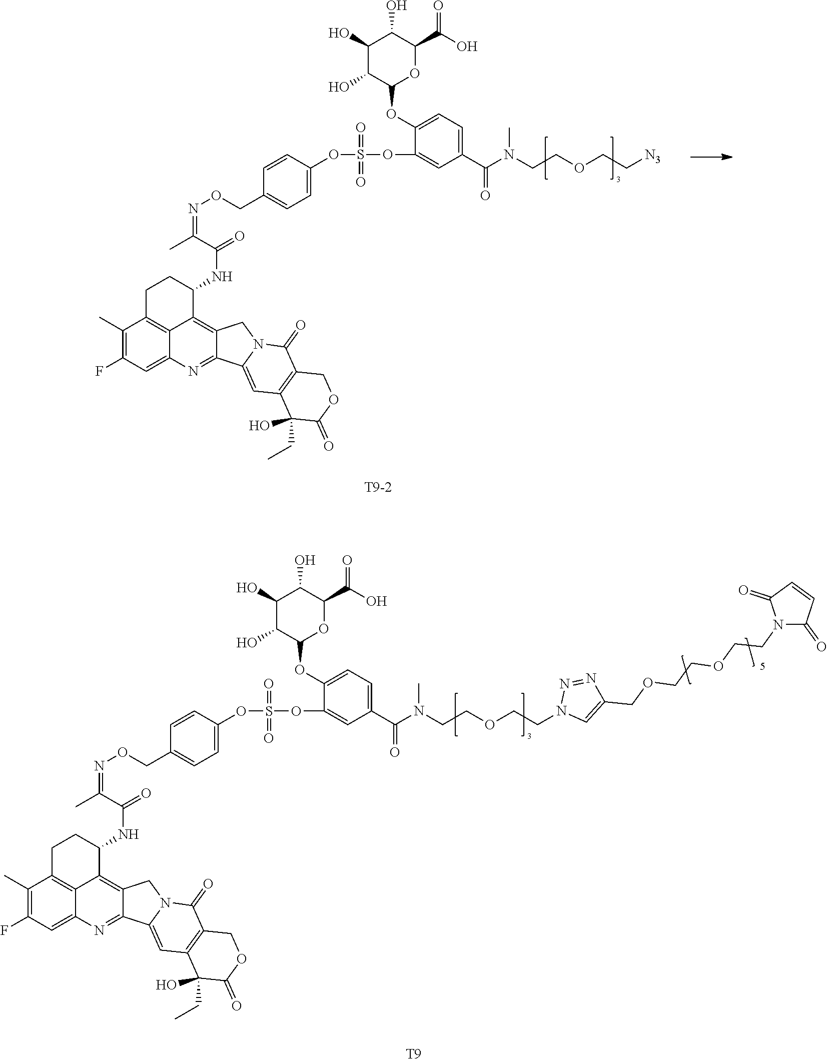

### Preparation of Compound T9-1

To a solution of pyruvic acid (1.2 mg, 0.014 mmol) in anhydrous DMF (0.5 mL) at room temperature under N₂ atmosphere was treated with EDCI (1-ethyl-3-(3-dimethylaminopropyl)carbodiimide, 2.7 mg, 0.014 mmol), N-hydroxysuccinimide (1.6 mg, 0.014 mmol) and stirred for 1 hour. The exatecan mesylate (5.0 mg, 0.009 mmol) in anhydrous DMF (1.0 mL), TEA (1.34 μL, 0.01 mmol) was added in reaction mixture and stirred for 22 hours. When the reaction was completed, the reaction mixture was purified by prep HPLC to obtain compound T9-1 (3.04 mg, 50.6%).

ESI-MS m/z: 506 (M-+1).

### Preparation of Compound T9-2

To a solution of compound T9-1 (2.2 mg, 0.004 mmol), Int-L4-3 (7.3 mg, 0.008 mmol) in anhydrous DMF (0.7 mL) at room temperature under N₂ atmosphere was treated with TEA (1.2 μL, 0.009 mmol) and stirred for 2.5 hours. When the reaction was completed, the mixture was purified by prep HPLC to obtain compound T9-1 (2.32 mg, 39%).

ESI-MS m/z: 1233 (M⁺).

### Preparation of Compound T9

To a solution of compound T9-2 (1.87 mg, 0.002 mmol), Mal-1 (1.82 mg, 0.004 mmol) in degassed DMSO (0.4 mL), distilled water (0.2 mL) at room temperature under N₂ atmosphere was treated with CuBr (1.3 mg, 0.009 mmol) and stirred for 20 minutes. The reaction mixture was purified by prep HPLC to obtain compound T9 (1.71 mg, 69%).

ESI-MS m/z: 1632 (M⁺).

### Example 29: Preparation of Compound T10

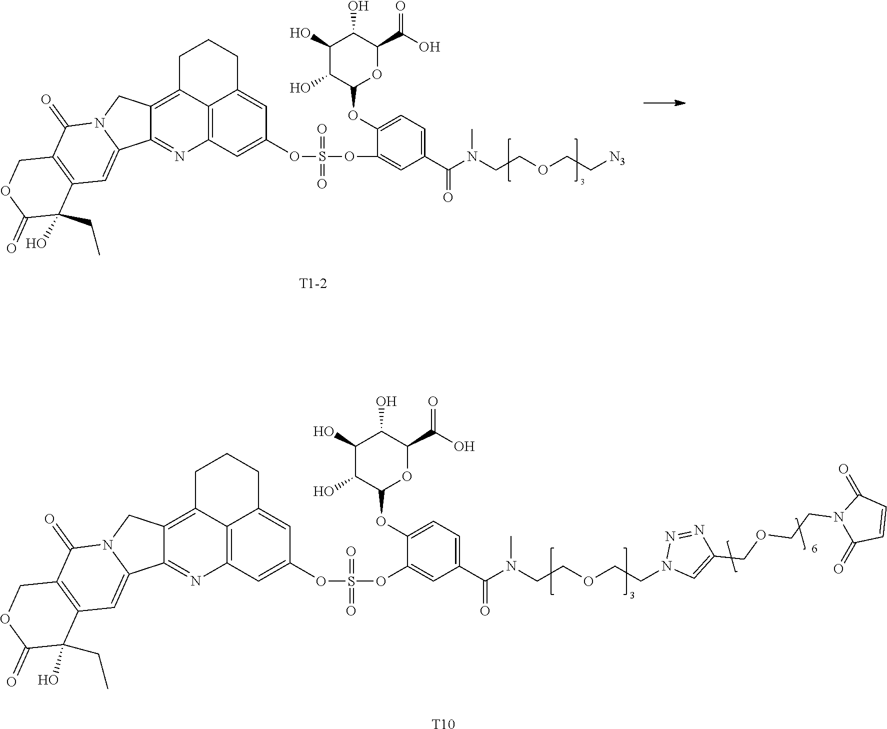

### Preparation of Compound T10

T10 was obtained by performing a reaction in a similar method as described in Example 28 compound T9 (11.4 mg, 82%).

ESI-MS m/z: 1411 (M⁺+1).

### Example 30: Preparation of Compound T26

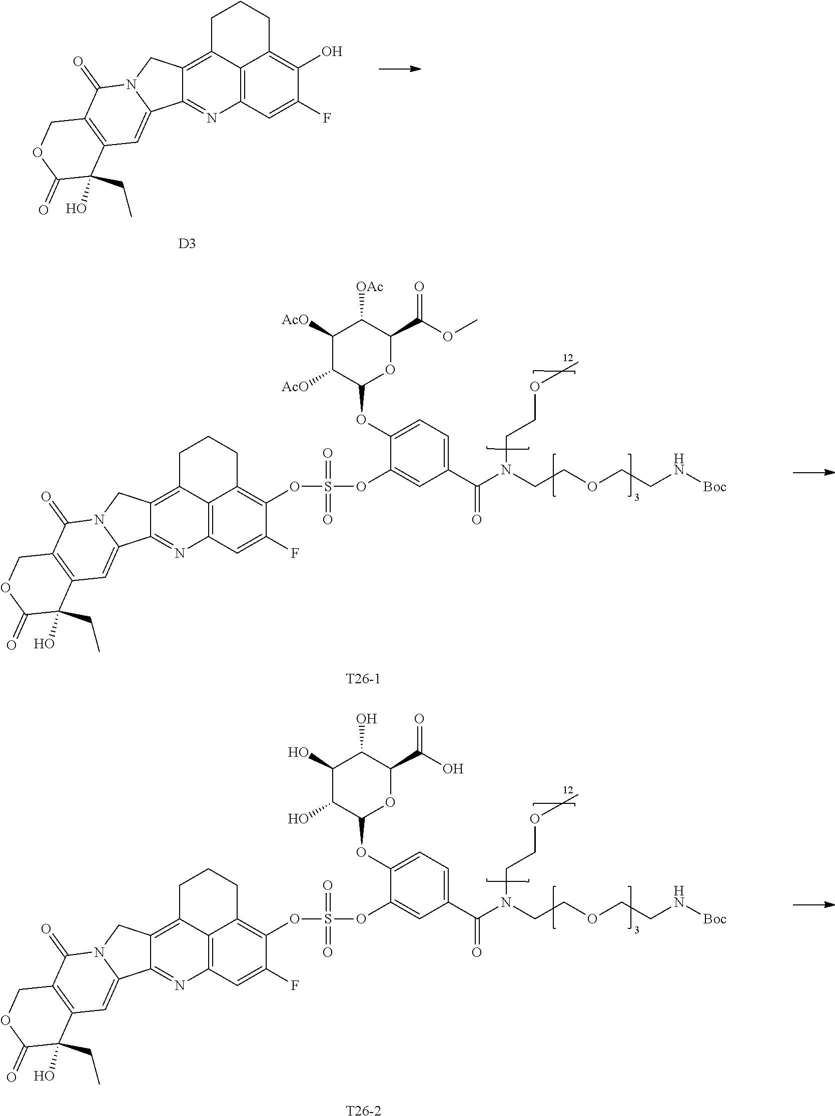

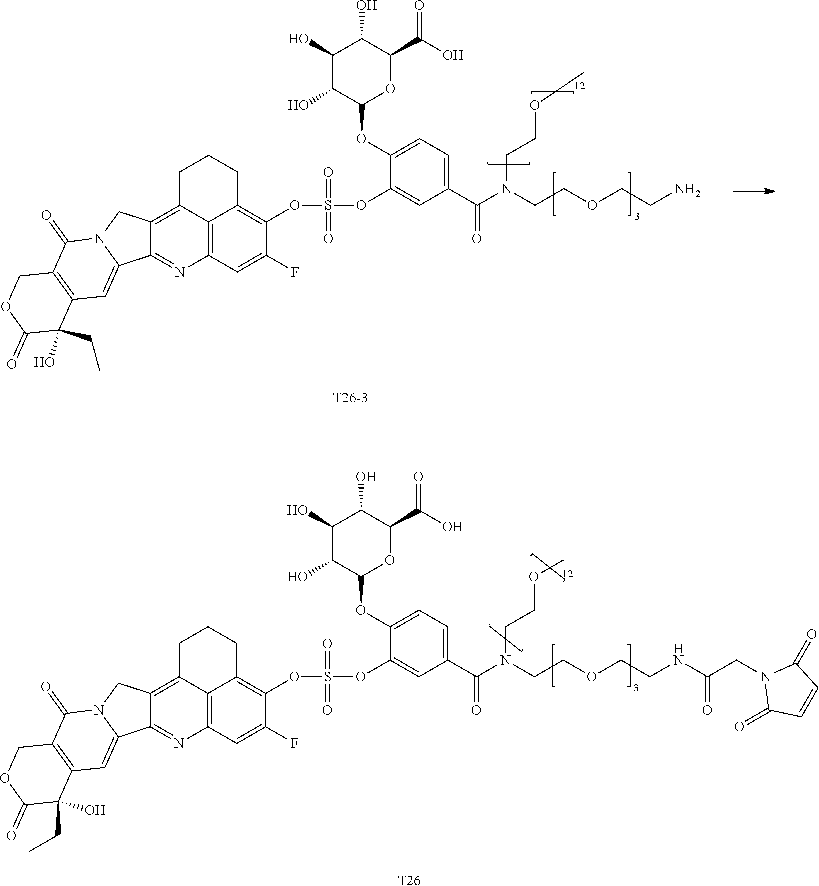

### Preparation of Compound T26-1

T26-1 was obtained by performing a reaction in a similar method as described in Example 22 compound T4-1 (36.3 mg, 51.5%).

ESI-MS m/z: 1772 (M⁺+1).

### Preparation of Compound T26-2

T26-2 was obtained by performing a reaction in a similar method as described in Example 22 compound T4-2 (14.4 mg, 43%).

ESI-MS m/z: 1632 (M⁺+1).

### Preparation of Compound T26-3

To a solution of T26-2 (14.4 mg, 0.009 mmol) in anhydrous DCM (300 uL) at 0° C. was treated with TFA (50 uL) and stirred for 3 hours. The reaction mixture was concentrated under reduced pressure. The residue was washed with Ether and vacuum dried to obtained compound T26-3 (13.6 mg, 93.9%).

ESI-MS m/z: 1532 (M⁺+1 free form).

### Preparation of Compound T26

To a solution of T26-3 (8.44 mg, 0.0051 mmol) in DMF (410 uL) at room temperature under N₂ atmosphere was treated with DIPEA (1.34 uL, 0.0077 mmol), solution of Maleimidoacetic acid N-hydroxysuccinimide ester (BlD, CAS: 55750-61-3, 1.16 mg, 0.0046 mmol) in DMF (100 uL) and stirred for 3 hours. The reaction mixture was purified by prepHPLC to obtain compound T26 (5.04 mg, 58.9%).

ESI-MS m/z: 1669 (M⁺+1).

### Example 31: Preparation of Compound T42

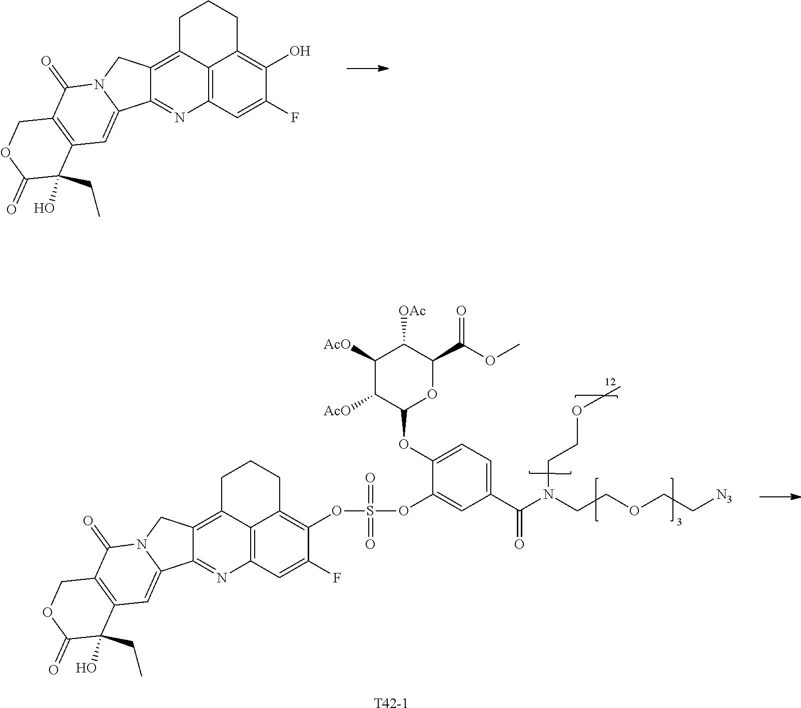

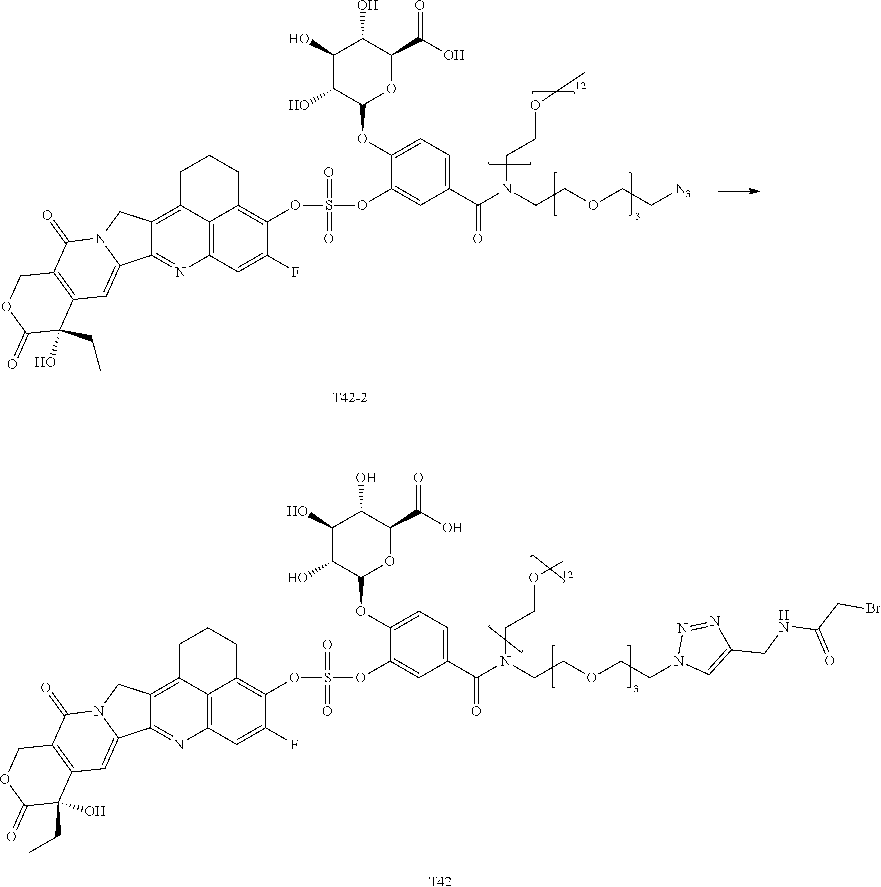

### Preparation of Compound T42-1

T42-1 was obtained by performing a reaction in a similar method as described in Example 22 compound T4-1 (44.8 mg, 53.7%).

ESI-MS m/z: 1698 (M⁺+1).

### Preparation of Compound T42-2

T42-2 was obtained by performing a reaction in a similar method as described in Example 22 compound T4-2 (22.3 mg, 54.2%).

ESI-MS m/z: 1558 (M⁺+1).

### Preparation of Compound T42

T42 was obtained by performing a reaction in a similar method as described in Example 22 compound T4 (21 mg, 58.8%).

ESI-MS m/z: 1734 (M⁺+1).

### Example 32: Preparation of Compound T11 to T25, T27 to T41, and T43 to T46

Compounds were obtained by performing a reaction in a similar method as described in Examples 29-31.

TABLE 2

| | | Ana- |
|---|---|---|
| Com- | | lytical |
| pounds | Structure | Data |
| T11 | 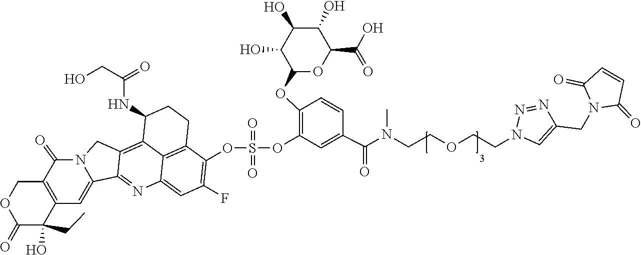 | ESI-MS m/z: 1237 (M⁺). |
| T12 | 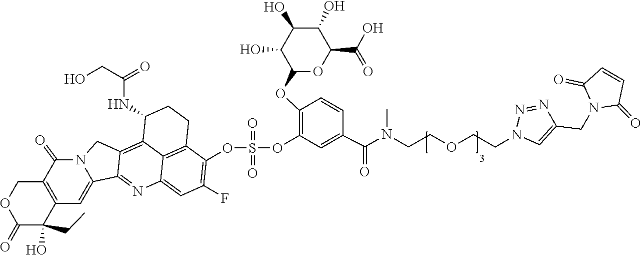 | ESI-MS m/z: 1238 (M⁺ + 1). |
| T13 | 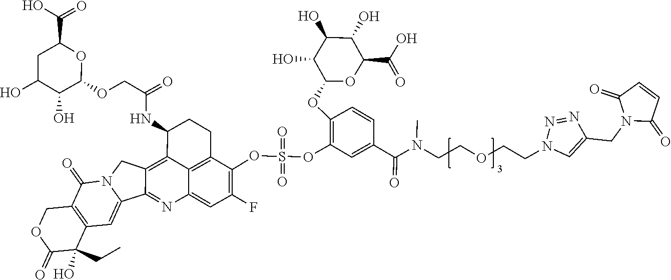 | ESI-MS m/z: 1414 (M⁺ + 1). |
| T14 | 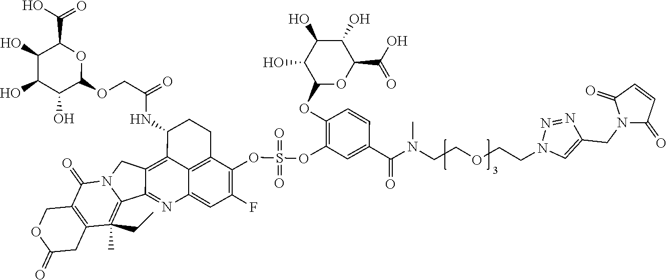 | ESI-MS m/z: 1414 (M⁺ + 1). |
| T15-1 | 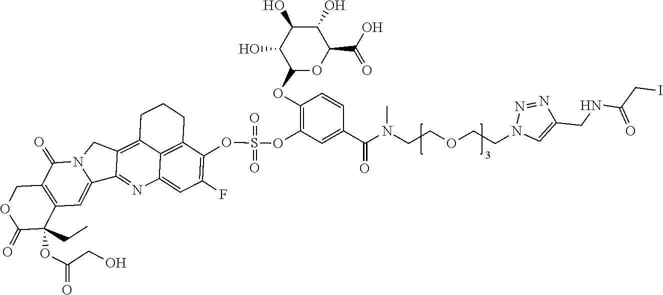 | ESI-MS m/z: 1311 (M⁺ + 1). |
| T15-2 | 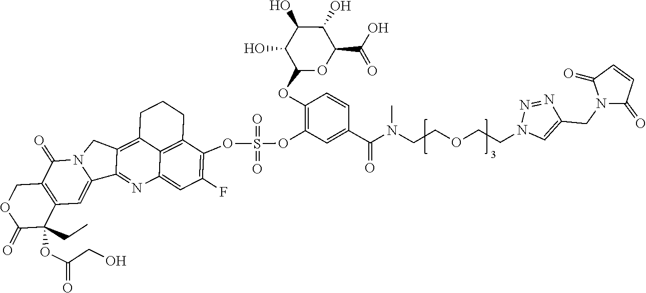 | ESI-MS m/z: 1223 (M⁺ + 1). |
| T16-1 | 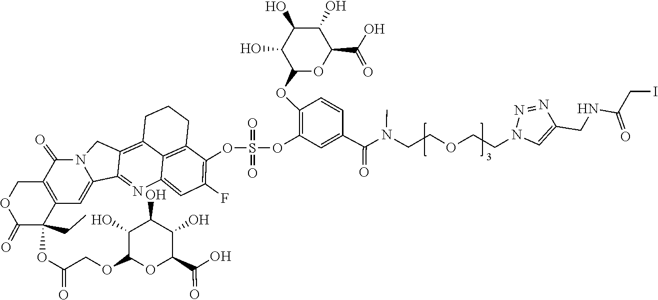 | ESI-MS m/z: 1487 (M⁺ + 1). |
| T16-2 | 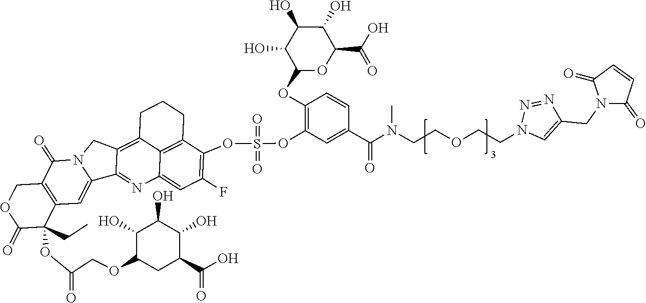 | ESI-MS m/z: 1399 (M⁺ + 1) |
| T17 | 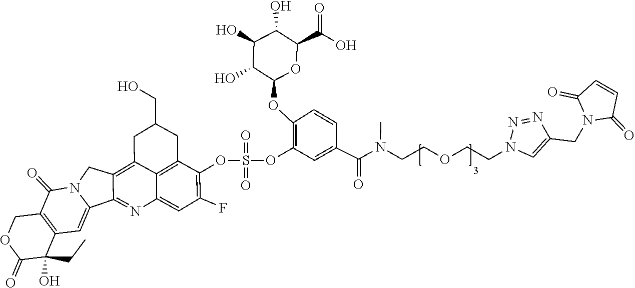 | ESI-MS m/z: 1195 (M⁺ + 1) |
| T18 | 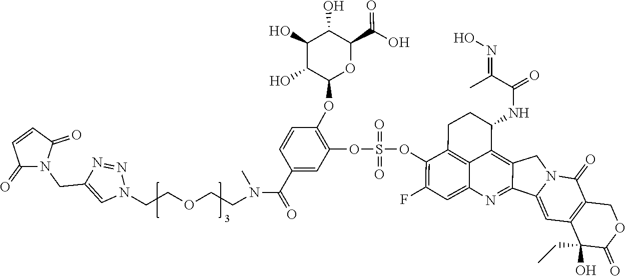 | ESI-MS m/z: 1264 (M⁺) |
| T19 | 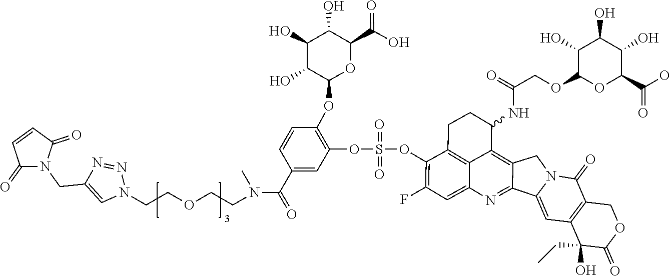 | ESI-MS m/z: 1414 (M⁺ + 1) |
| T20 | 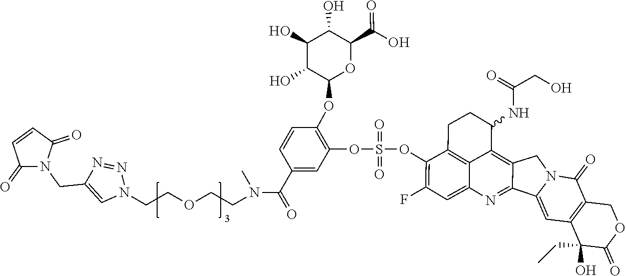 | ESI-MS m/z: 1238 (M⁺ + 1) |
| T21 | 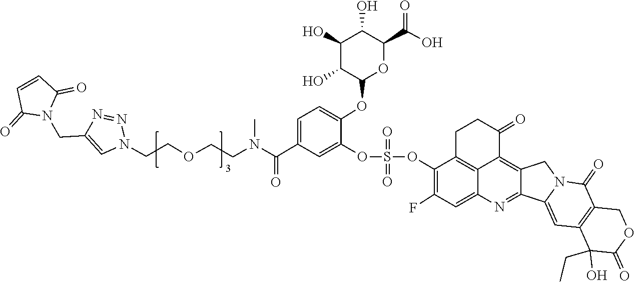 | ESI-MS m/z: 1179 (M⁺ + 1) |
| T22 | 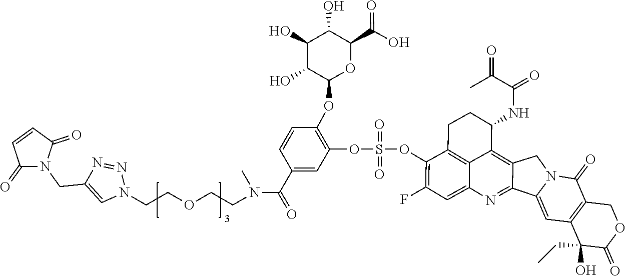 | ESI-MS m/z: 1249 (M⁺) |
| T23 | 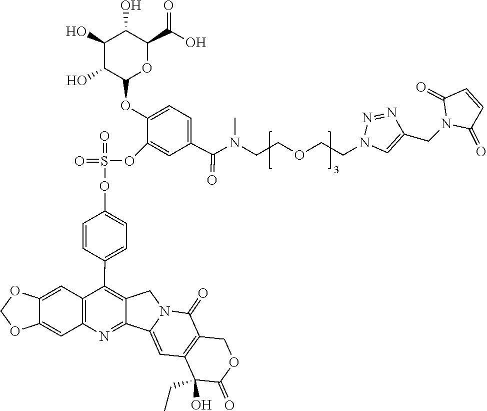 | ESI-MS m/z: 1227 (M⁺ + 1) |
| T24 | 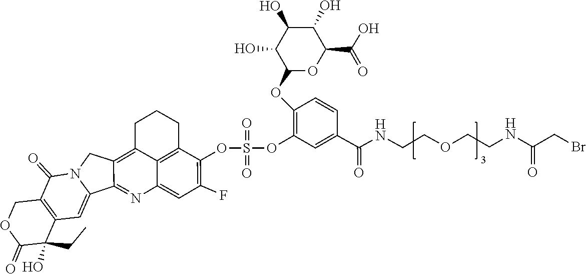 | ESI-MS m/z: 1110 (M⁺ + 1) |
| T25 | 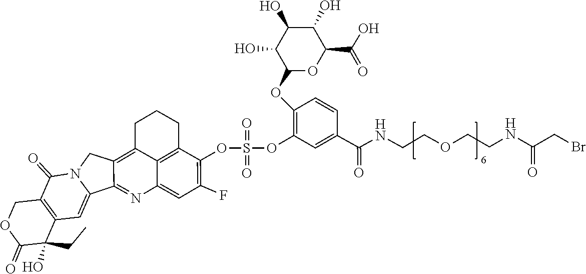 | ESI-MS m/z: 1243 (M⁺ + 1) |
| T26 | 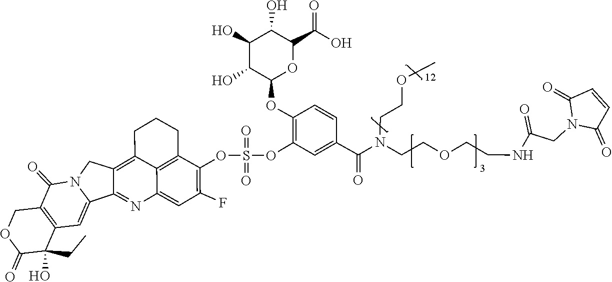 | ESI-MS m/z: 1669 (M⁺ + 1) |
| T27 | 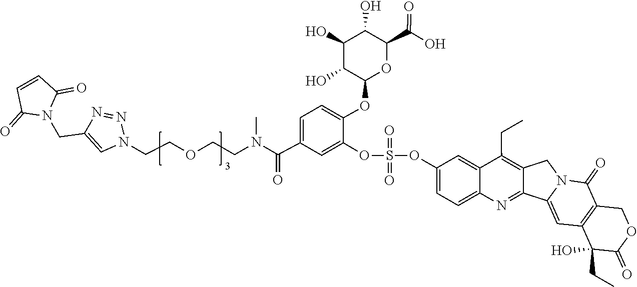 | ESI-MS m/z: 1135 (M⁺ + 1) |
| T28 | 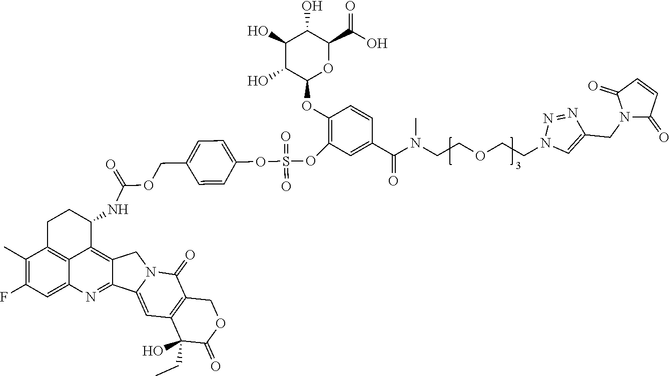 | ESI-MS m/z: 1328 (M⁺ + 1) |
| T29 | 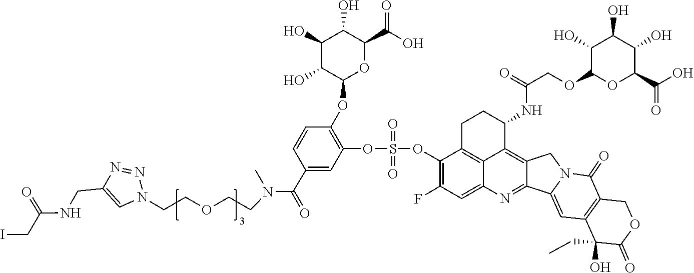 | ESI-MS m/z: 1502 (M⁺ + 1) |
| T30 | 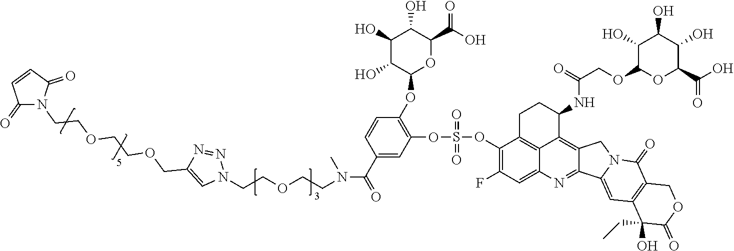 | ESI-MS m/z: 1678 (M⁺ + 1) |
| T31 | 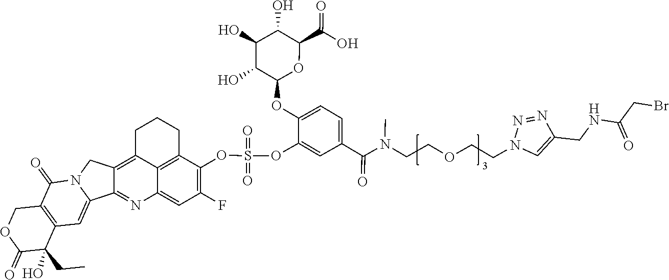 | ESI-MS m/z: 1205 (M⁺ + 1) |
| T32 | 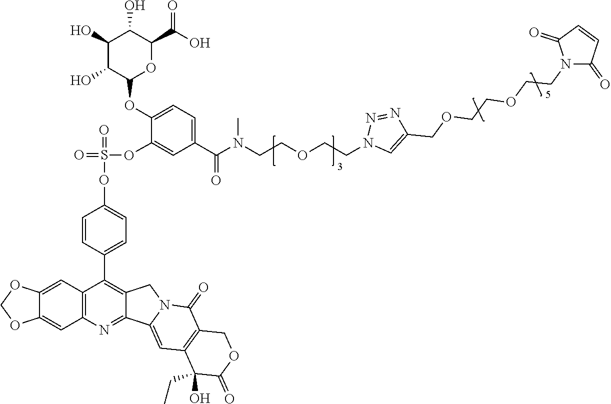 | ESI-MS m/z: 1491 (M⁺ + 1) |
| T33 | 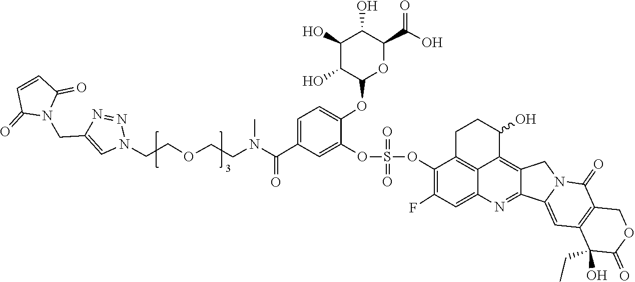 | ESI-MS m/z: 1181 (M⁺ + 1) |
| T34 | 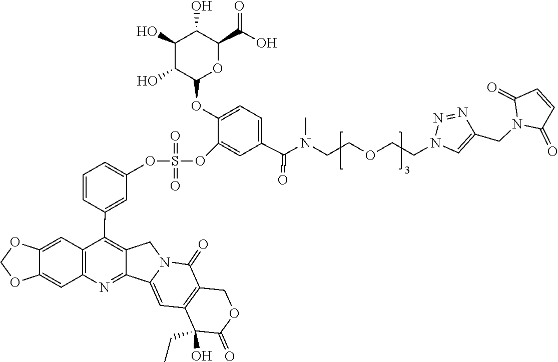 | ESI-MS m/z: 1227 (M⁺ + 1) |
| T35 | 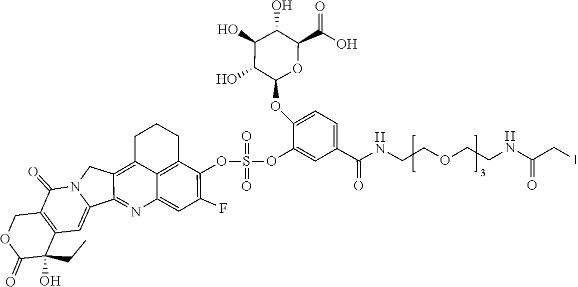 | ESI-MS m/z: 1290 (M⁺ + 1) |
| T36 | 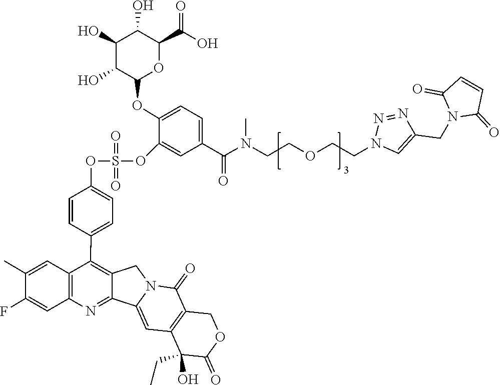 | ESI-MS m/z: 1215 (M⁺ + 1) |
| T37 | 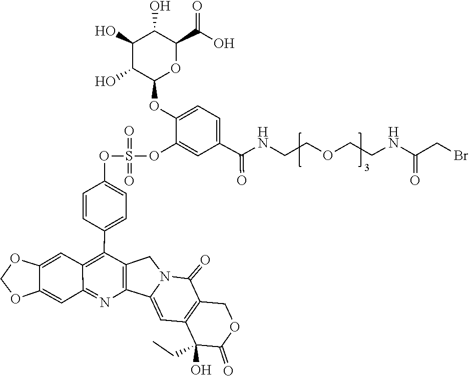 | ESI-MS m/z: 1172 (M⁺ + 1) |
| T38 | 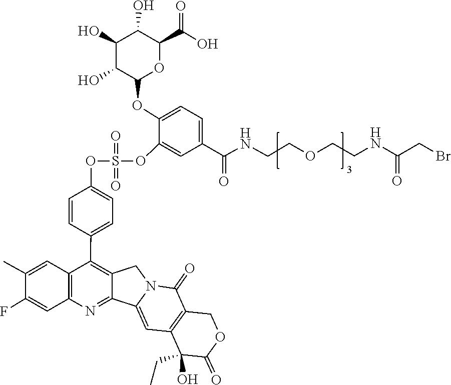 | ESI-MS m/z: 1160 (M⁺ + 1) |
| T39 | 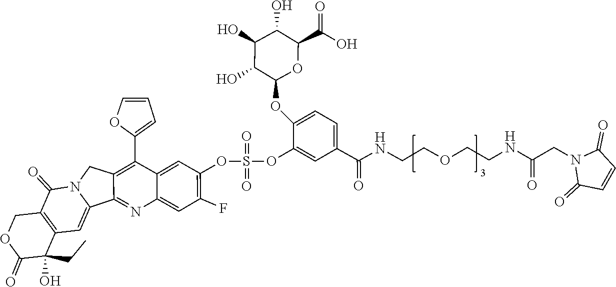 | ESI-MS m/z: 1153 (M⁺ + 1) |
| T40 | 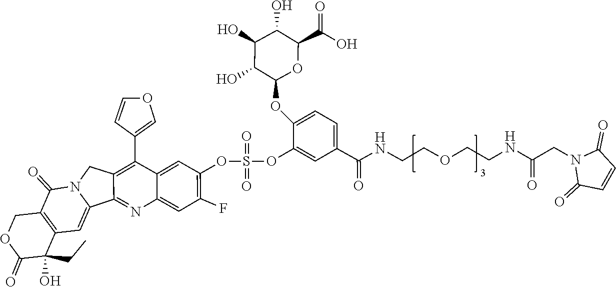 | ESI-MS m/z: 1153 (M⁺ + 1) |
| T41 | 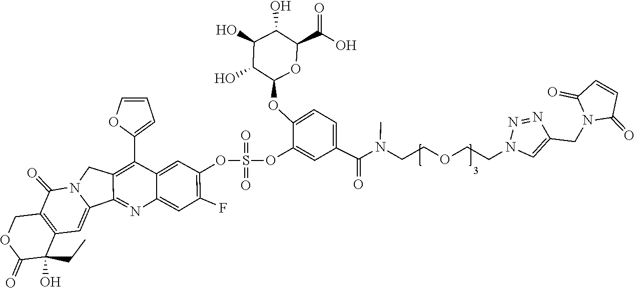 | ESI-MS m/z: 1191 (M⁺ + 1) |
| T42 | 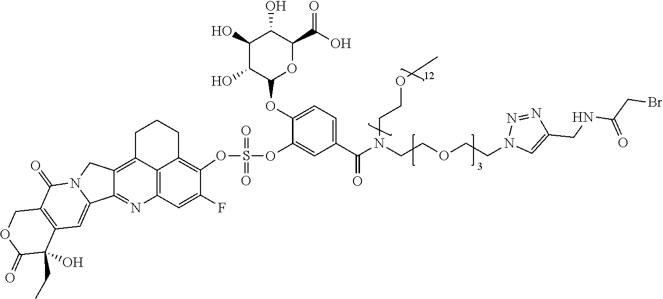 | ESI-MS m/z: 1734 (M⁺ + 1) |
| T43 | 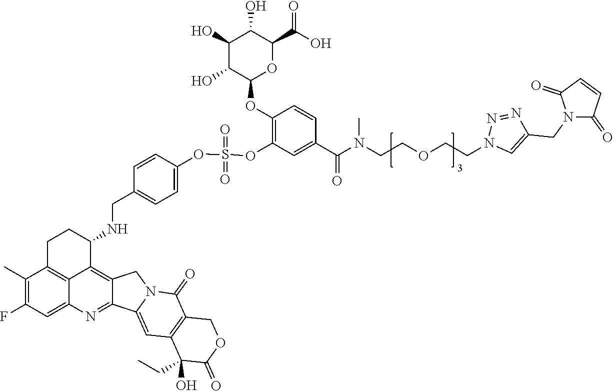 | ESI-MS m/z: 1284 (M⁺ + 1) |
| T44 | 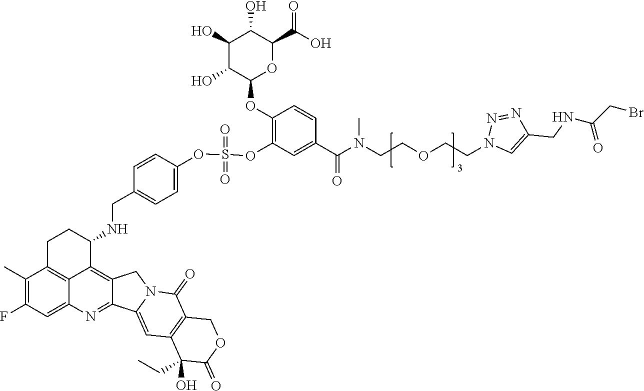 | ESI-MS m/z: 1325 (M⁺ + 1) |
| T45 | 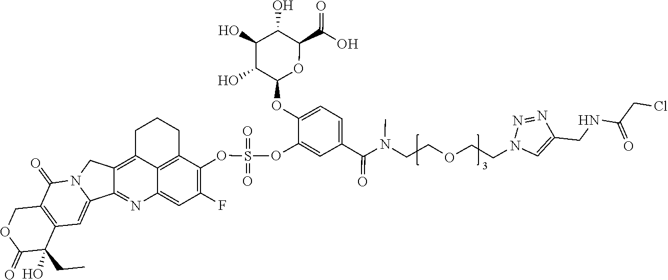 | ESI-MS m/z: 1161 (M⁺ + 1) |
| T46 | 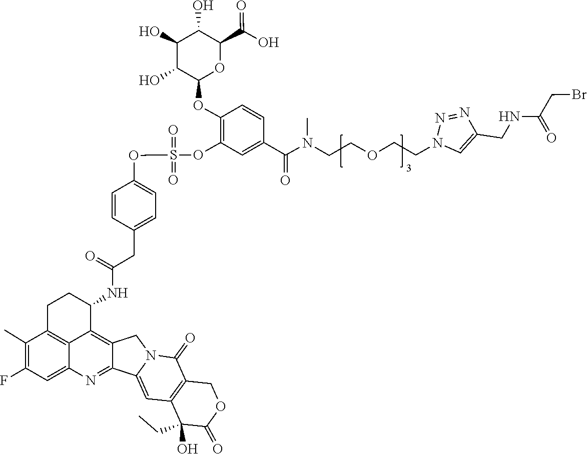 | ESI-MS m/z: 1353 (M⁺ + 1) |
| T47 | 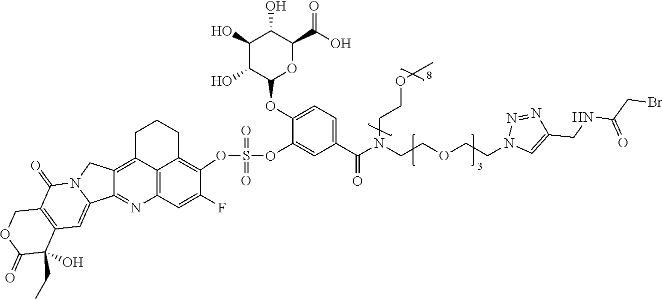 | ESI-MS m/z: 1558 (M⁺ + 1) |

### Example 33: In Vitro Analysis of Drugs

The drugs (compounds D1-D6, D21, D25, D27, D28, D35-D37, D46, D47, D48, D49 and controls DXd, FL118 and SN38) were evaluated on NCI-N87, MDA-MB-468, SK-BR-3, HCT116, and JIMT-1 cancer cells. The cancer cells were seeded in 96-well plates at a density of 2,000 to 5,000 cells per well in 100 L of medium and cultured for 22 to 26 hours. The series of compound was diluted by serial dilution of 1:4˜1:8 from 2 μM to 1 pM with DMSO. The diluted drugs were added to triplicate wells of 96-well plates at 50 μL per well. All assays were performed, by repeating up to 3 times and the results were obtained in up to 3 independent experiments. The plates were incubated for 6 days at 37° C. in a humidified 5% CO₂-in-air atmosphere. Cell viability was determined by the MTT assay. The 15 μL of 3-(4,5-dimethylthiazol-2-yl)-2,5-diphenyltetrazolium bromide (MTT) dye dissolved in PBS buffer solution (5 mg/mL) was added to each well of the plates. The formazans formed by reduction of the MTT dye by mitochondrial oxidoreductases in the living cells were dissolved in DMSO, the formazans measured using the absorbance at 550 nm. IC₅₀ (half maximal inhibitory concentration) was generated using a sigmoidal dose-response nonlinear regression curve fit (GraphPad software Inc.). Results of in vitro analysis of the drugs are shown in the Table 3 below.

TABLE 3

| | Drugs | | | | |
|---|---|---|---|---|---|
| | IC₅₀ ± SD (nM) | | | | |
| cell | SK-BR3 | NCI-N87 | JIMT-1 | HCT116 | MDA-MB-468 |
| expression | (Breast) | (Gastric) | (Breast) | (Colon) | (TNBC) |
| level | 21.8 × 10⁵ | 13.2 × 10⁵ | 1.70 × 10⁵ | 0.20 × 10⁵ | 0.003 × 10⁵ |
| D1 | 0.77 ± 0.06 | 3.60 ± 1.02 | 23.2 ± 10.6 | 6.29 ± 0.87 | 0.58 ± 0.22 |
| D2 | 0.62 ± 0.03 | 1.72 ± 0.42 | 14.8 ± 7.02 | 2.04 ± 0.14 | 0.33 ± 0.09 |
| D3 | 0.70 ± 0.23 | 1.64 ± 0.94 | N/A | N/A | 0.44 ± 0.22 |
| D4 | 12.9 ± 2.88 | N/A | N/A | N/A | N/A |
| D5 | 21.3 ± 6.85 | N/A | N/A | N/A | N/A |
| D6 | 12.4 ± 1.97 | N/A | N/A | N/A | N/A |
| D21 | 0.61 ± 0.03 | 1.49 ± 0.53 | N/A | N/A | 0.38 ± 0.14 |
| D25 | 2.81 ± 0.43 | 27.6 ± 8.26 | N/A | N/A | 4.49 ± 0.76 |
| D27 | 1.79 ± 0.34 | 6.52 ± 0.45 | N/A | N/A | 0.86 ± 0.11 |
| D28 | 5.31 ± 0.55 | 12.7 ± 4.20 | N/A | N/A | 2.45 ± 0.37 |
| D35 | N/A | 5.57 | N/A | N/A | 1.71 |
| D36 | 0.91 ± 0.13 | 1.19 ± 0.02 | N/A | N/A | 0.52 ± 0.29 |
| D37 | 0.89 ± 0.06 | 1.19 ± 0.25 | N/A | N/A | 0.36 ± 0.23 |
| D46 | 1.73 ± 0.19 | 2.20 ± 0.13 | N/A | N/A | 0.92 ± 0.12 |
| D47 | 1.80 | 2.13 | N/A | N/A | 0.96 |
| D48 | 1.06 ± 0.15 | 1.59 ± 0.09 | N/A | N/A | 0.40 ± 0.11 |
| D49 | 1.79 ± 0.24 | 6.11 ± 0.19 | N/A | N/A | 0.83 ± 0.01 |
| DXd | 1.87 ± 0.49 | 7.12 ± 1.75 | N/A | N/A | 0.82 ± 0.30 |
| FL118 | 0.86 ± 0.10 | 1.04 ± 0.18 | N/A | N/A | 0.37 ± 0.17 |
| SN38 | 1.82 ± 1.21 | 6.52 ± 1.55 | N/A | N/A | 0.68 ± 0.14 |

### Example 34: Conjugation Method 1: (Random Conjugation, Mal-Conjugation Protocol)

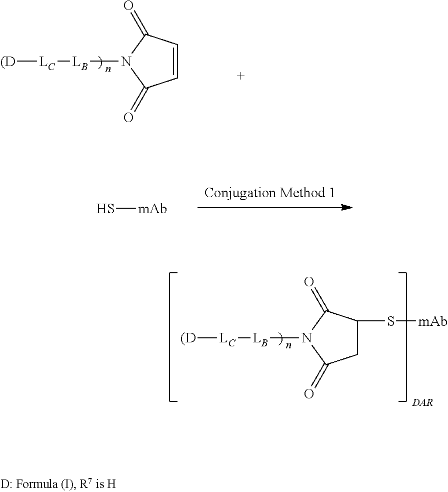

An antibody (70 μM Ab) dissolved in 1×PBS at pH 7.4 was treated with 20 molar excess of TCEP (tris(2-carboxyethyl)phosphine hydrochloride) in 25 mM tris buffer (pH 9.0) with 5 mM EDTA (ethylenediaminetetraacetic acid). After incubation at 37° C. for 3 hours, resulting solution and linker-drug (15 molar excess relative to antibody) were mixed in DMSO containing up to 10% v/v of 1×PBS, and 5 mM EDTA (pH 7.4). After the reaction was incubated for 2 hours at room temperature. N-acetyl cysteine was added with same molar relative to linker-drug into resulting solution and incubated for 0.5 hour at room temperature to quench excess linker-drug. Resulting solution was loaded and eluted through PD-10 column to remove excess linker-drug intermediate and other impurities. And the buffer was exchanged by elution through Vivaspin column (MWCO, 30 kDa) to PBS (pH 6.5) by repeating 3 times.

TABLE 4
Certain Exemplary ADCs Obtained by Conjugation Method 1 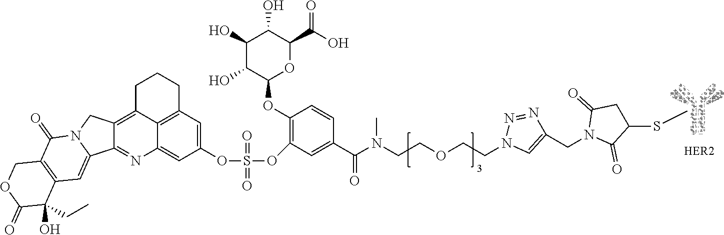
T1-AB1 (7.8) 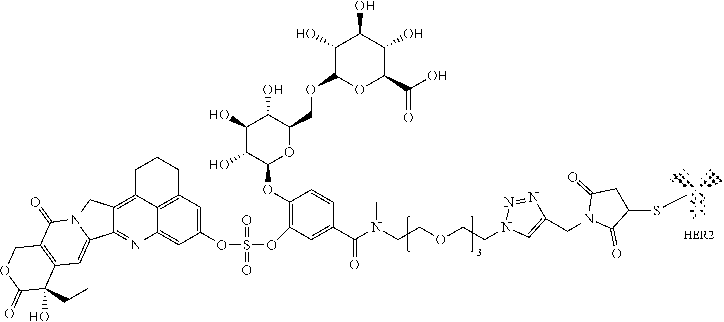
T2-AB1 (7.3) 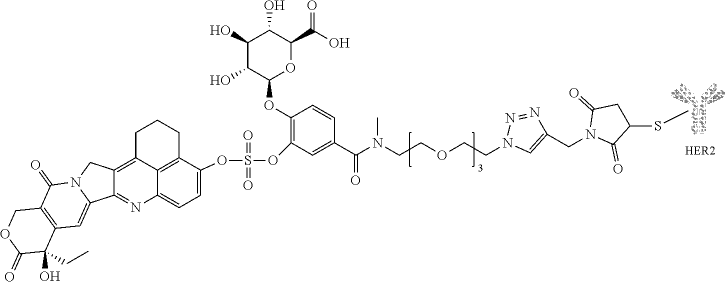
T3-AB1 (7.4) 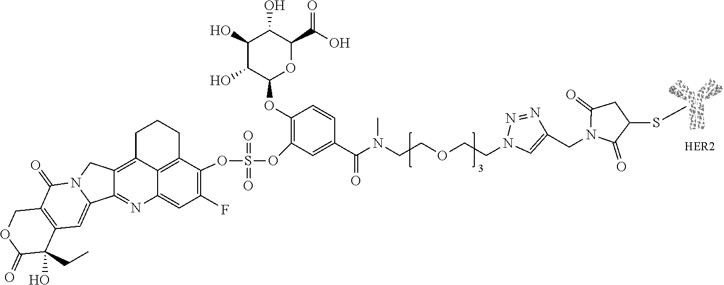
T4-AB1 (7.4) 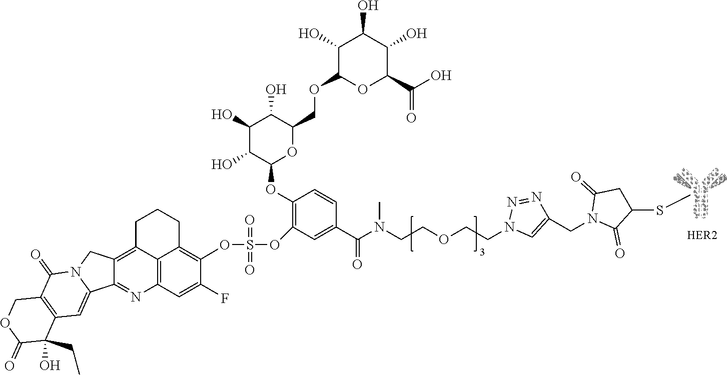
T6-AB1 (7.8) 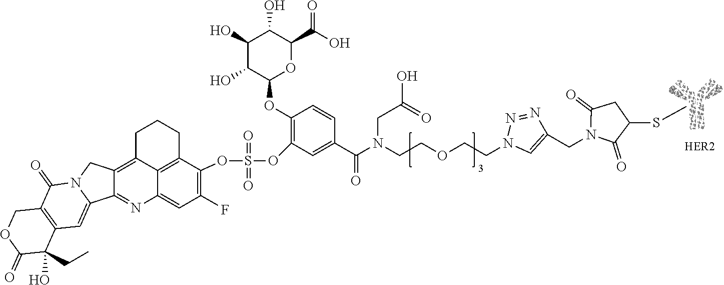
T7-AB1 (7.5) 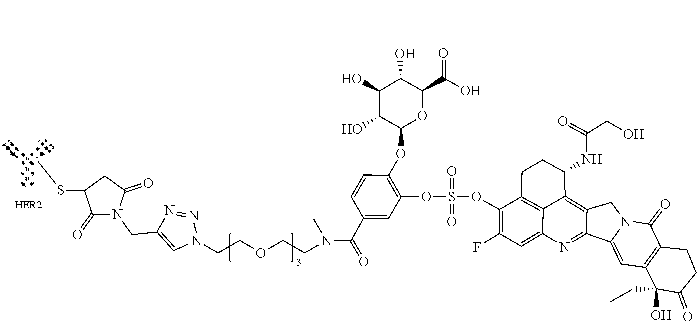
T11-AB1 (7.9) 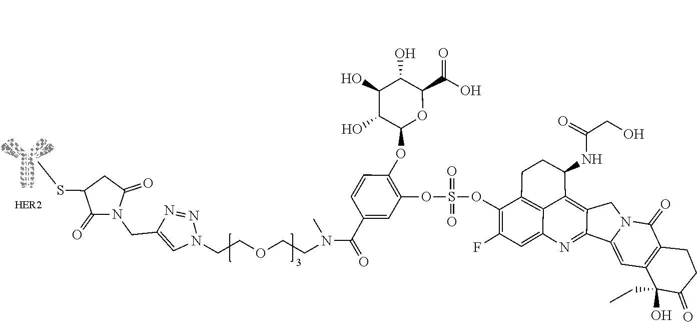
T12-AB1 (8.0) 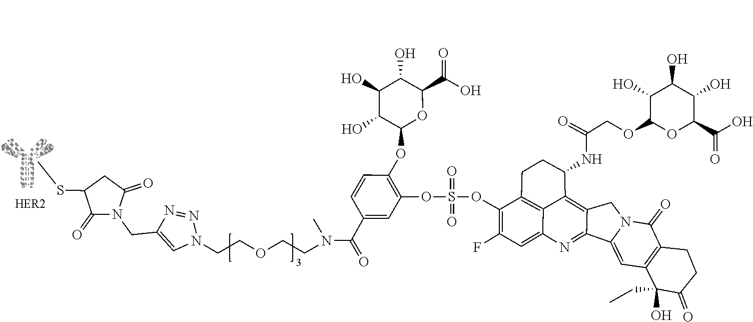
T13-AB1 (6.9) 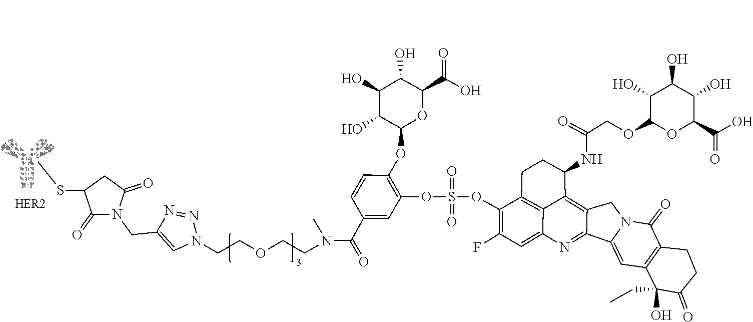
T14-AB1 (6.9) 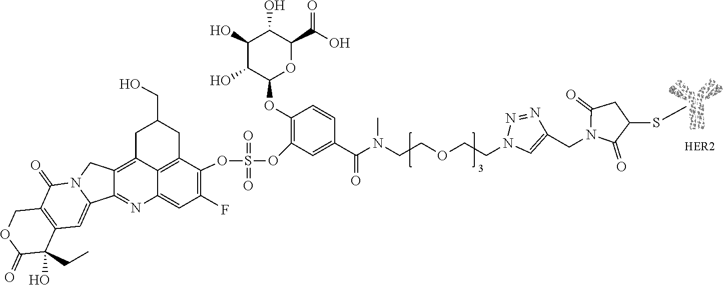
T17-AB1 (8.0) 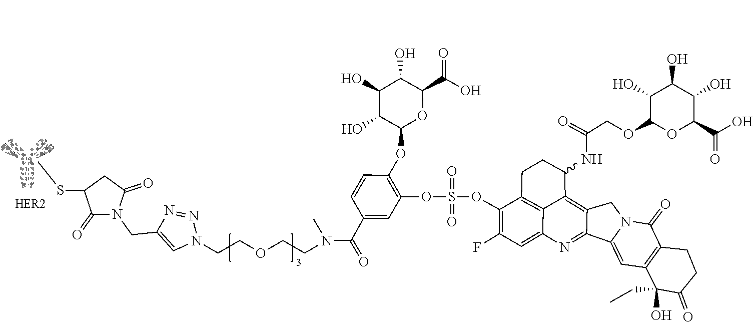
T19-AB1 (7.6) 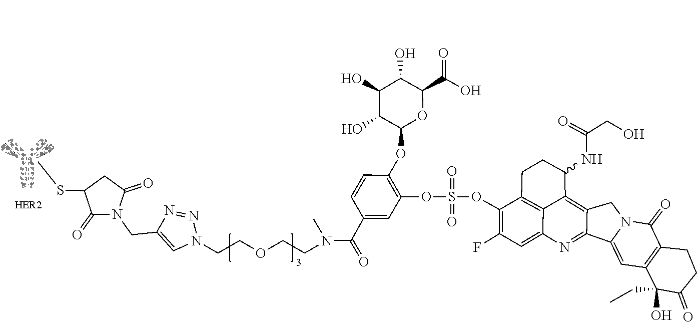
T20-AB1 (6.2) 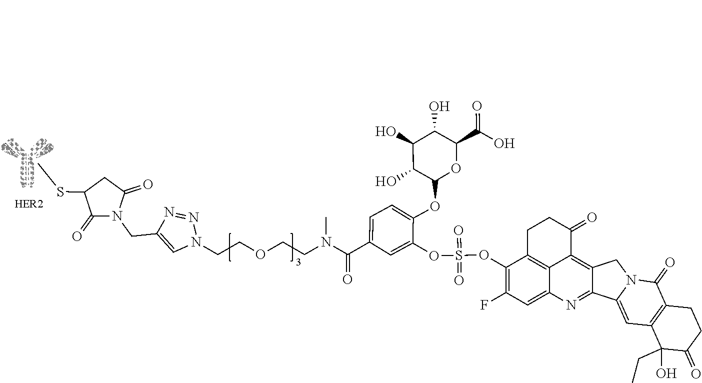
T21-AB1 (7.8) 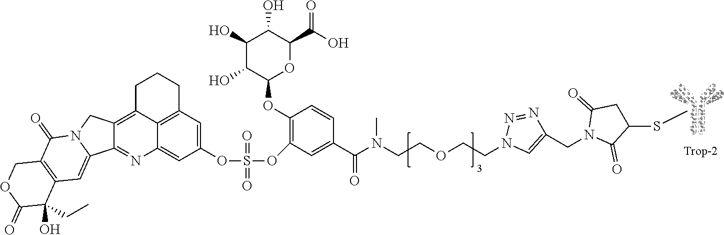
T1-AB2 (7.9) 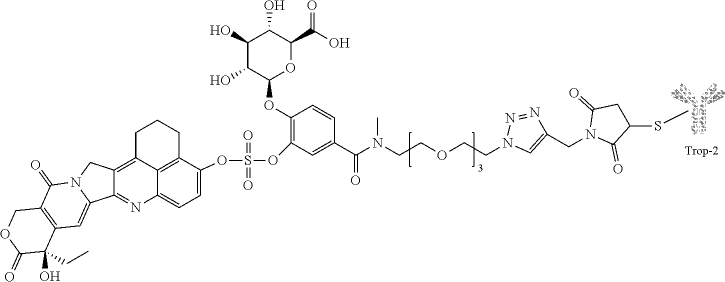
T3-AB2 (7.9) 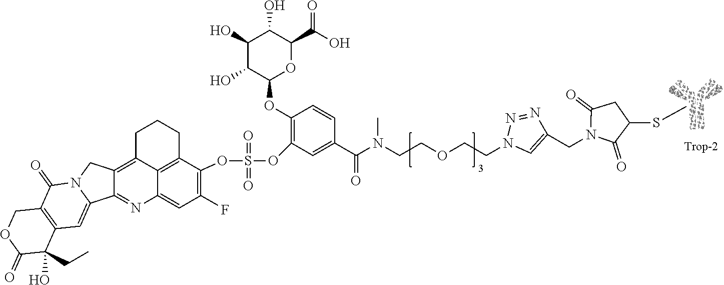
T4-AB2 (7.3) 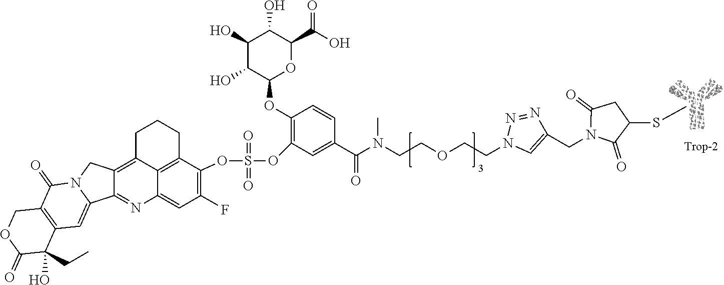
T4-AB2 (8.0) 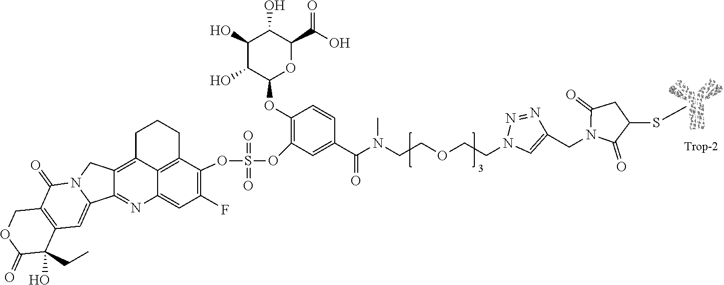
T4-AB2 (4.3) 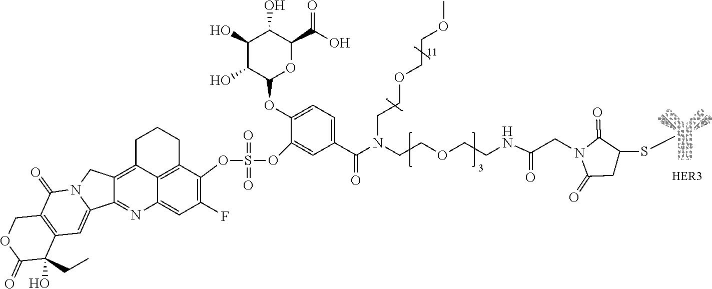
T26-AB3 (7.6) 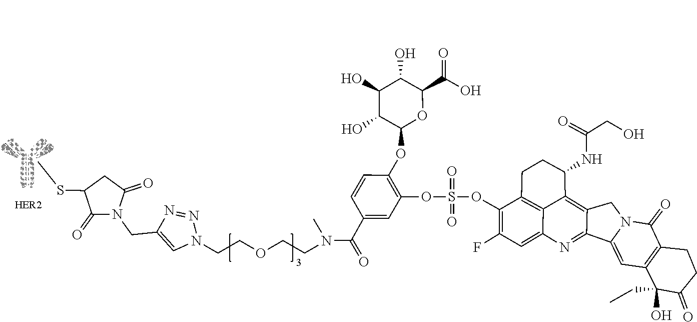
T11-AB1 (7.9) 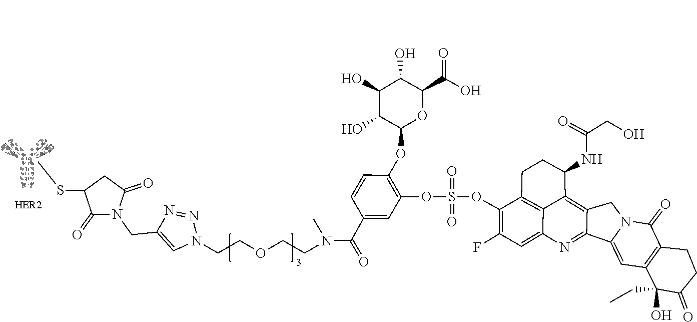
T12-AB1 (8.0) 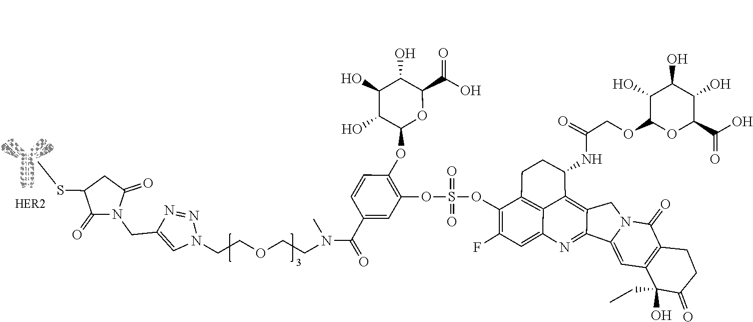
T13-AB1 (6.9) 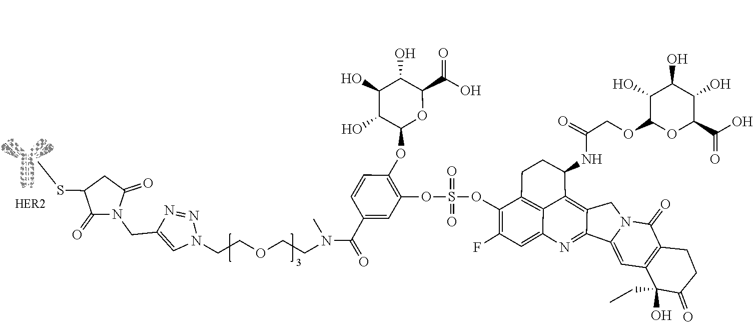
T14-AB1 (6.9) 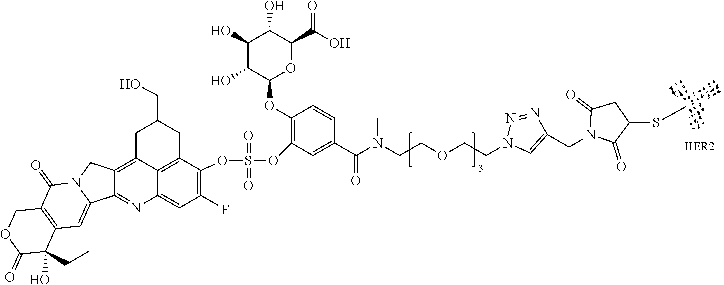
T17-AB1 (8.0) 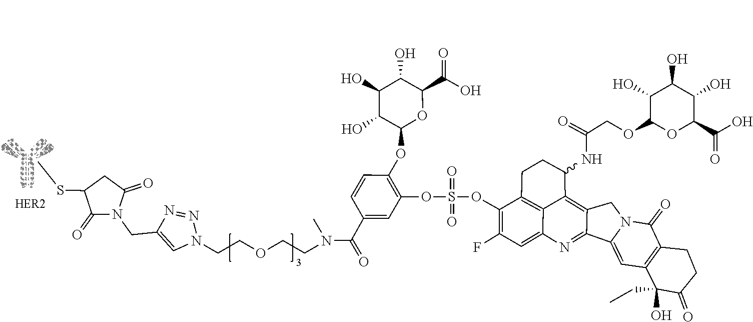
T19-AB1 (7.6) 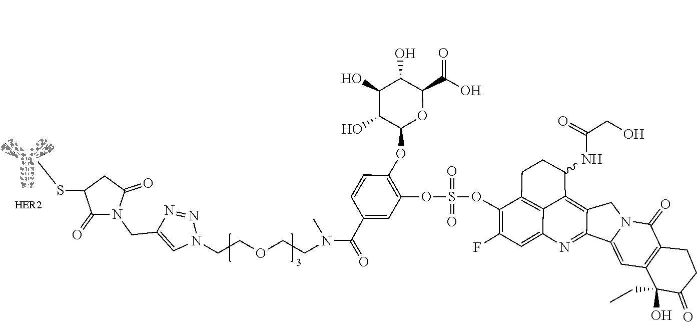
T20-AB1 (6.2) 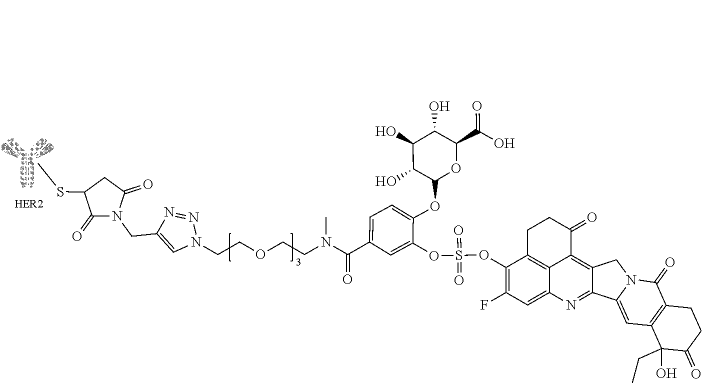
T21-AB1 (7.8)
AB1: HER2
AB2: Trop-2(Sacituzumab)
AB3: HER3(Patritumab)

### Example 35: Conjugation Method 2: (Random Conjugation, Mal-Ring Opening Protocol)

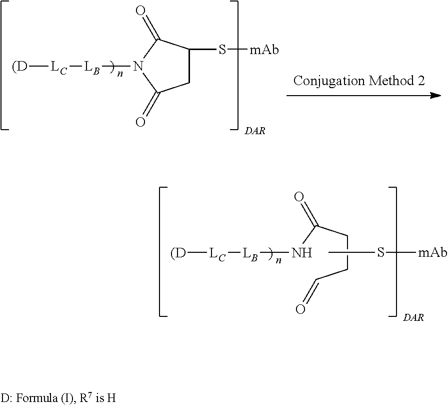

Optionally, an antibody drug conjugate prepared by method as described in Example 34 may perform Mal-ring opening by the following method: The antibody drug conjugate was incubated in borate buffer (pH 9.2) at 30° C. for 15 hours to hydrolyze the maleimide ring. The resulting solution was loaded and eluted through PD-10 column to remove excess linker-drug intermediate and other impurities. And the buffer was exchanged by elution through a Vivaspin column (MWCO, 30 kDa) to PBS (pH 6.5) by repeating 3 times.

TABLE 5
Certain Exemplary ADCs Obtained by Conjugation Method 2 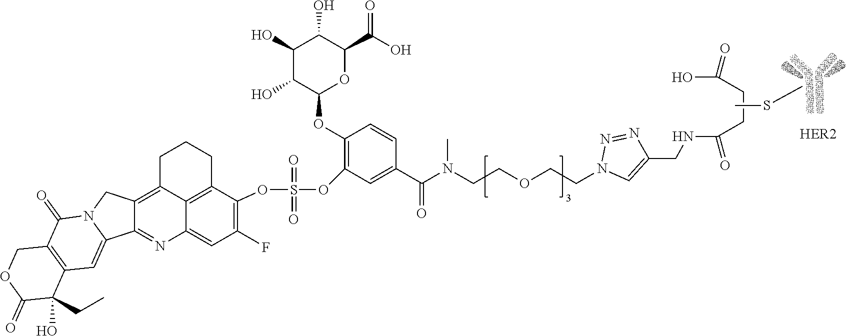
T4(RO)-AB1 (7.9) 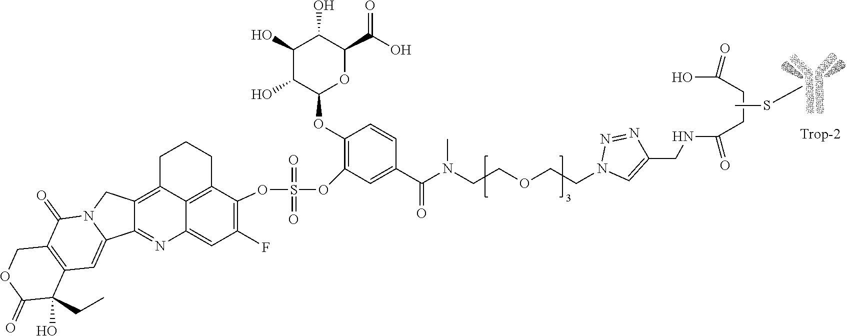
T4(RO)-AB2 (7.5) 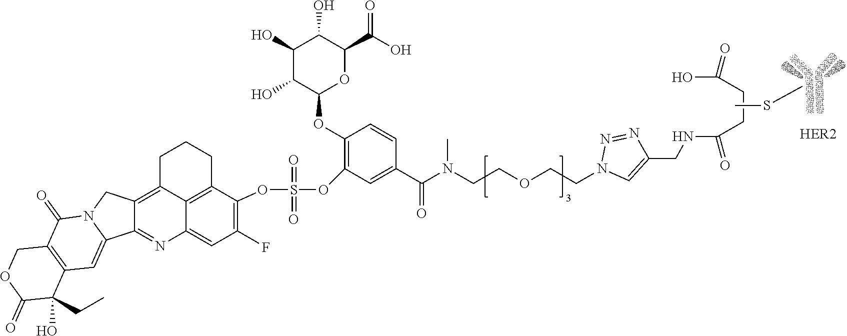
T4(RO)-AB1 (7.9)
AB1: HER2
AB2: Trop-2(Sacituzumab)
AB3: HER3(Patritumab)

### Example 36: Conjugation Method 3: (Random Conjugation, IAA-Conjugation Protocol)

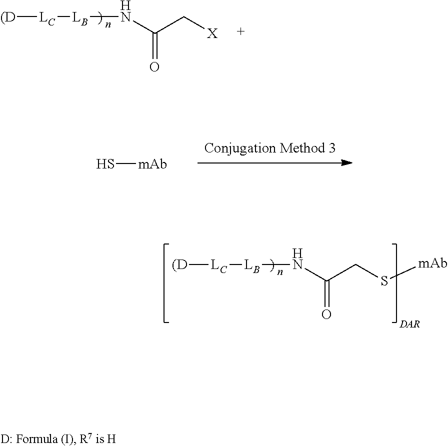

An antibody (70 μM Ab) dissolved in 1×PBS at pH 7.4 was treated with 20 molar excess of TCEP in 25 mM Tris pH 9.0 with 5 mM EDTA. After incubation at 37° C. for 3 hours, the resulting mixture and linker-drug (11 molar excess relative to antibody) were mixed in DMSO containing up to 10% v/v of 1×PBS, and 5 mM EDTA (pH 7.4). And then 0.3 M pH 9.0 borate buffer was added. After mixing, the resulting solution had pH 8.88±0.01. After the reaction was incubated for 2 hours at 37° C., the resulting solution was loaded and eluted through PD-10 column to remove excess linker-drug intermediate and other impurities. And the buffer was exchanged by elution through Vivaspin column (MWCO, 30 kDa) to PBS (pH 6.5) by repeating 3 times.

TABLE 6
Certain Exemplary ADCs Obtained by Conjugation Method 3 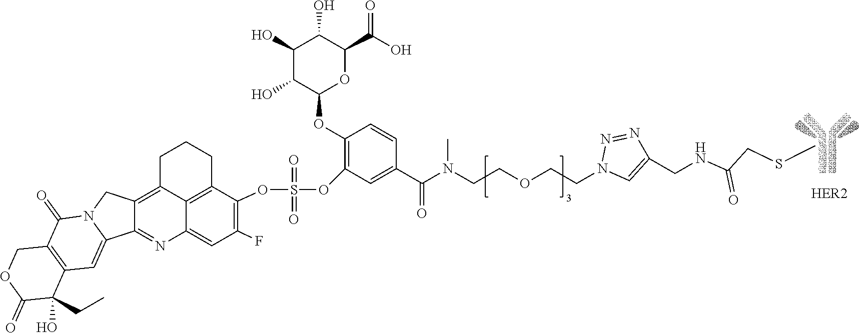
T5-AB1 (7.4) 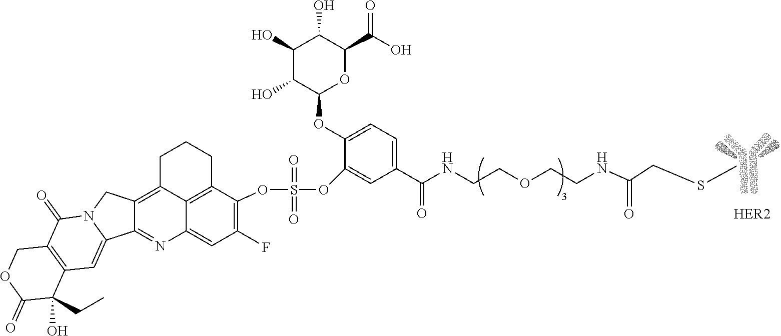
T24-AB1 (7.5) 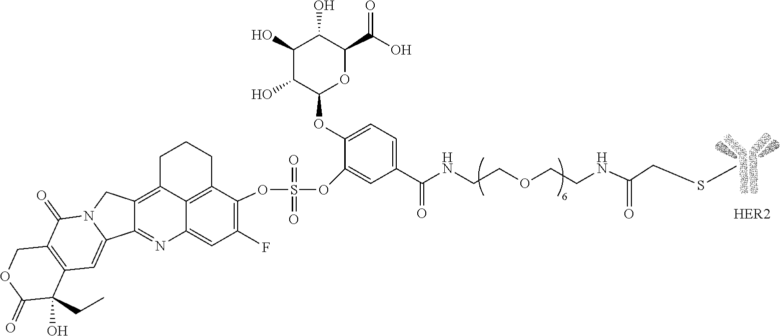
T25-AB3 (6.1) 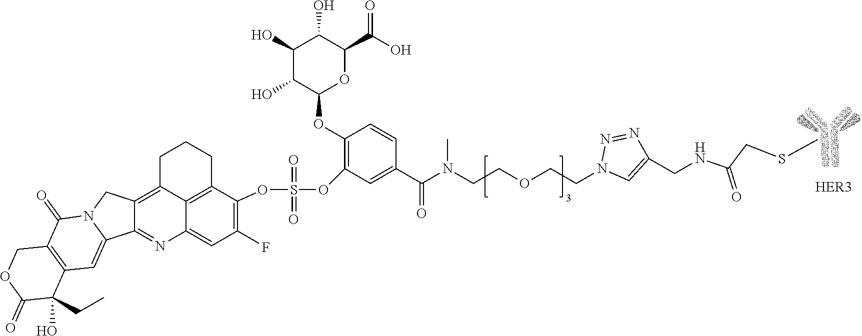
T31-AB3 (7.3) 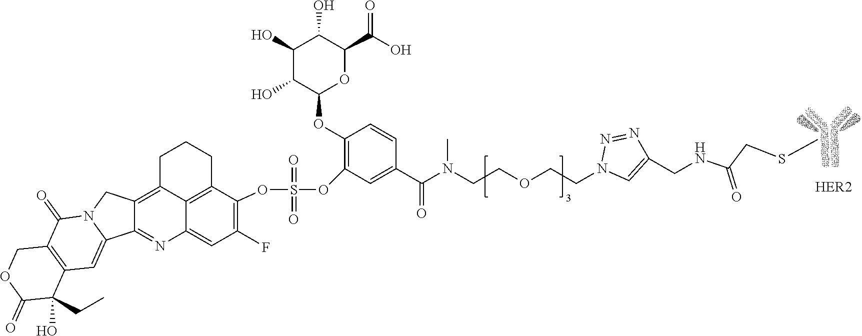
T5-AB1 (7.4) 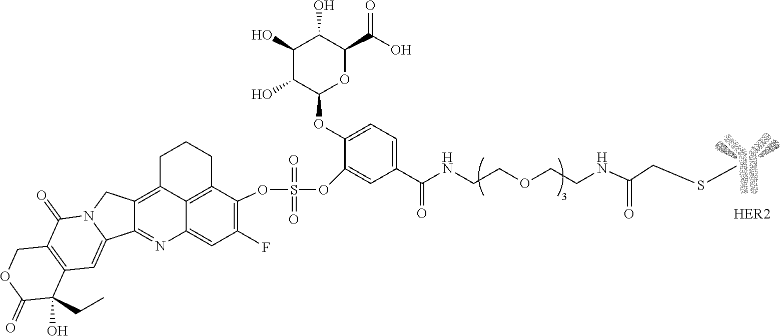
T24-AB1 (7.5) 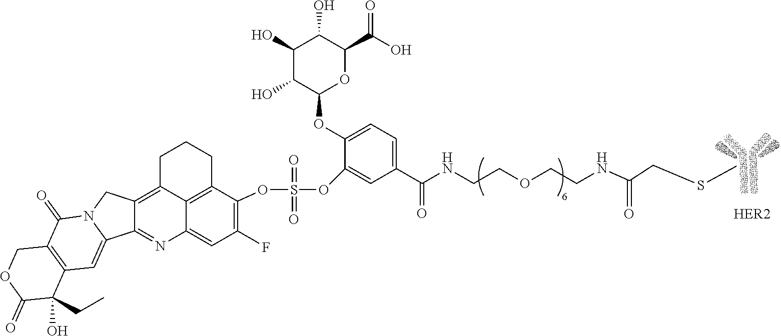
T25-AB1 (6.1)
AB1: HER2
AB2: Trop-2(Sacituzumab)
AB3: HER3(Patritumab)

### Example 37: In Vitro Analysis of Antibody-Drug Conjugates (1)

The antibody-drug conjugates (ADCs) were evaluated on NCI-N₈₇, MDA-MB-468, SK-BR-3, HCT116, and JIMT-1 cancer cells. The cancer cells were seeded in 96-well plates at a density of 2,000 to 5,000 cells per well in 100 μL of medium, and cultured for 22 to 26 hours. The ADCs were diluted by serial dilutions of 1:3-1:10 from 5 μM to 0.01 pM with media. The diluted ADCs were added to triplicate wells of 96-well plates at 50 μL per well. All assays were performed, by repeating nth and the results were obtained in nth independent experiments. The plates were incubated for 6 days at 37° C. in a humidified 5% C02-in-air atmosphere. Cell viability was determined by the MTT assay. The 15 μL of MTT dye dissolved in PBS buffer solution (5 mg/mL) was added to each well of the plates. The formazans formed by reduction of the MTT dye by mitochondrial oxidoreductases in the living cells were dissolved in DMSO. The formazans were measured using the absorbance at 550 nm. IC₅₀ was generated using a sigmoidal dose-response nonlinear regression curve fit (GraphPad software Inc.).

Results of in vitro analysis of the ADCs are shown in the Table 7 below.

TABLE 7

| | ADCs (DAR) | | | | |
|---|---|---|---|---|---|
| cell | IC₅₀ ± SD (nM) | | | | |
| expression | SK-BR3 | NCI-N87 | JIMT-1 | HCT116 | MDA-MB-468 |
| level | (21.8 × 10⁵) | (13.2 × 10⁵) | (1.7 × 10⁵) | (0.2 × 10⁵) | (0.003 × 10⁵) |
| T1-AB1 (7.8) | 0.09 ± 0.05 | N/A | N/A | N/A | N/A |
| T2-AB1 (7.3) | 0.22 ± 0.07 | N/A | N/A | N/A | N/A |
| T3-AB1 (7.4) | 0.11 ± 0.02 | N/A | N/A | N/A | N/A |
| T4-AB1 (7.4) | 0.06 ± 0.02 | 0.22 ± 0.06 | N/A | N/A | N/A |
| T4(RO)-AB1 (7.9) | 0.05 | 0.12 | N/A | N/A | 48.63 |
| T5-AB1 (7.4) | 0.07 ± 0.02 | 0.14 ± 0.02 | N/A | N/A | 13.79 ± 2.49 |
| T6-AB1 (7.8) | 0.56 ± 0.05 | N/A | N/A | N/A | N/A |
| T7-AB1 (7.5) | 0.08 ± 0.01 | 0.16 ± 0.04 | N/A | N/A | N/A |
| T11-AB1 (7.9) | 0.33 ± 0.24 | 0.56 ± 0.12 | N/A | N/A | 771.70 ± 390.75 |
| T12-AB1 (8.0) | 0.14 ± 0.05 | N/A | N/A | N/A | 889.40 ± 470.37 |
| T13-AB1 (6.9) | 0.19 ± 0.06 | N/A | N/A | N/A | 822.40 ± 348.75 |
| T14-AB1 (6.9) | 0.17 ± 0.05 | N/A | N/A | N/A | 474.55 ± 252.51 |
| T17-AB1 (8.0) | 0.06 ± 0.01 | 0.46 ± 0.12 | N/A | N/A | 131.30 ± 0.71 |
| T19-AB1 (7.6) | 0.25 | N/A | N/A | N/A | 180.20 |
| T20-AB1 (6.2) | 0.34 | N/A | N/A | N/A | 179.20 |
| T21-AB1 (7.8) | 0.06 ± 0.02 | N/A | N/A | N/A | 132.50 ± 6.80 |
| T24-AB1 (7.5) | N/A | 0.16 | N/A | N/A | 40.55 |
| T25-AB1 (6.1) | N/A | 0.22 | N/A | N/A | 58.36 |
| A1-AB1 (7.7) | 0.10 ± 0.03 | 0.42 ± 0.14 | N/A | N/A | 57.74 ± 22.32 |
| A2-AB1 (7.4) | 0.52 ± 0.12 | 2.18 ± 2.07 | N/A | N/A | 40.18 ± 4.73 |
| A3-AB1 (3.5) | 0.12 ± 0.09 | 0.07 ± 0.03 | N/A | N/A | 23.20 ± 4.98 |
| A4-AB1 (8.1) | 0.39 ± 0.03 | 1.65 ± 1.12 | N/A | N/A | 3.59 ± 1.72 |

AB1: HER2
A1: Comparative Example 1 (Mal-GGFG-DXd, DAIICHI SANKYO, Lot No.: 376438)
A2: Comparative Example 2 (Mal-bG_OHPAS-SN38(3 + 0PEG))
A3: Comparative Example 3 (MCC-DM1, Roche, Manufacture's serial No. N1026B12)
A4: Comparative Example 4 (Mal-bG_OHPAS-Exatecan(3 + 0PEG)-carbamate)
RO: Mal-Ring Opening

### Example 38. In Vitro Analysis of Antibody-Drug Conjugates (2)

The antibody-drug conjugates (ADCs) were evaluated on Calu-6, NCI-N87, BxPC3, HaCaT, and Pane-1 cancer cells. The cancer cells were seeded in 96-well plates at a density of 2,000 to 5,000 cells per well in 100 μL of medium, and cultured for 22 to 26 hours. The ADCs were diluted by serial dilutions of 1:5˜1:8 from 3 μM to 0.06 pM with media. The diluted ADCs were added to triplicate wells of 96-well plates at 50 μL per well. All assays were performed, by repeating nth and the results were obtained in nth independent experiments. The plates were incubated for 6 days at 37° C. in a humidified 5% C02-in-air atmosphere. Cell viability was determined by the MTT assay. The 15 μL of MTT dye dissolved in PBS buffer solution (5 mg/mL) was added to each well of the plates. The formazans formed by reduction of the MTT dye by mitochondrial oxidoreductases in the living cells were dissolved in DMSO. The formazans were measured using the absorbance at 550 nm. IC₅₀ was generated using a sigmoidal dose-response nonlinear regression curve fit (GraphPad software Inc.).

Results of in vitro analysis of the ADCs are shown in the Table 8 below.

TABLE 8

| | ADCs (DAR) | | | | |
|---|---|---|---|---|---|
| cell | IC₅₀ ± SD (nM) | | | | |
| expression | Calu-6 | NCI-N87 | BxPC3 | HaCaT | Panc-1 |
| level | (0.0025 × 10⁵) | (2.1 × 10⁵) | (7.3 × 10⁵) | (15.1 × 10⁵) | (0.002 × 10⁵) |
| T1-AB2 (7.9) | 133 ± 23.3 | 49.5 ± 38.3 | 0.66 ± 0.17 | 0.33 ± 0.03 | N/A |
| T3-AB2 (7.9) | 58.5 | 24.1 | 0.41 | 0.30 ± 0.01 | N/A |
| T4-AB2 (7.3) | 19.24 | N/A | 0.166 ± 0.06 | N/A | N/A |
| T4-AB2 (8.0) | N/A | N/A | N/A | 0.14 | 120 ± 8.49 |
| T4-AB2 (4.3) | N/A | 7.26 ± 4.98 | 0.23 ± 0.04 | N/A | 161 ± 35.4 |
| T4(RO)-AB2 (7.5) | N/A | 0.22 | 0.107 | N/A | 897 |
| A1-AB2 (7.9) | N/A | 60.0 ± 45.8 | 0.12 ± 0.04 | N/A | 832 ± 434 |
| B1 (4.5) | N/A | 379 ± 8.70 | 0.29 ± 0.17 | N/A | 388 |

AB2: Trop-2(Sacituzumab)
A1: Comparative Example 1 (Mal-GGFG-DXd, DAIICHI SANKYO, Lot No.: 376438)
B1: Comparative Example 2 (Dato-DXd, DS-1062, Lot No.: 281948)

### Example 39. In Vitro Analysis of Antibody-Drug Conjugates (3)

The antibody-drug conjugates (ADCs) were evaluated on BxPC3, Detroit-551, DLD-1, MDA-MB-231 and MDA-MB-453 cancer cells. The cancer cells were seeded in 96-well plates at a density of 1,500 to 4,000 cells per well in 100 μL of medium, and cultured for 22 to 26 hours. The ADCs were diluted by serial dilutions of 1:5˜1:8 from 5 μM to 0.06 pM with media. The diluted ADCs were added to triplicate wells of 96-well plates at 50 μL per well. All assays were performed, by repeating nth and the results were obtained in nth independent experiments. The plates were incubated for 6 days at 37° C. in a humidified 5% C02-in-air atmosphere. Cell viability was determined by the MTT assay. The 15 μL of MTT dye dissolved in PBS buffer solution (5 mg/mL) was added to each well of the plates. The formazans formed by reduction of the MTT dye by mitochondrial oxidoreductases in the living cells were dissolved in DMSO. The formazans were measured using the absorbance at 550 nm. IC₅₀ was generated using a sigmoidal dose-response nonlinear regression curve fit (GraphPad software Inc.).

Results of in vitro analysis of the ADCs are shown in the Table 9 below.

TABLE 9

| | ADCs (DAR) | | | | |
|---|---|---|---|---|---|
| cell | IC₅₀ ± SD (nM) | | | | |
| expression | BxPC3 | Detroit-551 | DLD-1 | MDA-MB-231 | MDA-MB-453 |
| level | (1.54 × 10⁴) | (0.20 × 10⁴) | (2.36 × 10⁴) | (15.1 × 10⁵) | (0.002 × 10⁵) |
| T31-AB3 (7.3) | 5.57 ± 0.94 | 162 ± 151 | N/A | N/A | N/A |
| T26-AB3 (7.6) | 2.61 ± 1.32 | 74.5 ± 57.8 | N/A | N/A | 0.42 |
| A1-AB3 (7.6) | 105 ± 28.4 | 1,566 ± 269 | 558 | >1,000 | 2.00 |

AB3: HER3(Patritumab)
A1: Comparative Example 1 (Mal-GGFG-DXd, DAIICHI SANKYO, Lot No.: 376438)

### Example 40. Cellular Uptake

Cellular inhibition study was preformed via a similar method as described in Example 34. Results of the cellular uptake studies for the ADCs are shown in the Table 10 below. These results show that ADCs exhibits selective uptake in HER2-positive cells.

TABLE 10

| | ADCs (DAR) | | | | |
|---|---|---|---|---|---|
| | IC₅₀ ± SD (nM) | | | Selectivity (Fold) | |
| cell expression | SK-BR3 | NCI-N87 | MDA-MB-468 | MDA-MB-468/ | MDA-MB-468/ |
| level | (21.8 × 10⁵) | (13.2 × 10⁵) | (0.003 × 10⁵) | SK-BR3 | NCI-N87 |
| T4(RO)-AB1 (7.9) | 0.05 | 0.12 | 48.63 | 972.60 | 405.25 |
| T5-AB1 (7.4) | 0.07 ± 0.02 | 0.14 ± 0.02 | 13.79 ± 2.49 | 197.00 | 98.50 |
| T11-AB1 (7.9) | 0.33 ± 0.24 | 0.56 ± 0.12 | 771.70 ± 390.75 | 2338.48 | 1378.04 |
| T12-AB1 (8.0) | 0.14 ± 0.05 | N/A | 889.40 ± 470.37 | 6352.86 | N/A |
| T13-AB1 (6.9) | 0.19 ± 0.06 | N/A | 822.40 ± 348.75 | 4328.42 | N/A |
| T14-AB1 (6.9) | 0.17 ± 0.05 | N/A | 474.55 ± 252.51 | 2791.47 | N/A |
| T17-AB1 (8.0) | 0.06 ± 0.01 | 0.46 ± 0.12 | 131.30 ± 0.71 | 2188.33 | 285.43 |
| T19-AB1 (7.6) | 0.25 | N/A | 180.20 | 720.80 | N/A |
| T20-AB1 (6.2) | 0.34 | N/A | 179.20 | 527.06 | N/A |
| T21-AB1 (7.8) | 0.06 ± 0.02 | N/A | 132.50 ± 6.80 | 2208.33 | N/A |
| T24-AB1 (7.5) | N/A | 0.16 | 40.55 | N/A | 253.44 |
| T25-AB1 (6.1) | N/A | 0.22 | 58.36 | N/A | 265.27 |
| A1-AB1 (7.7) | 0.10 ± 0.03 | 0.42 ± 0.14 | 57.74 ± 22.32 | 577.40 | 137.48 |
| A2-AB1 (7.4) | 0.52 ± 0.12 | 2.18 ± 2.07 | 40.18 ± 4.73 | 77.27 | 18.43 |
| A3-AB1 (3.5) | 0.12 ± 0.09 | 0.07 ± 0.03 | 23.20 ± 4.98 | 193.33 | 331.43 |
| A4-AB1 (8.1) | 0.39 ± 0.03 | 1.65 ± 1.12 | 3.59 ± 1.72 | 9.05 | 2.18 |

AB1: HER2
A1: Comparative Example 1 (Mal-GGFG-DXd, DAIICHI SANKYO, Lot No.: 376438)
A2: Comparative Example 2 (Mal-bG_OHPAS-SN38(3 + 0PEG))
A3: Comparative Example 3 (MCC-DM1, Roche, Manufacture's serial No. N1026B12)
A4: Comparative Example 4 (Mal-bG_OHPAS-Exatecan(3 + 0PEG)-carbamate)_
RO: Mal-Ring Opening

### Example 41: Enzymatic Cleavage Assay Evaluation of Linker-Drug

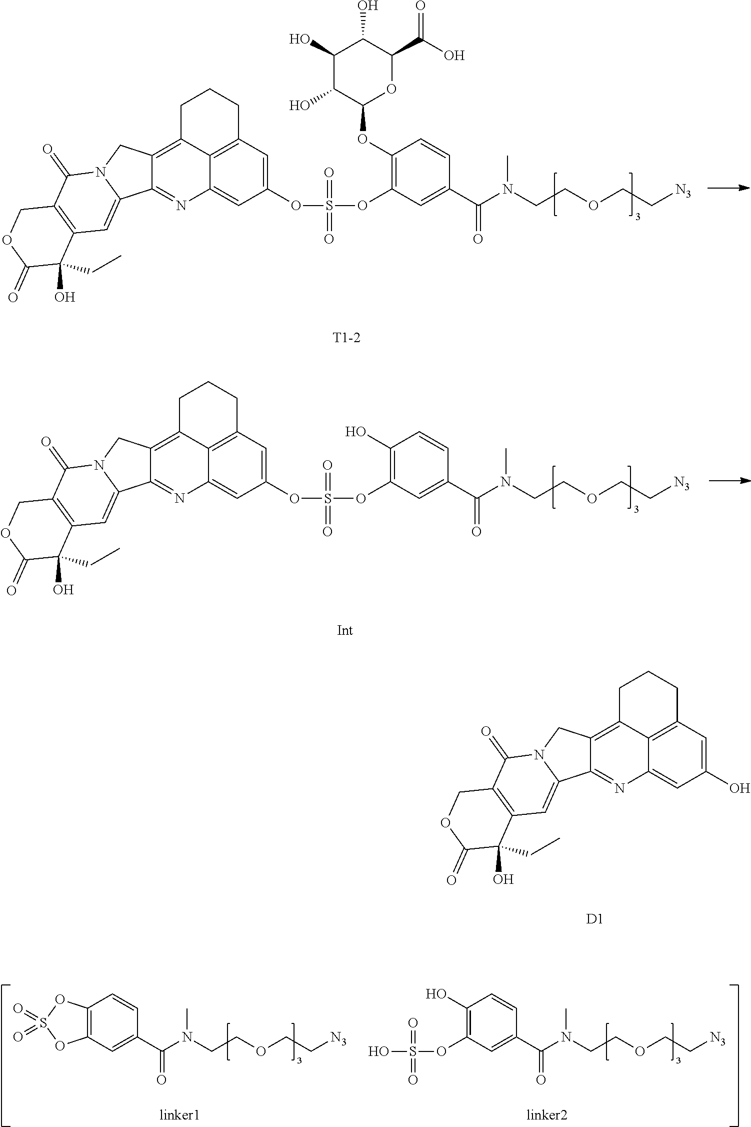

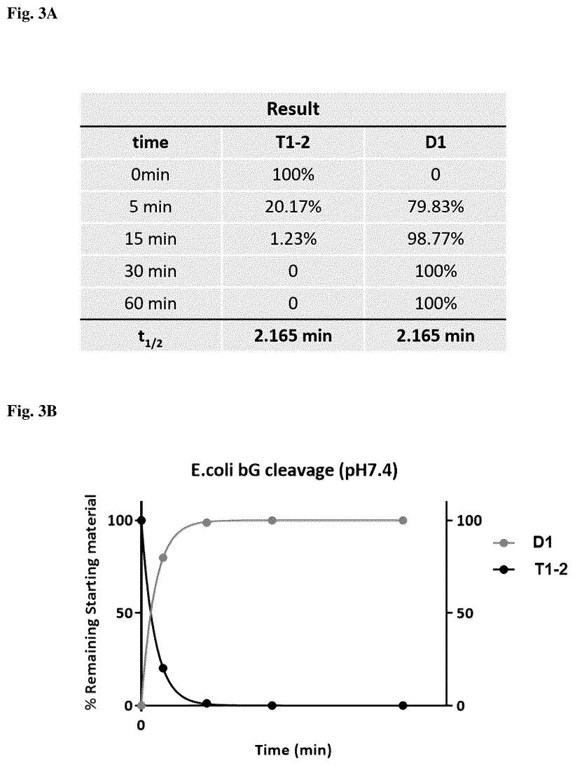

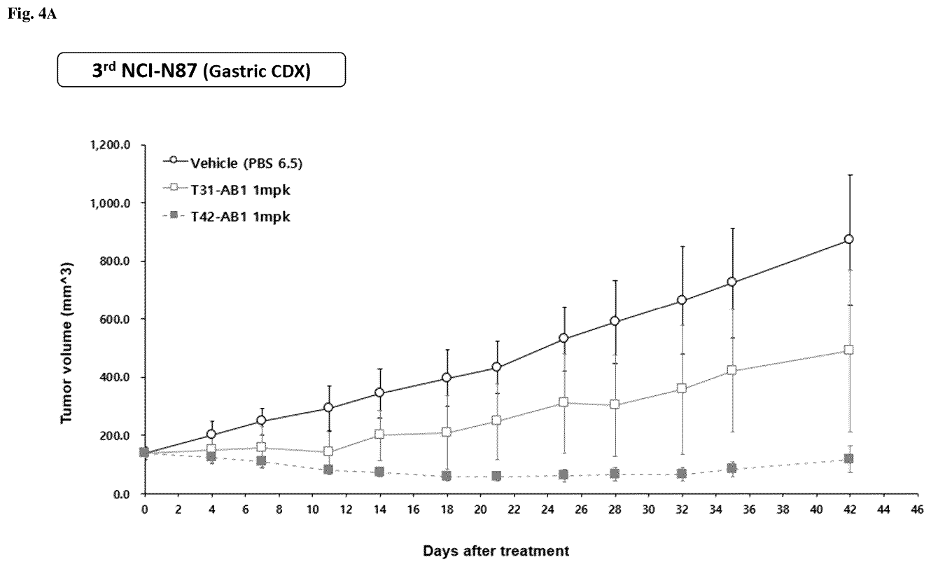

The reactivity of compound T1-2 to β-glucuronidase was evaluated. Compound T1-2 was dissolved in DMSO at a concentration of 10 mM. MPS was dissolved in PBS (phosphate buffered saline, pH 7.4) at the concentration of 500 μM. Premixed solution was prepared by mixing PBS buffer solution (790 μL), 500 μM MPS solution (200 L), and 10 mM compound T1-2 solution (10 μL). An enzyme reaction solution of compound T1-2 was prepared by adding 1 mg/mL enzyme solution (18 μL) to the premixed solution (882 μL), and then the reaction was initiated in the incubator set as 37° C. Beta-glucuronidase enzyme from Escherichia coli (Sigma G7396) was used for the reaction mixture comprising compound T1-2. The enzyme reaction solutions were taken in an aliquot 0 minute (prior to the reaction), 5, 15, 30, and 60 minutes after the reaction in each of 20 μL, and both D1 by an enzyme reaction and remaining compound T1-2 was quantitatively analyzed using HPLC. Test results above were shown in FIGS. 3A-3B, and the hydrolysis half-life by enzyme of compound T1-2 was confirmed to be 2.165 minutes.

### Example 42: In Vivo Efficacy

### 1st Method

In vivo efficacy studies (1^{st}) were performed in a target-expressing xenograft model using the NCI-N87 cell line. The human gastric cancer NCI-N87 xenograft was established in 6-week-old male BALB/c nude mice by implanting 5×10⁶ cells subcutaneously (SC) into their right flanks. When group mean tumor volumes reached approximately 184±22 mm³, the mice were randomized into groups to receive test agents (ADCs), comparative agent (ADC; A1-AB1), or vehicle control (PBS, pH 6.5). The ADCs or PBS were administered intravenously (IV, single) by tail vein injection (1.0 mg/kg). Tumor size and body weight were monitored twice weekly using caliper measurement with tumor sizes calculated as: (long×short×short)/2.

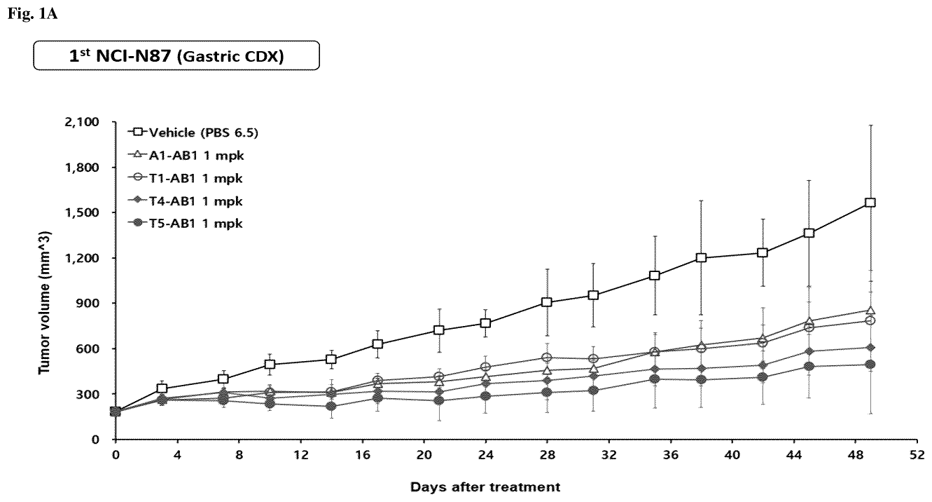

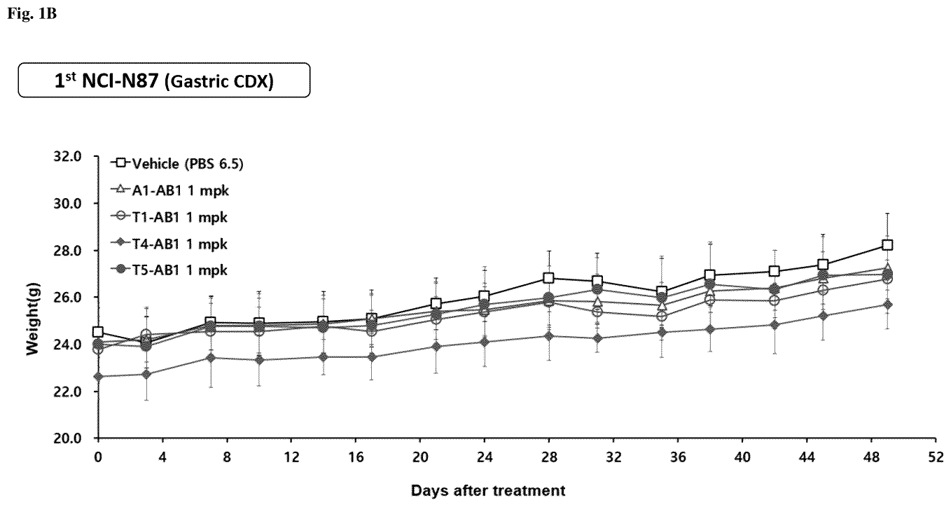

Results of in vivo efficacy of the ADCs are shown in the FIGS. 1A-1B.

### 2^{nd} Method

In vivo efficacy studies (2^{nd}) were performed via a similar method as described in 1^{st} Method. When group mean tumor volumes reached approximately 155±11 mm³, the mice were randomized into groups to receive test agents (ADCs), comparative agent (ADC; A1-AB1), or vehicle control (PBS, pH 6.5). The ADCs (test agents) were administered at 1.0 mg/kg, 2.0 mg/kg, and 4.0 mg/kg. Tumor size and body weight were monitored twice weekly using caliper measurement with tumor sizes calculated as: (long×short×short)/2.

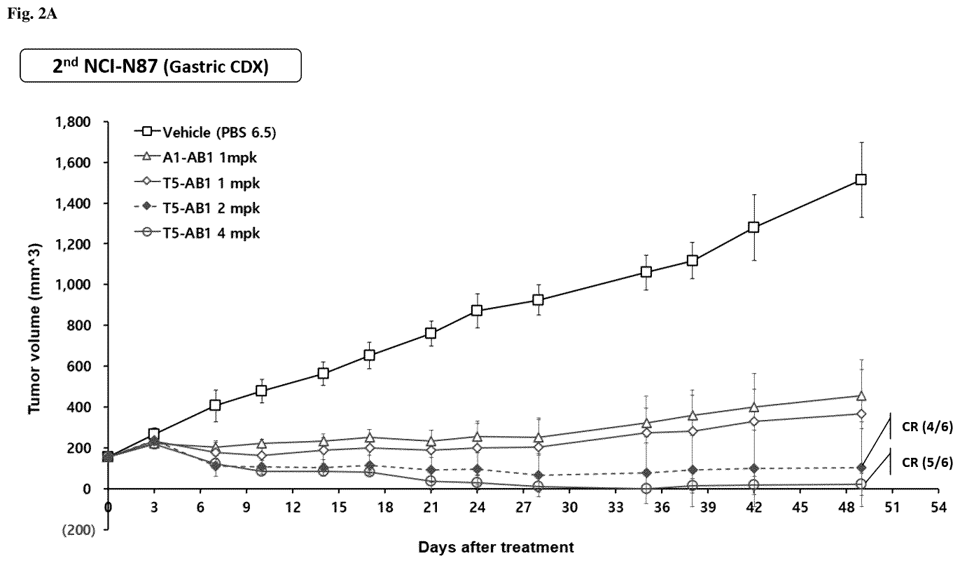

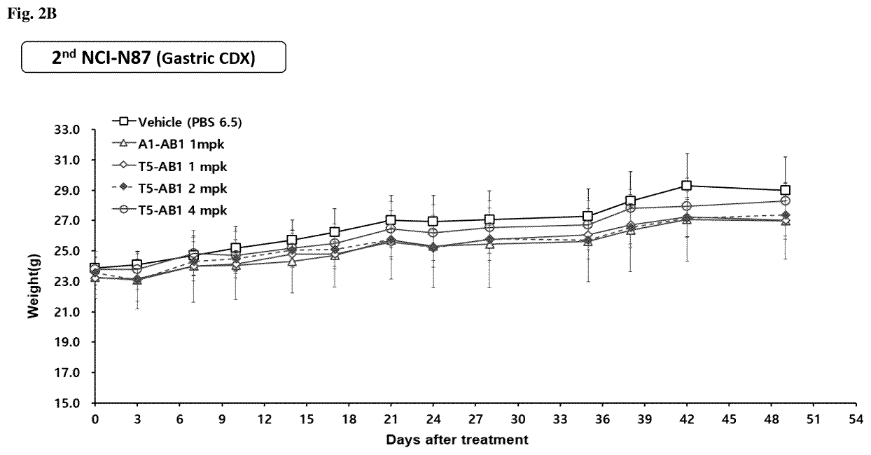

Results of in vivo efficacy of the ADCs are shown in the FIGS. 2A-2B.

### 3^{rd} Method

In vivo efficacy studies (3^{rd}) were performed via a similar method as described in 1^{st} Method. When group mean tumor volumes reached approximately 150±20 mm³, the mice were randomized into groups to receive test agents (ADCs), or vehicle control (PBS, pH 6.5). The ADCs or PBS were administered intravenously (IV, single) by tail vein injection (1.0 mg/kg). Tumor size and body weight were monitored twice weekly using caliper measurement with tumor sizes calculated as: (long×short×short)/2.

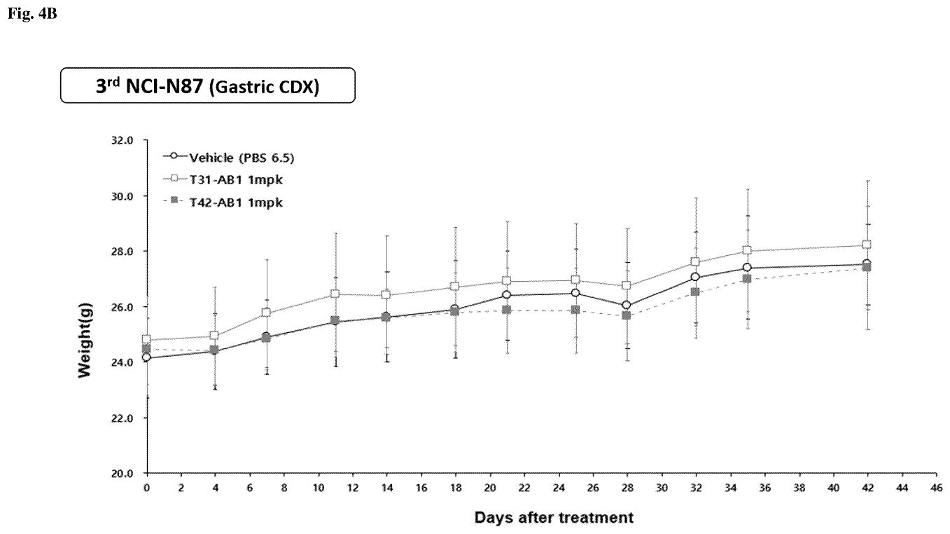

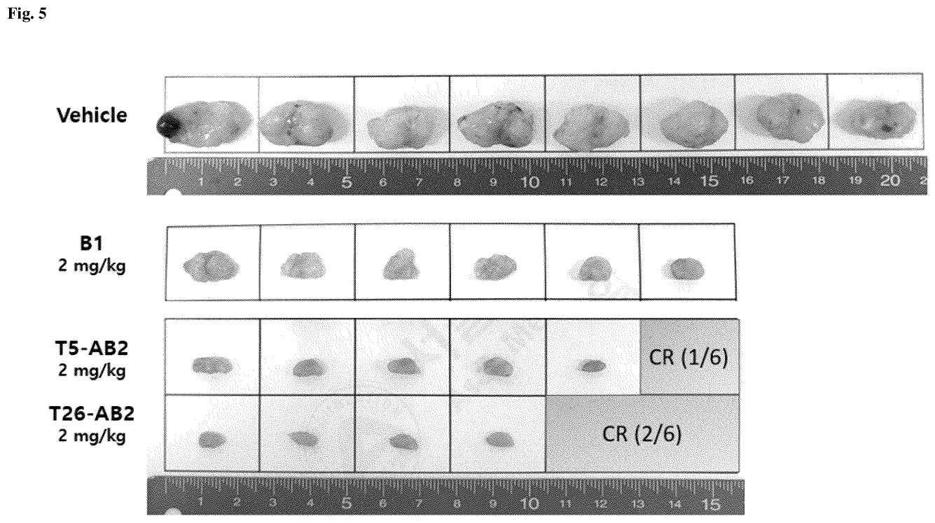

Results of in vivo efficacy of the ADCs are shown in the FIGS. 4A-4B.

### 4^{th} Method

In vivo efficacy studies (4^{th}) were performed via a similar method as described in 1^{st} Method. When group mean tumor volumes reached approximately 199.05±16.38 mm³, the mice were randomized into groups to receive test agents (ADCs), or vehicle control (PBS, pH 6.5). The ADCs comparative agent (ADC; B1), or vehicle control (PBS, pH 6.5) were administered intravenously (IV, single) by tail vein injection (2.0 mg/kg). Tumor size and body weight were monitored twice weekly using caliper measurement with tumor sizes calculated as: (long×short×short)/2.

1. A compound of Formula (I): 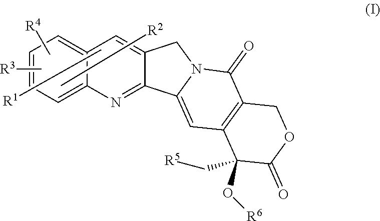
or a pharmaceutically acceptable salt thereof, wherein:
R¹ is selected from —(CH₂)ₜ(Ar¹¹)_{y}OR⁷—, —(CH₂)ₜNHC(═O)(CH₂)ₜOR⁷, —NHC(═O)CR₅═N—OR⁷, —NHC(═O)(CH₂)ₜ(Ar¹¹)_{y}OR⁷, —NH(CH₂)ₜ(Ar¹¹)_{y}OR⁷, and —NHC(═O)-alkyl-OR⁷;
R⁷ is H, -L^{C}-L^{B}-L^{A}, or -L^{C}-L^{B}-L^{D}-TM;
Ar¹¹ is aryl or heteroaryl;
y is 0 or 1;
L^{C} is a cleavage group;
L^{B} is a spacer group;
L^{A} is a reactive group;
L^{D} is a coupling group;
TM is a targeting moiety;
t is independently at each occurrence an integer from 0-5;
R², R³, and R⁴ are each independently selected from H, halo, hydroxy, cyano, nitro, amino, hydroxyamino, aminoacyl, amido, imino, alkyl, heteroalkyl, alkenyl, alkynyl, acyl, acyloxy, carboxyl, alkoxy, hydroxyalkyl, cycloalkyl, heterocycloalkyl, aryl, heteroaryl, (CH₂)Glyc, (CH₂)ₜN(R⁸)₂, (CH₂)ₜC(═O)(CH₂)ₜOR⁸, (CH₂)ₜNHC(═O)(CH₂)OR⁸, NHC(═O)C(═O)R⁸, NH(CH₂)ₜOR⁸, and (CH₂)ₜNHOR⁸; or any two of R¹, R², R³, and R⁴ combine with those carbons to which there are attached to complete an optionally substituted carbocyclyl or heterocyclyl, wherein the carbocyclyl or heterocyclyl, when substituted, is substituted with at least one R¹ or R⁹ substituent;
R⁵ is selected from H, halo, hydroxy, cyano, nitro, amino, hydroxyamino, aminoacyl, amido, imino, alkyl, heteroalkyl, alkenyl, alkynyl, acyl, acyloxy, alkoxy, hydroxyalkyl, cycloalkyl, heterocycloalkyl, aryl, heteroaryl, (CH₂)ₜGlyc, (CH₂)ₜNHC(═O)(CH₂)ₜOR⁸, NHC(═O)C(═O)R⁸, and (CH₂)ₜNHOR⁸;
Glyc is a monosaccharide, disaccharide, or oligosaccharide;
R⁶ is selected from H, C(═O)(CH₂)ₜGlyc, C(═O)(CH₂)OR′, phosphonic acid (—P(═O)(OH)₂), sulfonic acid (—SO₃H), and C(═O)-alkyl;
each R⁸ is independently selected from H, alkyl, acyl, cycloalkyl, heterocyclyl, aryl, heteroaryl, and Glyc; and
R⁹ is selected from H, halo, hydroxy, carboxy, oxo, cyano, nitro, hydroxyamino, amino, aminoacyl, amido, imino, alkyl (e.g., hydroxyalkyl), heteroalkyl, alkenyl, alkynyl, acyl, acyloxy, alkoxy, cycloalkyl, heterocyclyl, aryl, heteroaryl, (CH₂)ₜAr¹¹OR⁸, (CH₂)ₜNH(CH₂)OR⁸, (CH₂)ₜGlyc, (CH₂)ₜNHC(═O)(CH₂)ₜOR⁸, —NHC(═O)CR⁸═N—OR⁸, NHC(═O)C(═O)R⁸, and (CH₂)ₜNHOR⁸.

2-3. (canceled)

4. The compound of claim 1, wherein at least one pair of R¹, R², R³, and R⁴ combine to complete an optionally substituted 5- or 6-membered carbocyclyl or heterocyclyl.

5. The compound of claim 1, wherein:
R¹ is selected from —(CH₂)ₜ(Ar¹¹)_{y}OR⁷—, —(CH₂)ₜNHC(═O)(CH₂)ₜOR⁷, and —NHC(═O)CR₅═N—OR⁷;
R², R³, and R⁴ are each independently selected from H, halo, hydroxy, cyano, nitro, amino, hydroxyamino, aminoacyl, amido, imino, alkyl, heteroalkyl, alkenyl, alkynyl, acyl, acyloxy, alkoxy, hydroxyalkyl, cycloalkyl, heterocycloalkyl, aryl, heteroaryl, (CH₂)ₜGlyc, (CH₂)ₜNHC(═O)(CH₂)ₜOR⁸, NHC(═O)C(═O)R⁸, NH(CH₂)ₜOR⁸, and (CH₂)ₜNHOR⁸; or two of R², R³, and R⁴ are present on vicinal carbons and combine with those carbons to complete a carbocyclyl or heterocyclyl, which may bear the R¹ substituent; and
each R⁸ is independently selected from H, alkyl, acyl, cycloalkyl, heterocyclyl, aryl, and heteroaryl.

6. The compound of claim 1, wherein the compound has a structure of Formula (IIA) or (IIB): 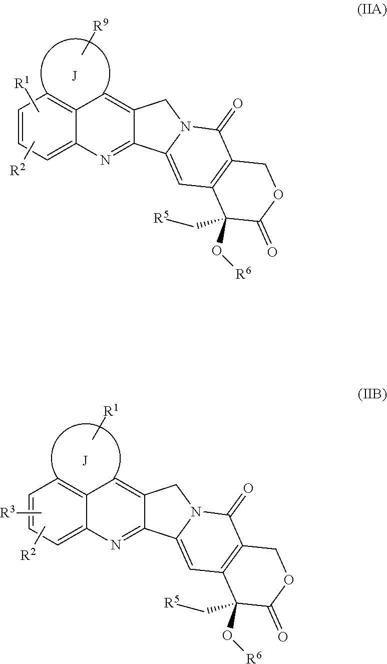
or a pharmaceutically acceptable salt thereof, wherein:
ring J is a 5-membered or 6-membered cycloalkenyl or heterocycloalkenyl.

7. (canceled)

8. The compound of claim 6, wherein the compound has a structure of Formula (III): 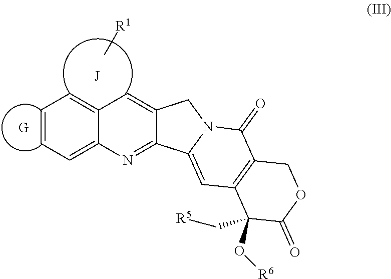
or a pharmaceutically acceptable salt thereof, wherein:
ring G is a 5- or 6-membered carbocyclyl or heterocyclyl ring.

9. The compound of claim 1, wherein R², R³, and R⁴ are each independently selected from H, halo, hydroxy, cyano, nitro, amino, hydroxyamino, aminoacyl, amido, imino, alkyl, heteroalkyl, alkenyl, alkynyl, acyl, acyloxy, alkoxy, hydroxyalkyl, cycloalkyl, heterocycloalkyl, aryl, heteroaryl, (CH₂)₁Glyc, (CH₂)ₜNHC(═O)(CH₂)ₜOR⁸, NHC(═O)C(═O)R⁸, NH(CH₂)ₜOR⁸, and (CH₂)ₜNHOR⁸.

10. The compound of claim 1, wherein the compound has a structure of Formula (IVA), (IVB), or (IVC): 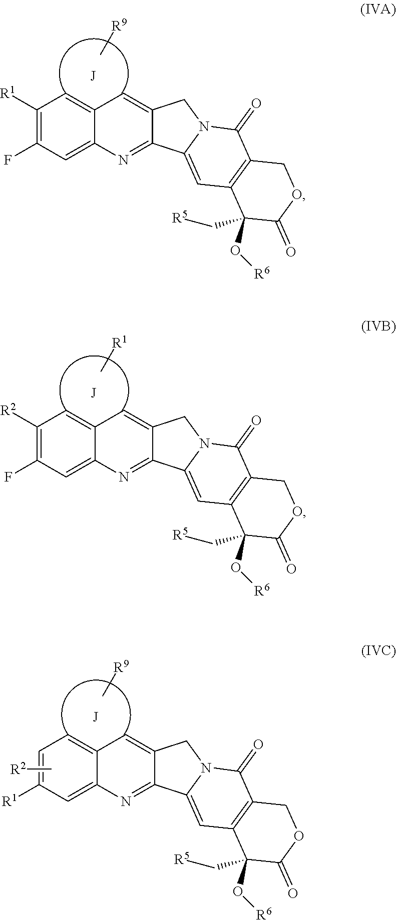
or a pharmaceutically acceptable salt thereof, wherein:
ring J is a 6-membered carbocyclyl or heterocyclyl.

11. (canceled)

12. The compound of claim 10, wherein the compound has a structure of Formula (VA): 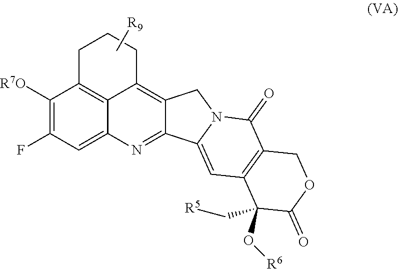
or a pharmaceutically acceptable salt thereof.

13. (canceled)

14. The compound of claim 1, wherein the compound has a structure of Formula (VIA) or (VIB): 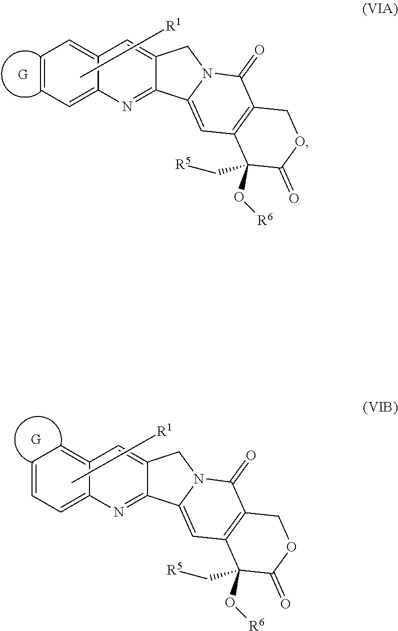
or a pharmaceutically acceptable salt thereof, wherein:
ring G is a 5- or 6-membered carbocyclyl or heterocyclyl.

15. (canceled)

16. The compound of claim 1, wherein the compound has a structure of Formula (VIIA) or (VIIB): 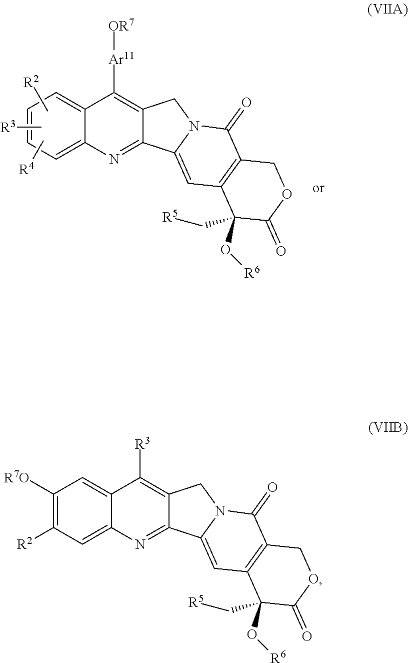
or a pharmaceutically acceptable salt thereof.

17. The compound of claim 16, wherein the compound has a structure of Formula (VIIIA): 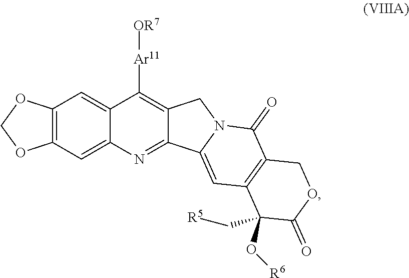
or a pharmaceutically acceptable salt thereof.

18-25. (canceled)

26. The compound of claim 1, wherein R⁷ is L^{C}-L^{B}-L^{A}.

27. The compound of claim 26, wherein L^{A} is selected from isocyanide, isothiocyanide, 2-pyridyl disulfide, haloacetamide (—NHC(═O)CH₂-halo), maleimide, diene, alkene, halide, tosylate, aldehyde, sulfonate, 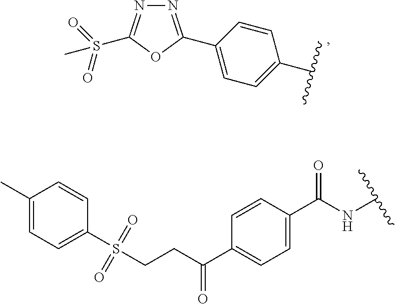 [SMILES: *c1ccc(-c2nnc(S(C)(=O)=O)o2)cc1 ; *NC(=O)c1ccc(C(=O)CCS(=O)(=O)c2ccc(C)cc2)cc1]
phosphonic acid (—P(═O)(OH)₂), ketone, C₈-C₁₀ cycloalkynyl, —OH, —NHOH, —NHNH₂, —SH, carboxylic acid, alkyne, azide, amino, sulfonic acid, an alkynone derivative (—C(O)C═C—Rₐ, wherein Rₐ is H or alkyl), and dihydrogen phosphate (—OP(═O)(OH)₂).

28-29. (canceled)

30. The compound of claim 1, wherein R⁷ is L^{C}-L^{B}-L^{D}-TM.

31. The compound of claim 26, wherein L^{C} is selected from 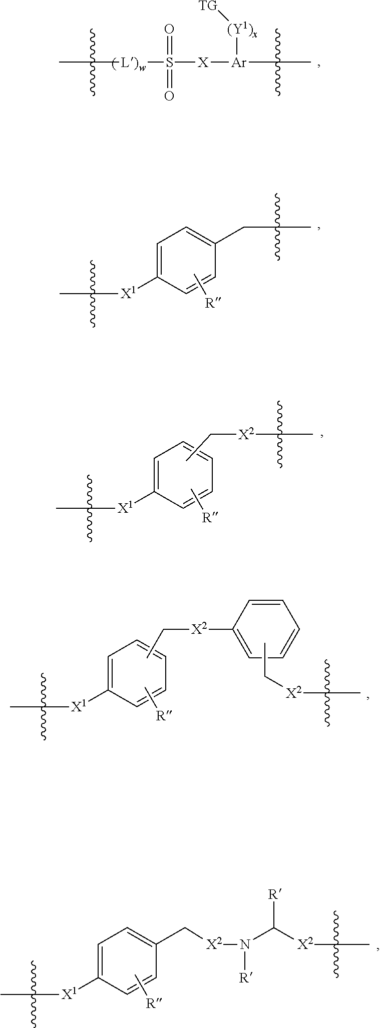
or a combination thereof,
wherein:
R′ is H or alkyl;
L′ is a spacer group attached to the SO₂ via a heteroatom selected from O, S, and N, preferably O or N, and selected such that cleavage of the bond between L′ and SO₂ promotes cleavage of the bond between L′ and the remainder of the compound;
w is 0 or 1;
x is 0 or 1;
X is selected from —O—, —CR^{b}₂, —S— and —NR^{a}—, where R^{a} and R^{b} are each independently at each occurrence H or alkyl;
Y¹ is —(CR^{b}₂)_{z}N(R^{a})—, —(CR^{b}₂)_{z}O—, or —(CR^{b}₂)_{z}S—, wherein z is an integer having a value of 0-5 and if z is 1-5, the N, O, or S atom is attached to TG, and wherein R^{a} and R^{b} are each independently for each occurrence H or alkyl;
at least one X is positioned in an ortho relationship or a para relationship to Y¹ on Ar;
Ar is aryl, heteroaryl, cycloalkyl, or heterocycloalkyl;
TG is a triggering group that, when activated, generates an O atom capable of reacting with the SO₂ to displace the remainder of the compound and form a 5-6-membered ring including X—SO₂ and the intervening atoms of Ar;
X¹ is —O— or NR′;
X² is —C(O), —C(O)O, —C(O)NR′—, or —NR′C(O)O—;
R″ is —SO₄, —(CH₂)ₜ(OCH₂CH₂)_{g}—R′, or —(CH₂)Glyc; and
g is an integer having a value of 0-10.

32-40. (canceled)

41. The compound of claim 30, wherein:
L^{C} is

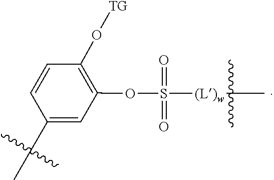

42-47. (canceled)

48. The compound of claim 41, wherein TG is a triggering group selected from —NO₂, —OC(═O)(CH₂)ᵣC(═O)R^{1A}, —NHOH, —BR^{1A}R^{1A}—NHNH₂ nitrobenzyl, 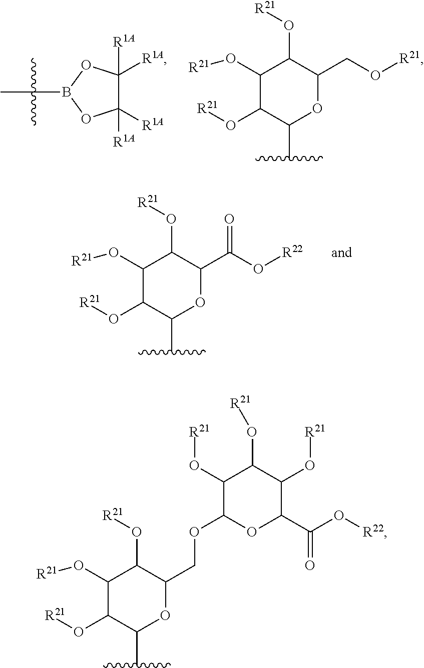
wherein:
R^{1A} is alkyl;
each R²¹ is independently hydrogen or acetyl;
R²² is hydrogen or C₁₋₆ alkyl; and
r is an integer from 0-5.

49-50. (canceled)

51. The compound of claim 26, wherein L^{B} is selected from a C₇-C₁₀₀ linear or branched, saturated or unsaturated alkylene moiety comprising at least two of the following:
(i) at least one moiety selected from —NH—, —C(═O), —O—, —S— and —P—;
(ii) at least one heteroarylene, wherein the heteroarylene is selected from 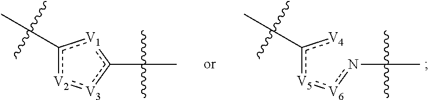
wherein:
V₁, V₂, and V₃ are independently selected from CH and N; and
V₄, V₅, and V₆ are each independently selected form C, CH, O, S, N, and NH;
preferably a triazolene;
provided that the heteroarylene may be fused to a saturated or unsaturated ring;
(iii) at least one amino acid moiety, sugar bond, peptide bond, or amide bond; and
(iv) one or more substitutents selected from the group consisting of C₁-C₂₀ alkyl, C₆-C₂₀ aryl C₁-C₈ alkyl, (CH₂)ₛCOOH, ((OCH₂CH₂)ₙ)ₚR′ and (CH₂)ₚNH₂, wherein each s and n independently is an integer having a value of 0 to 10, R′ is H or alkyl, and p is an integer having a value of 1 to about 10.

52. The compound of claim 26, wherein L^{B} comprises at least one amide bond and is selected from: 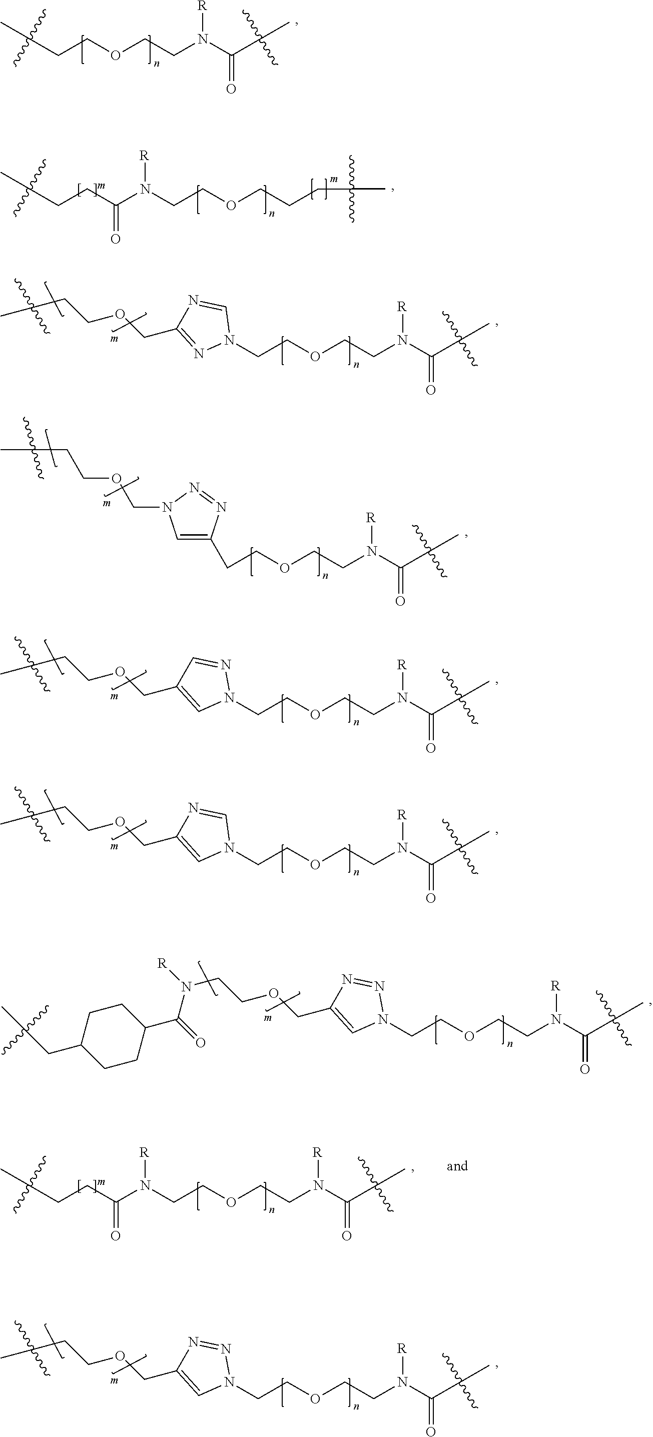
wherein each R is independently (CH₂)ₐₐR′;
aa is an integer from 0 to 10;
R′ is selected from H, hydroxy, aryl, cycloalkyl, nitro, amino, cyano, halo, (CH₂CH₂O)_{bb}R″, and C(═O)OR″;
R″ is selected from H, alkyl, and hydroxyl;
bb is an integer from 1 to 50;
n is an integer from 0 to 10; and
m is an integer from 0 to 10.

53-54. (canceled)

55. The compound of claim 26, wherein L^{B} is: 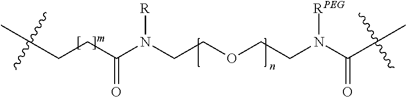
wherein
m is an integer from 0 to 10;
n is an integer from 0 to 10;
R is (CH₂)ₐₐR′;
aa is 0;
R′ is H;
R^{PEG} is 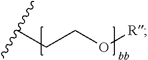
R″ is selected from H, alkyl, and hydroxyl; and
bb is an integer from 0 to 50.

56. (canceled)

57. The compound of claim 26, wherein L^{B} is: 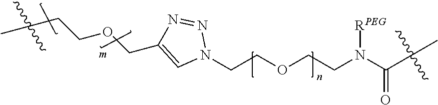
wherein
m is an integer from 0 to 10;
n is an integer from 0 to 10;
R^{PEG} is 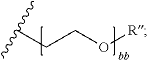
R″ is selected from H, alkyl, and hydroxyl; and
bb is an integer from 0 to 50.

58-59. (canceled)

60. The compound of claim 30, wherein L^{D} comprises a group that can be produced through a coupling reaction, e.g. the reaction of (a) a maleimide and a thiol; (b) a reaction between an azide and an alkyne, or (c) a haloacetamide and a thiol.

61. The compound of claim 60, wherein L^{D} comprises a linking unit formed from a precursor selected from isocyanide, isothiocyanide, 2-pyridyl disulfide, haloacetamide (e.g., —NHC(═O)CH₂-halo), maleimide, diene, alkene, halide, tosylate, aldehyde, sulfonate, 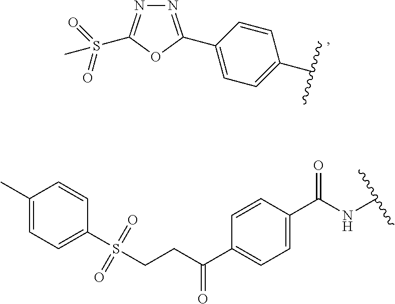 [SMILES: *c1ccc(-c2nnc(S(C)(=O)=O)o2)cc1 ; *NC(=O)c1ccc(C(=O)CCS(=O)(=O)c2ccc(C)cc2)cc1]
phosphonic acid (—P(═O)(OH)₂), ketone, C₈-C₁₀ cycloalkynyl, —OH, —NHOH, —NHNH₂, —SH, carboxylic acid, alkyne, azide, amino, sulfonic acid, an alkynone derivative (—C(O)C═C—Rₐ where Rₐ is H or alkyl), and dihydrogen phosphate (—OP(═O)(OH)₂).

62. The compound of claim 60, wherein L^{D} comprises a triazole, thiosuccinimide, tetrazole, thioacetamide, 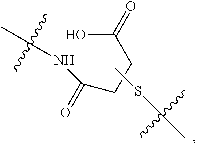
or thioether.

63-80. (canceled)

81. The compound of claim 1, wherein TM is selected from a nanoparticle, an immunoglobulin, a nucleic acid, a protein, an oligopeptide, a polypeptide, an antibody, a fragment of an antigenic polypeptide, or a repebody.

82-87. (canceled)

88. The compound of claim 1, wherein the compound is of one of the following structures, or is a pharmaceutically acceptable salt thereof:

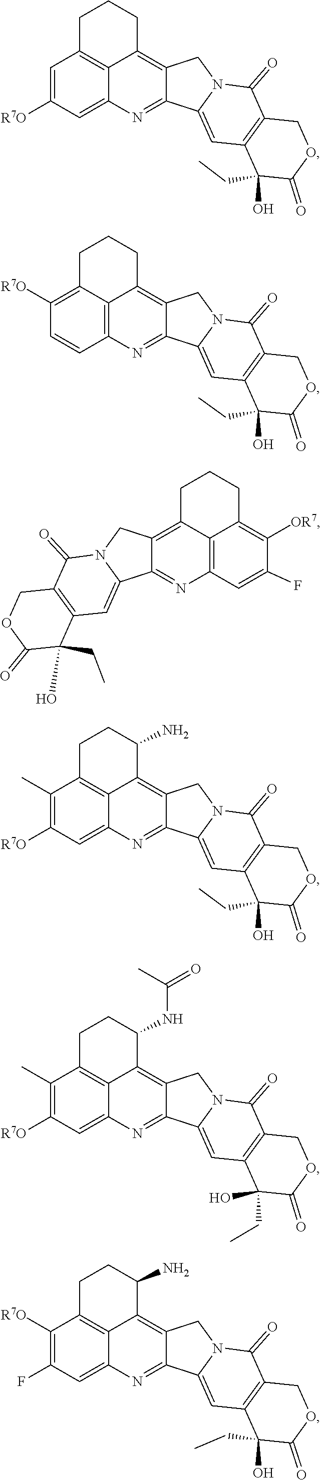

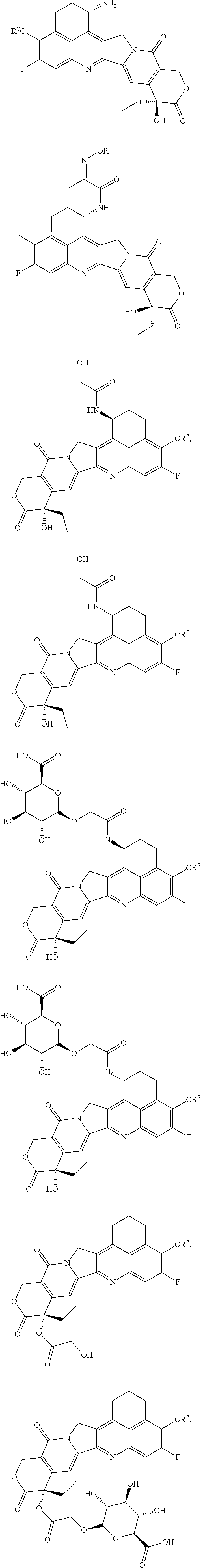

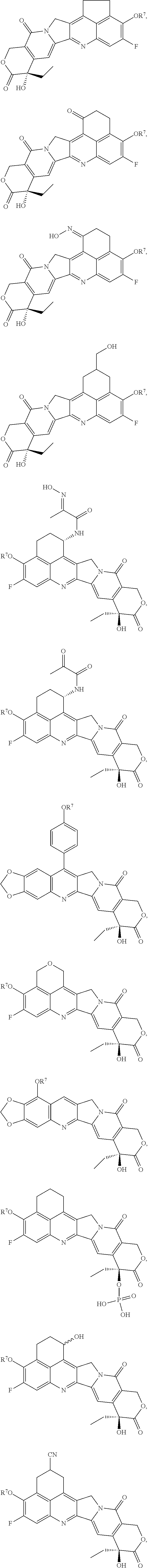

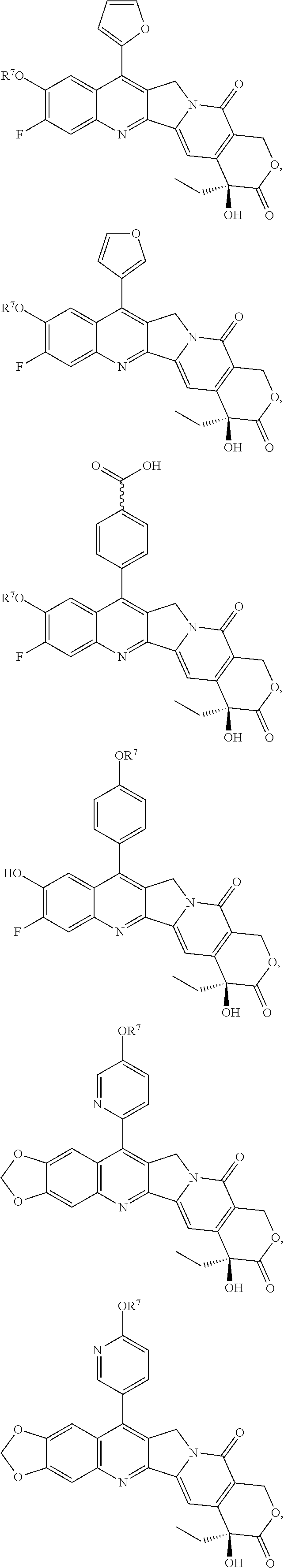

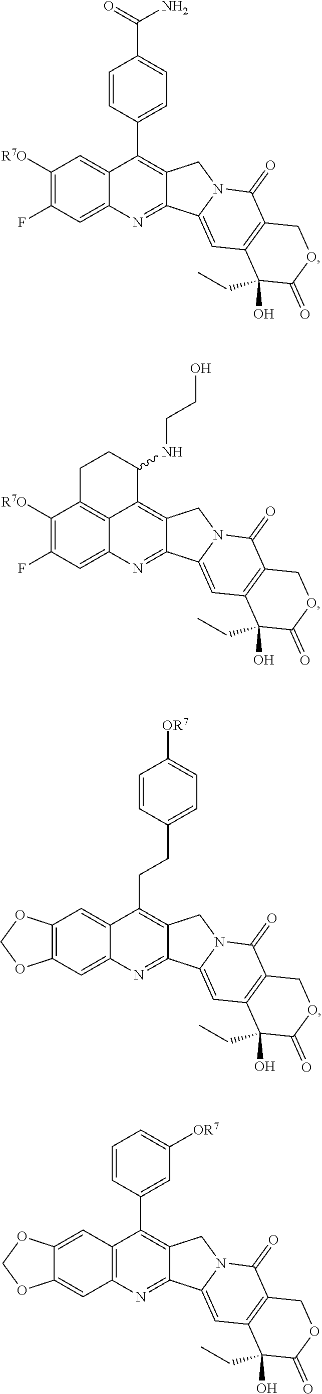

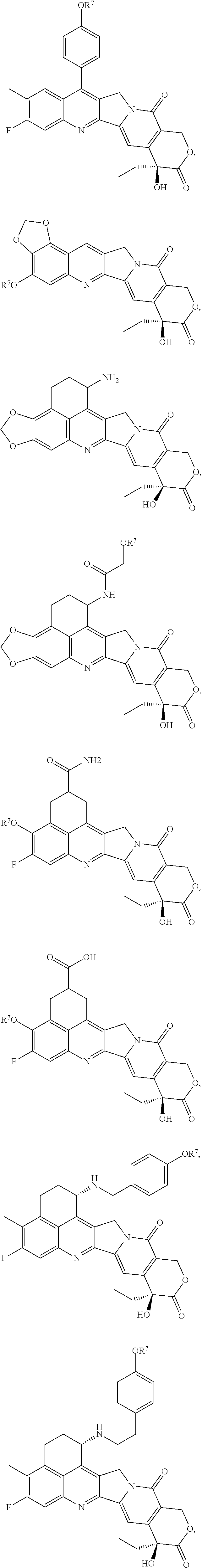

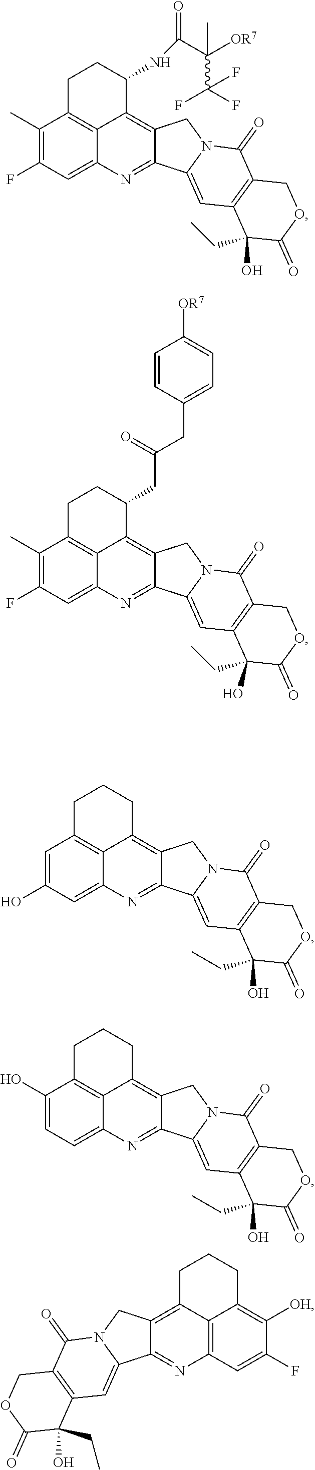

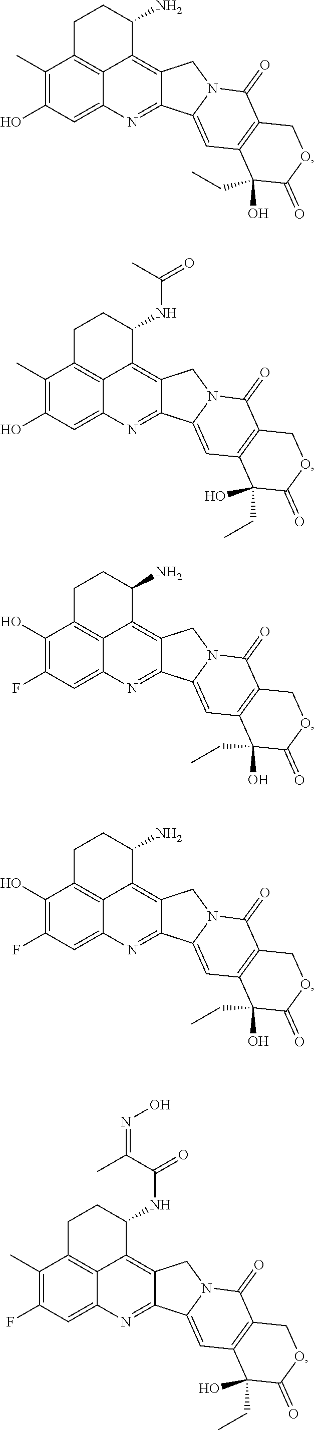 [SMILES: CC[C@@]1(O)C(=O)OCc2c1cc1n(c2=O)Cc2c-1nc1cc(O)c(C)c3c1c2[C@@H](N)CC3 ; CC[C@@]1(O)C(=O)OCc2c1cc1n(c2=O)Cc2c-1nc1cc(O)c(C)c3c1c2[C@@H](NC(C)=O)CC3 ; CC[C@@]1(O)C(=O)OCc2c1cc1n(c2=O)Cc2c-1nc1cc(F)c(O)c3c1c2[C@H](N)CC3 ; CC[C@@]1(O)C(=O)OCc2c1cc1n(c2=O)Cc2c-1nc1cc(F)c(O)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)/C(C)=N\O)CC3]

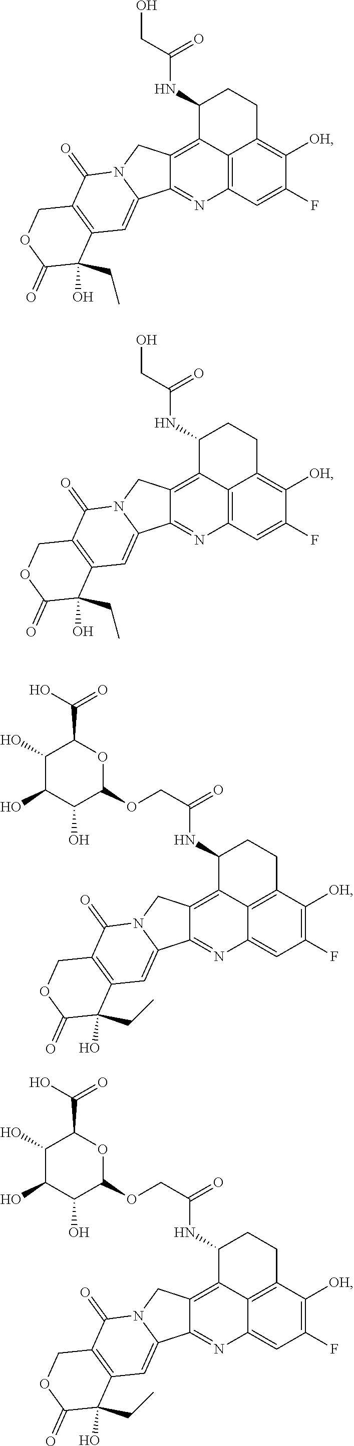

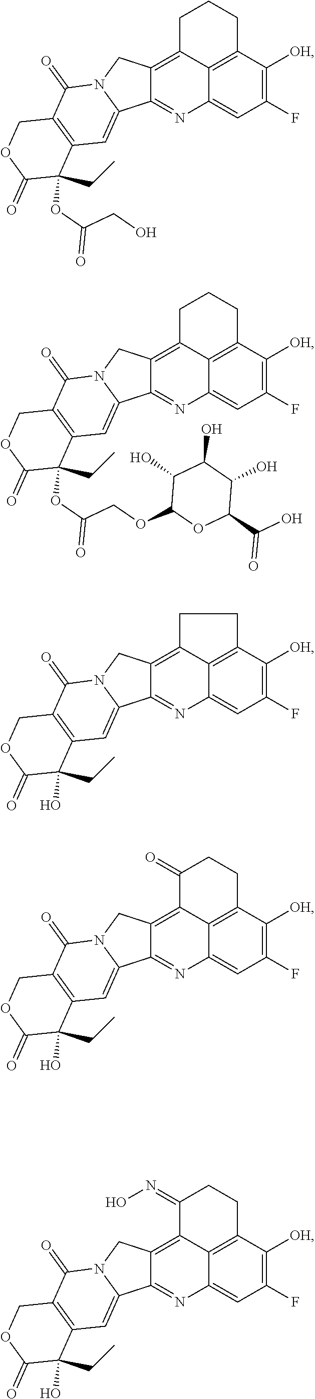

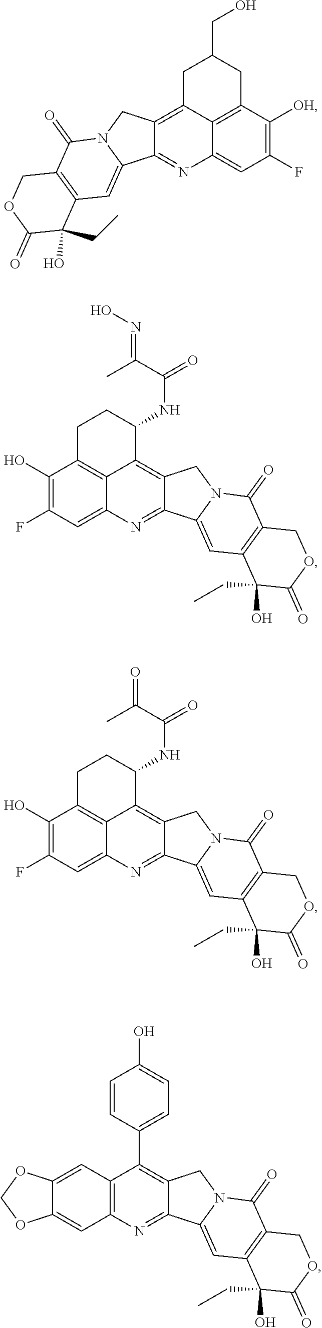 [SMILES: CC[C@@]1(O)C(=O)OCc2c1cc1n(c2=O)Cc2c-1nc1cc(F)c(O)c3c1c2CC(CO)C3 ; CC[C@@]1(O)C(=O)OCc2c1cc1n(c2=O)Cc2c-1nc1cc(F)c(O)c3c1c2[C@@H](NC(=O)/C(C)=N/O)CC3 ; CC[C@@]1(O)C(=O)OCc2c1cc1n(c2=O)Cc2c-1nc1cc(F)c(O)c3c1c2[C@@H](NC(=O)C(C)=O)CC3 ; CC[C@@]1(O)C(=O)OCc2c1cc1n(c2=O)Cc2c-1nc1cc3c(cc1c2-c1ccc(O)cc1)OCO3]

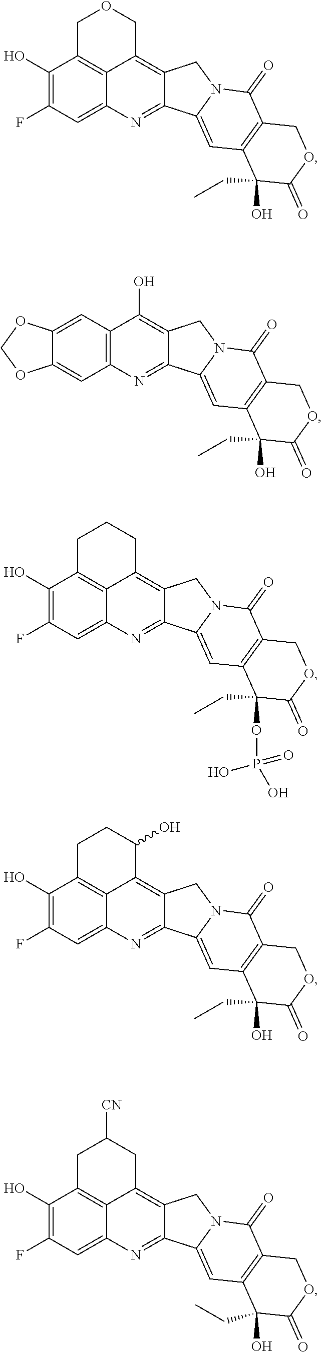 [SMILES: CC[C@@]1(O)C(=O)OCc2c1cc1n(c2=O)Cc2c-1nc1cc(F)c(O)c3c1c2COC3 ; CC[C@@]1(O)C(=O)OCc2c1cc1n(c2=O)Cc2c-1nc1cc3c(cc1c2O)OCO3 ; CC[C@@]1(OP(=O)(O)O)C(=O)OCc2c1cc1n(c2=O)Cc2c-1nc1cc(F)c(O)c3c1c2CCC3 ; CC[C@@]1(O)C(=O)OCc2c1cc1n(c2=O)Cc2c-1nc1cc(F)c(O)c3c1c2C(O)CC3 ; CC[C@@]1(O)C(=O)OCc2c1cc1n(c2=O)Cc2c-1nc1cc(F)c(O)c3c1c2CC(C#N)C3]

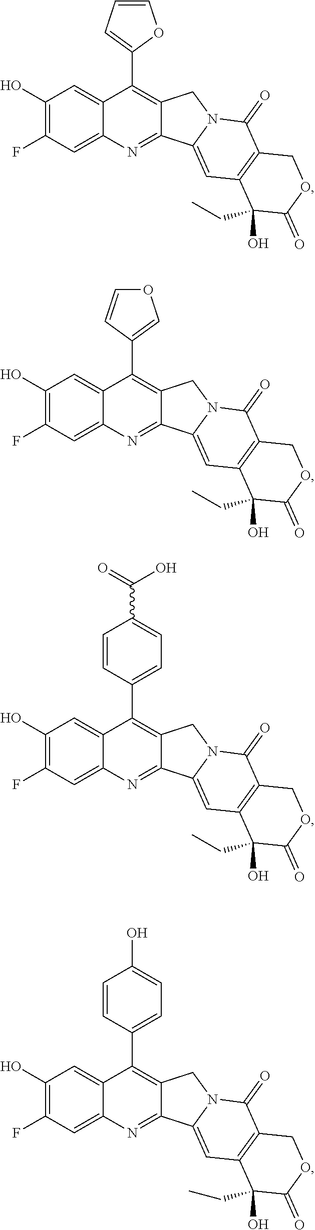 [SMILES: CC[C@@]1(O)C(=O)OCc2c1cc1n(c2=O)Cc2c-1nc1cc(F)c(O)cc1c2-c1ccco1 ; CC[C@@]1(O)C(=O)OCc2c1cc1n(c2=O)Cc2c-1nc1cc(F)c(O)cc1c2-c1ccoc1 ; CC[C@@]1(O)C(=O)OCc2c1cc1n(c2=O)Cc2c-1nc1cc(F)c(O)cc1c2-c1ccc(C(=O)O)cc1 ; CC[C@@]1(O)C(=O)OCc2c1cc1n(c2=O)Cc2c-1nc1cc(F)c(O)cc1c2-c1ccc(O)cc1]

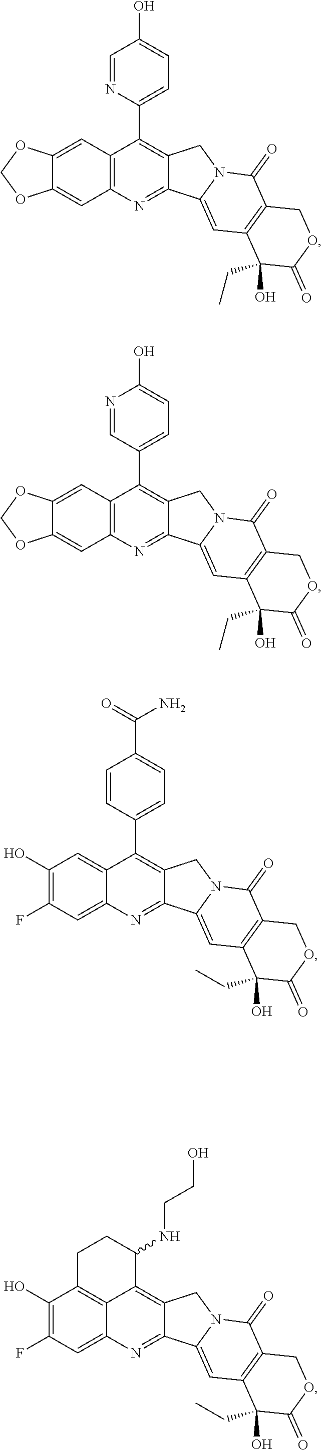 [SMILES: CC[C@@]1(O)C(=O)OCc2c1cc1n(c2=O)Cc2c-1nc1cc3c(cc1c2-c1ccc(O)cn1)OCO3 ; CC[C@@]1(O)C(=O)OCc2c1cc1n(c2=O)Cc2c-1nc1cc3c(cc1c2-c1ccc(O)nc1)OCO3 ; CC[C@@]1(O)C(=O)OCc2c1cc1n(c2=O)Cc2c-1nc1cc(F)c(O)cc1c2-c1ccc(C(N)=O)cc1 ; CC[C@@]1(O)C(=O)OCc2c1cc1n(c2=O)Cc2c-1nc1cc(F)c(O)c3c1c2C(NCCO)CC3]

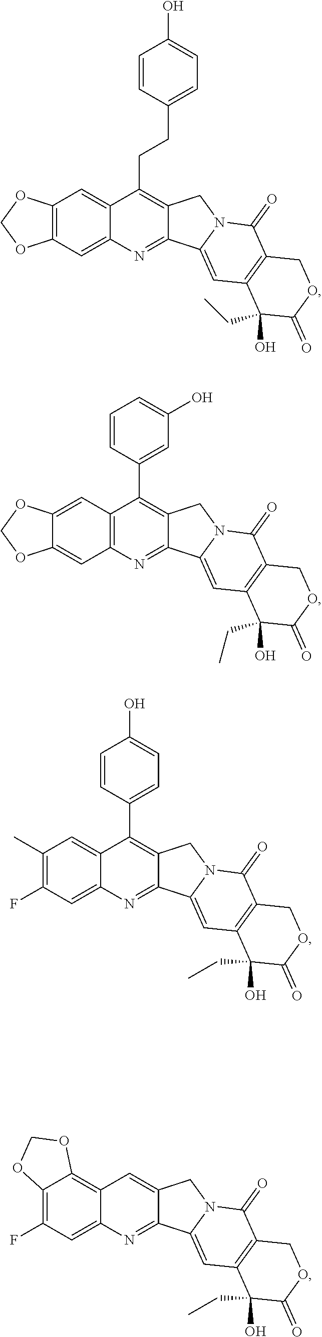 [SMILES: CC[C@@]1(O)C(=O)OCc2c1cc1n(c2=O)Cc2c-1nc1cc3c(cc1c2CCc1ccc(O)cc1)OCO3 ; CC[C@@]1(O)C(=O)OCc2c1cc1n(c2=O)Cc2c-1nc1cc3c(cc1c2-c1cccc(O)c1)OCO3 ; CC[C@@]1(O)C(=O)OCc2c1cc1n(c2=O)Cc2c-1nc1cc(F)c(C)cc1c2-c1ccc(O)cc1 ; CC[C@@]1(O)C(=O)OCc2c1cc1n(c2=O)Cc2cc3c4c(c(F)cc3nc2-1)OCO4]

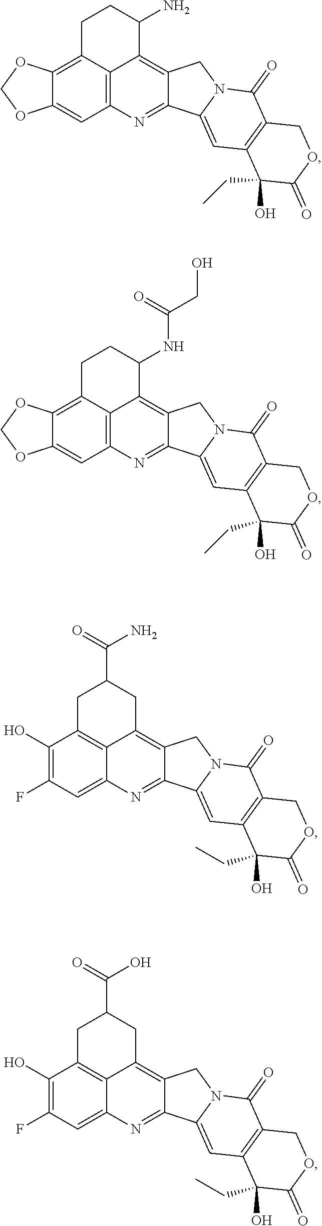 [SMILES: CC[C@@]1(O)C(=O)OCc2c1cc1n(c2=O)Cc2c-1nc1cc3c(c4c1c2C(N)CC4)OCO3 ; CC[C@@]1(O)C(=O)OCc2c1cc1n(c2=O)Cc2c-1nc1cc3c(c4c1c2C(NC(=O)CO)CC4)OCO3 ; CC[C@@]1(O)C(=O)OCc2c1cc1n(c2=O)Cc2c-1nc1cc(F)c(O)c3c1c2CC(C(N)=O)C3 ; CC[C@@]1(O)C(=O)OCc2c1cc1n(c2=O)Cc2c-1nc1cc(F)c(O)c3c1c2CC(C(=O)O)C3]

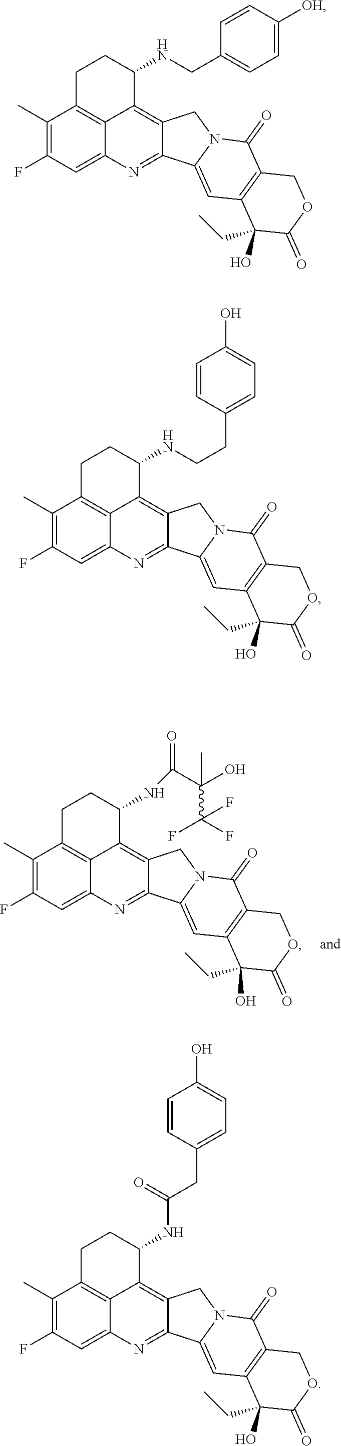 [SMILES: CC[C@@]1(O)C(=O)OCc2c1cc1n(c2=O)Cc2c-1nc1cc(F)c(C)c3c1c2[C@@H](NCc1ccc(O)cc1)CC3 ; CC[C@@]1(O)C(=O)OCc2c1cc1n(c2=O)Cc2c-1nc1cc(F)c(C)c3c1c2[C@@H](NCCc1ccc(O)cc1)CC3 ; CC[C@@]1(O)C(=O)OCc2c1cc1n(c2=O)Cc2c-1nc1cc(F)c(C)c3c1c2[C@@H](NC(=O)C(C)(O)C(F)(F)F)CC3 ; CC[C@@]1(O)C(=O)OCc2c1cc1n(c2=O)Cc2c-1nc1cc(F)c(C)c3c1c2[C@@H](NC(=O)Cc1ccc(O)cc1)CC3]

89-91. (canceled)

92. A pharmaceutical composition comprising the compound of claim 1, and a pharmaceutically acceptable carrier.

93. A method of treating a cancer, an autoimmune disease, or an inflammatory disease, comprising administering the compound or the pharmaceutical composition of claim 1 to a subject in need thereof.

94. The method of claim 93, wherein the cancer, autoimmune disease, or inflammatory disease is selected from leukemia, lymphoma, breast cancer, gastric cancer, colon cancer, ovarian cancer, bladder cancer, prostate cancer, glioma, lung cancer, bronchial cancer, colorectal cancer, pancreatic cancer, esophageal cancer, liver cancer, urinary bladder cancer, kidney cancer, renal pelvis cancer, oral cavity cancer, pharynx cancer, uterine corpus cancer, melanoma, B-cell mediated autoimmune diseases or inflammatory diseases, for example, systemic lupus erythematosus (SLE), rheumatoid arthritis (RA), idiopathic thrombocytopenic purpura (ITP), Waldenstrom's hypergammaglobulinemia, Sjogren's syndrome, multiple sclerosis (MS), and lupus nephritis.

95-97. (canceled)
